SEQUENCE LISTING (141 sequences)

SEQ ID NO: 1 - protein, 125 aa, Artificial Sequence
  VHH-72
QVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSS

SEQ ID NO: 2 - protein, 125 aa, Artificial Sequence
  VHH72-h1 humanized variant 1 of VHH-72
EVQLVESGGGLVQPGGSLRLSCAASGRTFSEYAMGWFRQAPGKEREFVATISWSGGSTYYTDSVKGRFTISRDNAKNTVYLQMNSLRPEDTAVYYCAAAGLGTVVSEWDYDYDYWGQGTLVTVSS

SEQ ID NO: 3 - protein, 125 aa, Artificial Sequence
  VHH72-h1(E1D) humanized variant 1(E1D) of VHH- 72
DVQLVESGGGLVQPGGSLRLSCAASGRTFSEYAMGWFRQAPGKEREFVATISWSGGSTYYTDSVKGRFTISRDNAKNTVYLQMNSLRPEDTAVYYCAAAGLGTVVSEWDYDYDYWGQGTLVTVSS

SEQ ID NO: 4 - protein, 125 aa, Artificial Sequence
  VHH72-S56A variant
QVQLQESGGGLVQAGGSLRLSCAASGRTFSEYAMGWFRQAPGKEREFVATISWSGGATYYTDSVKGRFTISRDNAKNTVYLQMNSLKPDDTAVYYCAAAGLGTVVSEWDYDYDYWGQGTQVTVSS

SEQ ID NO: 5 - protein, 125 aa, Artificial Sequence
  VHH72_h1(S56A) humanized variant 1of VHH72-S56A
EVQLVESGGGLVQPGGSLRLSCAASGRTFSEYAMGWFRQAPGKEREFVATISWSGGATYYTDSVKGRFTISRDNAKNTVYLQMNSLRPEDTAVYYCAAAGLGTVVSEWDYDYDYWGQGTLVTVSS

SEQ ID NO: 6 - protein, 125 aa, Artificial Sequence
  VHH72_h1(E1D)(S56A) humanized variant 1(E1D) of VHH72-S56A
DVQLVESGGGLVQPGGSLRLSCAASGRTFSEYAMGWFRQAPGKEREFVATISWSGGATYYTDSVKGRFTISRDNAKNTVYLQMNSLRPEDTAVYYCAAAGLGTVVSEWDYDYDYWGQGTLVTVSS

SEQ ID NO: 7 - protein, 5 aa, Artificial Sequence
  CDR1 of VHH-72 (or VHH72-S56A)
EYAMG

SEQ ID NO: 8 - protein, 17 aa, Artificial Sequence
  CDR2 of VHH-72
TISWSGGSTYYTDSVKG

SEQ ID NO: 9 - protein, 16 aa, Artificial Sequence
  CDR3 of VHH-72 (or VHH72-S56A)
AGLGTVVSEWDYDYDY

SEQ ID NO: 10 - protein, 17 aa, Artificial Sequence
  CDR2 of VHH-72-S56A
TISWSGGATYYTDSVKG

SEQ ID NO: 11 - protein, 125 aa, Artificial Sequence
  VHH72_h2 humanized variant 2 of VHH72
EVQLVESGGGLVQPGGSLRLSCAASGRTFSEYAMGWFRQAPGKEREFVATISWSGGSTYYADSVKGRFTISRDNAKNTVYLQMNSLRPEDTAVYYCAAAGLGTVVSEWDYDYDYWGQGTLVTVSS

SEQ ID NO: 12 - protein, 265 aa, Artificial Sequence
  bivalent fusion of VHH-72 with a (Gly4Ser)3 -linker
QVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSSGGGGSGGGGSGGGGSQVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSS

SEQ ID NO: 13 - protein, 369 aa, Artificial Sequence
  VHH-72 fused to human IgG1 Fc with a glycine- serine linker in between
QVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSSGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 388 aa, Artificial Sequence
  mouse VH signal sequence-VHH72-GSGGGGSGGGGS- hIgG1Hinge-hIgG1Fc
MGWSCIIFFLVATATGVHSQVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSSGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 388 aa, Artificial Sequence
  mouse VH signal sequence-VHH72-GSGGGGSGGGGS- hIgG1Hinge-hIgG2Fc
MGWSCIIFFLVATATGVHSQVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSSGSGGGGSGGGGSEPKSCDKTHTCPPCPAPELLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 379 aa, Artificial Sequence
  mouse VH signal sequence- VHH72-GSGGGGSGGGGS-hIgG2Hinge_ERKCCdel-hIgG2Fc
MGWSCIIFFLVATATGVHSQVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSSGSGGGGSGGGGSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 361 aa, Artificial Sequence
  D72-58 [VHH72_h1(E1D)_10GS_IgG1_LALA]
DVQLVESGGGLVQPGGSLRLSCAASGRTFSEYAMGWFRQAPGKEREFVATISWSGGSTYYTDSVKGRFTISRDNAKNTVYLQMNSLRPEDTAVYYCAAAGLGTVVSEWDYDYDYWGQGTLVTVSSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 369 aa, Artificial Sequence; identical to SEQ ID NO: 13
  D72-1 [VHH72-GS(G4S)2-hIgG1hinge-hIgG1Fc]

SEQ ID NO: 19 - protein, 359 aa, Artificial Sequence
  VHH72_h1_S56A-GS-hIgG1hinge-hIgG1Fc_LALAPG (D72-23)
EVQLVESGGGLVQPGGSLRLSCAASGRTFSEYAMGWFRQAPGKEREFVATISWSGGATYYTDSVKGRFTISRDNAKNTVYLQMNSLRPEDTAVYYCAAAGLGTVVSEWDYDYDYWGQGTLVTVSSGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 361 aa, Artificial Sequence
  VHH72_h1_E1D_S56A-(G4S)2-hIgG1hinge_EPKSCdel- hIgG1Fc_LALAPG_Kdel(D72-52)
DVQLVESGGGLVQPGGSLRLSCAASGRTFSEYAMGWFRQAPGKEREFVATISWSGGATYYTDSVKGRFTISRDNAKNTVYLQMNSLRPEDTAVYYCAAAGLGTVVSEWDYDYDYWGQGTLVTVSSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 493 aa, Artificial Sequence
  VHH72_ h1_E1D _S56A-(G4S)3-VHH72_h3_S56A-GS-hIgG1hinge_EPKSCdel-hIgG1Fc_LALAPG_ Kdel (D72-55)
DVQLVESGGGLVQPGGSLRLSCAASGRTFSEYAMGWFRQAPGKEREFVATISWSGGATYYTDSVKGRFTISRDNAKNTVYLQMNSLRPEDTAVYYCAAAGLGTVVSEWDYDYDYWGQGTLVTVSSGGGGSGGGGSGGGGSDVQLVESGGGLVQPGGSLRLSCAASGRTFSEYAMGWFRQAPGKEREFVATISWSGGATYYTDSVKGRFTISRDNAKNTVYLQMNSLRPEDTAVYYCAAAGLGTVVSEWDYDYDYWGQGTLVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 361 aa, Artificial Sequence
  VHH72_h1_E1D_S56A-(G4S)2-hIgG1hinge_EPKSCdel- hIgG1Fc_LALA_Kdel (361AA; D72-53 construct)
DVQLVESGGGLVQPGGSLRLSCAASGRTFSEYAMGWFRQAPGKEREFVATISWSGGATYYTDSVKGRFTISRDNAKNTVYLQMNSLRPEDTAVYYCAAAGLGTVVSEWDYDYDYWGQGTLVTVSSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 24 - protein, 1255 aa, Severe acute respiratory syndrome coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 25 - protein, 189 aa, Severe acute respiratory syndrome coronavirus 2
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELL

SEQ ID NO: 26 - protein, 183 aa, Severe acute respiratory syndrome coronavirus
TNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFE

SEQ ID NO: 27 - protein, 125 aa, Artificial Sequence
  VHH72-V100L
QVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTLVSEWDYDYDYWGQGTQVTVSS

SEQ ID NO: 28 - protein, 125 aa, Artificial Sequence
  VHH72-D61Q
QVQLQESGGGLVQAGGSLRLSCAASGRTFSEYAMGWFRQAPGKEREFVATISWSGGSTYYTQSVKGRFTISRDNAKNTVYLQMNSLKPDDTAVYYCAAAGLGTVVSEWDYDYDYWGQGTQVTVSS

SEQ ID NO: 29 - protein, 125 aa, Artificial Sequence
  VHH72-G55S
QVQLQESGGGLVQAGGSLRLSCAASGRTFSEYAMGWFRQAPGKEREFVATISWSGSSTYYTDSVKGRFTISRDNAKNTVYLQMNSLKPDDTAVYYCAAAGLGTVVSEWDYDYDYWGQGTQVTVSS

SEQ ID NO: 30 - protein, 125 aa, Artificial Sequence
  VHH72-S52A
QVQLQESGGGLVQAGGSLRLSCAASGRTFSEYAMGWFRQAPGKEREFVATIAWSGGSTYYTDSVKGRFTISRDNAKNTVYLQMNSLKPDDTAVYYCAAAGLGTVVSEWDYDYDYWGQGTQVTVSS

SEQ ID NO: 31 - protein, 125 aa, Artificial Sequence
  VHH72-W52aF
QVQLQESGGGLVQAGGSLRLSCAASGRTFSEYAMGWFRQAPGKEREFVATISFSGGSTYYTDSVKGRFTISRDNAKNTVYLQMNSLKPDDTAVYYCAAAGLGTVVSEWDYDYDYWGQGTQVTVSS

SEQ ID NO: 32 - protein, 125 aa, Artificial Sequence
  VHH72-W52aH
QVQLQESGGGLVQAGGSLRLSCAASGRTFSEYAMGWFRQAPGKEREFVATISHSGGSTYYTDSVKGRFTISRDNAKNTVYLQMNSLKPDDTAVYYCAAAGLGTVVSEWDYDYDYWGQGTQVTVSS

SEQ ID NO: 33 - protein, 125 aa, Artificial Sequence
  VHH72-V100A
QVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTAVSEWDYDYDYWGQGTQVTVSS

SEQ ID NO: 34 - protein, 125 aa, Artificial Sequence
  VHH72-W52aH-S56A
QVQLQESGGGLVQAGGSLRLSCAASGRTFSEYAMGWFRQAPGKEREFVATISHSGGATYYTDSVKGRFTISRDNAKNTVYLQMNSLKPDDTAVYYCAAAGLGTVVSEWDYDYDYWGQGTQVTVSS

SEQ ID NO: 35 - protein, 125 aa, Artificial Sequence
  VHH72-W52aF-S56A
QVQLQESGGGLVQAGGSLRLSCAASGRTFSEYAMGWFRQAPGKEREFVATISFSGGATYYTDSVKGRFTISRDNAKNTVYLQMNSLKPDDTAVYYCAAAGLGTVVSEWDYDYDYWGQGTQVTVSS

SEQ ID NO: 36 - protein, 125 aa, Artificial Sequence
  VHH72-MDoptA-T60W amino acid sequence- MDoptB refers to the second highest variant obtained by the Molecular Dynamics software
QVQLQESGGGLVQAGGSLRLSCAASGRTFSEYAMGWFRQAPGKEREFVATISWSGGSTYYWDSVKGRFTISRDNAKNTVYLQMNSLKPDDTAVYYCAAAGLGTVVSEWDYDYDYWGQGTQVTVSS

SEQ ID NO: 37 - protein, 125 aa, Artificial Sequence
  VHH72-MDoptA-T57Y
QVQLQESGGGLVQAGGSLRLSCAASGRTFSEYAMGWFRQAPGKEREFVATISWSGGSYYYTDSVKGRFTISRDNAKNTVYLQMNSLKPDDTAVYYCAAAGLGTVVSEWDYDYDYWGQGTQVTVSS

SEQ ID NO: 38 - protein, 125 aa, Artificial Sequence
  VHH72-MDoptA-T57W
QVQLQESGGGLVQAGGSLRLSCAASGRTFSEYAMGWFRQAPGKEREFVATISWSGGSWYYTDSVKGRFTISRDNAKNTVYLQMNSLKPDDTAVYYCAAAGLGTVVSEWDYDYDYWGQGTQVTVSS

SEQ ID NO: 39 - protein, 125 aa, Artificial Sequence
  VHH72-MDoptA-Y100fW
QVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDWDYDYWGQGTQVTVSS

SEQ ID NO: 40 - protein, 125 aa, Artificial Sequence
  VHH72-MDoptA-T57L
QVQLQESGGGLVQAGGSLRLSCAASGRTFSEYAMGWFRQAPGKEREFVATISWSGGSLYYTDSVKGRFTISRDNAKNTVYLQMNSLKPDDTAVYYCAAAGLGTVVSEWDYDYDYWGQGTQVTVSS

SEQ ID NO: 41 - protein, 125 aa, Artificial Sequence
  VHH72-MDoptA-G96Y
QVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAYLGTVVSEWDYDYDYWGQGTQVTVSS

SEQ ID NO: 42 - protein, 125 aa, Artificial Sequence
  VHH72-MDoptA-T57I
QVQLQESGGGLVQAGGSLRLSCAASGRTFSEYAMGWFRQAPGKEREFVATISWSGGSIYYTDSVKGRFTISRDNAKNTVYLQMNSLKPDDTAVYYCAAAGLGTVVSEWDYDYDYWGQGTQVTVSS

SEQ ID NO: 43 - protein, 125 aa, Artificial Sequence
  VHH72-MDoptA-V100I
QVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTIVSEWDYDYDYWGQGTQVTVSS

SEQ ID NO: 44 - protein, 125 aa, Artificial Sequence
  VHH72-MDoptA-T57H
QVQLQESGGGLVQAGGSLRLSCAASGRTFSEYAMGWFRQAPGKEREFVATISWSGGSHYYTDSVKGRFTISRDNAKNTVYLQMNSLKPDDTAVYYCAAAGLGTVVSEWDYDYDYWGQGTQVTVSS

SEQ ID NO: 45 - protein, 125 aa, Artificial Sequence
  VHH72-MDoptA-Y59W
QVQLQESGGGLVQAGGSLRLSCAASGRTFSEYAMGWFRQAPGKEREFVATISWSGGSTYWTDSVKGRFTISRDNAKNTVYLQMNSLKPDDTAVYYCAAAGLGTVVSEWDYDYDYWGQGTQVTVSS

SEQ ID NO: 46 - protein, 125 aa, Artificial Sequence
  VHH72-MDoptB-T60F
QVQLQESGGGLVQAGGSLRLSCAASGRTFSEYAMGWFRQAPGKEREFVATISWSGGSTYYFDSVKGRFTISRDNAKNTVYLQMNSLKPDDTAVYYCAAAGLGTVVSEWDYDYDYWGQGTQVTVSS

SEQ ID NO: 47 - protein, 125 aa, Artificial Sequence
  VHH72-MDoptB-T99V
QVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGVVVSEWDYDYDYWGQGTQVTVSS

SEQ ID NO: 48 - protein, 125 aa, Artificial Sequence
  VHH72-MDoptB-K64W
QVQLQESGGGLVQAGGSLRLSCAASGRTFSEYAMGWFRQAPGKEREFVATISWSGGSTYYTDSVWGRFTISRDNAKNTVYLQMNSLKPDDTAVYYCAAAGLGTVVSEWDYDYDYWGQGTQVTVSS

SEQ ID NO: 49 - protein, 125 aa, Artificial Sequence; identical to SEQ ID NO: 27
  VHH72-MDoptB-V100L

SEQ ID NO: 50 - protein, 125 aa, Artificial Sequence
  VHH72-MDoptC-F74R
QVQLQESGGGLVQAGGSLRLSCAASGRTFSEYAMGWFRQAPGKERERVATISWSGGSTYYTDSVKGRFTISRDNAKNTVYLQMNSLKPDDTAVYYCAAAGLGTVVSEWDYDYDYWGQGTQVTVSS

SEQ ID NO: 51 - protein, 125 aa, Artificial Sequence
  VHH72-MDoptC-G98P
QVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLPTVVSEWDYDYDYWGQGTQVTVSS

SEQ ID NO: 52 - protein, 125 aa, Artificial Sequence
  VHH72-MDoptC-V100L
QVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVLSEWDYDYDYWGQGTQVTVSS

SEQ ID NO: 53 - protein, 125 aa, Artificial Sequence
  VHH72-MDoptC-G98I
QVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLITVVSEWDYDYDYWGQGTQVTVSS

SEQ ID NO: 54 - protein, 125 aa, Artificial Sequence
  VHH72-MDoptC-T75R
QVQLQESGGGLVQAGGSLRLSCAASGRTFSEYAMGWFRQAPGKEREFVATISWSGGSRYYTDSVKGRFTISRDNAKNTVYLQMNSLKPDDTAVYYCAAAGLGTVVSEWDYDYDYWGQGTQVTVSS

SEQ ID NO: 55 - protein, 125 aa, Artificial Sequence
  VHH72-MDoptC-T75F
QVQLQESGGGLVQAGGSLRLSCAASGRTFSEYAMGWFRQAPGKEREFVATISWSGGSFYYTDSVKGRFTISRDNAKNTVYLQMNSLKPDDTAVYYCAAAGLGTVVSEWDYDYDYWGQGTQVTVSS

SEQ ID NO: 56 - protein, 125 aa, Artificial Sequence
  VHH72-MDoptC-T75N
QVQLQESGGGLVQAGGSLRLSCAASGRTFSEYAMGWFRQAPGKEREFVATISWSGGSNYYTDSVKGRFTISRDNAKNTVYLQMNSLKPDDTAVYYCAAAGLGTVVSEWDYDYDYWGQGTQVTVSS

SEQ ID NO: 57 - protein, 125 aa, Artificial Sequence
  VHH72-MDoptC-T50P
QVQLQESGGGLVQAGGSLRLSCAASGRTFSEYAMGWFRQAPGKEREFVAPISWSGGSTYYTDSVKGRFTISRDNAKNTVYLQMNSLKPDDTAVYYCAAAGLGTVVSEWDYDYDYWGQGTQVTVSS

SEQ ID NO: 58 - protein, 125 aa, Artificial Sequence
  VHH72-MDoptC-F47H
QVQLQESGGGLVQAGGSLRLSCAASGRTFSEYAMGWFRQAPGKEREHVATISWSGGSTYYTDSVKGRFTISRDNAKNTVYLQMNSLKPDDTAVYYCAAAGLGTVVSEWDYDYDYWGQGTQVTVSS

SEQ ID NO: 59 - protein, 125 aa, Artificial Sequence
  VHH72-MDoptC-T50Q
QVQLQESGGGLVQAGGSLRLSCAASGRTFSEYAMGWFRQAPGKEREFVAQISWSGGSTYYTDSVKGRFTISRDNAKNTVYLQMNSLKPDDTAVYYCAAAGLGTVVSEWDYDYDYWGQGTQVTVSS

SEQ ID NO: 60 - protein, 125 aa, Artificial Sequence
  VHH72-MDoptC-T60Y
QVQLQESGGGLVQAGGSLRLSCAASGRTFSEYAMGWFRQAPGKEREFVATISWSGGSTYYYDSVKGRFTISRDNAKNTVYLQMNSLKPDDTAVYYCAAAGLGTVVSEWDYDYDYWGQGTQVTVSS

SEQ ID NO: 61 - protein, 125 aa, Artificial Sequence
  VHH72-MDoptC-T60R
QVQLQESGGGLVQAGGSLRLSCAASGRTFSEYAMGWFRQAPGKEREFVATISWSGGSTYYRDSVKGRFTISRDNAKNTVYLQMNSLKPDDTAVYYCAAAGLGTVVSEWDYDYDYWGQGTQVTVSS

SEQ ID NO: 62 - protein, 214 aa, Artificial Sequence
  Light chain of S309 antibody
EIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 456 aa, Artificial Sequence
  Heavy chain of S309 antibody
DVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 64 - protein, 216 aa, Artificial Sequence
  CB6 light chain sequence
DIVMTQSPSSLSASVGDRVTITCRASQSISRYLNWYQQKPGKAPKLLIYAASSLQSGVPSRFSGSGSGTDFTLTISSLQPEDFATYYCQQSYSTPPEYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 449 aa, Artificial Sequence
  CB6 heavy chain sequence
EVQLVESGGGLVQPGGSLRLSCAASGFTVSSNYMSWVRQAPGKGLEWVSVIYSGGSTFYADSVKGRFTISRDNSMNTLFLQMNSLRAEDTAVYYCARVLPMYGDYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 96 aa, Artificial Sequence
  cov2_wuhan Spike protein RBD, with a deletion of the RBM loop
VLYNSASFSTFKCYGVSPTKLNDLCFTNVYADSFVIRGDEVRQIAPGQTGKIADYNYKLPDDFTGCVIAWNSNNLDPTNGVGYQPYRVVVLSFELL

SEQ ID NO: 67 - protein, 96 aa, Artificial Sequence
  GD-Pangolin Spike protein RBD, with a deletion of the RBM loop
VLYNSTSFSTFKCYGVSPTKLNDLCFTNVYADSFVVRGDEVRQIAPGQTGRIADYNYKLPDDFTGCVIAWNSNNLDPTNGVGYQPYRVVVLSFELL

SEQ ID NO: 68 - protein, 96 aa, Artificial Sequence
  RaTG13 Spike protein RBD, with a deletion of the RBM loop
VLYNSTSFSTFKCYGVSPTKLNDLCFTNVYADSFVITGDEVRQIAPGQTGKIADYNYKLPDDFTGCVIAWNSKHIDPTDGVGHQPYRVVVLSFELL

SEQ ID NO: 69 - protein, 96 aa, Artificial Sequence
  WIV1 Spike protein RBD, with a deletion of the RBM loop
VLYNSTSFSTFKCYGVSATKLNDLCFSNVYADSFVVKGDDVRQIAPGQTGVIADYNYKLPDDFTGCVLAWNTRNIDITNGIGYQPYRVVVLSFELL

SEQ ID NO: 70 - protein, 96 aa, Artificial Sequence
  LYRa11 Spike protein RBD, with a deletion of the RBM loop
VLYNSTSFSTFKCYGVSAIKLNDLCFSNVYADSFVVKGDDVRQIAPGQTGVIADYNYKLPDDFMGCVLAWNTRNIDTTNGIGYQPYRVVVLSFELL

SEQ ID NO: 71 - protein, 96 aa, Artificial Sequence
  RsSHC014 Spike protein RBD, with a deletion of the RBM loop
VLYNSTSFSTFKCYGVSATKLNDLCFSNVYADSFVVKGDDVRQIAPGQTGVIADYNYKLPDDFLGCVLAWNTNSKDTTAGVGHQPYRVVVLSFELL

SEQ ID NO: 72 - protein, 96 aa, Artificial Sequence
  Rs7327 Spike protein RBD domain, with a deletion of the RBM loop
VLYNSTSFSTFKCYGVSATKLNDLCFSNVYADSFVVKGDDVRQIAPGQTGVIADYNYKLPDDFMGCVLAWNTRNIDTTNGIGYQPYRVVVLSFELL

SEQ ID NO: 73 - protein, 96 aa, Artificial Sequence
  SARS-CoV-1 Spike protein RBD, with a deletion of the RBM loop
VLYNSTFFSTFKCYGVSATKLNDLCFSNVYADSFVVKGDDVRQIAPGQTGVIADYNYKLPDDFMGCVLAWNTRNIDTTTGIGYQPYRVVVLSFELL

SEQ ID NO: 74 - protein, 96 aa, Artificial Sequence; identical to SEQ ID NO: 71
  Rs4231 Spike protein RBD, with a deletion of the RBM loop

SEQ ID NO: 75 - protein, 96 aa, Artificial Sequence
  Rs4084 Spike protein RBD, with a deletion of the RBM loop
ILYNSTSFSTFKCYGVSATKLNDLCFSNVYADSFVVKGDDVRQIAPGQTGVIADYNYKLPDDFLGCVLAWNTNSKDTTAGVGHQPYRVVVLSFELL

SEQ ID NO: 76 - protein, 96 aa, Artificial Sequence
  Rp3 Spike protein RBD, with a deletion of the RBM loop
VLYNSTSFSTFKCYGVSPSKLIDLCFTSVYADTFLIRSSEVRQVAPGETGVIADYNYKLPDDFTGCVIAWNTAKQDPSVPVAYQATRVVVLSFELL

SEQ ID NO: 77 - protein, 96 aa, Artificial Sequence
  HKU3-1 Spike protein RBD, with a deletion of the RBM loop
VLYNSTSFSTFKCYGVSPSKLIDLCFTSVYADTFLIRSSEVRQVAPGETGVIADYNYKLPDDFTGCVIAWNTAKHDPNVPVAYQATRVVVLSFELL

SEQ ID NO: 78 - protein, 96 aa, Artificial Sequence
  ZXC21 Spike protein RBD, with a deletion of the RBM loop
VFYNSTSFSTFKCYGVSPSKLIDLCFTSVYADTFLIRFSEVRQVAPGQTGVIADYNYKLPDDFTGCVIAWNTAKQDPNVPLEYQATRVVVLSFELL

SEQ ID NO: 79 - protein, 96 aa, Artificial Sequence; identical to SEQ ID NO: 78
  ZC45 Spike protein RBD, with a deletion of the RBM loop

SEQ ID NO: 80 - protein, 96 aa, Artificial Sequence
  Rf1 Spike protein RBD, with a deletion of the RBM loop
VFYNSTSFSTFNCYGVSPSKLIDLCFTSVYADTFLIRFSEVRQVAPGQTGVIADYNYKLPDDFTGCVIAWNTAKQDQNVPLEYQATRVVVLSFELL

SEQ ID NO: 81 - protein, 85 aa, Artificial Sequence
  BM48-31 Spike protein RBD, with a deletion of the RBM loop
VLYNSSASFSTFQCYGVSPTKLNDLCFSSVYADYFVVKGDDVRQIAPAQTGVIADYNYKLPDDFTGCVIAWNTNSLDQSSGIGFQ

SEQ ID NO: 82 - DNA, 20 bp, Artificial Sequence
  Primer
ttacaaacattggccgcaaa

SEQ ID NO: 83 - DNA, 18 bp, Artificial Sequence
  Primer
gcgcgacattccgaagaa

SEQ ID NO: 84 - DNA, 20 bp, Artificial Sequence
  Primer
ccagtaatgtggacattgcc

SEQ ID NO: 85 - DNA, 23 bp, Artificial Sequence
  Primer
catcaacgaccttgtcttcagta

SEQ ID NO: 86 - DNA, 21 bp, Artificial Sequence
  Primer
gccattcatccacagttgaca

SEQ ID NO: 87 - DNA, 22 bp, Artificial Sequence
  Primer
catggtgctgacagtggagtct

SEQ ID NO: 88 - DNA, 21 bp, Artificial Sequence
  Primer
gggaactgtcaaagggtacag

SEQ ID NO: 89 - DNA, 22 bp, Artificial Sequence
  Primer
cccttcctacatcagtcctact

SEQ ID NO: 90 - DNA, 18 bp, Artificial Sequence
  Primer
ggccaggtcatcaccatt

SEQ ID NO: 91 - DNA, 24 bp, Artificial Sequence
  Primer
gagttgaatgtagtttcgtggatg

SEQ ID NO: 92 - protein, 125 aa, Artificial Sequence
  VHH2.50
QVQLQESGGGLVQAGGSLRLSCAASGRTFSSIAMGWFRQAPGKEREFVATISWSGGSTYYADSVKGRFTISRDNAKNTVYLQMNSLKPEDTAVYYCAAAGLGTVVSEWDYDYDYWGQGTQVTVSS

SEQ ID NO: 93 - protein, 125 aa, Artificial Sequence
  VHH3.17
QVQLQESGGGLVEAGGSLTLSCAASGRAFGDGAVGWFRQGPGRPREFVATVSWNGGGTYFAESVRGRFTISRDNAKNTVYLQMNNLKPEDTAVYYCALAGEGTVVSEWDYDYEYWGQGTQVTVSS

SEQ ID NO: 94 - protein, 125 aa, Artificial Sequence
  VHH3.77
QVQLQESGGGLVQAGGSLRLSCVSSGRAFGNGAVGWFRQGPGRPREFVATVSWNGGGTYFAESVRGRFTISRDNAKNTVYLQMNNLKPEDTAVYYCALAGEGTVVSEWDYDYEYWGQGTQVTVSS

SEQ ID NO: 95 - protein, 125 aa, Artificial Sequence
  VHH3.115
QVQLQESGGGLVQTGGSLRLSCAASGLTFSDIAMGWFRQAPGREREFVATISWSGGGTYYAEPVRGRFAISRDNAKNTVYLQMNSLKPEDTAVYYCAAAGAGTVVSEWDYDYDYWGQGTQVTVSS

SEQ ID NO: 96 - protein, 125 aa, Artificial Sequence
  VHH3.144
QVQLQESGGGLVQAGGSLRLSCVASGRAFGNGAVGWFRQAPGREREFVATVSWNGGGTYYAESVRGRFTISRDNAKSTVYLQMNSLKPEDTAVYYCASAGEGTVVSEWDYDYDYWGQGTQVTVSS

SEQ ID NO: 97 - protein, 125 aa, Artificial Sequence; identical to SEQ ID NO: 96
  VHH3BE4

SEQ ID NO: 98 - protein, 127 aa, Artificial Sequence
  VHH3.83
QVQLQESGGGLVQPGDSLRLSCVLSGGVFTSYAMGWFRQAPGKEREFLAAITFNSDATYYADSVKGRFTISRDNAKNTAYLQMNSLKPNDTAVYSCAAGGNHYNPQYYHDYDKYDHWGQGTQVTVSS

SEQ ID NO: 99 - protein, 121 aa, Artificial Sequence
  VHH3.36
QVQLQESGGGLVQAGDSLRLSCVVSGGTFSSYAMGWFRQAPGKERELVAAINWGGISVYYADSVKGRFTISRDNAENTVYLQMNSLKPEDTAVYYCAADPKGWSEWDMEYWGKGTQVTVSS

SEQ ID NO: 100 - protein, 121 aa, Artificial Sequence
  VHH3.47
QVQLQESGGGLVQAGDSLRLSCAASGHNFSTYAMAWFRQAPGTERELVAAISENDVMRYYADSVKGRFTISRDNAKNMAYLQMNSLKVEDTAVYLCAADPKGWSEWDMDYWGKGTQVTVSS

SEQ ID NO: 101 - protein, 121 aa, Artificial Sequence
  VHH3.55
QVQLQESGGGLVQAGGSLRLSCEVSGRTNDNYGVGWFRQVPGAERELVAAIRWSSISRYYKDSVKGRFTISRDNAKNTVYLQMDMLKPEDTAAYYCAADPAGWSEFGMEYWGKGTQVTVSS

SEQ ID NO: 102 - protein, 121 aa, Artificial Sequence
  VHH3.35
QVQLQESGGGLVQAGGSLTLSCEVSGRTNDNYGVGWFRQVPGAERELVAAIRWSSISRYYKDSVKGRFTISRDNAKNTVYLQMDMLKAEDTAVYYCAADPAGWSEFGMEYWGKGTQVTVSS

SEQ ID NO: 103 - protein, 121 aa, Artificial Sequence
  VHH3.29
QVQLQESGGGLVQAGGSLRLSCVASGRTFSSGGMGWFRQAPGQEREFVAGIGWAGLSSYYLDSVKGRFTISLDNAKNTVYLQMNMLKPEDTGVYYCAADDHGWSAAGMDYLGQGTQVTVSS

SEQ ID NO: 104 - protein, 122 aa, Artificial Sequence
  VHH3.38
QVQLQESGGGLVQAGGSLRLSCAASGGTFNNYAMAWFRQAPGQERELVAAMFWSGLPKYYADSVKGRFTISTDDAKNTVYLQMNGLKPEDTAVYYCAADSRGWSDVGGMDYWGKGTQVTVSS

SEQ ID NO: 105 - protein, 121 aa, Artificial Sequence
  VHH3.149
QVQLQESGGGLVQAGGSLKLSCAASGRTFSSYALGWFRQAPGTEREFVSAINWFGAPTYYADSVKGRFTISRDNAKNTVYLQMDNLKPEDTAVYYCAADSKGWDPQDMDYWGKGTQVTVSS

SEQ ID NO: 106 - protein, 134 aa, Artificial Sequence
  VHH3.39
QVQLQESGGGSVQPGDSLRLSCKASGITLGYYAIGWFRQAPGKEREEISCISSIDGSIYYTDSATGRFTISRDNAEHTVYLQMNNLKPEDTAVYYCALAIGYHTYRTDTPLYTHSCRENSFPTWGRGTQVTVSS

SEQ ID NO: 107 - protein, 122 aa, Artificial Sequence
  VHH3.89
QVQLQESGGGLVQPGGSLRLSCAASGFTLDYYAIGWFREVPGKEREGLSRIDSSDGSTYYADSVKGRFTISRDNTKNIVYLQMNNLKPEDTAVYYCATDPIIQGRNWYWTGWGQGTQVTVSS

SEQ ID NO: 108 - protein, 121 aa, Artificial Sequence
  VHH3.141
QVQLQESGGGLVQPGGSLRLSCVVSGELFSINGVGWYRQAPGKQRELVAAITEGGNTNYVDNVKGRFTISRDNAKNTVYLQMNSLKPEDTAVYYCNANLSFWSREYEYDYWGQGTQVTVSS

SEQ ID NO: 109 - protein, 128 aa, Artificial Sequence
  VHH3.151
QVQLQESGGGLVQPGGSLRLSCAASGFSSDYYAIGWFRQAPGKEREGVGCISSSDDSIYYADSVKGRFTISRDNGKNTVYLQMNSLKPEDTAVYHCATDWLRLCTIVSGTQVPPYDVWGQGTQVTVSS

SEQ ID NO: 110 - protein, 131 aa, Artificial Sequence
  VHH3BD9
QVQLQESGGGLVQPGGSLRLSPCAASGFTLLDRYAIGWVRQAPGKEREGVSCISSSDDSTYYARSVKGRFTISRDNAKNTVYLQMNSLKPEDTAVYYCATDWLRLCTVVANTEVPPYDLWGPQGTQVTVSS

SEQ ID NO: 111 - protein, 5 aa, Artificial Sequence
  CDR
SIAMG

SEQ ID NO: 112 - protein, 5 aa, Artificial Sequence
  CDR
DGAVG

SEQ ID NO: 113 - protein, 5 aa, Artificial Sequence
  CDR
NGAVG

SEQ ID NO: 114 - protein, 5 aa, Artificial Sequence
  CDR
DIAMG

SEQ ID NO: 115 - protein, 5 aa, Artificial Sequence
  CDR
TYAMA

SEQ ID NO: 116 - protein, 5 aa, Artificial Sequence
  CDR
NYGVG

SEQ ID NO: 117 - protein, 5 aa, Artificial Sequence
  CDR
SGGMG

SEQ ID NO: 118 - protein, 5 aa, Artificial Sequence
  CDR
NYAMA

SEQ ID NO: 119 - protein, 5 aa, Artificial Sequence
  CDR
SYALG

SEQ ID NO: 120 - protein, 17 aa, Artificial Sequence
  CDR
TISWSGGSTYYADSVKG

SEQ ID NO: 121 - protein, 17 aa, Artificial Sequence
  CDR
TVSWNGGGTYFAESVRG

SEQ ID NO: 122 - protein, 17 aa, Artificial Sequence
  CDR
TVSWNGGGTYYAEPVRG

SEQ ID NO: 123 - protein, 17 aa, Artificial Sequence
  CDR
TVSWNGGGTYYAESVRG

SEQ ID NO: 124 - protein, 17 aa, Artificial Sequence
  CDR
AITFNSDATYYADSVKG

SEQ ID NO: 125 - protein, 17 aa, Artificial Sequence
  CDR
AINWGGISVYYADSVKG

SEQ ID NO: 126 - protein, 17 aa, Artificial Sequence
  CDR
AISENDVMRYYADSVKG

SEQ ID NO: 127 - protein, 17 aa, Artificial Sequence
  CDR
AIRWSSISRYYKDSVKG

SEQ ID NO: 128 - protein, 17 aa, Artificial Sequence
  CDR
GIGWAGLSSYYLDSVKG

SEQ ID NO: 129 - protein, 17 aa, Artificial Sequence
  CDR
AMFWSGLPKYYADSVKG

SEQ ID NO: 130 - protein, 17 aa, Artificial Sequence
  CDR
AINWFGAPTYYADSVKG

SEQ ID NO: 131 - protein, 16 aa, Artificial Sequence
  CDR
AGEGTVVSEWDYDYEY

SEQ ID NO: 132 - protein, 16 aa, Artificial Sequence
  CDR
AGAGTVVSEWDYDYDY

SEQ ID NO: 133 - protein, 16 aa, Artificial Sequence
  CDR
AGEGTVVSEWDYDYDY

SEQ ID NO: 134 - protein, 18 aa, Artificial Sequence
  CDR
GGNHYNPQYYHDYDKYDH

SEQ ID NO: 135 - protein, 12 aa, Artificial Sequence
  CDR
DPKGWSEWDMEY

SEQ ID NO: 136 - protein, 12 aa, Artificial Sequence
  CDR
DPKGWSEWDMDY

SEQ ID NO: 137 - protein, 12 aa, Artificial Sequence
  CDR
DPAGWSEFGMEY

SEQ ID NO: 138 - protein, 12 aa, Artificial Sequence
  CDR
DDHGWSAAGMDY

SEQ ID NO: 139 - protein, 13 aa, Artificial Sequence
  CDR
DSRGWSDVGGMDY

SEQ ID NO: 140 - protein, 12 aa, Artificial Sequence
  CDR
DSKGWDPQDMDY

SEQ ID NO: 141 - protein, 17 aa, Artificial Sequence
  CDR2 of VHH-72- S52A-S56A mutant amino acid sequence
TIAWSGGATYYTDSVKG

(19) United States
(12) Patent Application Publication
(10) Pub. No.: US 2023/0227537 A1
(43) Pub. Date: Jul. 20, 2023

(54) CORONA VIRUS BINDERS

(71) Applicants: VIB VZW, Gent (BE); UNIVERSITEIT GENT, Gent (BE); BOARD OF REGENTS, THE UNIVERSITY OF TEXAS SYSTEM, Austin, TX (US); THE UNITED STATES OF AMERICA, AS REPRESENTED BY THE SECRETARY OF THE DEPARTMENT OF HEALTH AND HUMAN, Bethesda, MD (US); TRUSTEES OF DARTMOUTH COLLEGE, Hanover, NH (US); KATHOLIEKE UNIVERSITEIT LEUVEN, K.U.LEUVEN R&D, Leuven (BE); EXEVIR BIO BV, Gent (BE); VRIJE UNIVERSITEIT BRUSSEL, Brussel (BE)

(72) Inventors: Bert SCHEPENS, Drongen (BE); Xavier SAELENS, leper (BE); Nico CALLEWAERT, Nevele (BE); Dorien DE VLIEGER, Lotenhulle (BE); Loes VAN SCHIE, Gent (BE); Wim NERINCKX, Astene (BE); Kenny ROOSE, Gent (BE); Wander VAN BREEDAM, Antwerpen (BE); Hannah EECKHAUT, Oosterzele (BE); Daria FIJALKOWSKA, Evergem (BE); Chiara LONIGRO, Gent (BE); Sieglinde DE CAE, Lennik (BE); Bruno DOMBRECHT, Heusden (BE); Catelijne STORTELERS, Gent (BE); Johan NEYTS, Kessel-Lo (BE); Leen DELANG, Korbeek-Lo (BE); Suzanne KAPTEIN, DH Maastricht (NL); Joana DUARTE DA ROCHA PEREIRA, Sint- Agatha-Rode (BE); Barney GRAHAM, Rockville, MD (US); Jason MCLELLAN, Austin, TX (US); Daniel WRAPP, Chandler, AZ (US); Han REMAUT, Roosbeek (BE)

(21) Appl. No.: 17/760,300

(22) PCT Filed: Feb. 5, 2021
(86) PCT No.: PCT/EP2021/052885

§ 371 Date: Aug. 5, 2022

Related U.S. Application Data
(63) Continuation of application No. PCT/EP2020/077004, filed on Sep. 25, 2020.
(60) Provisional application No. 62/971,013, filed on Feb. 6, 2020.
(60) Provisional application No. 62/988,610, filed on Mar. 12, 2020.
(60) Provisional application No. 62/991,408, filed on Mar. 18, 2020.
(60) Provisional application No. 63/041,240, filed on Jun. 19, 2020.

(30) Foreign Application Priority Data
Dec. 23, 2020 (GB) 2020508.4
Jan. 13, 2021 (EP) 21151356.9

(51) Int. Cl. C07K 16/10; C12N 15/63; A61P 31/14

(52) U.S. Cl. CPC C07K 16/10; C12N 15/63; A61P 31/14; A61K 2039/505

(57) ABSTRACT

The present invention relates to the field of virology, more specifically to the field of zoonotic Coronaviruses. Specifically, the invention provides for binding agents specific for the spike protein receptor binding domain (RBD) of the SARS-Corona virus, more specifically for an epitope of the RBD present in a broad range of Sarbecoviruses and mutants thereof, even more specifically present in SARS-Cov and SARS-CoV-2 viruses. More specifically, the invention relates to compositions comprising antibodies capable of specifically binding and neutralizing SARS-Corona viruses. More specifically the invention relates to compositions comprising single domain antibodies, or specifically VHHs, and compositions comprising multivalent binding agents comprising IgG Fc fusions thereof, specifically VHH-Fc fusions thereof, even more specifically comprising heavy chain only VHH72-S56A-IgG1-Fc fusions, or compositions comprising any humanized form of any one thereof, and are capable of specifically binding and neutralizing SARS-Corona viruses, specifically SARS-Cov-2 virus. The compositions are useful in the diagnosis of Sarbecoviruses, and specifically SARS-CoV-2 virus, and in prophylactic and/or therapeutic treatment of a condition resulting from infections with Sarbecoviruses, specifically SARS-Corona or SARS-CoV-2 virus, or mutants thereof.

### STATEMENT REGARDING FEDERALLY SPONSORED R&D

This invention was made with government support under Grant no. R01 A1127521 awarded by the National Institutes of Health. The government has certain rights in the invention.

### REFERENCE TO SEQUENCE LISTING, TABLE, OR COMPUTER PROGRAM LISTING

The present application is being filed along with a Sequence Listing in electronic format. The Sequence Listing is provided as a file entitled 57460992_1.TXT, created and last modified on Apr. 14, 2023, which is 174 Kb in size. The information in the electronic format of the Sequence Listing is incorporated herein by reference in its entirety.

### FIELD OF THE INVENTION

The present invention relates to the field of virology, more specifically to the field of zoonotic Coronaviruses. Specifically, the invention provides for binding agents specific for the spike protein receptor binding domain (RBD) of the SARS-Corona virus, more specifically for an epitope of the RBD present in a broad range of Sarbecoviruses and mutants thereof, even more specifically present in SARS-Cov and SARS-CoV-2 viruses. More specifically, the invention relates to compositions comprising antibodies capable of specifically binding and neutralizing SARS-Corona viruses. More specifically the invention relates to compositions comprising single domain antibodies, or specifically VHHs, and compositions comprising multivalent binding agents comprising IgG Fc fusions thereof, specifically VHH-Fc fusions thereof, even more specifically comprising heavy chain only VHH72-S56A-IgG1-Fc fusions, or compositions comprising any humanized form of any one thereof, and are capable of specifically binding and neutralizing SARS-Corona viruses, specifically SARS-Cov-2 virus. The compositions are useful in the diagnosis of Sarbecoviruses, and specifically SARS-CoV-2 virus, and in prophylactic and/or therapeutic treatment of a condition resulting from infections with Sarbecoviruses, specifically SARS-Corona or SARS-CoV-2 virus, or mutants thereof.

### INTRODUCTION TO THE INVENTION

The Coronaviridae family has its name from the large spike protein molecules that are present on the virus surface and give the virions a crown-like shape. The coronavirus genomes are the largest among RNA viruses and the family has been classified into at least three primary genera (alpha, beta, and gamma). Coronaviruses thus represent a diverse family of large enveloped positive-stranded RNA viruses that infect a wide range of animals, a wide variety of vertebrate species, and humans. The spike (S) proteins of coronaviruses are essential for host receptor-binding and subsequent fusion of the viral and host cell membrane, effectively resulting in the release of the viral nucleocapsids in the host cell cytoplasm⁵³. Four coronaviruses, presumably from a zoonotic origin, are endemic in humans: HCoV-NL63 and HCoV-229E (α-coronaviruses) and HCoV-OC43 and HCoV-HKU1 (β-coronaviruses). In addition, 3 episodes of severe respiratory disease caused by β-coronaviruses have occurred since 2002. In the period 2002, severe acute respiratory syndrome virus (SARS), caused by SARS-CoV-1, emerged from a zoonotic origin (bats via civet cats as an intermediate species) spread across the globe and disappeared in 2004⁶⁶. Over 8000 SARS cases were reported with a mortality rate of approximately 10%. In 2012, Middle East respiratory syndrome (MERS) emerged in the Arabian Peninsula. MERS is caused by MERS-CoV, has been confirmed in over 2500 cases and has a case fatality rate of 34%⁶⁷.

Starting at the end of 2019, cases of severe acquired pneumonia were reported in the city of Wuhan (China) with a cluster of patients with connections to Huanan South China Seafood Market that is caused by a new β-coronavirus, known as SARS-CoV-2, given its genetic relationship with SARS-CoV-1⁶⁸, as the third zoonotic human coronavirus (CoV) of the century. Similar to severe acute respiratory syndrome coronavirus (SARS-CoV) and Middle East respiratory syndrome coronavirus (MERS-CoV) infections, patients exhibited symptoms of viral pneumonia including fever, difficulty breathing, and bilateral lung infiltration in the most severe cases (Gralinski L E and Menachery V D et al (2020) Viruses 12, 135).

Severe acute respiratory syndrome Coronavirus 2 (SARS-CoV-2) is the causative agent of COVID-19, a disease that has rapidly spread across the planet with devastating consequences¹. SARS-CoV-2 infections can be asymptomatic and mostly present with mild to moderately severe symptoms. However, in approximately 10% of patients, COVID-19 progresses to a more severe stage that is characterized by dyspnoea and hypoxemia, which may progress further to acute respiratory distress requiring often long-term intensive care and causing death in a proportion of patients. Most likely, the ongoing inflammation triggered by the innate recognition of the SARS-CoV-2 virus, and possibly also by immune complexes with antibodies from an ineffective immune response⁷⁶, contributes to severe disease progression.

The novel CoV (2019-nCoV or WUHAN-Corona or SARS-CoV-2 virus) was isolated from a single patient and subsequently verified in 16 additional patients⁵⁰⁻⁵². The 30.000 nucleotide 2019-nCoV (also designated herein as Wuhan-Corona virus, or SARS-CoV-2) genome was elucidated in record time (see the internet at: virological.org/t/novel-2019-coronavirus-genome/319 (accessed on 19 Jan. 2020). The first available sequence data placed the novel human pathogen SARS-CoV-2 in the Sarbecovirus subgenus of Coronaviridae, the same subgenus as the SARS virus. Although SARS-CoV-2 belongs to the same genus Betacoronavirus as SARS-CoV (lineage B) and MERS-CoV (lineage C), genomic analysis revealed greater similarity between SARS-CoV-2 and SARS-CoV, supporting its classification as a member of lineage B (from the International Committee on Taxonomy of Viruses). Among other betacoronaviruses, this virus is characterized by a unique combination of polybasic cleavage sites, a distinctive feature known to increase pathogenicity and transmissibility. A bat sarbecovirus, Bat CoV RaTG13, sampled from a Rhinolophus affinis horseshoe bat was reported to cluster with SARS-CoV-2 in almost all genomic regions with approximately 96% genome sequence identity, which lead to the conclusion that the COVID-19 outbreak, from SARS-Cov-2 with its proximity to RaTG13, originates from a bat transmission to humans. However, the bats' general biological differences from humans make it feasible that other mammalian species acted as intermediate hosts, in which SARS-CoV-2 obtained some or all of the mutations needed for effective human transmission. One of the suspected intermediate hosts, the Malayan pangolin, harbours coronaviruses showing high similarity to SARS-CoV-2 in the receptor-binding domain, which contains mutations believed to promote binding to the angiotensin-converting enzyme 2 (ACE2) receptor and demonstrates a 97% amino acid sequence similarity. SARS-CoV-1 and -2 both use angiotensin converting enzyme 2 (ACE2) as a receptor on human cells. SARS-CoV-2 binds ACE2 with a higher affinity than SARS-CoV-1²³.

The receptor binding domain (RBD) of the Spike protein of the bat coronavirus (RaTG13) also revealed to be highly similar, over 93%, to that of SARS-CoV-2 genome. On the other hand, relative to SARS-CoV, significant differences were observed in the sequence of the S gene of SARS-CoV-2, including three short insertions in the N-terminal domain, changes in four out of five of the crucial residues in the receptor-binding motif, and the presence of an unexpected furin cleavage site at the S1/S2 boundary of the SARS-CoV-2 spike glycoprotein, thereby differentiating SARS-CoV-2 from SARS-CoV and several SARS-related coronaviruses (SARSr-CoVs) (for an overview see 75).

The severe lung disease in COVID-19 patients seems to result from an overshooting inflammatory response⁶⁰. However, because even non-human primates do not fully replicate COVID-19, little information and no appropriate animal models were initially at hand to address this hypothesis⁶¹. Syrian hamsters (Mesocricetus auratus) have been proposed as a small animal model to study SARS-CoV-induced pathogenicity and the involvement of the immune response in aggravating lung disease. Their superiority as pre-clinical model is currently of interest to rationalize and assess the therapeutic benefit of new antivirals or immune modulators for the treatment of COVID-19 patients.

Antibodies protect against infectious diseases. Whereas prophylactic vaccines will expectedly become a cornerstone of controlling the pandemic, such vaccines will still leave a significant part of the population insufficiently protected. Indeed, immunity against coronaviruses can be short-lived, and, in the case of seasonal influenza, the other main respiratory virus of humankind, vaccine effectiveness rarely exceeds 60%². Especially the elderly, the section of the population that is most at risk of developing severe disease upon SARS-CoV-2 infection, tend to be protected less efficiently upon vaccination. Hence, passive antibody immunotherapy to suppress or even prevent viral replication in the lower airways will likely find an important place in rescuing patients who fall ill, even after safe and effective vaccines have become available. In such patients, immunoglobulin egress from the systemic circulation into the broncho-alveolar space is augmented due to the inflammation in the lower airways, and we hence can make use of systemic administration of the antibody. When using an IgG Fc-containing antibody construct, this comes with the strong advantage of long native circulatory half-life imparted by the FcRn-mediated recycling into the bloodstream of such antibodies³.

While the jury is still out whether antibodies could exacerbate inflammatory disease in COVID-19, it may be prudent in patients with aggravating COVID-19 disease to rely on a pure virus neutralization mechanism of action, and thus to engineer out effector functions from the antibody Fc domain. Evidence so far suggests that complement activation, including by immune complex formation, is the key pathway to be avoided⁴. Activation of complement receptor C5a on macrophages, e.g., leads to the production of the pro-inflammatory cytokines IL-6 and TNF, and an uncontrolled activation of this pathway may lead to a cytokine storm. In line with this, inhibition of complement activation as well as IL-6 receptor signaling blockage in COVID-19 patients with acute respiratory distress is likely beneficial, provided treated patients are carefully stratified according to their disease stages^{5,6}. The IgG Fc-LALA mutations are an effective and well-validated means to blunt antibody Fc-mediated effector functions⁷. These mutations obliterate FcγR-mediated effector functions, with only FcγRI interaction still detectable in vitro, be it with an extremely high ED₅₀ that is likely not physiologically relevant. The anticipated safety profile of an Fc-LALA molecule is also supported by the observation that a neutralizing human monoclonal directed against all four dengue virus serotypes, with introduced LALA mutations circumvented enhanced infection of human cells⁸. The trace of remaining FcγRI interaction can be further removed by an additional P329G mutation in the Fc (LALAPG)⁹.

Coronaviruses have lower mutation rates than other RNA viruses, especially influenza A viruses, and high rates of viral replication within hosts because of the 3′-to-5′ exoribonuclease activity associated with the non-structural protein nsp.14. Though, Severe acute respiratory syndrome Coronavirus 2 (SARS-CoV-2), spreads even more rapid across the planet since several viral mutants developed, with an increased infection potential. With SARS-CoV-2 vaccines being developed and administered within historically short periods, their coverage to also protect for these novel mutants cannot be anticipated. To combat disease, many antibodies currently under clinical development may provide for alternative treatment options which may, or may not cover future mutant viruses.

Passive antibody immunotherapy with broadly neutralizing molecules, to prevent or suppress viral replication in the lower airways, will thus find an important place in rescuing COVID-19 patients. Indeed, the early development of sufficient titers of neutralizing antibodies by the patient correlates with avoidance of progression to severe disease⁷⁷, and early administration of recombinant neutralizing antibodies or those present in high-titer convalescent plasma can avert severe disease⁷⁸⁻⁸⁰. A strong advantage of antibodies and antibody Fc-based fusions compared with small molecule drugs is their long circulatory half-life imparted by the FcRn-mediated recycling into the bloodstream, which provides for long term control of virus replication even after a single administration³.

So there remains a pressing need to learn more about this virus, particularly in the diagnostics, prophylaxis and/or treatment of this novel virus, and in particular novel mutants therefor, as the virus has now spread worldwide.

### SUMMARY OF THE INVENTION

The present invention provides for binding agents which can specifically bind to SARS-Corona (SARS-Cov or SARS-Cov-1) virus and 2019-nCorona virus (also called SARS-CoV-2 virus). More specifically we immunized a llama with prefusion stabilized spike (S) proteins of Severe Acute Respiratory Syndrome (SARS) and Middle East Respiratory Syndrome (MERS) coronavirus (CoV). These S proteins are antigenically diverse. We isolated a single domain antibody, named SARS VHH-72 (or further also designated herein as ‘VHH-72’, ‘VHH72’, ‘VHH72-wt’, ‘parental VHH72’, ‘WT-VHH’, or ‘nanobody-72’ (Nb72)), that potently neutralized SARS-CoV pseudotypes and is thus capable of preventing infection by this virus. Surprisingly, despite the antigenic divergence, SARS VHH-72 cross-reacted with SARS-CoV-2 S protein and also neutralized pseudotyped viruses. In addition, co-crystal structure analysis revealed that the SARS-CoV and SARS-CoV-2 cross-reactive single domain antibody bound to a conserved surface of the receptor-binding domain (RBD) of the spike protein, and yet prevented this RBD to bind to angiotensin converting enzyme 2 (ACE2), the known receptor of SARS-CoV-1 and SARS-CoV-2. CR3022 was also recently reported to be able to bind with purified recombinant 2019-nCoV RBD as determined by ELISA and bio-layer interferometry ss. However, CR3022 does not compete for finding of ACE2 to the SARS-CoV-2 RBD, whereas we observed a clear competition between ACE2 and SARS VHH-72 for binding with SARS RBD. In addition, CR3022 recognizes looped peptides in two domains, i.e. peptides ATSTGNYNYKYRYLRHGKLR and YTTTGIGYQPYRVVVLSFEL, which have the motif TXTGXXXXXYR in common, suggesting that this antibody recognizes linear epitopes in SARS CoV (patent application US2008/0014204; note CR3022 is named CR03-022 in this application). In contrast SARS VHH-72 interacts with a well-defined conformational epitope in the RBD of SARS CoV making close contact with Leu355, Tyr356, Ser358, Ser362, Thr363, F364, K365, C366, R426 and Y494, from the Spike protein of SARS-Cov-1, as depicted in SEQ ID NO:24. Said epitope corresponds to the epitope with residues L368, Y369, S371, S375, T376, F377, K378, C379 and Y508 of the Spike protein of SARS-Cov-2, as set forth in SEQ ID NO: 23. The binding agents specifically binding to said epitope as described herein specifically bind alternative RBD domain proteins of further Sarbecoviruses as well, as shown herein.

Based on the co-crystal structure of SARS-VHH72 with SARS-CoV-1 RBD, and the cryo-EM structure of the SARS-CoV-2 spike in the prefusion conformation²³, several variants of SARS-VHH72 were designed with superior binding characteristics such as improved kₒₙ rates and improved k_{off} rates, and/or a higher affinity for SARC-CoV-2 RBD, and thus with a further increased antiviral potential against the SARS-CoV-2 virus. One specific variant of VHH72 with superior binding and potency characteristics has been identified herein as the VHH72-S56A variant (as depicted in SEQ ID NO: 4) and was selected for further preclinical development in the bivalent format of an IgG Fc fusion as to provide for the VHH72 variant with optimal potency, efficacy and biophysical properties when administered as an Fc fusion to a subject. Said VHH72-S56A variant fused to a human IgG1 Fc domain showed an enhanced neutralization potency with SARS-CoV-1 or SARS-CoV-2 S protein in pseudotype assays, and even showed neutralization potency and efficacy in vivo upon injection with SARS-Cov-2 in Syrian hamsters.

Analysis of the binding site of VHH-72 in complex with the SARS-CoV-1 and/or SARS-CoV-2 RBD revealed that very conserved residues are bound by the VHH and may therefore provide for a cross-protection to other Coronaviruses as well as confer resistance to new SARS-CoV-2 mutant variants.

In the interest of strengthening the viral protection efficacy, multivalent or multispecific molecules comprising additional VHHs, wherein said additional VHHs or ISVDs may bind to the same epitope, an overlapping epitope, or a different non-competing epitope as VHH72, are envisaged herein. In the present application, several additional approaches are described as to provide for additional VHH72 family members and additional VHH families that bind and/or compete for the same conserved RBD binding site on the Spike protein, and wherein said additional VHHs of the same family as VHH72, or of different VHH families are further improved in binding and neutralization characteristics.

So in a first aspect the invention relates to a binding agent recognizing the Corona virus SARS-Cov-1 spike protein by binding to its RBD domain at least via the residues Leu355, Tyr356, Ser358, Ser362, Thr363, F364, K365, C366, R426 and Y494, from the Spike protein of SARS-Cov-1, as depicted in SEQ ID NO:24, or alternatively, further via the residue R426 as depicted in SEQ ID NO:24. Alternatively, binding agent can be defined as specifically recognizing the Corona virus SARS-Cov-2 spike protein by binding to its RBD domain at least via the residues or residues L368, Y369, S371, S375, T376, F377, K378, C379 and Y508 of the Spike protein of SARS-Cov-2, as set forth in SEQ ID NO: 23. Another embodiment relates to a binding agent specifically binding the Corona virus Spike protein, which binds to said binding site region in a competing mode with the binding agent specifically binding to those specific residues L368, Y369, S371, S375, T376, F377, K378, C379 and Y508 of the Spike protein of SARS-Cov-2, as set forth in SEQ ID NO: 23. Specifically, said competing binding agent specifically binds an epitope on the Spike protein comprising at least a part of the residues L368, Y369, S371, S375, T376, F377, K378, C379 and Y508 of the Spike protein of SARS-Cov-2, as depicted in SEQ ID NO:23, so as to provide an overlapping epitope, more specifically at least binding 30% of the residues, or at least 50% of the residues, or at least 80% of the residues, and/or specifically including residues K378, and/or F377.

In different embodiments, said binding agents may be a small molecule, a chemical, a peptide, a compound, a peptidomimetic, an antibody, an antibody mimetic, an active antibody fragment, an immunoglobulin single variable domain (ISVD), or a Nanobody.

In one embodiment, said binding agent specifically binding the RBD of the Spike protein as defined herein, in particular relates to polypeptides comprising an ISVD, said ISVD comprising 4 framework regions (FR) and 3 complementarity determining regions (CDR) according to the following structure: FR1-CDR1-FR2-CDR2-FR3-CDR3-FR4 (1); as depicted in VHH or Nanobodies for instance. In more specific embodiments, the CDRs are defined as CDR1 comprising SEQ ID NO: 7, or SEQ ID NO:111-119, or the sequence SYAMG, CDR2 comprising SEQ ID NO: 8, SEQ ID NO:10, SEQ ID NO:120-130, or SEQ ID NO:141, or the sequence TISWSGGGTYYAEPVRG, and CDR3 comprising SEQ ID NO: 9, or SEQ ID NO:131-140.

An alternative embodiment provides for said binding agents wherein the 3 CDRs are selected from those CDR1, CDR2, and CDR3 regions as depicted in SEQ ID NO: 1, SEQ ID NO:4, SEQ ID NO:27-61, or SEQ ID NO: 92-105, wherein the CDR regions may be annotated according to Kabat, MacCallum, IMGT, AbM, or Chothia, as further defined herein. A further specific embodiment relates to said binding agents described herein, wherein at least one ISVD comprises SEQ ID NO: 1, 4, 27-61, or SEQ ID NO: 92-105, or a sequence with at least 90% amino acid identity thereof, considered over the whole length of the ISVD and wherein CDRs are identical, or a humanized variant of any one thereof. A specifical embodiment relates to the binding agents as described herein, wherein at least one ISVD comprises a humanized variant as depicted in SEQ ID NO: 2, 3, 5, 6, or 11, or a further variant thereof.

In another embodiment, the binding agent as described herein comprises an ISVD which linked to an Fc domain or fused to an IgG Fc tail, which may be derived from a conventional antibody structure, or a variant thereof, such as for example an IgG, IgG1 or IgG2 Fc domain, or a variant thereof.

Another embodiment relates to said binding agent which is multivalent or multispecific binding agent, possibly with one or more ISVDs being identical or binding the same of different epitopes on the Spike protein. In a specific embodiment, the binding agent comprising a bivalent ISVD, potentially fused to an Fc domain. In a further specific embodiment said bivalent ISVD may comprise SEQ ID NO:12, or a humanized variant thereof. A further specific embodiment relates to said binding agent described herein, wherein said ISVD is fused to an IgG Fc domain in a monovalent or multivalent format, preferably resulting in a tetravalent binding agent.

In some embodiments, said binding agent as described herein comprises a bivalent ISVD-Fc domain fusion, wherein said binding agent comprises a sequence selected from the group of SEQ ID NO:13 to SEQ ID NO:22, or a further humanized variant thereof, with at least 90% identity thereof. In a specific embodiment, the binding agent of the present invention consists of SEQ ID NO:22.

Another aspect of the invention relates to a nucleic acid molecule encoding any of the binding agents as described herein. Further embodiments relate to recombinant vectors comprising said nucleic acid molecule encoding the binding agent of the invention.

Another aspect of the invention relates to a complex comprising the Receptor binding domain of SARS-Corona virus as depicted in SEQ ID NO: 25 or SEQ ID NO: 26 and a binding agent specifically bound to said RBD, as described herein, more specifically said binding agent comprising the ISVD comprising any one of SEQ ID NOs: 1-6.

A further aspect relates to a host cell comprising the binding agent, the nucleic acid molecule, the recombinant vector, or the complex as described herein.

Another aspect relates to a pharmaceutical composition comprising the binding agent, the nucleic acid molecule, or the recombinant vector as described herein, optionally comprising a carrier, diluent or excipient.

An alternative aspect relates to the binding agent, the nucleic acid molecule, the recombinant vector, or the pharmaceutical composition as described herein, for use as a diagnostic. Or the binding agent, the nucleic acid molecule, the recombinant vector, or the pharmaceutical composition as described herein, for use in in vivo imaging.

Further aspects of the invention relate to the binding agent, the nucleic acid molecule, the recombinant vector, or the pharmaceutical composition as described herein, for use as a medicament.

Specifically, the binding agent, the nucleic acid molecule, the recombinant vector, or the pharmaceutical composition as described herein, are envisaged for use in prophylactic or therapeutic treatment of a subject with a coronavirus infection, more specifically a β-coronavirus infection, even more specifically, an infection from a zoonotic sarbecovirus, such as SARS-Corona virus infection, such as a SARS-CoV-2 virus infection, or a SARS-CoV-2 mutant virus infection, or for treatment of COVID-19. With prophylactic treatment is meant administration of the binding agent to the subject prior to illness or viral infection. Said prophylactic use of the binding agents may involve a treatment with a dose in a range of 0.5 mg/kg-25 mg/kg, preferably between 2 mg/kg and 20 mg/kg. Another embodiment relates to said binding agents as described herein for use in therapeutic treatment of SARS-Corona virus infection, more specifically for use in the treatment of 2019-nCorona (or SARS Cov-2) virus infection.

In a specific embodiment, with SARS-CoV-2 mutant virus infection, is meant a SARS-CoV-2 virus with a mutation in the Spike protein, preferably in the RBD domain, even more preferably comprising the specific mutation of N439K, S477N, E484K, N501Y, and/or D614G, as set forth in SEQ ID NO:23.

An alternative embodiment relates to the binding agent, or the pharmaceutical composition as described herein, are envisaged for use in prophylactic or therapeutic treatment of a subject with a coronavirus infection, said treatment comprising administration of a dose of 0.5 mg/kg-25 mg/kg of said binding agent or pharmaceutical composition. More specifically, administration may be envisaged intravenous, interperitoneally, subcutaneous, intranasal, or via inhalation.

A final aspect of the invention relates to the use of the binding agent as described herein, or a labelled form thereof, for detection of a viral particle or a viral Spike protein from a virus selected from the group of viruses belonging to clade 1a, 1b, 2 and/or 3 of bat SARS-related sarbecoviruses. More specifically, from the group of SARS-Cov-2, GD-Pangolin, RaTG13, WIV1, LYRa11, RsSHC014, Rs7327, SARS-CoV-1, Rs4231, Rs4084, Rp3, HKU3-1, or BM48-31 viruses.

### DESCRIPTION OF THE FIGURES

The drawings described are only schematic and are non-limiting. In the drawings, the size of some of the elements may be exaggerated and not drawn on scale for illustrative purposes.

FIG. 1. SARS VHH-72 binds to SARS CoV S RBD but not to the N-terminal domain of SARS CoV S.

Wells of microtiter plates (type II, F96 Maxisorp, Nuc) were coated overnight at 4° C., with 100 ng recombinant SARS-CoV S-2P protein (with foldon)(top), SARS-CoV RBD (middle) or SARS-CoV NTD (N-terminal domain, bottom). The coated plates were blocked with 5% milk powder in PBS. Dilution series of the indicated VHHs were added to the wells. Binding was detected by incubating the plates sequentially with mouse anti-Histidine Tag antibody (MCA1396, Abd Serotec) followed by horseradish peroxidase (HRP)-linked anti-mouse IgG (1/2000, NXA931, GE Healthcare). After washing, 50 μL of TMB substrate (Tetramethylbenzidine, BD OptETA) was added to the plates and the reaction was stopped by addition of 50 μL of 1 M H₂SO4. The absorbance at 450 nM was measured with an iMark Microplate Absorbance Reader (Bio Rad).

FIG. 2. Surface plasmon resonance (SPR) sensorgrams for the binding of SARS VHH-72 to immobilized SARS CoV RBD (top), WIV1-CoV RBD (middle) and 2019-nCoV RBD (bottom).

FIG. 3. Crystal structure of VHH72 binding to SARS-Cov RBD.

A. Crystal structure of SARS CoV RBD in complex with SARS VHH-72 (shown in blue) revealing the epitope-paratope interactions. The top left panel shows that SARS VHH-72 binds an epitope of the RBD that is distal from the ACE2 (the SARS CoV receptor) binding interface (shown in red). The bottom left panel is a close-up image of the interactions between the indicated amino acid such as the salt bridge between Asp61 in SARS VHH-72 and Arg426 in SARS CoV RBD. Top right depicts the clash between ACE2 bound to the SARS CoV RBD and the CDR-distal framework of SARS VHH-72 and ACE2. B. Sequence variation mapped onto the SARS CoV RBD crystal structure in complex with SARS VHH-72, illustrating the conservation of the conformational epitope.

FIG. 4: RBD-ACE2 binding is blocked by VHH72.

Octet neutralization assay. Diagram depicts the ligands/analytes. Blue curve shows association between SARS RBD and ACE2 (blocked by VHH72 in lower purple curve).

FIG. 5. Alignment of the amino acid sequences of the Receptor-binding domain of SARS-CoV and 2019-nCoV.

The residues in SARS-CoV RBD that are directly involved in the interaction with SARS VHH-72 are underlined. The residues in 2019-nCoV RBD that are underlined are identical to the corresponding residues in SARS RBD that are directly involved in interaction with SARS VHH-72. The amino acid residue in bold in 2019-nCoV RBD differs from the corresponding amino acid residue in SARS-CoV RBD that is involved in direct interaction with SARS VHH-72.

FIG. 6. VHH-72 prevents binding of ACE2 to the RBD of SARS-CoV (SARS-CoV RBD) and 2019-nCoV (2019-nCoV RBD-SD1).

Octet-based competition assay. The graph shows the association of the RBDs with their respective receptors in the presence of VHH-55 (MERS RBD-specific) and VHH-72 (SARS-CoV RBD- and 2019-nCoV RBD-specific).

FIG. 7. VSV-coronavirus spike pseudotype neutralization assay.

Vesicular stomatitis virus (VSV) reporter viruses encoding firefly luciferase and pseudotyped with spike proteins of 2019-nCoV, SARS-CoV or MERS-CoV (as indicated above each graph) were used in a pseudotype neutralization assay using VHH-72 (nb72), VHH-55 (nb55), GFP-binding protein (GBP=a nanobody that binds to GFP) or VHH-72 fused to human IgG1 Fc (nb72Fc). Preimmune and postimmune serum derived from the immunized llama that was used to isolate the VHHs from was also included. A-C. VSV pseudotypes were preincubated for 30 minutes with a serial dilution of cell supernatant derived from HEK293 cells that were transiently transfected with an expression construct for secretion of GBP or nb72Fc. VSV pseudotypes were also preincubated for 30 minutes with serial dilutions of llama pre-or postimmune serum or with PBS as indicated. D-F. VSV pseudotypes were preincubated for 30 minutes with a serial dilution of purified VHH-72 or VHH-55 or with PBS as indicated. After incubation, the pseudotype samples were transferred to a monolayer of VeroE6 cells, seeded in wells of a 96-well microtiter plate. Sixteen hours after incubation at 37 degrees Celsius, the supernatant was removed and the cells were lysed with 100 microliter of lysis buffer. Ten microliter of the lysate was then mixed with luciferine substrate and luciferase buffer and the luciferase signal (RLU) was measured in a Promega Glomax multi plate reader. Data points depict the measured luminescence signals. NI: not infected.

FIG. 8. Viral RNA levels in hamster lungs after prophylactic treatment with VHH72-Fc antibody or human plasma.

(A) Schematic representation of SARS-CoV-2 inoculation schedule. WT hamster strains were intranasally inoculated with 2×10⁶ of passage 6 SARS-CoV-2 (BetaCov/Belgium/GHB-03021/2020). On the indicated days post inoculation (d.p.i.), organs and blood were collected to determine viral RNA levels. (B) Viral RNA levels in hamsters after treatment with purified VHH72-Fc binding agents or convalescent SARS-CoV-2 plasma. Hamsters were either left untreated (IC, infection control, n=5) or treated with a bivalent VHH72-Fc antibody (VHH-72-Fc, n=4), convalescent plasma (patient #2, n=4) or negative control plasma (patient #3 NC, negative control, n=4) and sacrificed on day 4 p.i. Viral RNA levels were determined in the lungs, normalized against f-actin and fold-changes were calculated using the 2^{(-ΔΔCq)} method compared to the mean of IC. The data shown are means±SEM. Statistical significance between groups was calculated by the nonparametric two-tailed Mann-Whitney U-test (ns P>0.05,*P<0.05).

FIG. 9. Model of VHH72 in complex with SARS-CoV-2 spike protein RBD domain.

VHH72 at top-right; RBD at bottom.

FIG. 10. Zoom-In near S56A of a VHH72-S56A/RBD model.

VHH72 at top-right and RBD at bottom as in FIG. 9. S56A, W52a, V100 and V100a of VHH72; and Y369, F377 and P384 of RBD indicated as sticks. Although S56's OH group resides in a minor depression of the RBD that is apparently polar (nearby presence of backbone carbonyls from L368, Y369, S371 and F374, not shown), this S56A mutant was selected due to its relative proximity to RBD's Y369 which we suspected to be in an “up” conformational position in contradistinction to the “down” position as observed in many SARS-RBD crystal structures. After in vitro observation of improved binding, a subsequent molecular dynamics run (using Gromacs with Amber) instead unexpectedly suggests S56A to be in hydrophobic interaction with VHH72's V100 and V100a, and RBD's Y369 and F377.

FIG. 11. Zoom-in near T60W of a VHH72-T60W/RBD model.

VHH72 at top-right and RBD at bottom as in FIG. 9. T60W, F47 and Y58 of VHH72; and D437, V503 and Y508 of RBD indicated as sticks.

FIG. 12. Coomassie blue staining of SDS-PAGE gels containing Pichia pastoris culture supernatants expressing different VHH-IgG Fc fusion constructs.

The constructs expressed for each sample lane are indicated in the figure.

FIG. 13. Coomassie blue staining of SDS-PAGE gels containing Pichia pastoris culture supernatants expressing different VHH-IgG Fc fusion constructs.

The constructs expressed for each sample lane are indicated in the figure.

FIG. 14. Coomassie blue staining of SDS-PAGE gels containing Pichia pastoris culture supernatants expressing different VHH-IgG Fc fusion constructs.

The constructs expressed for each sample lane are indicated in the figure.

FIG. 15. Coomassie blue staining of SDS-PAGE gels containing HEK293-S culture supernatants expression different VHH-IgG Fc fusion constructs.

The constructs expressed for each sample lane are indicated in the figure.

FIG. 16. Western blot images from the SDS-PAGE samples containing HEK293-S culture supernatants expression different VHH-IgG Fc fusion constructs.

The constructs expressed for each sample lane are indicated in the figure, and as shown in SDS-PAGE in FIG. 15. The antibodies used in the left panels specifically bind VHH, and the antibodies used in the right panels specifically bind the human Fc part of the antibodies.

FIG. 17. Binding of VHH72-Fc to immobilized SARS-CoV-2 RBD as determined by BLI.

Association and dissociation rates are comparable for two different linkers tested (hIgG1 hinge without or with an additional (GGGGS)x2 linker).

FIG. 18. Binding of VHH72-Fc to immobilized SARS-CoV-2 RBD as determined by BLI.

VHH72-T60W variant has improved binding to compared to parental VHH72, whereas VHH72-W52aH binds less well.

FIG. 19. Binding of VHH72-Fc to immobilized SARS-CoV-2 RBD as determined by BLI.

Comparison of binding of VHH72-D61Q and VHH72-V100L variants with parental VHH72.

FIG. 20. Binding of VHH72-Fc to immobilized SARS-CoV-2 RBD as determined by BLI.

Variant VHH72-S56A has a slower dissociation rate compared with parental VHH72.

FIG. 21. Binding of VHH72-Fc to immobilized SARS-CoV-2 RBD as determined by BLI.

Calculated dissociation constants of VHH72-Fc parental and VHH72-Fc variant constructs based on BLI measurements.

FIG. 22. Binding of VHH72-Fc to immobilized SARS-CoV-2 RBD as determined by BLI.

FIG. 23. Binding of the SARS VHH-72 variants to cells expressing the SARS-CoV (grey) or SARS-CoV-2 (black) Spike proteins.

The bars represent the AF633 mean fluorescence intensity (MFI) of GFP expressing cells (GFP⁺) divided by the MFI of GFP negative cells (GFP⁻).

FIG. 24. VHH72 and VHH72(S56A) bind to a conserved epitope on SARS-CoV-2 RBD. a. RBD as surface-view with the VHH72 epitope indicated in yellow, for which PDBePISA¹ predicts residues 368-379, 381-385, 404, 405, 407, 408, 435-437, 503, 504 and 508. Right; as calculated by FastContact, averaged from 30 Molecular Dynamics snapshots. RBD as surface view with the epitope indicated in thresholds of calculated electrostatic plus desolvation free energy (kcal/mol) per residue by FastContact^{2,3}. The epitope shows a prominent hot-spot consisting of Lys378 and Phe377. Red: −9.8 (K378); orange: −4.27 (F377); yellow: −2.21/−0.96 (Y369, A372, S375, T376, C379, V407, R408, Y508); green: −0.71/−0.30 (S371, F374, P384, K386, W436, N437, V503); blue: −0.27/−0.13 (L368, S373, T385, R403, A411, Q414, N439, N501, G502, G504, Y505). b. Location of VHH72 on the SARS-CoV-2 pre-fusion spike protein. VHH72 (rainbow cartoon, C-terminus as red sphere, top left) on the RBD of chain C (magenta cartoon, top right) from spike's 2-RBDs ‘up’ state in its u152q quadruple mutant (A570L, T572I, F855Y, N856I) structure pdb-entry 6×2b⁴. Small, sideways-binding, and with its C-terminus pointing far outwards, a VHH72-Fc construct can easily follow the wide movements of an ‘up’ RBD on the spike protein. c. The epitope of VHH72 is occluded in the RBD-closed state of 5ARS-CoV-2 spike pre-fusion protein. Apex-view of intact wild-type SARS-CoV-2 pre-fusion closed state spike trimer pdb-entry 6×r8⁵, showing only the three RBDs. Chain A, grey-surface; chain B, cyan-cartoon; chain C, magenta-cartoon. The VHH72 epitope according to PDBePISA is indicated in yellow with the Lys378 and Phe377 hot-spot is shown in red and orange. d. Comparison of VHH72 (top) in complex with SARS-CoV-1 RBD (bottom) PDB entry 6WAQ (chain D)) with a [homology-model of VHH72-S56A binding to] SARS-CoV-2 RBD (model obtained from the I-TASSER server³), zoomed-in to the zone near VHH72Ser56. Residues Ser52, Trp52a, Ser53, Ser56 and Val100 of VHH72, residues Tyr352, Tyr356, Asn357, Ser358, Thr359 (this NXT sequence bears an N-glycan, not shown) and Ala371 of SARS-CoV-1 RBD, and SARS-CoV-2 RBD residues Tyr365, Tyr369, Asn370, Ser371, Ala372 (no NXT) and Pro384 are shown as sticks. Left: VHH72/SARS-CoV-1 RBD. Tyr356 and Tyr352 are pointing downward in a groove-like depression of the RBD. Right: VHH72/SARS-CoV-2 RBD. In this I-TASSER RBD model, the corresponding Tyr356 and Tyr369 are pointing upward. The Tyr369 upward conformation appears to be preferred as a result of the nearby Pro384 in SARS-CoV-2 RBD (Ala371 in SARS-CoV-1 RBD). Tyr369 then resides in a small cavity of VHH72 and is surrounded by Ser52, Trp52a, Ser53, Ser56 and Val100. The hydroxyl group of VHH72 Ser56 is oriented towards the centre of the aromatic group of SARS-CoV-2 RBDTyr369. Figures prepared with Pymol (The PyMOL Molecular Graphics System, Open Source Version 2.3. Schrödinger, LLC).

FIG. 25. Binding affinity determination of monovalent humanized VHH72_S56A for SARS-CoV RBD. a. BLI sensorgram of different VHH72 variants binding to monomeric RBD from Sars-CoV-1 and Sars-CoV-2. KD values of VHH72 variants to Sars-CoV-2 RBD (biotinylated via Avi-tag) in 1:1 interaction. b. To assess the affinity of the VHH72 variants in a 1:1 interaction, the kinetic binding constant K_{D} of the monovalent affinity optimized variants VHH72(S56A into h1, and into h2) were assessed in BLI, comparing binding to monomeric SARS-CoV-2 RBD protein, and dimeric SARS-CoV-2 RBD-Fc-fusion. As reference, the humanized VHH72 h1 was included. The concentration range of VHHs was between 100 nM and 1.56 nM, and results were fitted according to 1:1 interaction.

FIG. 26. Monovalent VHH72_S56A binding and neutralization activity.

a. Flow cytometry analysis of the binding of VHH72WT, VHH72S56A, and, as a control GBP to 293T cells that were transiently transfected with a GFP expression vector combined with a SARS-CoV-2 expression vector. Binding of HIS-tagged VHHs was detected using a mouse monoclonal anti-HIS antibody and a AF647 conjugated donkey anti-mouse IgG antibody. Y-axis: median fluorescent intensity (MFI) of the AF647 fluorescence of the GFP-positive cells divided by the MFI of the GFP-negative cells. b. Flow cytometry analysis of binding of recombinant SARS-CoV-2 RBD-Fc fusion protein to VeroE6 cells in the presence of different concentrations of VHH-72 (moWT), VHH-72S56A (moS56A), or GBP. PBS and no RBD were also included as controls. Cells bound by SARS-CoV-2 RBD-Fc were detected using an AF488 conjugated donkey anti-mouse IgG antibody. The graph shows the mean t standard deviation (n=3) percentage of VeroE6 cells bound by SARS-CoV-2 RBD-Fc. c. SARS-CoV-2 spike pseudotyped GFP reporter vesicular stomatitis virus (VSV) neutralization assays. VHH-72 h1, VHH-72 h1-S56A, or GBP were added to the VSV reporter virus at the concentrations indicated in the X-axis prior to infection of VeroE6 cell monolayers. GFP fluorescence of the cells was measured 19 hours later. NI: not infected. The graph shows the mean t standard deviation (n=4) GFP MFI. d. ELISA that shows binding of VHH72-h1 and VHH72-h1(S56A) to immobilized SARS-CoV-1 RBD. GBP=GFP-binding protein=a VHH that is specific for green fluorescent protein. Binding of VHHs was detected using a hrp-conjugated rabbit anti-VHH monoclonal antibody. The graph shows the mean t standard deviation (n=3) O.D. at 450 nm. e. SARS-CoV-1 spike pseudotyped GFP reporter vesicular stomatitis virus (VSV) neutralization assays. VHH-72 h1, VHH-72 h1-S56A, or GBP were added to the VSV reporter virus at the concentrations indicated in the X-axis prior to infection of VeroE6 cell monolayers. GFP fluorescence of the cells was measured 19 hours later. NI: not infected. The graph shows the mean t standard deviation (n=4) GFP MFI.

FIG. 27. VHH72_S56A-Fc constructs have increased affinity for SARS-CoV-2 spike protein.

a. ELISA that demonstrates binding to immobilized SARS-CoV-2 spike of the indicated VHH-72-Fc constructs. Syn=synagis. Binding of VHH-Fc constructs was detected using a hrp-conjugated rabbit anti-human IgG antibody. The graph shows the mean±standard deviation (n=2) O.D. at 450 nm. b. ELISA that demonstrates binding to immobilized SARS-CoV-2 RBD-murine Fc fusion protein of the indicated VHH-72-Fc constructs. Syn=synagis. The graph shows the mean±standard deviation (n=2) O.D. at 450 nm. c and d. SARS-CoV-2 spike glycoprotein expressing HEK293T were assessed for binding efficiency of the VHH72_h1(E1D,S56A)_10GS_Fc hIgG1 LALA (PB9683; SEQ ID NO: 22) and VHH72_h1(E1D,S56A)_10GS_Fc hIgG1 (PB9587 in d). Binding was determined via incubation of the HEK293T cell line with test antibodies (1.22-5000 ng/mL, 4-fold dilutions) or hIgG1 isotype control (312.5-5000 ng/mL) followed by anti-human IgG PE-conjugated secondary antibody staining. Unstained and stained cells were analysed by flow cytometry. Data shown as Median Fluorescence Intensity (MFI) and % PE-bound cells+/−SEM of technical replicates. Non-linear four parameter curve fit was applied to generate curves of best fit where possible and EC50 calculated for MFI. e. Binding efficiency of VHH72_h1(E1D,S56A)-Fc hIgG1 LALA (PB9683) to recombinant SARS-CoV-2 RBD-SD1-hFc glycoprotein. Wells of microtiter plates (type II, F96 Maxisorp, Nuc) were coated overnight at 4° C. with 30 ng recombinant SARS-CoV-2 RBD-SD1-hFc. The coated plates were blocked with 3% BSA in PBS. Dilution series of the VHHs were added to the wells. After washing, serially diluted mAbs were added into wells and incubated for 1 h at RT. Binding was detected by incubating the plates with an HRP-conjugated rabbit anti-camelid VHH monoRAB antibody 96A3F5 (A01861-200, GenScript, 1:5000 dilution). After washing 50 μL of TMB substrate (Tetramethylbenzidine, BD OptETA) was added to the plates and the reaction was stopped by addition of 50 μL of 1 M H2SO4. The absorbance at 450 nM was measured with an iMark Microplate Absorbance Reader (Bio Rad). Curve fitting was performed using nonlinear regression (Graphpad 8.0). f. Competition of VHH72_h1(E1D,S56A)-Fc hIgG1 LALA (PB9683) of the binding of monovalent VHH72_h1(E1D,S56A) sequence optimized (SO) to recombinant SARS-CoV-2 RBD glycoprotein.

FIG. 28. Neutralization of VSV pseudotyped with SARS-CoV-1 and -2 spike.

a. Vesicular stomatitis virus (VSV) GFP reporter virus pseudotyped with SARS-CoV-1 spike neutralization assays. Serial dilutions of the indicated VHH-Fc constructs were added to the VSV reporter virus at the concentrations indicated in the X-axis prior to infection of VeroE6 cell monolayers. GFP fluorescence of the cells was measured 19 hours later. The graph shows the mean±standard deviation (n=3) normalized GFP MFI. D72-2: VHH72-GS-hIgG1hinge-hIgG1Fc, D72-16: VHH72_h1-GS-hIgG1hinge-hIgG1Fc, D72-22: VHH72_h1_S56A-GS-hIgG1hinge-hIgG1Fc, D72-15: VHH72-GS-hIgG1hinge-hIgG1Fc_LALAPG, D72-17: VHH72_h1-GS-hIgG1hinge-hIgG1Fc_LALAPG, D72-23: VHH72_h1_S56A-GS-hIgG1hinge-hIgG1Fc_LALAPG, b-d. VSV SARS-CoV-2 spike pseudotype virus neutralization assay, tested using VHH72_h1(E1D,S56A)_10GS_Fc hIgG1 LALA (PB9683), VHH72_h1(E1D,S56A)_10GS_IgG1_LALAPG (PB9590), VHH72_h1(E1D,S56A)_10GS_IgG4_FALA (PB9677), VHH72_h1(E1D, S56A)_10GS_IgG1 (PB9587), and as a reference the original wild-type VHH72-Fc is included¹⁰. GFP readout, normalized.

FIG. 29. SARS-CoV-2 plaque reduction neutralization assay.

A SARS-CoV-2 plaque reduction neutralization assay was performed with 3-fold serial dilutions of the indicated VHH-Fc fusion constructs. Approximately 70 plaque forming units of SARS-CoV-2 were incubated for 1 h at 37 degrees Celsius and then transferred to confluent VeroE6 cells monolayers in wells of a 24-well plate. The cells were overlayed with methylcellulose and incubated for 72 h at 37 degrees Celsius. The overlay was removed, the cells fixed with 3.7% paraformaldehyde and stained with 0.5% crystal violet. Data points in the graph represent the number of plaques and are representative of one experiment that was repeated once. PB9682 and VHH23-Fc is a negative control VHH-Fc fusion.

FIG. 30. ACE2 competition assays.

Left: Inhibition of SARS-CoV-2 RBD-mFc protein binding to ACE-2 expressed on VeroE6 cells determined by flow cytometry. The VHH72_h1(E1D)_S56A-10GS-hIgG1Fc_LALAPG (D72-52; PG mutant as compared to PB9683) showed competition of ACE2 with an IC50 of 198.6 ng/mL, vs the prototype VHH72-Fc IC50 of 505 ng/mL.

Right: Competition of ACE2 binding to SARS-CoV-2 spike RBD domain was assessed in a competition Alphascreen with recombinant human ACE2-mFc protein bound to SARS-CoV-2 RBD protein biotinylated through the Avi-tag. The IC50 of the VHH72_h1(E1D)S56A-10GS-hIgG1Fc_LALA (PB9683) in this assay setup is 15.4 ng/ml (186 pM). In this assay the prototype VHH72-Fc showed an IC50 of 34 ng/ml.

FIG. 31. Tetravalent VHH72-Fc has Increased affinity for SARS-CoV-2 RBD.

a. Biolayer interferometry (BLI) sensogram measuring apparent binding affinity of VHH72_h1_hFc, (VHH72_h1)2_hFc, VHH72_h1_E1D_S56A-hFc_ΔEPKC-LALAPG-ΔK, and tetravalent (VHH72_h1_E1D_S56A)2-hFc_ΔEPKC-LALAPG-ΔK to immobilized SARS-CoV-2 RBD-mFc. Black lines represent double reference-subtracted data and the fit of the data to a 1:1 binding curve is colored red. b. A SARS-CoV-2 plaque reduction neutralization assay was performed with 3-fold serial dilutions of the indicated VHH-Fc fusion constructs. Approximately 70 plaque forming units of SARS-CoV-2 were incubated for 1 h at 37 degrees Celsius and then transferred to confluent VeroE6 cells monolayers in wells of a 24-well plate. The cells were overlayed with methylcellulose and incubated for 72 h at 37 degrees Celsius. The overlay was removed, the cells fixed with 3.7% paraformaldehyde and stained with 0.5% crystal violet. Data points in the graph represent the number of plaques and are representative of one experiment that was repeated once. Batch D72-52 corresponds to the construct: VHH72_h1(E1D, S56A)-10GS-hIgG1Fc_LALAPG and batch D72-55 to the tetravalent counterpart: VHH72_h3_S56A-(G₄S)₃-VHH72_h3_S56A-GS-hIgG1Fc_LALAPG.

FIG. 32. SARS-CoV-2 plaque reduction neutralization assay.

An assay was performed as described for FIG. 29. The constructs compared herein revealed that E1D modification, combined with truncation of the human IgG1 hinge and deletion of the C-terminal lysine residue does not affect VHH72-Fc affinity for RBD and SARS-CoV-2 neutralizing activity.

FIG. 33. Neutralization activities in a SARS-CoV-2 live virus assay.

D72-51 (VHH72_h1(E1D)S56A-10GS-hIgG1hinge_EPKSCdel-hIgG1Fc_LALAPG) and D72-52 (VHH72_h1(E1D)_S56A-10GS-hIgG1hinge_EPKSCdel-hIgG1Fc_LALAPG_Kdel) containing hIgG1_LALAPG Fc showed PRNT50 of 164.8 ng/mL and 163.9 ng/ml, respectively.

FIG. 34. Prophylactic administration of VHH72-Fc constructs in bivalent or tetravalent (VHH-VHH72-Fc) formats protects Syrian hamsters against SARS-CoV-2 viral replication.

Golden Syrian hamsters were treated with bivalent D72-23 and tetravalent D72-13 VHH-Fcs at 20 mg/kg by intraperitoneal injection 24 h before challenge with 2.4×10⁶ TCID50 of passage 6 BetaCov/Belgium/GHB-03021/2020. Control animals received 20 mg/kg of Synagis (n=6 per group). Genomic SARS-CoV-2 RNA copies were determined by RT-qPCR in lungs, ileum and stool tissues taken at day 4 post infection. b. Infectious virus loads in the lungs (day 4 after infection). c. Severity score of lung damage and of dilated bronchi was assessed by micro CT scan on day 4 after the challenge. TCID50=50% tissue culture infectious dose. Statistical analysis was performed using non-parametric Mann Whitney U-test. **P<0.005, ***P<0.001. Dotted line represents lower limit of detection (LOD).

FIG. 35. Prophylactic administration of 4 mg/kg of bivalent VHH72-Fc protects hamsters against SARS-CoV-2 infection.

A, study outline. Gold Syrian hamsters received bivalent D72-23 (VHH72_S56A-Fc (LALAPG)) at 4 or 20 mg/kg by intraperitoneal injection one day prior to challenge (n=5). Control animals received Synagis at the dose of 20 mg/kg (n=6). Intranasal challenge was done with 2.4×10′ TCID50 of passage 6 BetaCov/Belgium/GHB-03021/2020. B, Viral genomic RNA copies in lung, ileum and stool samples determined by qPCR, and infectious virus in lungs and nasal swabs determined by titration, in samples of day 4 after challenge. The two hamsters that had received 20 mg/kg of D72-23 and displayed high virus loads in lungs and nasal swabs, had no VHH72-23 exposure. C, Cumulative lung histopathology score assessed by immune-histochemistry analysis (day 4).

FIG. 36. Therapeutic administration of VHH72-Fc protects hamsters against SARS-CoV-2 challenge Infection.

Infectious SARS-CoV-2 in lung of Syrian hamsters following prophylactic (day −1 post infection (p.i.)) or therapeutic (day 1 p.i.) IP treatment with D72-52/PB9590 and D72-55/PB9589 (7 or 1 mg/kg) or the control Ab Synagis (7 mg/kg). Challenge was done with 2.4×106 TCID50 of BetaCov/Belgium/GHB-03021/2020 (p6). A, Study outline. B, Infectious SARS-CoV-2 particles in the lung, and genomic SARS-CoV-2 RNA copies in lungs, ilium and stool samples collected at day 4. C, Histopathology analysis of day 4 lungs assessed by immunohistochemistry (left panel), showing cumulative lung damage score. Middle and right panel: General lung damage and the bronchi image scoring assessed by micro-CT analysis. Statistical analysis was performed using non-parametric Mann Whitney U-test.: **** P<0.0001; ***P<0.001; ** P<0.01; * P<0.05. Dotted line represents lower limit of detection (LOD).

FIG. 37. Effect of therapeutic administration of VHH72-Fc protects hamsters against SARS-CoV-2 infection in the upper and lower respiratory tract.

Anti-viral efficacy in Syrian hamsters following therapeutic IP treatment with D72-52/PB9590 and D72-55/PB9589 (20, 7 or 2 mg/kg) or the control Ab Synagis (20 mg/kg), or prophylactic treatment of D72-52 at 20 mg/kg. Challenge was done with 1×104 TCID50 of BetaCoV/Munich/BavPat1/2020 (p3).

A: Study outline; C: Lung pathology, scoring the % of affected lung region by macroscopic lesions. Significant reduction of macroscopic lesions by 7 mg/kg dose groups was observed compared to the control group. D-E: Body weight loss over time and % loss at endpoint day 4 in different treatments groups. No significant effect of treatment on body weight loss was observed compared to control group, with high variability between animals. B, F-I: Viral load in samples of upper and lower respiratory tract, analysed for viral genomic RNA copies by qPCR and infectious SARS-CoV-2 virus titration. B+F) lungs, G) bronchoalveolar lavage fluid (BALF), H) nasal turbinate, I) throat swabs day 1 and 2, J) correlation between infectious virus in throat and day 4 lung. LLOD of the assay is dependent of the weight of the tissue sample, indicated by dashed lines. Volumes of BALF were 1 mL per animal. TCID50=50% tissue culture infectious dose. Statistical analysis was performed using non-parametric Mann Whitney U-test. **** P<0.0001; ***P<0.001; ** P<0.01; * P<0.05.

FIG. 38. Pharmacokinetic profile in Syrian hamsters.

Serum exposure over time of VHH72_h1(E1D, S56A)_10GS_Fc hIgG1 LALA (D72-53, PB9683) following a single dose of 5 mg/kg by intraperitoneal (IP) and intravenous (IV) administration in healthy male hamsters (body weight range 90-108 g). Twelve animals were used per group, with each animal sampled for 3 timepoints (n=4 per timepoint). Sample bioanalysis was done in competition AlphaLISA (dynamic range 1.2-142.5 μg/mL).

FIG. 39. VHH72_S56A and humanized VHH72_S56A sequences with CDR annotations.

Amino acid Numbering according to Kabat. CDR annotations according to MacCallum, AbM, Chothia, Kabat and IMGT in grey labelled boxes corresponding to the sequences of VHH72_S56A (SEQ ID NO:4) and VHH72_h1(E1D, S56A) (SEQ ID NO:6). Humanisation substitutions in the FRs in bold; CDR substitution S56A in red bold.

FIG. 40. Therapeutic and prophylactic treatment with D72-53 (PB9683) protects hamsters against SARS-CoV-2 infection.

A: Infectious SARS-CoV-2 particles in lung of Syrian following prophylactic (day −1 p.i.) or therapeutic (day 1 p.i.) IP treatment with D72-53(batch PB9683) (7 or 2 mg/kg) or the control Ab Synagis (7 mg/kg). B: Genomic SARS-CoV-2 RNA copies in lungs of Syrian hamsters with D72-53 (PB9683), or the control Ab. C: Histopathology analysis of lungs of hamsters, showing cumulative lung damage score. Statistical analysis was performed using non-parametric Mann Whitney U-test.: **** P<0.0001; ***P<0.001; ** P<0.01; * P<0.05. Dotted line represents lower limit of detection (LOD). Outliers are indicated by different symbols. One animal in the prophylactic 7 mg/kg group did not have detectable levels of drug in sera, suggesting it was not exposed to drug.

FIG. 41. Therapeutic and prophylactic treatment with D72-53 (PB9683) protects hamsters against SARS-CoV-2 infection.

Left: Genomic SARS-CoV-2 RNA copies in lungs of Syrian hamsters intraperitoneally administered with D72-53 (PB9683), D72-58 (VHH72_h1_E1D-(G4S)2-hIgG1hinge_EPKSCdel-hIgG1Fc_LALA_K447del)) or the control Ab Synagis. Right: Infectious SARS-CoV-2 particles in lung of Syrian following therapeutic IP treatment at 4 mg/kg with D72-53 (PB9683), D72-58, or the control Ab (Synagis). Statistical analysis was performed using non-parametric Mann Whitney U-test. * P<0.01; * P<0.05. Dotted line represents lower limit of detection (LOD). TCID50=50% tissue culture infectious dose. Outliers are indicated by different symbols.

FIG. 42. VHH72_S56A-Fc binds to the RBD of a diverse range of Sarbecoviruses.

The construct D72-53 (VHH72_h1_E1D_S56A-(G₄S)₂-hIgG1hinge_EPKSCdel-hIgG1Fc_LALA_K477del) was used herein. a. Cladogram (UPGMA method) based on the RBD of SARS-CoV-1-related, SARS-CoV-2-related and clade 2 and clade 3 Bat SARS-related sarbecoviruses. The colored boxes indicate the RBD variants that are bound by D72-53 as determined by flow cytometry of either yeast cells that display the indicated RBD variants, or HEK293T cells that express SARS-CoV-1 spike proteins in which the RBD is substituted by the indicated RBD variants. The grey boxes indicate the RBD variants for which no binding of D72-53 could be observed. b. Analysis of the binding of VHH72_S56A-Fc (D72-53), S309, CB6 and Synagis antibodies to Saccharomyces cerevisiae cells that display the RBD of the indicated Sarbecoviruses. The graphs show the MFI of AF633 conjugated anti-human IgG that was used to detect the binding of dilution series of the tested antibodies to S. cerevisiae cells that express the RBD derived from the indicated Sarbecoviruses. c. Amino acid sequence alignment of the tested RBD variants. Amino acid residues that deviate from the SARS-CoV-2 RBD are shown in bold. The amino acid residues that make part of the VHH72 epitope are indicated in colors according to their binding energy as calculated by Molecular Dynamics followed by FastContact (7) analysis.

FIG. 43. The epitope of VHH72 is highly conserved in circulating SARS-CoV-2 viruses.

Mutations in SARS-CoV-2 RBD, their impact on VHH72 binding and RBD fold. The upper part of the plot depicts all missense mutations detected at least once across the RBD sequence (spike protein amino acid positions 330-518 of SEQ ID NO:23) in 240,239 SARS-CoV-2 genomes analyzed (analysis on Jan. 4 2021). Minor variants are ordered vertically, according to their frequency, represented by letter size and the number of observed cases. Letter color corresponds to an estimated impact of a given mutation on VHH72 binding in Δ kcal/mol. Red and blue case number highlights significantly enhanced or decreased VHH72 binding (p-value≤0.05), respectively. The lower part of the plot shows: i) epitopes of VHH72 (by PISA buried surface estimation⁷⁴), colored according to epitope's similarity to VHH72 (Jaccard score), ii) ACE2 binding site, iii) individual contributions of RBD residues to VHH72 binding in kcal/mol, iv) RBD residues with statistically relevant binding energy contribution (95% confidence based on 30 simulations).

FIG. 44. Surface representation of SARS-CoV-2 RBD with the FastContact binding energy color-indicated epitope of VHH72.

The locations of observed variant residues N439K, S477N, E484K and N501Y are indicated in magenta.

FIG. 45: Alignment of VHH amino acid sequences.

Top 5 sequences were identified as non-competing VHHs of VHH72 for binding to RBD. VHH72 and the remaining sequences aligned include VHH family member representatives showing full competition with VHH72 in binding the SARS-CoV-2 RBD and all have the capability of blocking ACE2 binding to the RBD. CDRs annotated according to Kabat are indicated. The 56 position, Ser in VHH72 and VHH50, and G in 3^{rd} generation VHH72-family members is underlined in VHH72. Boxed VHHs belong to the same family, as defined by the CDR3 sequence.

FIG. 46. Dose-dependent inhibition of VHH72 binding to SARS-CoV-2 RBD by VHHs from different families.

Competition Alphascreen with avi-tagged biotinylated SARS-CoV-2 RBD (0.5 nM final) and Flag-tagged VHH72 h1 S56A (0.6 nM). VHHs belonging to the same (super) family are indicated in boxes. VHH Numbering: VHH50 FIG. 47. Dose-dependent inhibition of ACE-2 binding to SARS-CoV-2 RBD by VHHs from different families.

Competition Alphascreen with avi-tagged biotinylated SARS-CoV-2 RBD (1 nM final) and human ACE-2-mFc (0.2 nM). VHHs belonging to the same (super) family are indicated in boxes.

FIG. 48. VHH2.50 is able to neutralize SARS-CoV-1 and -2 pseudotyped VSV viruses.

A and B. SARS-CoV-2 and -1 Spike pseudotyped VSV-dG were incubated with 20 μg/ml of the indicated VHHs for 30 minutes at RT and subsequently used to infect Vero E6 cells. Twenty hours after infection the cells were lysed and used for analysis of luciferase activity. Graphs A and B show the luciferase activity for each VHH tested for neutralizing activity against respectively SARS-CoV-2 and -1 pseudotyped VSV (n=4 for SARS-CoV-2, n=1 for SARS-CoV-1). C. SARS-CoV-2 Spike pseudotyped VSV-dG was incubated with dilution series of CoV-2_VHH50 (=VHH2.50, SEQ ID NO:92) and VHH72 for 30 minutes at RT and subsequently used to infect Vero E6 cells. Twenty hours after infection GFP expressed by infected cells was measured using a Tecan infinite 200 PRO plate reader.

FIG. 49. Identification of the VHHs present in the PE extracts can potently neutralize SARS-CoV-2 Spike pseudotyped VSV-dG.

SARS-CoV-2 Spike pseudotyped VSV-dG were incubated with 16-, 80-, or 400-fold diluted PE extracts for 30 minutes at RT and subsequently used to infect Vero E6 cells. Twenty hours after infection the cells were lysed and used for analysis of luciferase activity. The luciferase activity measured for the 16, 80- and 400-fold diluted PE samples grouped per VHH family is shown. Each VHH family is indicated by a F-number for one of its representative VHHs (F55 represents VHH3.55 family; F36: VHH3.36 family; F38: VHH3.38 family; F121: VHH3.121 family; F29: VHH3.29 family; F72sim: 3th generation VHHs classified in VHH72 family; F83: VHH3.83 family; F149: VHH3.149 family); PE_2_VHH50, periplasmic extract of VHH2.50.

FIG. 50. VHH72-12GS-Fc binding to SARS-CoV-2 mutant variants.

a. Composite overlay showing the locations of VHH72 (grey cartoon with transparent surface, centre-left) and ACE-2 (orange cartoon, top) versus SARS-CoV-2RBD (cyan cartoon, centre). Tyr369 of SARS-CoV-2 RBD is indicated and shown as purple sticks. The ACE-2 glycan sugars at N322 (clashing with VHH72) are shown as orange sticks; RBD glycan sugars at N343 are shown as cyan sticks. The emerging RBD variants at residues K417(->N), L452(->R), S477(->N), E484(->K) and N501(->Y) are indicated and shown as yellow sticks. Of these, only the backbone carbonyl of N501 is peripheral to VHH72. b. Binding of VHH72-12GS-Fc and mAb CB6 to SARS-CoV-1 spike with the RBD replaced by WT, N439K or N501Y RBD of SARS-CoV-2, expressed on the surface of 293Tcells. Data point represent the ratio of the mean fluorescence intensity (MFI) of untransfected GFP-negative cells over the transfected GFP-positive cells, as determined by flowcytometry.

FIG. 51. PK/PD in hamster challenge studies.

A-B, Correlation of day 4 serum concentrations of IP treated hamsters to the lung infectious viral load (TCID50) combined from hamster challenge studies at two different centres with two different SARS-CoV-2 isolates. Compounds: VHH72 h1 S46A-Fc fusions (bivalent D72-23, D72-52(PB9690), D72-53(PB9683), and tetravalent D72-55/PB9589). Limits of quantification are indicated by dotted lines. The median response of the control animals is indicated with striped line. C, Correlation between day 4 BALF and serum concentrations in hamsters challenged with SARS-CoV-2 Munich isolate treated therapeutically 4 h post infection. Regression: R2 0.6128, P<000.1 for combined bivalent and tetravalent formats.

FIG. 52. SDS PAGE analysis of the purified VHHs.

SDS-PAGE and Coomassie staining of the indicated purified VHHs produced by Pichia pastoris (top panel) or WK6 E. coli cells (bottom panel). Note the higher molecular weight band for VHH3.47 representing glycosylated protein.

FIG. 53. Binding of VHHs to the SARS-CoV-2 RBD, and Spike proteins of SARS-CoV-1 and SARS-CoV-2 by ELISA and BLI.

Binding of VHHs to the RBD of SARS-CoV-2 (B), the spike of SARS-CoV-2 (C), the spike of SARS-CoV-1 (D) and the negative control antigen, BSA (A). VHH72 was used as control. (E) Affinity measurements of VHHs at a single concentration (200 nM) to monomeric human Fc-fused SARS-CoV-2_RBD-SD1 captured by anti-human IgG Fc capture (AHC) biosensors (FortéBio). The graph shows the representative data of 1 of the duplicate measurements. VHH72_h1_S56A (labeled VHH72, this is VHH72 with an S56A substitution with increased affinity for SARS-CoV1 and -2 RBD) was used as reference. (F) Binding kinetics of VHH3.17, VHH3.77 and VHH3.115 to monomeric human Fc-fused SARS-CoV-2_RBD-SD1 captured by anti-human IgG Fc capture (AHC) biosensors (FortéBio).

FIG. 54. Binding of VHHs to the RBD of a diverse range of Sarbecoviruses.

(A) Cladogram (UPGMA method) based on the RBD of SARS-CoV-1-related, SARS-CoV-2-related and clade 2 and clade 3 Bat SARS-related Sarbecoviruses. (B) Flowcytometric analysis of the binding of VHHs to Saccharomyces cerevisiae cells that display the RBD of the indicated Sarbecoviruses. The graphs show for the tested RBD variants the ratio of the MFI of AF647 conjugated anti-mouse IgG antibody used to detect VHHs bound to the cells that express RBD (FITC conjugated anti-myc tag antibody positive) over that of cells that do not express RBD (FITC conjugated anti-myc tag antibody negative). The GFP binding VHH (GBP) was used as a negative control antibody and VHH72_h1_S56A (VHH72) was used as reference. All VHHs except VHH3.83 were tested at 10 μg/ml. VHH3.83 was tested at 100 μg/ml.

FIG. 55. Binding of VHH3.38 and VHH3.83 to the RBD of a diverse range of Sarbecoviruses.

Flowcytometric analysis of the binding of VHH3.38 and VHH3.83 to the indicated RBDs at 100, 1 and 0.01 μg/ml. PBS was used as negative control and VHH72_h1_S56A (VHH72) was used as reference. The graphs show for the indicated RBD variants the ratio of the MFI of AF647 conjugated anti-mouse IgG antibody used to detect VHHs bound the cells that express RBD (FITC conjugated anti-myc tag antibody positive) over that of cells that do not express RBD (FITC conjugated anti-myc tag antibody negative).

FIG. 56. Visualization of the conserved surface patches on the RBD among clade 1, 2 and 3 Sarbecoviruses.

(A) Surface representation of the SARS-CoV-2 RBD with the VHH72 epitope indicated according to the display color scheme that indicates the binding energy (kcal/mol) of the interaction between VHH72 and the respective RBD residues. The binding energy of each amino acid of the VHH72 footprint on the SARS-CoV-2 RBD was calculated by FastContact and molecular dynamics based on the crystal structure of the VHH72/SARS-CoV-1 complex^{10, 14}. (B) Surface representation of the conserved surface patches on the RBD of Sarbecoviruses. The RBD protein conservation of the Sarbecoviruses tested FIG. 55. Was visualized using Scop3D (Vermeire et al, 2015 Proteomics, 15(8):1448-52) and PyMol (DeLano, 2002). Red to blue represents highly to not conserved.

FIG. 57. the selected VHHs compete with VHH72 for binding to the SARS-CoV-2 RBD.

(A) The selected VHHs can bind to monomeric SARS-CoV-2 RBD captured by the S309 antibody but fail to bind SARS-CoV-2 RBD captured by VHH72-Fc. The graph shows the average (n=2+variation) binding (OD405) of the selected VHHs and two additional RBD specific VHHs (non-competing VHH1 and 2) and an irrelevant GFP binding VHH (GBP) at 0.5 ug/ml to RBD that was captured by either coated VHH72-Fc or coated S309. VHH72_h1_S56A (VHH72) at 10 ug/ml was included as reference. (B) Surface representation of the SARS-CoV-2 RBD (white surface) bound by VHH72 and the S309 antibody (Pinto et al. 2020, Nature, 583), both shown in black cartoon representation. (C) Schematic set-up of the BLI competition experiment. VHH72-Fc was loaded on anti-human Fc biosensor tip and subsequently dipped into a solution containing SARS-CoV-1-muFc (Sino Biological) until saturation was achieved. Next, the tips were dipped into a solution containing the VHHs that are under investigation. These VHHs will either bind or bind not to VHH72-Fc captured RBD and will respectively increase or not increase the BLI-signal over time. In contrast, VHHs that compete with VHH72 for the binding of RBD might displace the captured RBD-muFc from the VHH72-Fc coated tips and will hence lower the BLI signal over time. (D) The selected VHHs displace the RBD-muFc form the VHH72-Fc coated tips. As controls buffer, VHH72_h1_S56A (VHH72) were used. The graphs show the BLI signal overtime starting from the moment the tips were dipped in the solution containing the VHHs that are under investigation.

FIG. 58. K378N substitution in the SARS-CoV-2 RBD severely affects the binding of VHH3.38 and VHH3.83.

Dilutions series of VHH3.8 (A) and VH3.83 (B) were used to stain HEK293 cells transfected with a GFP expression vector in combination with a non-coding expression vector (GFP) or an expression vector for the SARS-CoV-1 spike in which the RBD was replaced by the either WT 5ARS-CoV-2 RBD (WT) or the SARS-CoV-2 RBD in which K378 was replaced by N (K378N). Bound VHHs were detected with a mouse anti-HIS-tag antibody and a AF647 conjugated anti-mouse IgG antibody. The graphs show the ratio of AF647 MFI of transfected (GFP⁺) cells over that of non-transfected cells (GFP⁻).

FIG. 59. The selected VHHs can potently neutralize VSV-delG pseudotyped with the SARS-CoV-2 spike protein.

(A) Neutralization of SARS-CoV-2 pseudotyped VSV by VHHs produced by P. pastoris. VHH72_h1_S56A (VHH72) was included as a reference. The graphs show the GFP fluorescence intensity of triplicate dilutions series (n=3±SEM), each normalized to the lowest and highest GFP fluorescence intensity value of that dilution series. (B) Neutralization of SARS-CoV-2 pseudotyped VSV by VHHs3.83 and VHH3.E4 produced by E. coli. The graphs show the GFP fluorescence (n=1) normalized to the lowest and highest GFP FI value of each dilution series.

FIG. 60. The selected VHHs can potently neutralize VSV-delG pseudotyped with the SARS-CoV-1 spike protein.

Neutralization of SARS-CoV-1 spike pseudotyped VSV by VHHs produced by P. pastoris. The irrelevant GFP binding VHH (GBP) and non-infected cells (NI) were included as controls and VHH72_h1_S56A (VHH72) was included as a reference. The graphs show the mean (n=2 t variation) GFP fluorescence intensity.

FIG. 61. The selected VHHs prevent binding of RBD to VeroE6 target cells expressing the ACE2 spike receptor.

The graph shows the binding of RBD-muFc (Sino Biological) that was pre-incubated with the indicated VHHs to VeroE6 cells (these cells express an ACE2 receptor that can be recognized by SARS-CoV-2 spike, RBD and viruses) as detected by an AF647 conjugated anti-mouse IgG antibody via flowcytometry. As controls VeroE6 cells not treated with RBD (noRBD) and VeroE6 cells stained with RBD-muFc that was pre-incubated with PBS or an irrelevant control VHH (GBP) were used. VHH72_h1_S56A was used as reference next to 2 VHHs that do not compete with VHH72 for RBD binding (non VHH72-competing VHHs) The bars represent one single analysis per VHH. The controls, PBS and noRBD were tested in duplicate.

FIG. 62. Sorting of yeast cells from the RBD-variant-yeast-display library that exhibit diminished binding by VHH72, VHH3.38, VHH3.83 and VHH3.55.

(A) Flowcytometric analysis of the binding of VHH72_h1_S56A (upper graph) and VHH3.38, VHH3.55 and VHH3.83 (lower graph) to yeast cells expressing myc-tagged WT SARS-CoV-2 RBD at their surface. The graphs show for each indicated concentration of the tested VHHs, the ratio of MFI of the AF594 conjugated antibody that was used to detect VHH binding on RBD⁺ (myc-tag⁺) cells over that of the RBD-(myc-tag) yeast cells. The dotted line indicates the concentration of the VHHs that was selected for the scanning of the RDB yeast-display libraries. (B) Sorting of the RBD yeast-display libraries for yeast cells that present with diminished binding by VHH72_h1_S56A, VHH3.83, VHH3.38 and VHH3.55. The dot plots show the binding of the indicated VHHs and anti-myc tag antibody to one of the 2 libraries of the RBD-variants-displaying yeast cells. For each VHH the percentage of yeast cells that display diminished VHH binding and fall into the “escape” gate for sorting and subsequent deep sequence analysis is indicated in the plots.

FIG. 63. Outlining of the epitopes of VHH72, VHH3.38, VHH3.83 and VHH3.55 based on the deep mutational scanning.

(A) Indication of the RBD amino acid positions that significantly affect the binding of VHH72_h1_S56A (VHH72), VHH3.38, VHH3.83 and VHH3.55 as identified by deep mutational scanning. The SARS-CoV-2 RBD amino acid sequence is shown. In the upper line (SARS-CoV-2 RBD) the amino acids involved in the binding of VHH72 as determined by FastContact and molecular dynamics based on the crystal structure of the VHH72 in complex with the SARS-CoV-1 are indicated following the color code depicted in panel C. In the second line (SARS-CoV-2 RBD) the RBD amino acids that define the VHH72 footprint are indicated in bold. In the third (Escape VHH72), fourth (Escape VHH3.83), fifth (Escape VHH3.55) and sixth (Escape VHH3.38) line the VHH72 footprint is indicated in bold and the amino acid positions involved in the binding of the respective VHHs as identified by the deep mutational scanning are indicated in underlined bold. (B) The profile of the RBD amino acid positions involved in the binding of VHH72_h1_S56A, VHH3.38, VHH3.55 and VHH3.83 as determined by deep mutational scanning (black lines) overlaps among the VHHs and with the VHH72 epitope on the SARS-CoV-2 RBD based on FastContact and modeling (orange bars). (C) A schematic representation of the color code that indicates the binding energy (kcal/mol) calculated for each amino acid of the VHH72 footprint on the SARS-CoV-2 RBD by FastContact and molecular dynamics based on the crystal structure of the VHH72/SARS-CoV-1 complex^{10, 14}. (D) RBD Surface representation of the VHH72 epitope (according to the color code in panel (C), VHH72 footprint (blue) and the RBD amino acids (in red) involved in the binding of the indicated VHHs as identified by deep mutational scanning.

FIG. 64. Representation of the amino acids involved in the binding of VHH72-h1_S56A, VHH3.38, VHH3.83 and VHH3.55 as identified by deep mutational scanning that locate outside the VHH72 footprint.

(A) Indication of the RBD amino acid positions that significantly affect the binding of VHH72_h1_S56A (VHH72), VHH3.38, VHH3.83 and VHH3.55 as identified by deep mutational scanning but locate outside the VHH72 footprint. The displayed sequence represents the RBD amino acid sequence. In the upper line (SARS-CoV-2 RBD) the amino acids involved in the binding of VHH72 as determined by FastContact and molecular dynamics based on the crystal structure of the VHH72 in complex with the SARS-CoV-1 are indicated following the color code depicted in panel C of FIG. 63. In the second line (SARS-CoV-2 RBD) the RBD amino acids that form the VHH72 footprint are indicated in bold. In the third (Escape VHH72), fourth (Escape VHH3.83), fifth (Escape VHH3.55) and sixth (Escape VHH3.38) line the VHH72 footprint is indicated in bold. The amino acid positions involved in the binding of the respective VHHs as identified by the deep mutational scanning and locate in or outside the VHH72 footprint are respectively indicated in underlined bold and underlined italic. (B) RBD Surface and cartoon representations of the RBD with the VHH72 footprint indicated in blue. The RBD amino acid positions involved in the binding of VHH72_h1_S56A as identified by deep mutational scanning that locate within or outside the VHH72 footprint are respectively indicated in red and green. A cartoon representation of VHH72 bound to the RBD is shown in orange. (C) RBD Surface and cartoon representations of the RBD with the VHH72 footprint indicated in blue. The amino acid positions involved in the binding of VHH3.38 epitope as identified by deep mutational scanning that fall in or outside the VHH72 footprint are respectively indicated in red and green. The RBD amino acid C361 that forms a disulfide bond with C336 is indicated in orange. (D) RBD Surface and cartoon representations of the RBD with the VHH72 footprint indicated in blue. The amino acid positions involved in the binding of VHH3.55 epitope as identified by deep mutational scanning that fall in or outside the VHH72 footprint are respectively indicated in red and green. The RBD amino acid C525 that forms a disulfide bond with C391 is indicated in orange.

FIG. 65. Structural studies of the SC2-VHH3.38 complex. (A, B) Electron potential map (grey mesh) and build in structural model (cartoon representation) of the 3D cryoEM reconstruction of the SC2-VHH3.38 complex shown in side (A) or top (B) view. The reconstruction shows density for the SC2 trimer (blue, cyan and violet for the three protomers) as well as three copies of the VHH3.38 (yellow; labeled 3.38). The SC2 receptor binding domain, N-terminal domain and stem region are labelled RBD, NTD and S2, respectively. (C) Close-up view of the VHH3.38 binding site in the SC2-VHH3.38 complex (cryoEM electron potential map shown as grey mesh). The nanobody binds the SC2 RBD, covering a binding surface comprising the binding epitope subject of claim 1 (residues S368, Y369, S371, S375, T376, F377, K378, C379 and Y508; shown in green and in stick representation). (D) Top view of the SC2-VHH3.38 complex, colored as in panel A, with the SC2 trimer shown as molecular surface and the VHH3.38 molecules as secondary structure cartoon. Shown in green is the binding epitope of claim 1. The picture shows the 3-RBD up orientation of the SC2-VHH3.38 complex. (E, F) Close-up views of the SC2 RBD (shown as a molecular surface) in complex with VHH3.38 (yellow, cartoon representation). Shown in green (panel E) or red (panel F) are, respectively, the residues that comprise the VHH binding epitope as defined in claim 3, and the residues identified in the deep mutational scanning experiment as site for mutants that escape VHH3.38 binding.

FIG. 66. Comparison of SC2 conformational states and the SC2-VHH3.38 complex.

(A) Shown from left to right are the molecular surfaces of 3D structures of the SC2 spike trimer in closed or “3-RBD down” conformation (PDB: 6ZGI), the open or “1 RBD-up” conformation (PDB: 6ZGG) and the SC2-VHH3.38 complex (this application), which shows the RBD domains in a fully open, 3-RBD up confirmation. N-terminal domain, receptor-binding domain and stem region are colored cyan, blue and orange respectively. VHH3.38 is shown in red, as secondary structure cartoon. (B) Side (bottom) and close-up view (top) of the SC2-VHH3.38 complex and the superimposition with the structure of the SARS-CoV-2 RBD in complex with human Ace2 (PDB: 7dmu). RBD, VHH3.38 and Ace2 are colored blue, red and cyan respectively.

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein. The invention together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, N.Y. (2012); and Ausubel et al., Current Protocols in Molecular Biology (Supplement 114), John Wiley & Sons, New York (2016), for definitions and terms of the art. Unless defined otherwise, all technical and scientific terms used herein have the same meaning as commonly understood by one of ordinary skill in the art (e.g. in molecular biology, biochemistry, structural biology, and/or computational biology).

‘Nucleotide sequence’, “DNA sequence” or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, and RNA. It also includes known types of modifications, for example, methylation, “caps” substitution of one or more of the naturally occurring nucleotides with an analog. By “nucleic acid construct” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes comprising non-native nucleic acid sequences, and the like. “Coding sequence” is a nucleotide sequence, which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances. With a “chimeric gene” or “chimeric construct” or “chimeric gene construct” is meant a recombinant nucleic acid sequence in which a promoter or regulatory nucleic acid sequence is operatively linked to, or associated with, a nucleic acid sequence that codes for an mRNA, such that the regulatory nucleic acid sequence is able to regulate transcription or expression of the associated nucleic acid coding sequence. The regulatory nucleic acid sequence of the chimeric gene is not operatively linked to the associated nucleic acid sequence as found in nature. An “expression cassette” comprises any nucleic acid construct capable of directing the expression of a gene/coding sequence of interest, which is operably linked to a promoter of the expression cassette. Expression cassettes are generally DNA constructs preferably including (5′ to 3′ in the direction of transcription): a promoter region, a polynucleotide sequence, homologue, variant or fragment thereof operably linked with the transcription initiation region, and a termination sequence including a stop signal for RNA polymerase and a polyadenylation signal. It is understood that all of these regions should be capable of operating in biological cells, such as prokaryotic or eukaryotic cells, to be transformed. The promoter region comprising the transcription initiation region, which preferably includes the RNA polymerase binding site, and the polyadenylation signal may be native to the biological cell to be transformed or may be derived from an alternative source, where the region is functional in the biological cell. Such cassettes can be constructed into a “vector”.

The terms “protein”, “polypeptide”, and “peptide” are interchangeably used further herein to refer to a polymer of amino acid residues and to variants and synthetic analogues of the same. A “peptide” may also be referred to as a partial amino acid sequence derived from its original protein, for instance after tryptic digestion. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. This term also includes posttranslational modifications of the polypeptide, such as glycosylation, phosphorylation and acetylation. Based on the amino acid sequence and the modifications, the atomic or molecular mass or weight of a polypeptide is expressed in (kilo)dalton (kDa). A “protein domain” is a distinct functional and/or structural unit in a protein. Usually a protein domain is responsible for a particular function or interaction, contributing to the overall role of a protein. Domains may exist in a variety of biological contexts, where similar domains can be found in proteins with different functions. By “isolated” or “purified” is meant material that is substantially or essentially free from components that normally accompany it in its native state. For example, an “isolated polypeptide” or “purified polypeptide” refers to a polypeptide which has been purified from the molecules which flank it in a naturally-occurring state, e.g., an antibody or nanobody as identified and disclosed herein which has been removed from the molecules present in the a sample or mixture, such as a production host, that are adjacent to said polypeptide. An isolated protein or peptide can be generated by amino acid chemical synthesis or can be generated by recombinant production or by purification from a complex sample.

The term “fused to”, as used herein, and interchangeably used herein as “connected to”, “conjugated to”, “ligated to” refers, in particular, to “genetic fusion”, e.g., by recombinant DNA technology, as well as to “chemical and/or enzymatic conjugation” resulting in a stable covalent link. The same applies for the term “inserted in”, wherein one nucleic acid or protein sequence part may be inserted in another sequence by fusing the two sequences genetically, enzymatically or chemically.

“Homologue”, “Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met, also indicated in one-letter code herein) occurs in both sequences to yield the number of matched positions, dividing the number of matched positions by the total number of positions in the window of comparison (i.e., the window size), and multiplying the result by 100 to yield the percentage of sequence identity. A “substitution”, or “mutation”, or “variant” as used herein, results from the replacement of one or more amino acids or nucleotides by different amino acids or nucleotides, respectively as compared to an amino acid sequence or nucleotide sequence of a parental protein or a fragment thereof. it is understood that a protein or a fragment thereof may have conservative amino acid substitutions which have substantially no effect on the protein's activity.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant”, “engineered” or “variant” refers to a gene or gene product that displays modifications in sequence, post-translational modifications and/or functional properties (i.e., altered characteristics) when compared to the wild-type gene or gene product. It is noted that naturally occurring mutants can be isolated; these are identified by the fact that they have altered characteristics when compared to the wild-type gene or gene product.

The term “molecular complex” or “complex” refers to a molecule associated with at least one other molecule, which may be a chemical entity. The term “associating with” refers to a condition of proximity between a chemical entity or compound, or portions thereof, and a binding pocket or binding site on a protein. The association maybe non-covalent—wherein the juxtaposition is energetically favored by hydrogen bonding or van der Waals or electrostatic interactions—or it may be covalent. The term “chemical entity” refers to chemical compounds, complexes of at least two chemical compounds, and fragments of such compounds or complexes. The chemical entity may be, for example, a ligand, a substrate, a phosphate, a nucleotide, an agonist, antagonist, inhibitor, antibody, a single domain antibody, drug, peptide, peptidomimetic, protein or compound.

As used herein, the term “crystal” means a structure (such as a three-dimensional (3D) solid aggregate) in which the plane faces intersect at definite angles and in which there is a regular structure (such as an internal structure) of the constituent chemical species. The term “crystal” refers in particular to a solid physical crystal form such as an experimentally prepared crystal. The term “co-crystal” as used herein refers to a structure that consist of two or more components that form a unique crystalline structure having unique properties, wherein the components may be atoms, ions or molecules. In the context of current application, a co-crystal comprising the RBD domain of a Corona virus S protein and the herein described Nanobody (VHH-72) is equivalent to a crystal of the RBD domain in complex with the herein described Nanobody. The term “crystallization solution” refers to a solution which promotes crystallization comprising at least one agent including a buffer, one or more salts, a precipitating agent, one or more detergents, sugars or organic compounds, lanthanide ions, a poly-ionic compound, and/or stabilizer.

The terms “suitable conditions” refers to the environmental factors, such as temperature, movement, other components, and/or “buffer condition(s)” among others, wherein “buffer conditions” refer specifically to the composition of the solution in which the molecules are present. A composition includes buffered solutions and/or solutes such as pH buffering substances, water, saline, physiological salt solutions, glycerol, preservatives, etc. for which a person skilled in the art is aware of the suitability to obtain optimal assay performance. Suitable conditions as used herein could also refer to suitable binding conditions, for instance when Nbs are aimed to bind a RBD. Suitable conditions as used herein could also refer to suitable crystallization or cryo-EM conditions, which may alternatively mean suitable conditions wherein the aimed structural analysis is expected. Suitable conditions may further relate to buffer conditions in which thermal stability assays can be performed.

The term “binding pocket” or “binding site” refers to a region of a molecule or molecular complex, that, as a result of its shape and charge, favourably associates with another chemical entity, compound, proteins, peptide, antibody or Nb. For antibody-related molecules, the term “epitope” or “conformational epitope” is also used interchangeably herein. The term “pocket” includes, but is not limited to cleft, channel or site. The RBD domain of a Corona virus herein described comprises a binding pocket or binding site which include, but is not limited to a Nanobody binding site. The term “part of a binding pocket/site” refers to less than all of the amino acid residues that define the binding pocket, binding site or epitope. For example, the atomic coordinates of residues that constitute part of a binding pocket may be specific for defining the chemical environment of the binding pocket, or useful in designing fragments of an inhibitor that may interact with those residues. For example, the portion of residues may be key residues that play a role in ligand binding, or may be residues that are spatially related and define a three-dimensional compartment of the binding pocket. The residues may be contiguous or non-contiguous in primary sequence.

“Binding” means any interaction, be it direct or indirect. A direct interaction implies a contact between the binding partners. An indirect interaction means any interaction whereby the interaction partners interact in a complex of more than two molecules. The interaction can be completely indirect, with the help of one or more bridging molecules, or partly indirect, where there is still a direct contact between the partners, which is stabilized by the additional interaction of one or more molecules. By the term “specifically binds,” as used herein is meant a binding domain which recognizes a specific target, but does not substantially recognize or bind other molecules in a sample. Specific binding does not mean exclusive binding. However, specific binding does mean that proteins have a certain increased affinity or preference for one or a few of their binders. The term “affinity”, as used herein, generally refers to the degree to which a ligand, chemical, protein or peptide binds to another (target) protein or peptide so as to shift the equilibrium of single protein monomers toward the presence of a complex formed by their binding. A “binding agent” relates to a molecule that is capable of binding to another molecules, wherein said binding is preferably a specific binding, recognizing a defined binding site, pocket or epitope. The binding agent may be of any nature or type and is not dependent on its origin. The binding agent may be chemically synthesized, naturally occurring, recombinantly produced (and purified), as well as designed and synthetically produced. Said binding agent may hence be a small molecule, a chemical, a peptide, a polypeptide, an antibody, or any derivatives thereof, such as a peptidomimetic, an antibody mimetic, an active fragment, a chemical derivative, among others.

The RBD domain of a Corona virus herein described comprises a binding pocket or binding site which include, but is not limited to a Nanobody binding site. The term “part of a binding pocket/site” refers to less than all of the amino acid residues that define the binding pocket, binding site or epitope. For example, the atomic coordinates of residues that constitute part of a binding pocket may be specific for defining the chemical environment of the binding pocket, or useful in designing fragments of an inhibitor that may interact with those residues. For example, the portion of residues may be key residues that play a role in ligand binding, or may be residues that are spatially related and define a three-dimensional compartment of the binding pocket. The residues may be contiguous or non-contiguous in primary sequence.

An “epitope”, as used herein, refers to an antigenic determinant of a polypeptide, constituting a binding site or binding pocket on a target molecule, such as Corona virus RBD domain, more particularly 2019-nCoV RBD domain. An epitope could comprise 3 amino acids in a spatial conformation, which is unique to the epitope. Generally, an epitope consists of at least 4, 5, 6, 7 such amino acids, and more usually, consists of at least 8, 9, 10 such amino acids. Methods of determining the spatial conformation of amino acids are known in the art, and include, for example, X-ray crystallography and multi-dimensional nuclear magnetic resonance. A “conformational epitope”, as used herein, refers to an epitope comprising amino acids in a spatial conformation that is unique to a folded 3-dimensional conformation of a polypeptide. Generally, a conformational epitope consists of amino acids that are discontinuous in the linear sequence but that come together in the folded structure of the protein. However, a conformational epitope may also consist of a linear sequence of amino acids that adopts a conformation that is unique to a folded 3-dimensional conformation of the polypeptide (and not present in a denatured state). In protein complexes, conformational epitopes consist of amino acids that are discontinuous in the linear sequences of one or more polypeptides that come together upon folding of the different folded polypeptides and their association in a unique quaternary structure. Similarly, conformational epitopes may here also consist of a linear sequence of amino acids of one or more polypeptides that come together and adopt a conformation that is unique to the quaternary structure. The term “conformation” or “conformational state” of a protein refers generally to the range of structures that a protein may adopt at any instant in time. One of skill in the art will recognize that determinants of conformation or conformational state include a protein's primary structure as reflected in a protein's amino acid sequence (including modified amino acids) and the environment surrounding the protein. The conformation or conformational state of a protein also relates to structural features such as protein secondary structures (e.g., α-helix, β-sheet, among others), tertiary structure (e.g., the three dimensional folding of a polypeptide chain), and quaternary structure (e.g., interactions of a polypeptide chain with other protein subunits). Posttranslational and other modifications to a polypeptide chain such as ligand binding, phosphorylation, sulfation, glycosylation, or attachments of hydrophobic groups, among others, can influence the conformation of a protein. Furthermore, environmental factors, such as pH, salt concentration, ionic strength, and osmolality of the surrounding solution, and interaction with other proteins and co-factors, among others, can affect protein conformation. The conformational state of a protein may be determined by either functional assay for activity or binding to another molecule or by means of physical methods such as X-ray crystallography, NMR, or spin labeling, among other methods. For a general discussion of protein conformation and conformational states, one is referred to Cantor and Schimmel, Biophysical Chemistry, Part I: The Conformation of Biological. Macromolecules, W.H. Freeman and Company, 1980, and Creighton, Proteins: Structures and Molecular Properties, W.H. Freeman and Company, 1993.

The term “antibody” refers to an immunoglobulin (Ig) molecule or a molecule comprising an immunoglobulin (Ig) domain, which specifically binds with an antigen. ‘Antibodies’ can further be intact immunoglobulins derived from natural sources or from recombinant sources and can be immunoreactive portions of intact immunoglobulins. The term “active antibody fragment” refers to a portion of any antibody or antibody-like structure that by itself has high affinity for an antigenic determinant, or epitope, and contains one or more CDRs accounting for such specificity. Non-limiting examples include immunoglobulin domains, Fab, F(ab)′2, scFv, heavy-light chain dimers, immunoglobulin single variable domains, Nanobodies (or VHH antibodies), domain antibodies, and single chain structures, such as a complete light chain or complete heavy chain.

The term “antibody fragment” and “active antibody fragment” as used herein refer to a protein comprising an immunoglobulin domain or an antigen binding domain capable of specifically binding a RBD present in the Spike protein of the SARS-CoV-2 virus. Antibodies are typically tetramers of immunoglobulin molecules. The term “immunoglobulin (Ig) domain”, or more specifically “immunoglobulin variable domain” (abbreviated as “IVD”) means an immunoglobulin domain essentially consisting of four “framework regions” which are referred to in the art and herein below as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and herein below as “complementarity determining region 1” or “CDR1”; as “complementarity determining region 2” or “CDR2”; and as “complementarity determining region 3” or “CDR3”, respectively. Thus, the general structure or sequence of an immunoglobulin variable domain can be indicated as follows: FR1-CDR1-FR2-CDR2-FR3-CDR3-FR4. It is the immunoglobulin variable domain(s) (IVDs) that confer specificity to an antibody for the antigen by carrying the antigen-binding site. Typically, in conventional immunoglobulins, a heavy chain variable domain (VH) and a light chain variable domain (VL) interact to form an antigen binding site. In this case, the complementarity determining regions (CDRs) of both VH and VL will contribute to the antigen binding site, i.e. a total of 6 CDRs will be involved in antigen binding site formation. In view of the above definition, the antigen-binding domain of a conventional 4-chain antibody (such as an IgG, IgM, IgA, IgD or IgE molecule; known in the art) or of a Fab fragment, a F(ab′)2 fragment, an Fv fragment such as a disulphide linked Fv or a scFv fragment, or a diabody (all known in the art) derived from such conventional 4-chain antibody, with binding to the respective epitope of an antigen by a pair of (associated) immunoglobulin domains such as light and heavy chain variable domains, i.e., by a VH-VL pair of immunoglobulin domains, which jointly bind to an epitope of the respective antigen. An immunoglobulin single variable domain (ISVD) as used herein, refers to a protein with an amino acid sequence comprising 4 Framework regions (FR) and 3 complementary determining regions (CDR) according to the format of FR1-CDR1-FR2-CDR2-FR3-CDR3-FR4. An “immunoglobulin domain” of this invention refers to “immunoglobulin single variable domains” (abbreviated as “ISVD”), equivalent to the term “single variable domains”, and defines molecules wherein the antigen binding site is present on, and formed by, a single immunoglobulin domain. This sets immunoglobulin single variable domains apart from “conventional” immunoglobulins or their fragments, wherein two immunoglobulin domains, in particular two variable domains, interact to form an antigen binding site. The binding site of an immunoglobulin single variable domain is formed by a single VH/VHH or VL domain. Hence, the antigen binding site of an immunoglobulin single variable domain is formed by no more than three CDR's. As such, the single variable domain may be a light chain variable domain sequence (e.g., a VL-sequence) or a suitable fragment thereof; or a heavy chain variable domain sequence (e.g., a VH-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit). In one embodiment of the invention, the immunoglobulin single variable domains are heavy chain variable domain sequences (e.g., a VH-sequence); more specifically, the immunoglobulin single variable domains can be heavy chain variable domain sequences that are derived from a conventional four-chain antibody or heavy chain variable domain sequences that are derived from a heavy chain antibody. For example, the immunoglobulin single variable domain may be a (single) domain antibody (or an amino acid sequence that is suitable for use as a (single) domain antibody), a “dAb” or dAb (or an amino acid sequence that is suitable for use as a dAb) or a Nanobody (as defined herein, and including but not limited to a VHH); other single variable domains, or any suitable fragment of any one thereof. In particular, the immunoglobulin single variable domain may be a Nanobody (as defined herein) or a suitable fragment thereof. Note: Nanobody®, Nanobodies® and Nanoclone® are registered trademarks of Ablynx N.V. (a Sanofi Company). For a general description of Nanobodies, reference is made to the further description below, as well as to the prior art cited herein, such as e.g. described in WO2008/020079. “VHH domains”, also known as VHHs, VHH domains, VHH antibody fragments, and VHH antibodies, have originally been described as the antigen binding immunoglobulin (Ig) (variable) domain of “heavy chain antibodies” (i.e., of “antibodies devoid of light chains”; Hamers-Casterman et al (1993) Nature 363: 446-448). The term “VHH domain” has been chosen to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHHs and Nanobody, reference is made to the review article by Muyldermans (Reviews in Molecular Biotechnology 74: 277-302, 2001), as well as to the following patent applications, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx N.V.; WO 01/90190 by the National Research Council of Canada; WO 03/025020 (=EP 1433793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V. As described in these references, Nanobody (in particular VHH sequences and partially humanized Nanobody) can in particular be characterized by the presence of one or more “Hallmark residues” in one or more of the framework sequences. For numbering of the amino acid residues of an IVD different numbering schemes can be applied. For example, numbering can be performed according to the AHo numbering scheme for all heavy (VH) and light chain variable domains (VL) given by Honegger, A. and Plückthun, A. (J. Mol. Biol. 309, 2001), as applied to VHH domains from camelids. Alternative methods for numbering the amino acid residues of VH domains, which can also be applied in an analogous manner to VHH domains, are known in the art. For example, the delineation of the FR and CDR sequences can be done by using the Kabat numbering system as applied to VHH domains from camelids in the article of Riechmann, L. and Muyldermans, S., 231(1-2), J Immunol Methods. 1999. It should be noted that—as is well known in the art for V_{H} domains and for VHH domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. The total number of amino acid residues in a VH domain and a VHH domain will usually be in the range of from 110 to 120, often between 112 and 115. It should however be noted that smaller and longer sequences may also be suitable for the purposes described herein. Determination of CDR regions may also be done according to different methods, such as the designation based on contact analysis and binding site topography as described in MacCallum et al., J. Mol. Biol. (1996) 262, 732-745. Or alternatively the annotation of CDRs may be done according to AbM (AbM is Oxford Molecular Ltd.'s antibody modelling package as described on http://www.bioinf.org.uk/abs/index.html), Chothia (Chothia and Lesk, 1987; Mol Biol. 196:901-17), Kabat (Kabat et al., 1991; 5^{th} edition, NIH publication 91-3242), and IMGT (LeFranc, 2014; Frontiers in Immunology. 5 (22): 1-22). These annotations differ slightly, but each intend to comprise the regions of the loops involved in binding the target.

VHHs or Nbs are often classified in different sequences families or even superfamilies, as to cluster the clonally related sequences derived from the same progenitor during B cell maturation (Deschaght et al. 2017. Front Immunol. 10; 8:420). This classification is often based on the CDR sequence of the Nbs, and wherein for instance each Nb family is defined as a cluster of (clonally) related sequences with a sequence identity threshold of the CDR3 region. Within a single VHH family defined herein, the CDR3 sequence is thus identical or very similar in amino acid composition, preferably with at least 80% identity, or at least 85% identity, or at least 90% identity in the CDR3 sequence, resulting in Nbs of the same family binding to the same binding site, having the same effect.

Immunoglobulin single variable domains such as Domain antibodies and Nanobody® (including VHH domains) can be subjected to humanization, i.e. increase the degree of sequence identity with the closest human germline sequence. In particular, humanized immunoglobulin single variable domains, such as Nanobody® (including VHH domains) may be immunoglobulin single variable domains in which at least one amino acid residue is present (and in particular, at least one framework residue) that is and/or that corresponds to a humanizing substitution (as defined further herein). Potentially useful humanizing substitutions can be ascertained by comparing the sequence of the framework regions of a naturally occurring VHH sequence with the corresponding framework sequence of one or more closely related human VH sequences, after which one or more of the potentially useful humanizing substitutions (or combinations thereof) thus determined can be introduced into said VHH sequence (in any manner known per se, as further described herein) and the resulting humanized VHH sequences can be tested for affinity for the target, for stability, for ease and level of expression, and/or for other desired properties. In this way, by means of a limited degree of trial and error, other suitable humanizing substitutions (or suitable combinations thereof) can be determined by the skilled person. Also, based on what is described before, (the framework regions of) an immunoglobulin single variable domain, such as a Nanobody® (including VHH domains) may be partially humanized or fully humanized. Humanized immunoglobulin single variable domains, in particular Nanobody®, may have several advantages, such as a reduced immunogenicity, compared to the corresponding naturally occurring VHH domains. By humanized is meant mutated so that immunogenicity upon administration in human patients is minor or non-existent. The humanizing substitutions should be chosen such that the resulting humanized amino acid sequence and/or VHH still retains the favourable properties of the VHH, such as the antigen-binding capacity. Based on the description provided herein, the skilled person will be able to select humanizing substitutions or suitable combinations of humanizing substitutions which optimize or achieve a desired or suitable balance between the favourable properties provided by the humanizing substitutions on the one hand and the favourable properties of naturally occurring VHH domains on the other hand. Such methods are known by the skilled addressee. A human consensus sequence can be used as target sequence for humanization, but also other means are known in the art. One alternative includes a method wherein the skilled person aligns a number of human germline alleles, such as for instance but not limited to the alignment of IGHV3 alleles, to use said alignment for identification of residues suitable for humanization in the target sequence. Also a subset of human germline alleles most homologous to the target sequence may be aligned as starting point to identify suitable humanisation residues. Alternatively, the VHH is analyzed to identify its closest homologue in the human alleles and used for humanisation construct design. A humanisation technique applied to Camelidae VHHs may also be performed by a method comprising the replacement of specific amino acids, either alone or in combination. Said replacements may be selected based on what is known from literature, are from known humanization efforts, as well as from human consensus sequences compared to the natural VHH sequences, or the human alleles most similar to the VHH sequence of interest. As can be seen from the data on the VHH entropy and VHH variability given in Tables A-5-A-8 of WO 08/020079, some amino acid residues in the framework regions are more conserved between human and Camelidae than others. Generally, although the invention in its broadest sense is not limited thereto, any substitutions, deletions or insertions are preferably made at positions that are less conserved. Also, generally, amino acid substitutions are preferred over amino acid deletions or insertions. For instance, a human-like class of Camelidae single domain antibodies contain the hydrophobic FR2 residues typically found in conventional antibodies of human origin or from other species, but compensating this loss in hydrophilicity by other substitutions at position 103 that substitutes the conserved tryptophan residue present in VH from double-chain antibodies. As such, peptides belonging to these two classes show a high amino acid sequence homology to human VH framework regions and said peptides might be administered to a human directly without expectation of an unwanted immune response therefrom, and without the burden of further humanisation. Indeed, some Camelidae VHH sequences display a high sequence homology to human VH framework regions and therefore said VHH might be administered to patients directly without expectation of an immune response therefrom, and without the additional burden of humanization.

Suitable mutations, in particular substitutions, can be introduced during humanization to generate a polypeptide with reduced binding to pre-existing antibodies (reference is made for example to WO 2012/175741 and WO2015/173325), for example at at least one of the positions: 11, 13, 14, 15, 40, 41, 42, 82, 82a, 82b, 83, 84, 85, 87, 88, 89, 103, or 108. The amino acid sequences and/or VHH of the invention may be suitably humanized at any framework residue(s), such as at one or more Hallmark residues (as defined below) or at one or more other framework residues (i.e. non-Hallmark residues) or any suitable combination thereof. Depending on the host organism used to express the amino acid sequence, VHH or polypeptide of the invention, such deletions and/or substitutions may also be designed in such a way that one or more sites for posttranslational modification (such as one or more glycosylation sites) are removed, as will be within the ability of the person skilled in the art. Alternatively, substitutions or insertions may be designed so as to introduce one or more sites for attachment of functional groups (as described herein), for example to allow site-specific pegylation.

In some cases, at least one of the typical Camelidae hallmark residues with hydrophilic characteristics at position 37, 44, 45 and/or 47 is replaced (see WO2008/020079 Table A-03). Another example of humanization includes substitution of residues in FR 1, such as position 1, 5, 11, 14, 16, and/or 28; in FR3, such as positions 73, 74, 75, 76, 78, 79, 82b, 83, 84, 93 and/or 94; and in FR4, such as position 10 103, 104, 108 and/or 111 (see WO2008/020079 Tables A-05-A08; all numbering according to the Kabat). Humanization typically only concerns substitutions in the FR and not in the CDRs, as this could/would impact binding affinity to the target and/or potency.

As used herein, a “therapeutically active agent” means any molecule that has or may have a therapeutic effect (i.e. curative or prophylactic effect) in the context of treatment of a disease (as described further herein). Preferably, a therapeutically active agent is a disease-modifying agent, which can be a cytotoxic agent, such as a toxin, or a cytotoxic drug, or an enzyme capable of converting a prodrug into a cytotoxic drug, or a radionuclide, or a cytotoxic cell, or which can be a non-cytotoxic agent. Even more preferably, a therapeutically active agent has a curative effect on the disease. The binding agent or the composition, or pharmaceutical composition of the invention may act as a therapeutically active agent, when beneficial in treating patients infected with corona virus infections, such as SARS Corona virus or patients suffering from COVID-19. The binding agent may include an agent comprising a variant VHH-72 ISVD, preferably an improved variant binding to the same binding region of the RBD, and more preferably a humanized variant thereof, and may contain or be coupled to additional functional groups, advantageous when administrated to a subject. Examples of such functional groups and of techniques for introducing them will be clear to the skilled person, and can generally comprise all functional groups and techniques mentioned in the art as well as the functional groups and techniques known per se for the modification of pharmaceutical proteins, and in particular for the modification of antibodies or antibody fragments, for which reference is for example made to Remington's Pharmaceutical Sciences, 16th ed., Mack Publishing Co., Easton, Pa. (1980). Such functional groups may for example be linked directly (for example covalently) to the ISVD or active antibody fragment, or optionally via a suitable linker or spacer, as will again be clear to the skilled person. One of the most widely used techniques for increasing the half-life and/or reducing immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). For example, for this purpose, PEG may be attached to a cysteine residue that naturally occurs in a immunoglobulin single variable domain of the invention, a immunoglobulin single variable domain of the invention may be modified so as to suitably introduce one or more cysteine residues for attachment of PEG, or an amino acid sequence comprising one or more cysteine residues for attachment of PEG may be fused to the N- and/or C-terminus of an ISVD or active antibody fragment of the invention, all using techniques of protein engineering known per se to the skilled person. Another, usually less preferred modification comprises N-linked or O-linked glycosylation, usually as part of co-translational and/or post-translational modification, depending on the host cell used for expressing the antibody or active antibody fragment. Another technique for increasing the half-life of a binding domain may comprise the engineering into bifunctional or bispecific domains (for example, one ISVD or active antibody fragment against the target RBD of Corona virus and one against a serum protein such as albumin or Surfactant Protein A (SpA)—which is a surface protein abundantly present in the lungs aiding in prolonging half-life)) or into fusions of antibody fragments, in particular immunoglobulin single variable domains, with peptides (for example, a peptide against a serum protein such as albumin). In yet another example, the variant ISVD of the invention can be fused to an immunoglobulin Fc domain such as an IgA Fc domain or an IgG Fc domain, such as for example IgG1, IgG2 or IgG4 Fc domains. Examples are further shown in the experimental section and are also depicted in the sequence listing.

The term “compound” or “test compound” or “candidate compound” or “drug candidate compound” as used herein describes any molecule, either naturally occurring or synthetic that is designed, identified, screened for, or generated and may be tested in an assay, such as a screening assay or drug discovery assay, or specifically in the method for identifying a compound capable of neutralizing Corona virus, specifically 2019-Corona virus infections. As such, these compounds comprise organic and inorganic compounds. For high-throughput purposes, test compound libraries may be used, such as combinatorial or randomized libraries that provide a sufficient range of diversity. Examples include, but are not limited to, natural compound libraries, allosteric compound libraries, peptide libraries, antibody fragment libraries, synthetic compound libraries, fragment-based libraries, phage-display libraries, and the like. Such compounds may also be referred to as binding agents; as referred to herein, these may be “small molecules”, which refers to a low molecular weight (e.g., <900 Da or <500 Da) organic compound. The compounds or binding agents also include chemicals, polynucleotides, lipids or hormone analogs that are characterized by low molecular weights. Other biopolymeric organic test compounds include small peptides or peptide-like molecules (peptidomimetics) comprising from about 2 to about 40 amino acids and larger polypeptides comprising from about 40 to about 500 amino acids, such as antibodies, antibody mimetics, antibody fragments or antibody conjugates.

As used herein, the terms “determining,” “measuring,” “assessing,”, “identifying”, “screening”, and “assaying” are used interchangeably and include both quantitative and qualitative determinations. “Similar” as used herein, is interchangeable for alike, analogous, comparable, corresponding, and -like or alike, and is meant to have the same or common characteristics, and/or in a quantifiable manner to show comparable results i.e. with a variation of maximum 20%, 10%, more preferably 5%, or even more preferably 1%, or less.

The term “subject”, “individual” or “patient”, used interchangeably herein, relates to any organism such as a vertebrate, particularly any mammal, including both a human and another mammal, for whom diagnosis, therapy or prophylaxis is desired, e.g., an animal such as a rodent, a rabbit, a cow, a sheep, a horse, a dog, a cat, a lama, a pig, or a non-human primate (e.g., a monkey). The rodent may be a mouse, rat, hamster, guinea pig, or chinchilla. In one embodiment, the subject is a human, a rat or a non-human primate. Preferably, the subject is a human. In one embodiment, a subject is a subject with or suspected of having a disease or disorder, in particular a disease or disorder as disclosed herein, also designated “patient” herein. However, it will be understood that the aforementioned terms do not imply that symptoms are present.

The term “treatment” or “treating” or “treat” can be used interchangeably and are defined by a therapeutic intervention that slows, interrupts, arrests, controls, stops, reduces, or reverts the progression or severity of a sign, symptom, disorder, condition, or disease, but does not necessarily involve a total elimination of all disease-related signs, symptoms, conditions, or disorders. Therapeutic treatment is thus designed to treat an illness or to improve a person's health, rather than to prevent an illness. Treatment may also refer to a prophylactic treatment which relates to a medication or a treatment designed and used to prevent a disease from occurring.

### DETAILED DESCRIPTION

In a first aspect of the invention, a binding agent is disclosed, which specifically interacts with the Receptor binding domain present in the spike protein of the Corona virus, specifically the SARS-CoV-1 virus and the SARS-Cov-2 Corona virus. Binding between the agent and the spike protein results in a neutralization of the infection capacity of the Corona virus. In a particular embodiment the invention provides a binding agent specifically binding the Corona virus spike protein at an epitope comprising amino acid residues Leu355, Tyr356, Ser358, Ser362, Thr363, F364, K365, C366 and Y494 wherein the sequence of said spike protein is set forth in SEQ ID NO:24. In another particular embodiment the invention provides a binding agent specifically binding the Corona virus spike protein at an epitope comprising amino acid residues Leu355, Tyr356, Ser358, Ser362, Thr363, F364, K365, C366, Y494 and R426 wherein the sequence of said spike protein is set forth in SEQ ID NO:24. Comparison of the Spike of SARS-CoV-1 and -2, as well as structural comparison and further cryo-EM analysis revealed that the epitope as defined herein on the SARS-CoV-1 Spike corresponds to binding to the same epitope of SARS-Cov-2 Spike defined by a conformational epitope formed by the residues L368, Y369, S371, S375, T376, F377, K378, C379 and Y508 as set forth in SEQ ID NO: 23, which is the sequence of the SARS-Cov-2 Spike protein. Moreover, the structural analysis further demonstrates that said epitope as defined herein, specifically binding the binding agents as defined herein, in particular VHH72, is occluded in the closed spike conformation that is the dominant one on the native virus⁸¹. Even in the ‘1-RBD-up’ conformation that can bind the ACE2 receptor, the epitope is positioned such that human monoclonal antibodies cannot easily reach it. Possibly because of this, amidst hundreds of antibodies against other regions of the spike, very few human antibodies thus bind to an epitope that substantially overlaps the VHH72 epitope⁸². Moreover, the epitope is comprised of residues that form crucial packing contacts between the protomers of the trimeric spike. SARS-CoV-2 viruses with mutations in this epitope so far remain extremely rare. Consistently, none of the emerging and rapidly spreading viral variant's RBD mutations affect the VHH72 binding site. Antibodies that cross-neutralize SARS-CoV-1 and -2 and other viruses of the Sarbecovirus subgenus, as is the case for the binding agents of the present invention, are thus rare and the present binding agents comprising said ISVDs are thereby unique.

Another embodiment relates to a binding agent specifically binding the Corona virus Spike protein, which is defined as a binding agent competing for the epitope as defined herein, or competing with VHH72 binding to the RBD epitope. With ‘competing’ is meant that the binding of VHH72 to the Spike protein as depicted in SEQ ID NO:23 is reduced with at least 30%, or at least 50%, or preferably at least 80% in strength in the presence of said competing binding agent. More specifically, said competing binding agent specifically binds an epitope on the Spike protein comprising at least three, at least four, at least five, at least 6 or more of the residues L368, Y369, S371, S375, T376, F377, K378, C379 and Y508 of the Spike protein of SARS-Cov-2, as depicted in SEQ ID NO:23, so as to provide an overlapping epitope, more specifically at least binding to 2 of its residues, or at least to 3, or at least 4, or at least 6 of its residues. In a specific embodiment the competing binding agent specifically binds to residues K378, Y369 and F377.

In another specific embodiment the competing binding agent specifically binds to residues K378, Y369 and F377 as depicted in SEQ ID NO:23, and said competing binding agent competes for ACE2 receptor binding to the Spike protein and/or RBD domain.

In another specific embodiment said competing binding agent is also capable of binding to the SARS-CoV-1 Spike protein, as depicted in SEQ ID NO:24.

The need for improved variants of VHH72 with superior binding characteristics such as improved Kₒₙ rates and improved K_{off} rates, resulted in the identification of VHH72-S56A variant, with a serine to alanine mutation at position 56 (Kabat numbering) as a building block, and alternatively, humanized variants thereof, such as VHH72_h1_E1D_S56A. The S56A mutation was shown to result in a higher affinity for SARS-CoV-1 and -2 spike and receptor-binding domain and an approximately 5-7 fold higher authentic SARS-CoV-2 neutralizing activity when fused to a human IgG1 Fc (see examples). The in vivo efficacy of said S56A mutation has been analysed in a hamster model for SARS-Cov2 herein, as compared to the humanized variant of VHH72-Fc, and revealed to be superior to the VHH72 formats not comprising the S56A mutant. However, any alternative VHH building blocks, as disclosed herein, with similar or improved binding and neutralization properties that compete for or bind to the same RBD epitope as VHH72, and fused to an Fc domain are envisaged herein in any such combination or variant as discussed herein for VHH72 or vHH72S56A. Typically, any further humanization efforts, as described herein may also be used to generate more clinically relevant forms of for instance the VHHs ISVDs identified herein by SEQ ID NOs: 27 to 61, or SEQ ID NOs:92 to 105.

Thus in another specific embodiment the binding agent is a polypeptide binder, containing at least one ISVD, which is further defined by its binding residues or paratopic residues, and herein limited to the sequence of its CDRs. As shown in the structural examples, the CDRs regions confer the binding characteristics of the ISVDs and thus comprise one of the following CDR1, CDR2, and CDR3 combinations:
CDR1 consisting of a SEQ ID NO: 7; CDR2 of SEQ ID NO: 8 or 10; and CDR3 of SEQ ID NO: 9, or
CDR1 consisting of a SEQ ID NO: 111; CDR2 of SEQ ID NO: 120; and CDR3 of SEQ ID NO: 9, or
CDR1 consisting of a SEQ ID NO: 112; CDR2 of SEQ ID NO: 121; and CDR3 of SEQ ID NO: 131, or
CDR1 consisting of a SEQ ID NO: 113; CDR2 of SEQ ID NO: 121; and CDR3 of SEQ ID NO: 131, or
CDR1 consisting of a SEQ ID NO: 114; CDR2 of the sequence TISWSGGGTYYAEPVRG; and CDR3 of SEQ ID NO: 132, or
CDR1 consisting of a SEQ ID NO: 113; CDR2 of SEQ ID NO: 123; and CDR3 of SEQ ID NO: 133, or
CDR1 consisting of the sequence SYAMG; CDR2 of SEQ ID NO: 124; and CDR3 of SEQ ID NO: 134, or
CDR1 consisting of a the sequence SYAMG; CDR2 of SEQ ID NO: 125; and CDR3 of SEQ ID NO: 135, or
CDR1 consisting of a SEQ ID NO: 115; CDR2 of SEQ ID NO: 126; and CDR3 of SEQ ID NO: 136, or
CDR1 consisting of a SEQ ID NO: 116; CDR2 of SEQ ID NO: 127; and CDR3 of SEQ ID NO: 137, or
CDR1 consisting of a SEQ ID NO: 117; CDR2 of SEQ ID NO: 128; and CDR3 of SEQ ID NO: 138, or
CDR1 consisting of a SEQ ID NO: 118; CDR2 of SEQ ID NO: 129; and CDR3 of SEQ ID NO: 139, or
CDR1 consisting of a SEQ ID NO: 119; CDR2 of SEQ ID NO: 130; and CDR3 of SEQ ID NO: 140.

In another specific embodiment the binding polypeptide comprises an ISVD comprising the CDR1, CDR2, and CDR3 selected from a specific ISVDs selected from the group of SEQ ID NO: 1, SEQ ID NO:4, or SEQ ID NO:27-61, or SEQ ID NO:92-105, wherein said CDR sequences are defined by any one of the annotations as provided by Kabat, MacCallum, IMGT, AbM, or Chothia, as described herein, and as exemplified for VHH72-S56A in FIG. 39.

In a more specific embodiment, said binding agents comprising one or more ISVDs is defined by the full length sequence of the ISVD, wherein said sequence is selected from the group of SEQ ID NO: 1 to 6, 11, 27 to 61 and 92 to 105, or a sequence with at least 90% identity thereof, or at least 95% identity thereof, wherein said difference in identity, or variability, is limited to the FR residues, or any humanized variant thereof, wherein said humanized variant is a functional orthologue, i.e. a binding agent still retaining the same binding site specificity and capability to compete with ACE2 binding to the RBD.

In another specific embodiment, said binding agent comprises one or more ISVDs which belong to the VHH72 family, and are defined by an ISVD comprising ISVD comprising the CDR1, CDR2, and CDR3 selected from a specific ISVDs selected from the group of SEQ ID NO: 1, SEQ ID NO:4, or SEQ ID NO:27-61, or SEQ ID NO:92-97, wherein said CDR sequences are defined by any one of the annotations as provided by Kabat, MacCallum, IMGT, AbM, or Chothia, as described herein, and as exemplified for VHH72-S56A in FIG. 39, and as exemplified for Kabat annotation for SEQ ID NO:92-97 in Table 6.

In another specific embodiment, said binding agent comprises one or more ISVDs which belong to a different VHH family than the VHH72 family, and have been shown to bind exactly the same epitope, and are defined by an ISVD comprising ISVD comprising the CDR1, CDR2, and CDR3 selected from a specific ISVDs selected from the group of SEQ ID NO: 98 (VHH3.83), SEQ D NO:101 (VHH3.55), SEQ ID NO:102 (VHH3.35), and SEQ ID NO:104 (VHH3.38), wherein said CDR sequences are defined by any one of the annotations as provided by Kabat, MacCallum, IMGT, AbM, or Chothia, as described herein, and as exemplified for VHH72-S56A in FIG. 39, and as exemplified for Kabat annotation for SEQ ID NO: 98, 101, 102, and 104 in Table 6.

In another specific embodiment, said binding agent comprises one or more ISVDs which belong to a different VHH family than the VHH72 family, and have been shown to compete for the same epitope as VHH72, and are defined by an ISVD comprising ISVD comprising the CDR1, CDR2, and CDR3 selected from a specific ISVDs selected from the group of SEQ ID NO: 99 (VHH3.36), SEQ D NO:100 (VHH3.47), SEQ ID NO:103 (VHH3.29), and SEQ ID NO:105 (VHH3.149), wherein said CDR sequences are defined by any one of the annotations as provided by Kabat, MacCallum, IMGT, AbM, or Chothia, as described herein, and as exemplified for VHH72-S56A in FIG. 39, and as exemplified for Kabat annotation for SEQ ID NO: 99, SEQ D NO:100, SEQ ID NO:103, and SEQ ID NO:105, in Table 6.

Another embodiment relates to said protein binding agents wherein the at least one or more ISVD is bound or fused to an Fc domain, wherein with Fc domain is meant the fragment crystallizable region (Fc region) of an antibody, which is the tail region known to interact with cell surface receptors called Fc receptors and some proteins of the complement system. Said Fc domain is composed of two identical protein fragments, derived from the second and third constant domains of the antibody's two heavy chains. All conventional antibodies comprise an Fc domain, hence, the Fc domain fusion may comprise an Fc domain derived from or as a variant of the IgG, IgA and IgD antibody Fc regions, even more specifically an IgG1, IgG2 or IgG4. The hinge region of IgG2, may be replaced by the hinge of human IgG1 to generate SARS VHH-72 fusion constructs, and vice versa. Additional linkers that are used to fuse SARS VHH-72 to the IgG1 and IgG2 Fc domains comprise (G₄S)₂₋₃. In addition, Fc variants with known half-live extension may be used such as the M257Y/S259T/T261E (also known as YTE) or the LS variant (M428L combined with N434S). These mutations increase the binding of the Fc domain of a conventional antibody to the neonatal receptor (FcRn).

In a particular embodiment, the binding agent of the invention comprising one or more immunoglobulin single variable domains are in a “multivalent” or “multispecific” form and are formed by bonding, chemically or by recombinant DNA techniques, together two or more identical or variant monovalent ISVDs. Said multivalent forms may be formed by connecting the building block directly or via a linker, or through fusing the with an Fc domain encoding sequence. Non-limiting examples of multivalent constructs include “bivalent” constructs, “trivalent” constructs, “tetravalent” constructs, and so on. An example of such a bivalent construct is herein further described in the appended examples section. The immunoglobulin single variable domains comprised within a multivalent construct may be identical or different. In another particular embodiment, the immunoglobulin single variable domains of the invention are in a “multi-specific” form and are formed by bonding together two or more immunoglobulin single variable domains, of which at least one with a different specificity. Non-limiting examples of multi-specific constructs include “bi-specific” constructs, “tri-specific” constructs, “tetra-specific” constructs, and so on. To illustrate this further, any multivalent or multi-specific (as defined herein) ISVD of the invention may be suitably directed against two or more different epitopes on the same RBD of Corona virus antigen, or may be directed against two or more different antigens, for example against the Corona RBD and one as a half-life extension against Serum Albumin or SpA. Multivalent or multi-specific ISVDs of the invention may also have (or be engineered and/or selected for) increased avidity and/or improved selectivity for the desired Corona RBD interaction, and/or for any other desired property or combination of desired properties that may be obtained by the use of such multivalent or multi-specific immunoglobulin single variable domains. Upon binding the Corona RBD, said multi-specific binding agent or multivalent ISVD may have an additive or synergistic impact on the binding and neutralization of Corona virus, such as SARS-Corona or 2019-novel Corona virus. In another embodiment, the invention provides a polypeptide comprising any of the immunoglobulin single variable domains according to the invention, either in a monovalent, multivalent or multi-specific form. Thus, polypeptides comprising monovalent, multivalent or multi-specific nanobodies are included here as non-limiting examples.

Particularly, a single ISVD as described herein may be fused at its C-terminus to an IgG Fc domain, resulting in a SARS-Cov-2 binding agents of bivalent format wherein two of said VHH72_S56A IgG Fcs, or humanized forms thereof, form a heavy chain only-antibody-type molecule through disulfide bridges in the hinge region of the IgG Fc part. Said humanized forms thereof, include but are not limited to the IgG humanization variants known in the art, such as C-terminal deletion of Lysine, alteration or truncation in the hinge region, LALA or LALAPG mutations as described herein, among other substitutions in the IgG sequence. In a specific embodiment, said SARS-Cov-2 binding agents comprise the amino acid sequence as depicted in SEQ ID NO: 13 to 22, or a variant with at least 90% identity thereof.

In particular, the amino acid sequence of SEQ ID NO:18 provides for the construct that is composed of the VHH72 building block, linked via a GS(G4S)2-linker to the human IgG1 hinge sequence, which is further connected to the Fc part of the human IgG1. This protein sequence provides for the prototype or wild-type VHH72-Fc as also described in¹⁰. The amino acid sequence of SEQ ID NO:17 (as used herein as D72-58 batch) provides for the construct that is composed of the VHH72_h1(E1D) humanized variant of VHH72 as building block, linked via a 10GS-linker to the human IgG1 hinge sequence containing a deletion (EPKSC), which is further connected to the Fc part of the human IgG1, containing the LALA mutation for reduced Fcγ receptor binding, and with the C-terminal lysine deleted. So in fact, the Prelead sequence provides for a fully optimized humanization variant of SEQ ID NO:18. The amino acid sequence of SEQ ID NO:22 (as used herein as PB9683 batch and also representing the Lead molecule) provides for the construct that is composed of the VHH72_h1(E1D) building block (identical to the building block of SEQ ID NO:17), containing a mutation in the CDR2 region, S56A (according to Kabat), linked via a 10GS-linker to the human IgG1 hinge sequence containing a deletion (EPKSC), which is further connected to the Fc part of the human IgG1, containing the LALA mutation for reduced Fcγ receptor binding, and with the C-terminal lysine deleted. So, the lead protein batch as used herein provides for a humanized variant of VHH72-Fc that is identical to the Prelead, with the exception for the improved S56A mutation.

In yet another aspect, the invention provides a nucleic acid molecule encoding a SARS-CoV-2 binder as described herein. In yet another embodiment the invention provides a recombinant vector comprising the nucleic acid molecule as described herein. Said vectors may include a cloning or expression vector, as well as a delivery vehicle such as a viral, lentiviral or adenoviral vector. The term “vector”, “vector construct,” “expression vector,” “recombinant vector” or “gene transfer vector,” as used herein, is intended to refer to a nucleic acid molecule capable of transporting another nucleic acid molecule to which it has been linked. More particular, said vector may include any vector known to the skilled person, including any suitable type, but not limited to, for instance, plasmid vectors, cosmid vectors, phage vectors, such as lambda phage, viral vectors, even more particular a lentiviral, adenoviral, AAV or baculoviral vectors, or artificial chromosome vectors such as bacterial artificial chromosomes (BAC), yeast artificial chromosomes (YAC), or P1 artificial chromosomes (PAC). Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments. The construction of expression vectors for use in transfecting cells is also well known in the art, and thus can be accomplished via standard techniques (see, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion, Austin, Tex.). Furthermore, an alternative embodiment relates to the use of said nucleic acid molecule, expression cassette, or vector described herein encoding said binding agent of the present invention, for production as an intrabody. An intracellular antibody or “intrabody” is an antibody or an active fragment of an antibody that is heterologously expressed within a designated intracellular compartment, a process which is made possible through the in-frame incorporation of intracellular trafficking signals. Intrabodies exert their functions upon exquisitely specific interaction with target antigens. This results in interruption or modification of the biological functions of the target protein. An intrabody can be expressed in any shape or form such as an intact IgG molecule or a Fab fragment. More frequently, intrabodies are used in genetically engineered antibody fragment format and structures of scFv intrabodies, single domain intrabodies, or bispecific tetravalent intradiabodies. For a review see Zhu, and Marasco, 2008 (Therapeutic Antibodies. Handbook of Experimental Pharmacology 181. _c Springer-Verlag Berlin Heidelberg). The binding agents comprising an ISVD as described herein, possibly encoded by a nucleic acid molecule or expression cassette are present on a vector as described herein, resulting in an intrabody upon expression within a suitable host system, could also serve as a tool, as a diagnostic, for in vivo imaging, or as well as a therapeutic, when an applicable form of gene delivery is identified. A skilled person is aware about the currently applied methodologies of administration and delivery (also see Zhu and Marasco 2008).

Where said binding agent is provided as a nucleic acid or a vector, it is particularly envisaged that the modulator is administered through gene therapy. ‘Gene therapy’ as used herein refers to therapy performed by the administration to a subject of an expressed or expressible nucleic acid. For such applications, the nucleic acid molecule or vector as described herein allow for production of the binding agent within a cell. A large set of methods for gene therapy are available in the art and include, for instance (adeno-associated) virus mediated gene silencing, or virus mediated gene therapy (e.g. US 20040023390; Mendell et al 2017, N Eng J Med 377:1713-1722). A plethora of delivery methods are well known to those of skill in the art and include but are not limited to viral delivery systems, microinjection of DNA plasmids, biolistics of naked nucleic acids, use of a liposome. In vivo delivery by administration to an individual patient occurs typically by systemic administration (e.g., intravenous, intraperitoneal infusion or brain injection; e.g. Mendell et al 2017, N Eng J Med 377:1713-1722). Where said binding agent is provided as a nucleic acid or a vector, it is more particularly also envisaged that the modulator is administered through delivery methods and vehicles that comprise nanoparticles or lipid-based delivery systems such as artificial exosomes, which may also be cell-specific, and suitable for delivery of the binding agents or multi-specific binding agents as intrabodies or in the form of DNA to encode said binding agent or modulator.

One further aspect of the invention provides for a host cell comprising the ISVD or active antibody fragment of the invention. The host cell may therefore comprise the nucleic acid molecule encoding said ISVD. Host cells can be either prokaryotic or eukaryotic. The host cell may also be a recombinant host cell, which involves a cell which has been genetically modified to contain an isolated DNA molecule, nucleic acid molecule encoding the ISVD of the invention. Representative host cells that may be used to produce said ISVDs, but are not limited to, bacterial cells, yeast cells, plant cells and animal cells. Bacterial host cells suitable for production of the binding agents of the invention include Escherichia spp. cells, Bacillus spp. cells, Streptomyces spp. cells, Erwinia spp. cells, Klebsiella spp. cells, Serratia spp. cells, Pseudomonas spp. cells, and Salmonella spp. cells. Yeast host cells suitable for use with the invention include species within Saccharomyces, Schizosaccharomyces, Kluyveromyces, Pichia (e.g. Pichia pastoris), Hansenula (e.g. Hansenula polymorpha), Yarowia, Schwaniomyces, Schizosaccharomyces, Zygosaccharomyces and the like. Saccharomyces cerevisiae, S. carlsbergensis and K. lactis are the most commonly used yeast hosts, and are convenient fungal hosts. Animal host cells suitable for use with the invention include insect cells and mammalian cells (most particularly derived from Chinese hamster (e.g. CHO), and human cell lines, such as HeLa). Exemplary insect cell lines include, but are not limited to, Sf9 cells, baculovirus-insect cell systems (e.g. review Jarvis, Virology Volume 310, Issue 1, 25 May 2003, Pages 1-7). Alternatively, the host cells may also be transgenic animals.

### Crystal Complexes

Another aspect of the invention relates to a complex comprising the RBD of Corona virus and a binding agent as described herein. In a further embodiment, said complex is of a crystalline form. The crystalline allows to further use said the atomic details of the interactions in said complex as a molecular template to design molecules that will recapitulate the key features of the RBD-binding agent interfaces. In the light of recent developments in computational docking and in pharmacophore building, the isolation of small compounds that can mimic protein-protein interface is becoming a realistic strategy.

A specific embodiment is thus related to the crystal comprising the SARS-Corona RBP as depicted in SEQ ID NO: 26 and the binding agent depicted in SEQ ID NO: 1, and characterized in that the crystal is:
i) a crystal between SEQ ID NO: 26 and SEQ ID NO: 1 in the space group P3₁21, with the following crystal lattice constants: a=88.8 Å±5%, b=88.8 Å±5%, c=200.8 Å±5%, α=90, β=90, γ=120°.

Said crystal has a three-dimensional structure wherein the crystal i) comprises an atomic structure characterized by the coordinates of PDB 6WAQ (deposited on 2020/03/25 to the RCSB Protein Database; released on 2020/04/01 as Version 1.0) or a subset of atomic coordinates thereof.

A binding site, consisting of a subset of atomic coordinates, present in the crystal i) as defined herein, wherein said binding site consists of the amino acid residues: Leu355, Tyr356, Ser358, Ser362, Thr363, F364, K365, C366 and Y494, or Leu355, Tyr356, Ser358, Ser362, Thr363, F364, K365, C366, Y494 and R426 as set forth in SEQ ID NO:24 and wherein said amino acid residues represent the binding agent's SARS-Corona virus RBP, more particularly 2019-nCoV RBP.

Another specific embodiment thus relates to a computer-assisted method of identifying, designing or screening for a neutralizing agent of the Corona virus RBP domain wherein said neutralizing agent is a binding agent selected from the group consisting of a small molecule compound, a chemical, a peptide, a peptidomimetic, an antibody mimetic, an ISVD, an antibody or antibody fragment, and comprising:
i. introducing into suitable computer program parameters defining the three-dimensional structure of said binding site,
ii. creating a three-dimensional structure of a test compound in said computer program;
iii. displaying a superimposing model of said test compound on the three-dimensional model of the binding site; and
iv. assessing whether said test compound model fits spatially and chemically into a binding site.

Said binding site as described herein is also referred to herein as the epitope of the invention. Moreover, the epitope here refers to specific residues in the RBD of the Spike protein of SARS-Corona virus of which Spike protein sequence is depicted in SEQ ID NO: 24. These residues are in ‘in contact’ with the binding agent. In particular, where the epitope is described as disclosed herein ‘contact’ is defined herein as closer than 4 Å, as closer than 5 Å, as closer than 6 Å or as closer than 7 Å from any residue (or atom) belonging to the nanobody (VHH-72 or also designated herein as SARS VHH-72, or a variant thereof) or any other binding agent of interest specifically binding to the RBD in SARS-Corona or 2019-novel Corona virus, in particular any of said binding agents binding to the same epitope, and with a certain potential to outcompete the ACE2 receptor for binding to the RBD of said Spike protein.

### Rational Drug Design

Using a variety of known modelling techniques, the crystal structures of the present application can be used to produce models for evaluating the interaction of compounds with SARS-Corona virus or 2019-novel Corona virus, in particular with the RBD, or vice versa evaluating the design of novel epitope-mimicking compounds and their interaction with the binding agents of the invention. As used herein, the term “modelling” includes the quantitative and qualitative analysis of molecular structure and/or function based on atomic structural information and interaction models. The term “modelling” includes conventional numeric-based molecular dynamic and energy minimisation models, interactive computer graphic models, modified molecular mechanics models, distance geometry and other structure-based constraint models. Molecular modelling techniques can be applied to the atomic coordinates of the SARS-Corona virus or 2019-novel Corona virus RBD domain to derive a range of 3D models and to investigate the structure of binding sites, such as the binding sites with chemical entities. These techniques may also be used to screen for or design small and large chemical entities which are capable of binding the SARS-Corona virus or 2019-novel Corona virus RBD domain, or with the ISVDs disclosed herein, and may modulate the neutralization of SARS-Corona virus or 2019-novel Corona virus. Such a screen may employ a solid 3D screening system or a computational screening system. Such modelling methods are to design or select chemical entities that possess stereochemical complementary to identified binding sites or pockets in the RBD domain. By “stereochemical complementarity” it is meant that the compound makes a sufficient number of energetically favourable contacts with the RBD domain as to have a net reduction of free energy on binding to the RBD domain. By “stereochemical similarity” it is meant that the compound makes about the same number of energetically favourable contacts with the RBD domain set out by the coordinates shown in Appendixes I. Stereochemical complementarity is characteristic of a molecule that matches intra-site surface residues lining the groove of the receptor site as enumerated by the coordinates set out in the Protein database entry provided for the complex of the present invention, for instance the PDB 6WAQ. By “match” we mean that the identified portions interact with the surface residues, for example, via hydrogen bonding or by non-covalent Van der Waals and Coulomb interactions (with surface or residue) which promote dissolvation of the molecule within the site, in such a way that retention of the molecule at the binding site is favoured energetically. It is preferred that the stereochemical complementarity is such that the compound has a K_{d} for the binding site of less than 10¹M, more preferably less than 10⁻⁵ M and more preferably 10⁻⁶ M. In a most particular embodiment, the K_{d} value is less than 10⁻⁸ M and more particularly less than 10⁻⁹ M.

A number of methods may be used to identify chemical entities possessing stereochemical complementarity to the structure or substructures of the RBD binding domain. For instance, the process may begin by visual inspection of a selected binding site in the RBD domain on the computer screen based on the coordinates in PDB 6WAQ generated from the machine-readable storage medium. Alternatively, selected fragments or chemical entities may then be positioned in a variety of orientations, or docked, within the selected binding site. Modelling software is well known and available in the art. This modelling step may be followed by energy minimization with standard available molecular mechanics force fields. Once suitable chemical entities or fragments have been selected, they can be assembled into a single compound. In one embodiment, assembly may proceed by visual inspection of the relationship of the fragments to each other on the three-dimensional image displayed on a computer screen in relation to the atomic coordinates of selected binding site or binding pocket in the RBD binding site. This is followed by manual model building, typically using available software. Alternatively, fragments may be joined to additional atoms using standard chemical geometry. The above-described evaluation process for chemical entities may be performed in a similar fashion for chemical compounds.

Databases of chemical structures are available from a number of sources including Cambridge Crystallographic Data Centre (Cambridge, U.K.), Molecular Design, Ltd., (San Leandro, Calif.), Tripos Associates, Inc. (St. Louis, Mo.), Chemical Abstracts Service (Columbus, Ohio), the Available Chemical Directory (Symyx Technologies, Inc.), the Derwent World Drug Index (WDI), BioByteMasterFile, the National Cancer Institute database (NCI), Medchem Database (BioByte Corp.), ZINC docking database (University of California, Sterling and Irwin, J. Chem. Inf. Model, 2015), and the Maybridge catalogue. Once an entity or compound has been designed or selected by the above methods, the efficiency with which that entity or compound may bind to the RBD domain or binding site can be tested and optimised by computational evaluation. For example, a compound that has been designed or selected to function as a RBD domain binding compound must also preferably traverse a volume not overlapping that occupied by the binding site when it is bound to the native RBD domain. An effective SARS-Corona virus or 2019-novel Corona virus RBD binding compound must preferably demonstrate a relatively small difference in energy between its bound and free states (i.e. a small deformation energy of binding). Thus, the most efficient RBD binding compound should preferably be designed with a deformation energy of binding of not greater than about 10 kcal/mole, particularly, not greater than 7 kcal/mole. RBD binding compounds may interact with, for instance but not limited to, the RBD domain in more than one conformation that are similar in overall binding energy. In those cases, the deformation energy of binding is taken to be the difference between the energy of the free compound and the average energy of the conformations observed when the compound binds to the protein. Further, a compound designed or selected as binding to the RBD domain may be further computationally optimised so that in its bound state it would preferably lack repulsive electrostatic interaction with the target protein.

Once a RBD domain or SARS-Corona (SARS-CoV-1) virus or SARS-CoV-2 virus or mutant SARS-CoV-2 virus binding compound has been optimally selected or designed, as described above, substitutions may then be made in some of its atoms or side groups to improve or modify its binding properties. Generally, initial substitutions are conservative, i.e. the replacement group will have approximately the same size, shape, hydrophobicity and charge as the original group. Preferred conservative substitutions are those fulfilling the criteria defined for an accepted point mutation in Dayhoff et al., Atlas of Protein Sequence and Structure, 5, pp. 345-352 (1978 & Supp.), which is incorporated herein by reference. Examples of conservative substitutions are substitutions including but not limited to the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine. It should, of course, be understood that components known in the art to alter conformation should be avoided. Such substituted chemical compounds may then be analysed for efficiency of fit to the RBD domain by the same computer methods described above.

Specific computer software is available in the art to evaluate compound deformation energy and electrostatic interaction. The screening/design methods may be implemented in hardware or software, or a combination of both. However, preferably, the methods are implemented in computer programs executing on programmable computers each comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design. Each program is preferably implemented in a high-level procedural or object-oriented programming language to communicate with a computer system. However, the programs can be implemented in assembly or machine language, if desired. In any case, the language may be compiled or interpreted language. Each such computer program is preferably stored on a storage medium or device (e.g., ROM or magnetic diskette) readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein.

### Compounds

The term “compound” or “test compound” or “candidate compound” or “drug candidate compound” as used herein describes any molecule, either naturally occurring or synthetic that may be tested in an assay, such as a screening assay or drug discovery assay, or specifically in the method for identifying a compound capable of binding and neutralizing SARS-Corona virus or 2019-novel Corona virus. As such, these compounds comprise organic and inorganic compounds. The compounds may be small molecules, chemicals, peptides, antibodies or ISVDs or active antibody fragments.

Compounds of the present invention include both those designed or identified using a screening method of the invention and those which are capable of binding and neutralizing SARS-Corona virus or 2019-novel Corona virus as defined above. Compounds capable of binding and neutralizing SARS-Corona virus or 2019-novel Corona virus may be produced using a screening method based on use of the atomic coordinates corresponding to the 3D structure of the RBD-VHH-72 complex as presented herein. The candidate compounds and/or compounds identified or designed using a method of the present invention may be any suitable compound, synthetic or naturally occurring, preferably synthetic. In one embodiment, a synthetic compound selected or designed by the methods of the invention preferably has a molecular weight equal to or less than about 5000, 4000, 3000, 2000, 1000 or more preferably less than about 500 daltons, or is preferably a peptide. A compound of the present invention is preferably soluble under physiological conditions. Such compounds can comprise functional groups necessary for structural interaction with proteins, particularly hydrogen bonding, and typically include at least an amine, carbonyl, hydroxyl or carboxyl group, preferably at least two of the functional chemical groups. The compound may comprise cyclical carbon or heterocyclic structures and/or aromatic or polyaromatic structures substituted with one or more of the above functional groups. Compounds can also comprise biomolecules including peptides, saccharides, fatty acids, steroids, purines, pyrimidines, derivatives, structural analogues, or combinations thereof. Compounds may include, for example: (1) peptides such as soluble peptides, including Ig-tailed fusion peptides and members of random peptide libraries and combinatorial chemistry-derived molecular libraries made of D- and/or L-configuration amino acids; (2) phosphopeptides (e.g. members of random and partially degenerate, directed phosphopeptide libraries, (3) antibodies (e.g., polyclonal, monoclonal, humanized, anti-idiotypic, chimeric, and single chain antibodies, nanobodies as well as Fab, (Fab)₂, Fab expression library and epitope-binding fragments of antibodies); (4) non-immunoglobulin binding proteins such as but not restricted to avimers, DARPins and lipocalins; (5) nucleic acid-based aptamers; and (6) small organic and inorganic molecules.

Synthetic compound libraries are commercially available from, for example, Maybridge Chemical Co. (Tintagel, Cornwall, UK), AMRI (Budapest, Hungary) and ChemDiv (San Diego, Calif.), Specs (Delft, The Netherlands), ZINC15 (Univ. of California). In addition, numerous means are available for random and directed synthesis of a wide variety of organic compounds and biomolecules, including expression of randomized oligonucleotides. Alternatively, libraries of natural compounds in the form of bacterial, fungal, plant and animal extracts can be readily produced. In addition, natural or synthetic compound libraries and compounds can be readily modified through conventional chemical, physical and biochemical means and may be used to produce combinatorial libraries. In addition, numerous methods of producing combinatorial libraries are known in the art, including those involving biological libraries; spatially addressable parallel solid phase or solution phase libraries; synthetic library methods requiring deconvolution; the “one-bead one-compound” library method; and synthetic library methods using affinity chromatography selection. The biological library approach is limited to polypeptide or peptide libraries, while the other four approaches are applicable to polypeptide, peptide, nonpeptide oligomer, or small molecule libraries of compounds. Compounds also include those that may be synthesized from leads generated by fragment-based drug design, wherein the binding of such chemical fragments is assessed by soaking or co-crystallizing such screen fragments into crystals provided by the invention and then subjecting these to an X-ray beam and obtaining diffraction data. Difference Fourier techniques are readily applied by those skilled in the art to determine the location within the RBD structure at which these fragments bind, and such fragments can then be assembled by synthetic chemistry into larger compounds with increased affinity for SARS-Corona virus or 2019-novel Corona virus. Further, compounds identified or designed using the methods of the invention can be a peptide or a mimetic thereof. The isolated peptides or mimetics of the invention may be conformationally constrained molecules or alternatively molecules which are not conformationally constrained such as, for example, non-constrained peptide sequences. The term “conformationally constrained molecules” means conformationally constrained peptides and conformationally constrained peptide analogues and derivatives. In addition, the amino acids may be replaced with a variety of uncoded or modified amino acids such as the corresponding D-amino acid or N-methyl amino acid. Other modifications include substitution of hydroxyl, thiol, amino and carboxyl functional groups with chemically similar groups. With regard to peptides and mimetics thereof, still other examples of other unnatural amino acids or chemical amino acid analogues/derivatives can be introduced as a substitution or addition. Also, a peptidomimetic may be used. A peptidomimetic is a molecule that mimics the biological activity of a peptide but is no longer peptidic in chemical nature. By strict definition, a peptidomimetic is a molecule that no longer contains any peptide bonds (that is, amide bonds between amino acids). However, the term peptide mimetic is sometimes used to describe molecules that are no longer completely peptidic in nature, such as pseudo-peptides, semi-peptides and peptoids. Whether completely or partially non-peptide, peptidomimetics for use in the invention, provide a spatial arrangement of reactive chemical moieties that closely resembles the three-dimensional arrangement of active groups in the peptide on which the peptidomimetic is based.

For instance a peptide or peptidomimetic may be designed as to mimic the 3 dimensional structure of the epitope described herein; and could possibly serve as an immunogen or vaccine, serving as an artificial antigen to present the conformational epitope to the immune system of a subject. Alternatively, a screening method is disclosed which screens for artificial peptide antigen molecules that specifically bind the ISVDs of the invention, as to produce a novel vaccine comprising said peptide, optionally presented in a suitable scaffold structure.

Typically, as a result of this similar active-site geometry, peptidomimetics has effects on biological systems which are similar to the biological activity of the peptide. There are sometimes advantages for using a mimetic of a given peptide rather than the peptide itself, because peptides commonly exhibit two undesirable properties: (1) poor bioavailability; and (2) short duration of action. Peptide mimetics offer an obvious route around these two major obstacles, since the molecules concerned are small enough to be both orally active and have a long duration of action. There are also considerable cost savings and improved patient compliance associated with peptide mimetics, since they can be administered orally compared with parenteral administration for peptides. Furthermore, peptide mimetics are generally cheaper to produce than peptides. Naturally, those skilled in the art will recognize that the design of a peptidomimetic may require slight structural alteration or adjustment of a chemical structure designed or identified using the methods of the invention. In general, chemical compounds or peptides identified or designed using the binding agents of the invention can be synthesized chemically and then tested for ability to bind and neutralize or the SARS-Corona virus or 2019-novel Corona virus, or the ISVDs of the invention, using any of the methods described herein. The peptides or peptidomimetics of the present invention can be used in assays for screening for candidate compounds which bind to selected regions or selected conformations of SARS-Corona virus or 2019-novel Corona virus. Binding can be either by covalent or non-covalent interactions, or both. Examples of non-covalent interactions include electrostatic interactions, van der Waals interactions, hydrophobic interactions and hydrophilic interactions.

### Pharmaceutical Compositions

A further aspect provides for a pharmaceutical composition comprising said binding agent or nucleic acid molecule, or recombinant vector as provided herein, optionally comprising a carrier, diluent or excipient. A “carrier”, or “adjuvant”, in particular a “pharmaceutically acceptable carrier” or “pharmaceutically acceptable adjuvant” is any suitable excipient, diluent, carrier and/or adjuvant which, by themselves, do not induce the production of antibodies harmful to the individual receiving the composition nor do they elicit protection. By “pharmaceutically acceptable” is meant a material that is not biologically or otherwise undesirable, i.e., the material may be administered to an individual along with the compound without causing any undesirable biological effects or interacting in a deleterious manner with any of the other components of the pharmaceutical composition in which it is contained. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. Preferably, a pharmaceutically acceptable carrier or adjuvant enhances the immune response elicited by an antigen. Suitable carriers or adjuvantia typically comprise one or more of the compounds included in the following non-exhaustive list: large slowly metabolized macromolecules such as proteins, polysaccharides, polylactic acids, polyglycolic acids, polymeric amino acids, amino acid copolymers and inactive virus particles. The term “excipient”, as used herein, is intended to include all substances which may be present in a pharmaceutical composition and which are not active ingredients, such as salts, binders (e.g., lactose, dextrose, sucrose, trehalose, sorbitol, mannitol), lubricants, thickeners, surface active agents, preservatives, emulsifiers, buffer substances, stabilizing agents, flavouring agents or colorants. A “diluent”, in particular a “pharmaceutically acceptable vehicle”, includes vehicles such as water, saline, physiological salt solutions, glycerol, ethanol, etc. Auxiliary substances such as wetting or emulsifying agents, pH buffering substances, preservatives may be included in such vehicles. A pharmaceutically effective amount of polypeptides, or conjugates of the invention and a pharmaceutically acceptable carrier is preferably that amount which produces a result or exerts an influence on the particular condition being treated. For therapy, the pharmaceutical composition of the invention can be administered to any patient in accordance with standard techniques. The administration can be by any appropriate mode, including orally, parenterally, topically, nasally, ophthalmically, intrathecally, intracerebroventricularly, sublingually, rectally, vaginally, and the like. Still other techniques of formulation as nanotechnology and aerosol and inhalant are also within the scope of this invention. The dosage and frequency of administration will depend on the age, sex and condition of the patient, concurrent administration of other drugs, counter-indications and other parameters to be taken into account by the clinician. The pharmaceutical composition of this invention can be lyophilized for storage and reconstituted in a suitable carrier prior to use. When prepared as lyophilization or liquid, physiologically acceptable carrier, excipient, stabilizer need to be added into the pharmaceutical composition of the invention (Remington's Pharmaceutical Sciences 22nd edition, Ed. Allen, Loyd V, Jr. (2012). The dosage and concentration of the carrier, excipient and stabilizer should be safe to the subject (human, mice and other mammals), including buffers such as phosphate, citrate, and other organic acid; antioxidant such as vitamin C, small polypeptide, protein such as serum albumin, gelatin or immunoglobulin; hydrophilic polymer such as PVP, amino acid such as amino acetate, glutamate, asparagine, arginine, lysine; glycose, disaccharide, and other carbohydrate such as glucose, mannose or dextrin, chelate agent such as EDTA, sugar alcohols such as mannitol, sorbitol; counterions such as Na+, and/or surfactant such as TWEEN™, PLURONICS™ or PEG and the like. The preparation containing pharmaceutical composition of this invention should be sterilized before injection. This procedure can be done using sterile filtration membranes before or after lyophilization and reconstitution. The pharmaceutical composition is usually filled in a container with sterile access port, such as an i.v. solution bottle with a cork.

Another aspect relates to the binding agents, nucleic acid molecules or pharmaceutical compositions of the present invention, for use as a medicine. More specifically the binding agents, nucleic acid molecules or pharmaceutical compositions of the present invention, for use in prophylaxis to prevent viral infection of a subject. Alternatively, the binding agents, nucleic acid molecules or pharmaceutical compositions of the present invention, for use in treatment of a subject with a coronavirus infection, such as patients with COVID19 disease. Specific embodiments relate to the binding agents of the invention for use to treat mammals suffering from Corona virus infection, more specifically for use in the treatment of mammals, such as humans, for the treatment 2019-novel Corona virus infection. In a specific embodiment, the binding agent nucleic acid molecules or pharmaceutical compositions of the present invention, are used for treatment of an infection with a SARS-Corona virus mutant, specifically a newly appearing Spike protein mutant, such as for instance, but not limited to the mutants at position N439, S477, E484, N501 or D614, as in SEQ ID NO:23, depicting the SARS-CoV-2 spike protein amino acid sequence.

With regards to the mutation of D to G at position 614 and S to N at position 477 for secondary structure prediction shows no changes in secondary structure while remaining in the coil region, whereas the mutation of N to Y at position 501 changes from coil structure to extended strand. N501Y mutation has a higher affinity to human ACE2 protein compared to D614G and S477N based on a docking study. D614G spike mutation was identified to exist between the two hosts based on a comparison of SARS-CoV-2 derived between the mink and human. Further research is needed on the link between the mink mutation N501T and the mutation N501Y in humans, which has evolved as a separate variant.

A further specific embodiment relates to prophylactic treatment, preferably with a single dose of the binding agent in the range of 0.5 mg/kg to 25 mg/kg. Alternatively, a therapeutic treatment with a single dose of the binding agent in the range of 0.5 mg/kg to 25 mg/kg is envisaged.

Further embodiments provide for a treatment using the binding agent or the pharmaceutical composition wherein the subject is administered via intravenous injection, subcutaneous injection, or intranasally. Alternatively inhalation and pulmonary delivery is in scope.

Another embodiment of the invention relates to a method to treatment of a subject by administering the binding agents as described herein to said subject in a therapeutically effective amount, for inhibition, prevention, and/or curing said subject of a corona virus infection. Said method of treatment may specifically relate to a prophylactic and/or therapeutic treatment of a condition resulting from infections with SARS-Corona virus.

A final aspect relates to the use of the binding agent described herein in a detection method of for detecting a viral particle or the Spike protein by binding to the binding site of the RBD of said viral Spike protein as described herein. Said method maybe an in vitro method, or alternatively the use of a sample of a subject comprising the viral protein or particle. Analyzing a sample may be done using a labelled variant of the binding agent as described herein, said label may be a detectable label, and/or a tag. So with a label or tag, as used herein, it is referred herein to detectable labels or tags allowing the detection and/or quantification of the viral particle or protein or binding agent as described herein, and is meant to include any labels/tags known in the art for these purposes. Particularly preferred, but not limiting, are affinity tags, such as chitin binding protein (CBP), maltose binding protein (MBP), glutathione-S-transferase (GST), poly(His) (e.g., 6×His or His6), biotin or streptavidin, such as Strep-tag®, Strep-tag II® and Twin-Strep-tag®; solubilizing tags, such as thioredoxin (TRX), poly(NANP) and SUMO; chromatography tags, such as a FLAG-tag; epitope tags, such as V5-tag, myc-tag and HA-tag; fluorescent labels or tags (i.e., fluorochromes/-phores), such as fluorescent proteins (e.g., GFP, YFP, RFP etc.) and fluorescent dyes (e.g., FITC, TRITC, coumarin and cyanine); luminescent labels or tags, such as luciferase, bioluminescent or chemiluminescent compounds (such as luminal, isoluminol, theromatic acridinium ester, imidazole, acridinium salts, oxalate ester, dioxetane or GFP and its analogs); phosphorescent labels; a metal chelator; and (other) enzymatic labels (e.g., peroxidase, alkaline phosphatase, beta-galactosidase, urease or glucose oxidase); radioisotopes. Also included are combinations of any of the foregoing labels or tags. Technologies for generating labelled polypeptides and proteins are well known in the art. A binding agent comprising the ISVD-containing binder of the invention, coupled to, or further comprising a label or tag allows for instance immune-based detection of said bound viral particle. Immune-based detection is well known in the art and can be achieved through the application of numerous approaches. These methods are generally based upon the detection of a label or marker, such as described above. See, for example, U.S. Pat. Nos. 3,817,837; 3,850,752; 3,939,350; 3,996,345; 4,277,437; 4,275,149 and 4,366,241. In the case where multiple antibodies are reacted with a single array, each antibody can be labelled with a distinct label or tag for simultaneous detection. Yet another embodiment may comprise the introduction of one or more detectable labels or other signal-generating groups or moieties, or tags, depending on the intended use of the labelled or tagged binding agent of the present invention. Other suitable labels will be clear to the skilled person, and for example include moieties that can be detected using NMR or ESR spectroscopy. Such labelled ISVD-based binding agents as disclosed herein may for example be used for in vitro, in vivo or in situ assays (including immunoassays known per se such as ELISA, RIA, EIA and other “sandwich assays”, etc.) as well as in vivo imaging purposes, depending on the choice of the specific label.

A specific embodiment discloses the use of the binding agent, optionally in a labelled form, for detection of a virus or Spike protein of said virus, wherein said virus is selected from the group of clade 1a, 1b, 2 and/or clade 3 bat SARS-related sarbecovirsues, such as SARS-Cov-2, GD-Pangolin, RaTG13, WIV1, LYRa11, RsSHC014, Rs7327, SARS-CoV-1, Rs4231, Rs4084, Rp3, HKU3-1, or BM48-31 viruses.

In another alternative aspect of the invention, any of the binding agents described herein, optionally with a label, or any of the nucleic acid molecules encoding said agent, or any of the compositions, or vectors as described herein may as well be used as a diagnostic, or in detection of a corona virus, as described herein. Diagnostic methods are known to the skilled person and may involve biological samples from a subject. Also in vitro methods may be in scope for detection of viral protein or particles using the binding agents as described herein. Finally, the binding agents as described herein, optionally labelled, may also be suitable for use in in vivo imaging.

It is to be understood that although particular embodiments, specific configurations as well as materials and/or molecules, have been discussed herein for methods, samples and biomarker products according to the disclosure, various changes or modifications in form and detail may be made without departing from the scope of this invention. The following examples are provided to better illustrate particular embodiments, and they should not be considered limiting the application. The application is limited only by the claims.

### EXAMPLES

### Example 1. Isolation of SARS VHH-72

A llama was immunized subcutaneously two times with SARS-CoV S protein, two times with MERS-CoV S protein, a 5^{th} time with SARS-CoV S protein and a 6^{th} time with both SARS-CoV and MERS-CoV S protein. The recombinant S proteins were stabilized in the prefusion conformation⁵². After the immunization, peripheral blood lymphocytes were isolated from the llama and an immune VHH-displaying phagemid library of approximately 3×10⁸ clones was constructed. SARS CoV S-specific VHHs were selected by 2 rounds of bio-panning of the recombinant phages on purified recombinant foldon containing SARS CoS that was immobilized to a well of a microtiter plate using an anti-foldon monoclonal antibody. Foldon-specific phages were removed by prior panning of the phage library on human respiratory syncytial virus-derived DS-Cav1 containing a C-terminal foldon⁵³. Next periplasmic extracts were prepared from individual phagemid clones obtained after the panning and the specificity of the VHHs in these extracts was evaluated in a SARS CoV S protein binding by ELISA. One of the selected VHH displayed strong binding to the SARS CoV S protein that was retained for further analysis was named herein SARS VHH-72. The sequence of SARS VHH-72 is depicted in SEQ ID NO: 1.

### Example 2. Binding of SARS VHH-72 to SARS CoV S, SARS CoV RBD, WIV1 CoV RBD and 2019-nCoV RBD

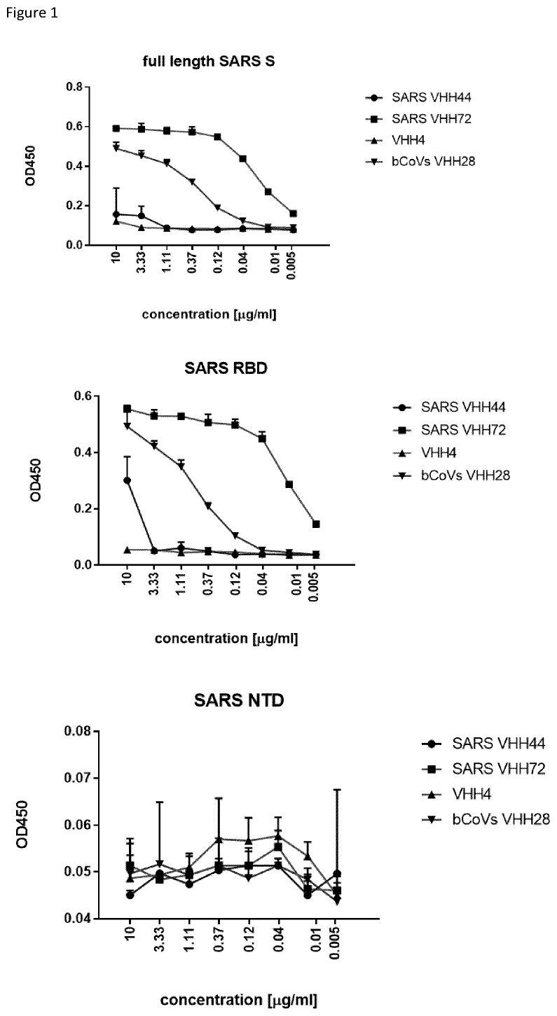

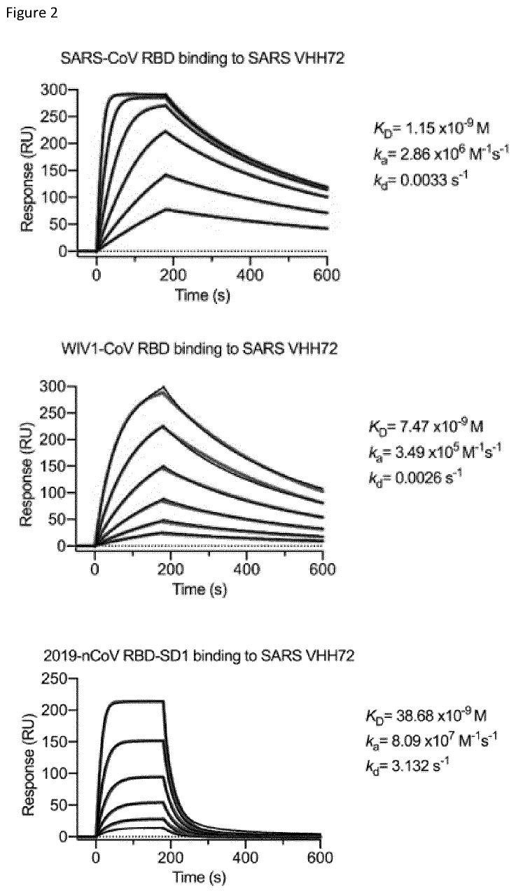

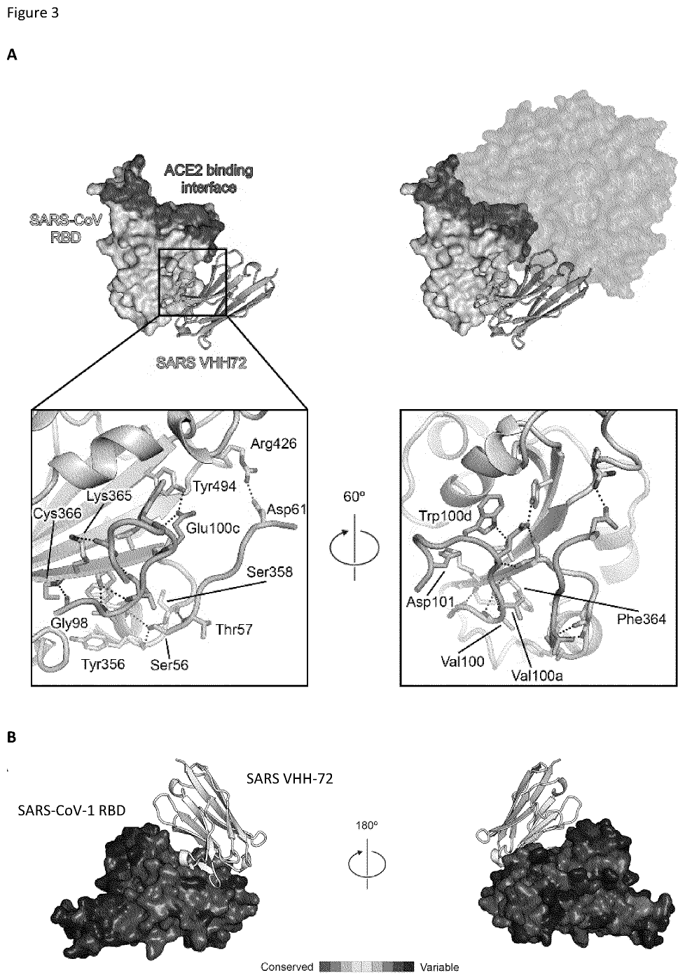

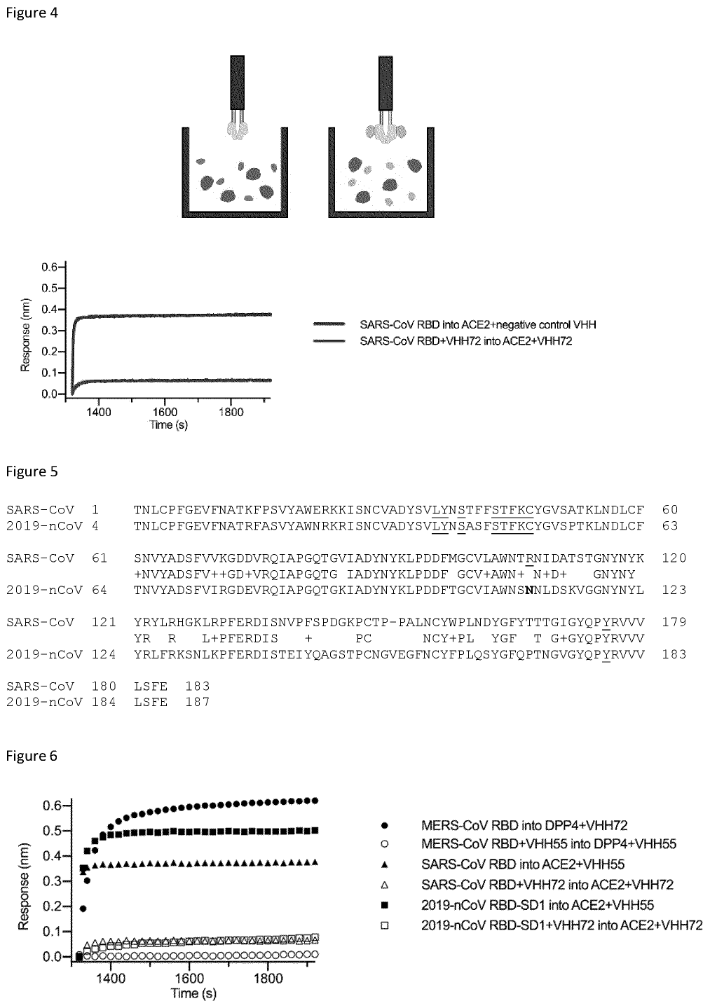

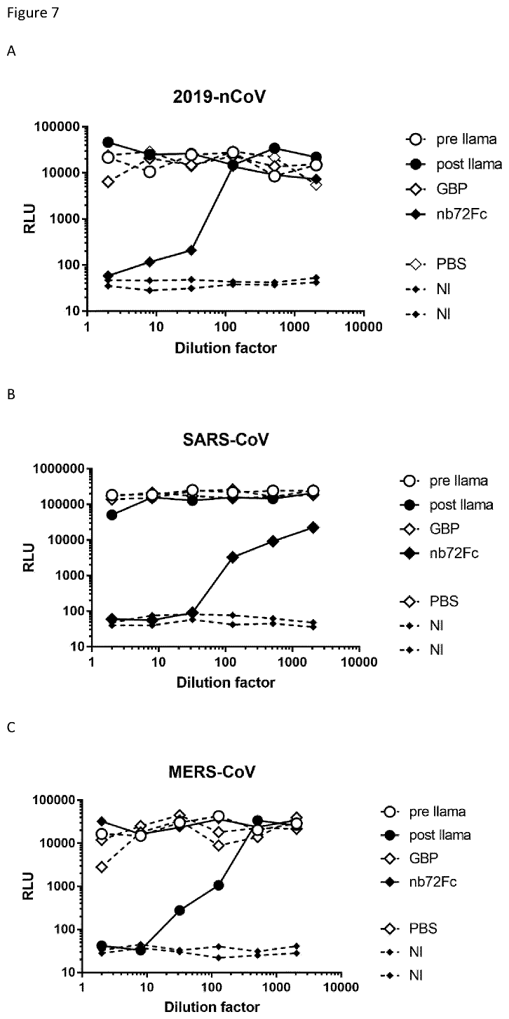

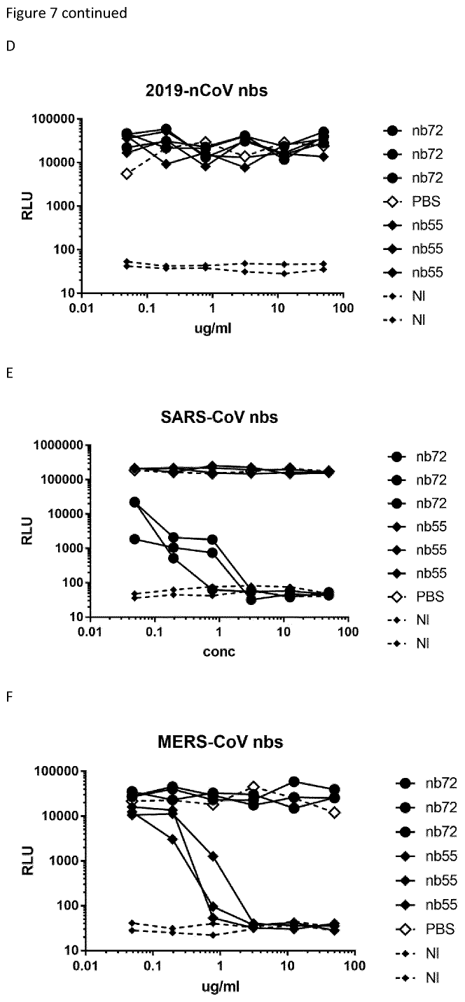

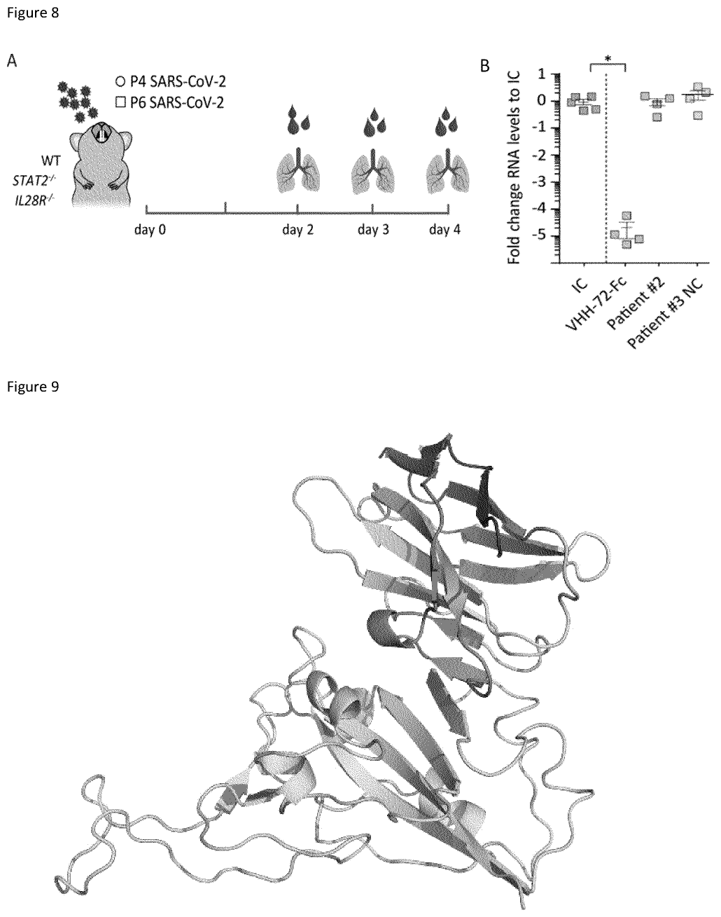

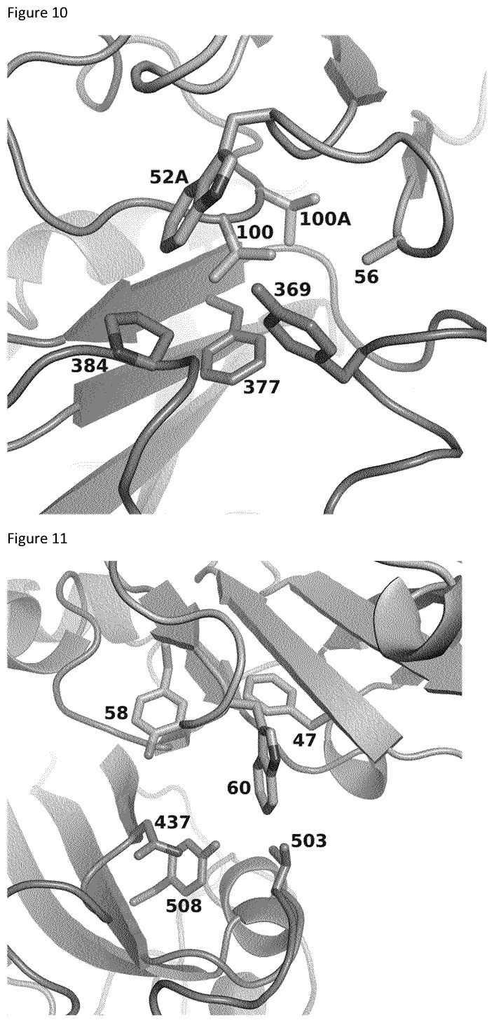

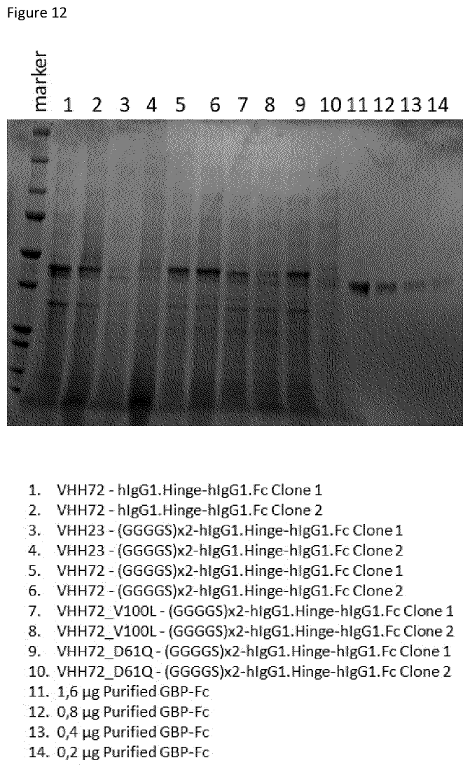

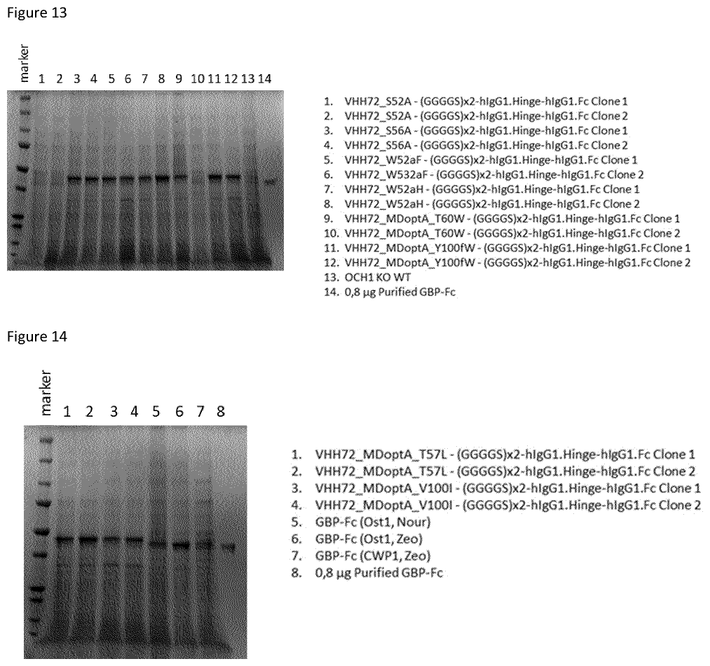

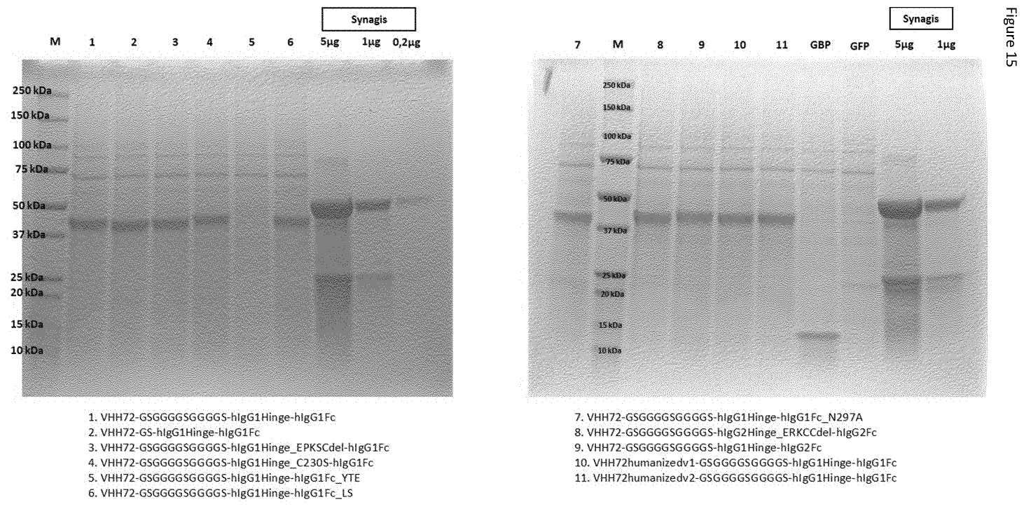

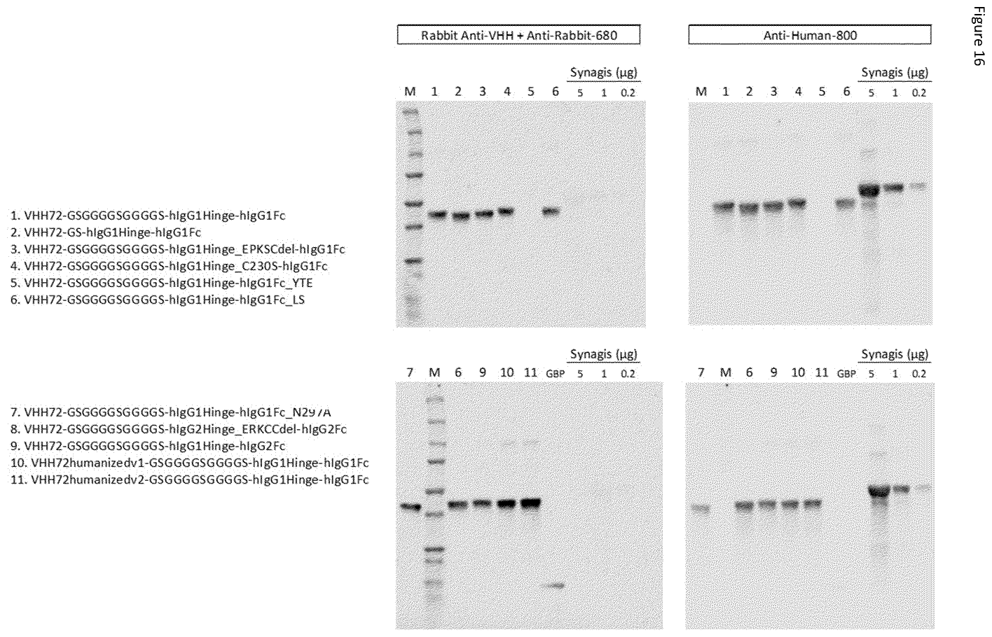

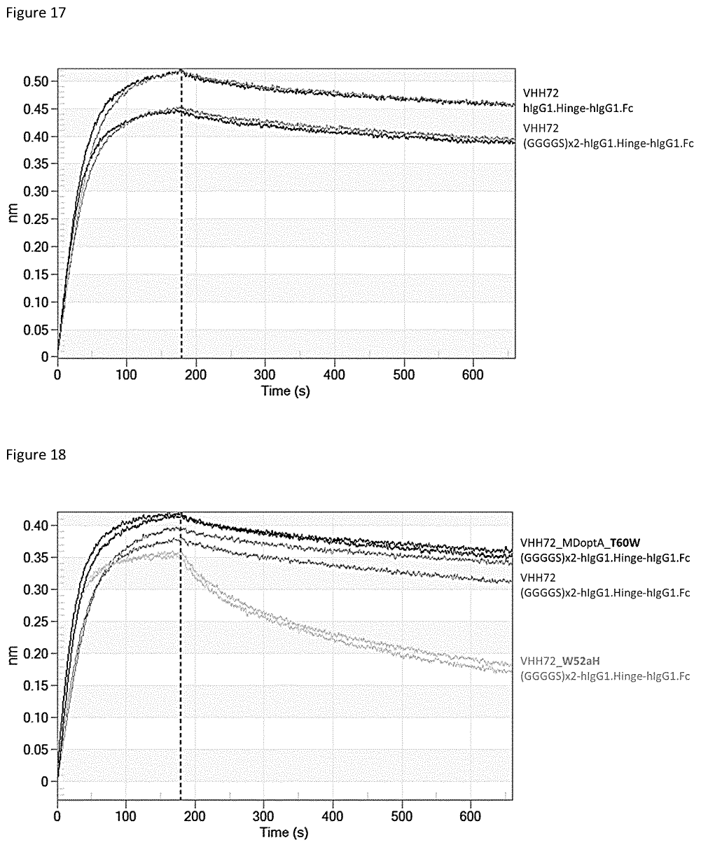

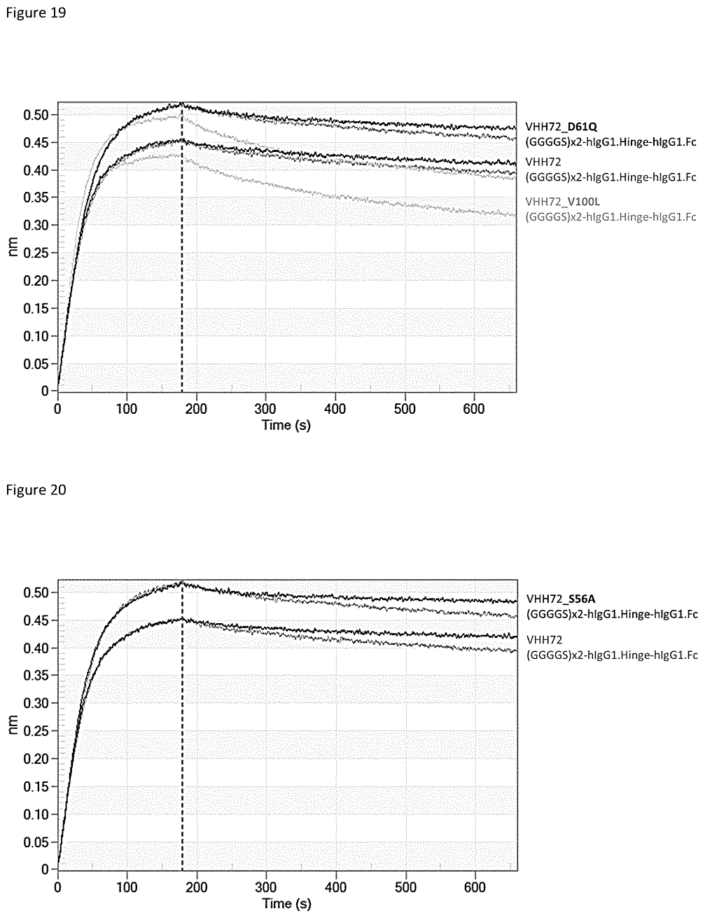

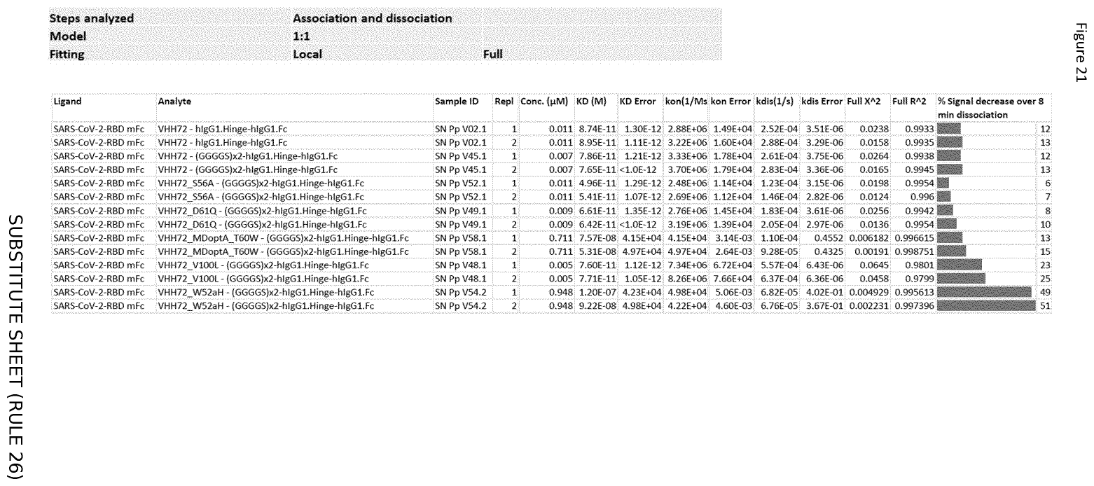

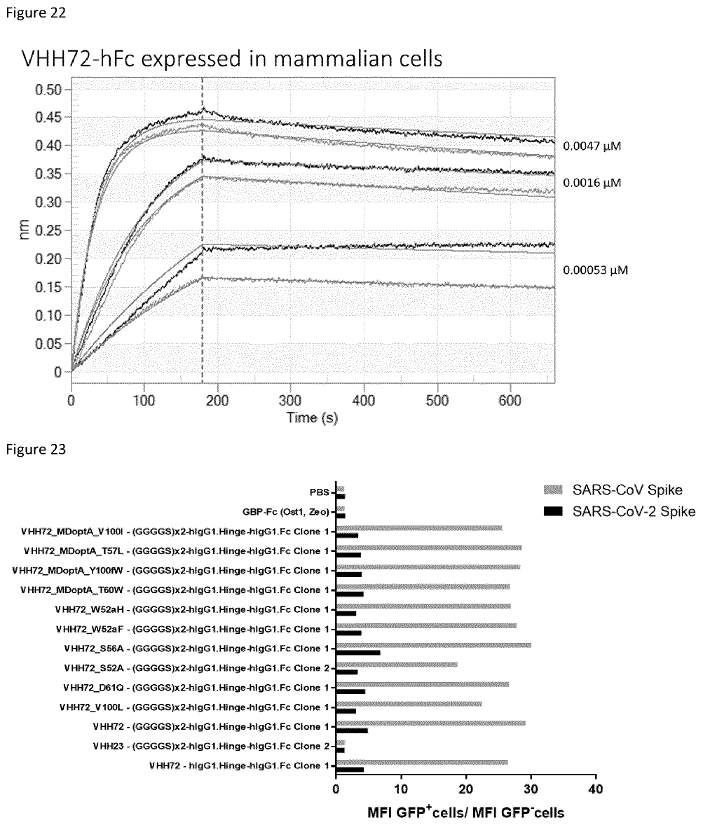

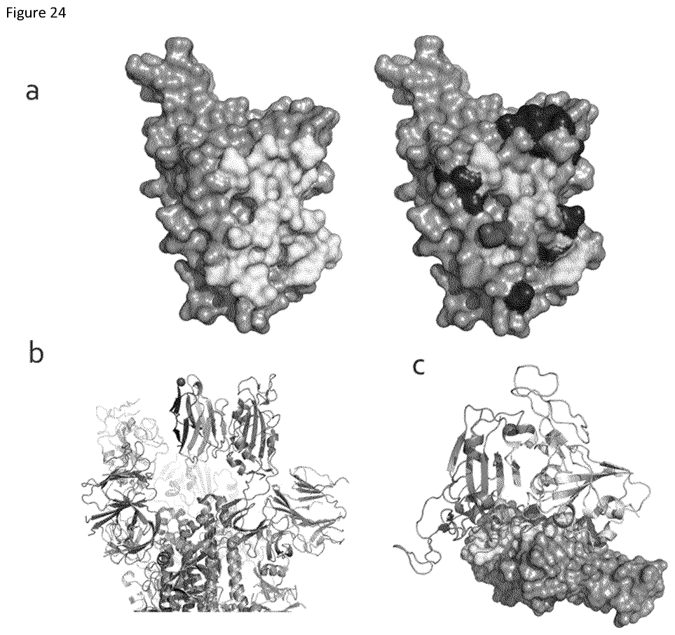

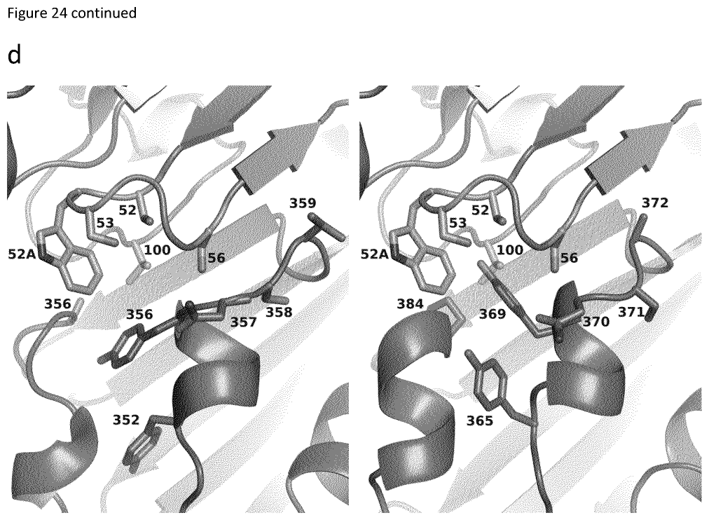

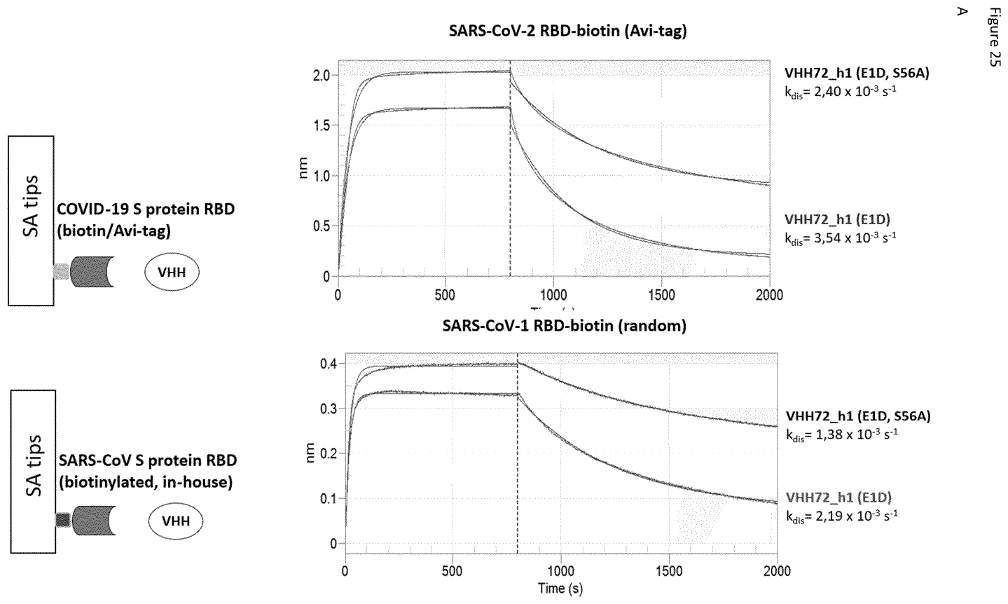

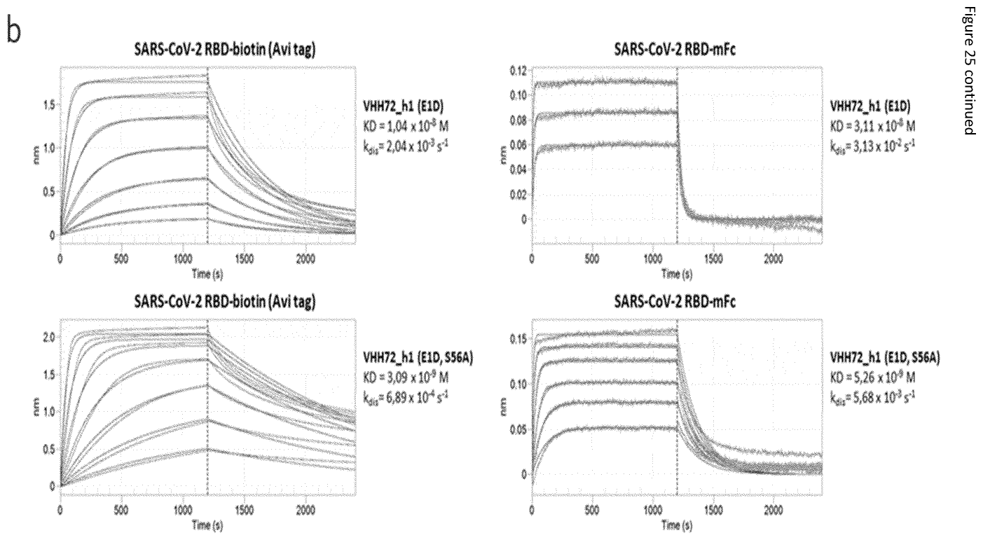

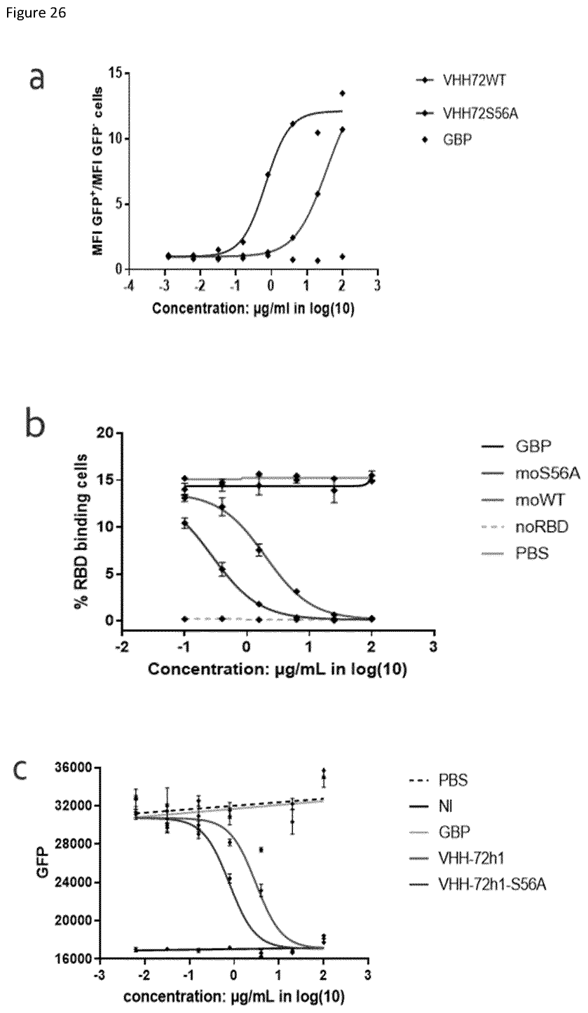

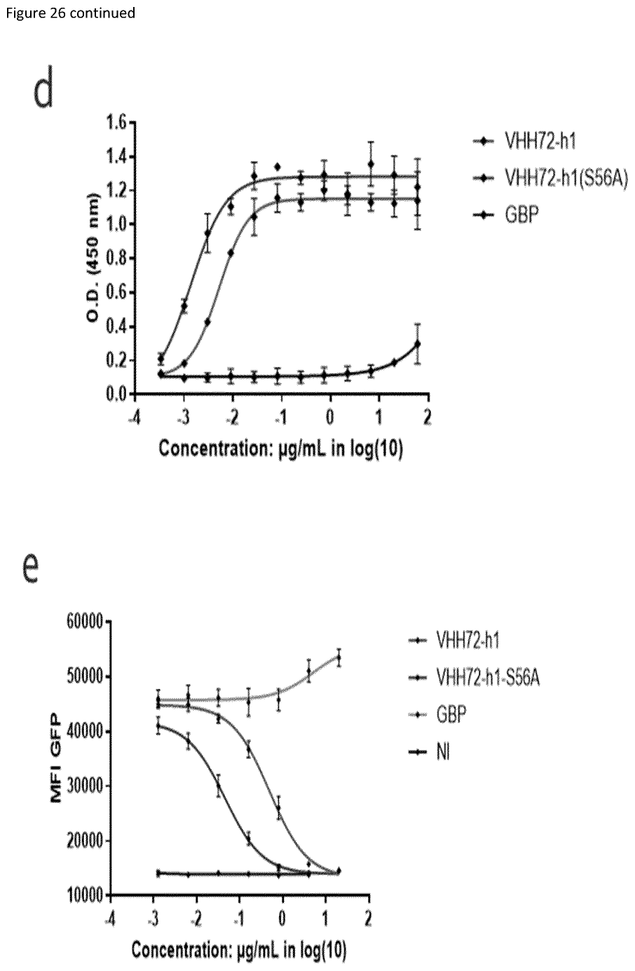

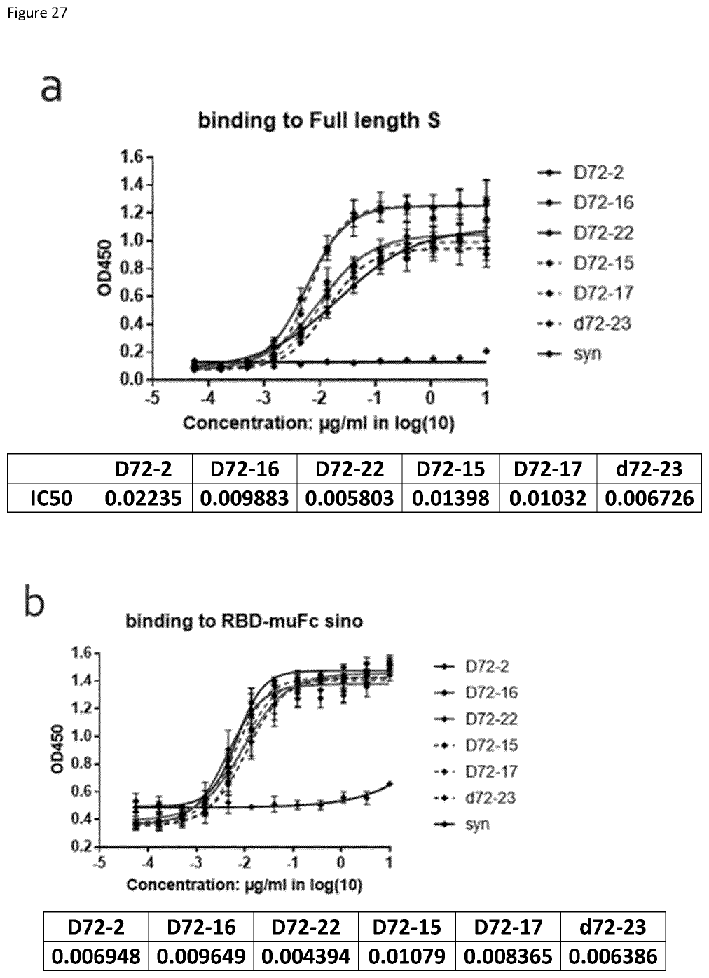

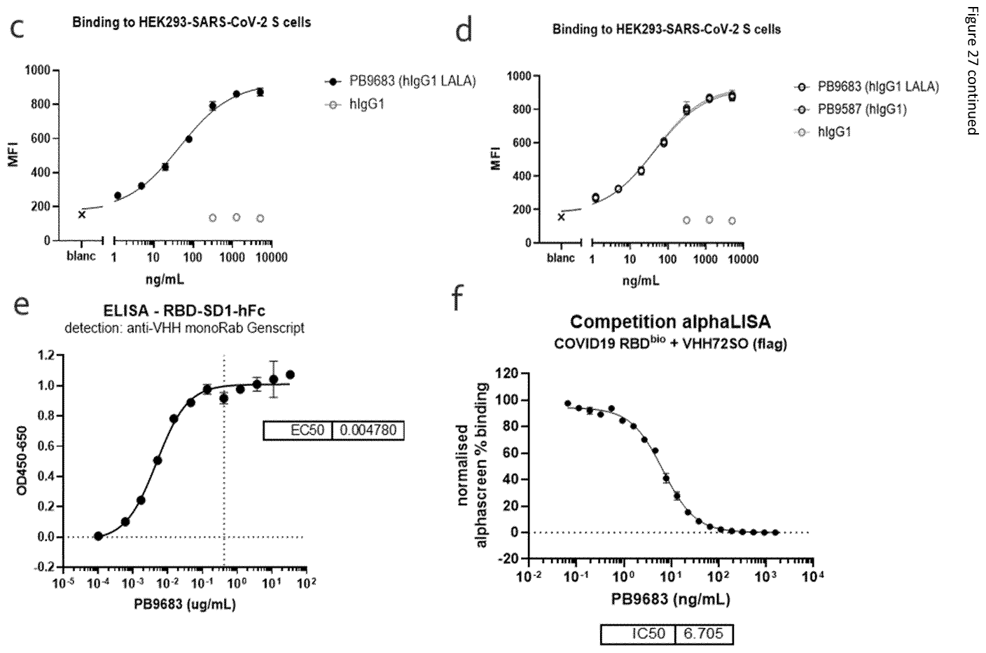

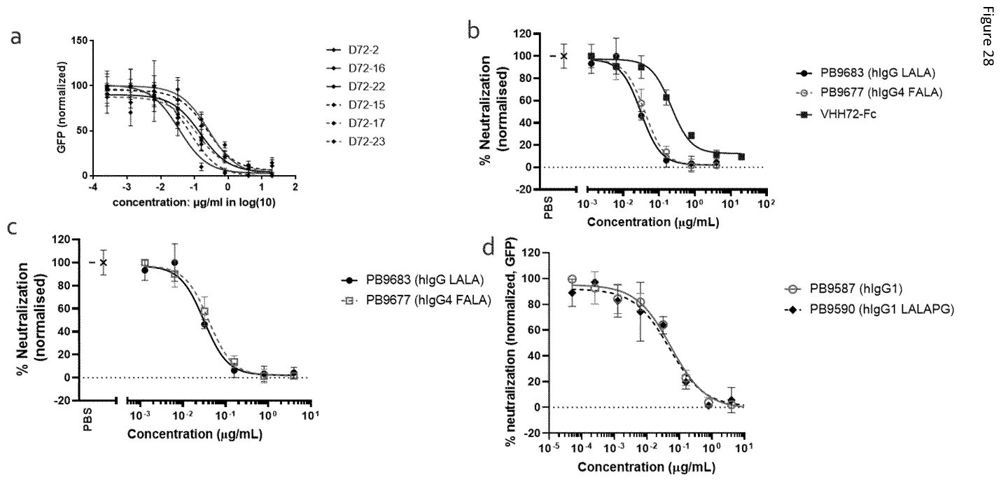

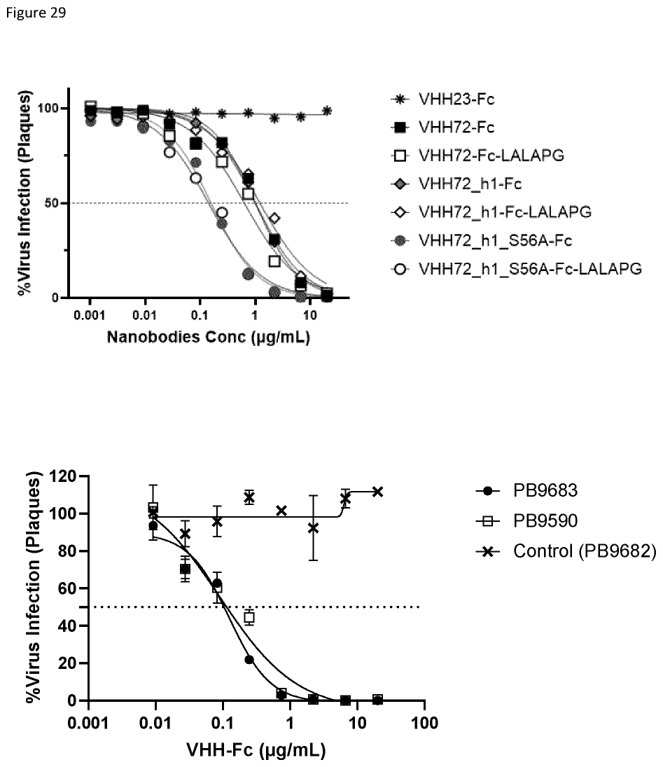

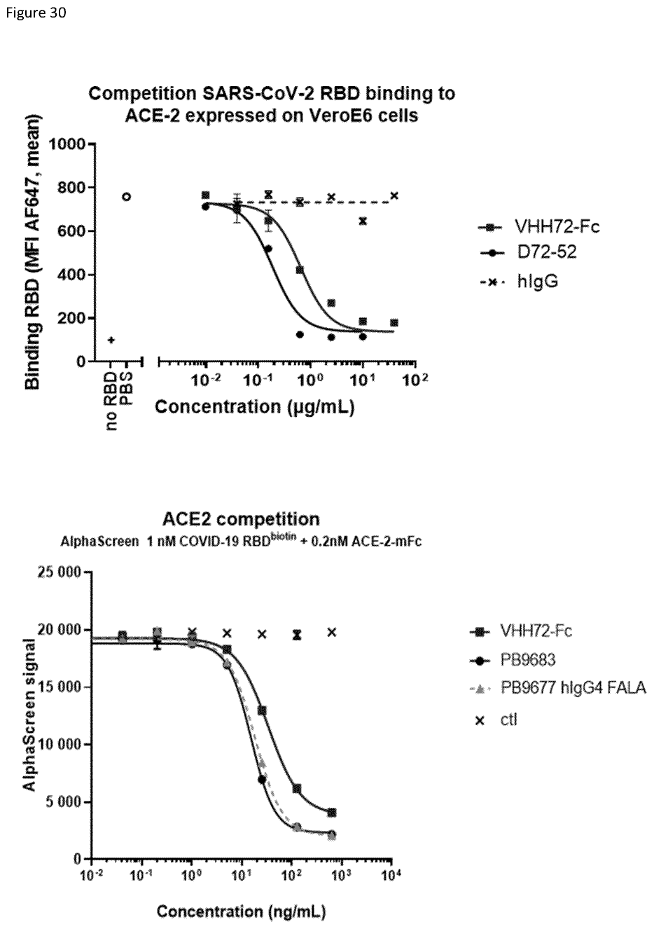

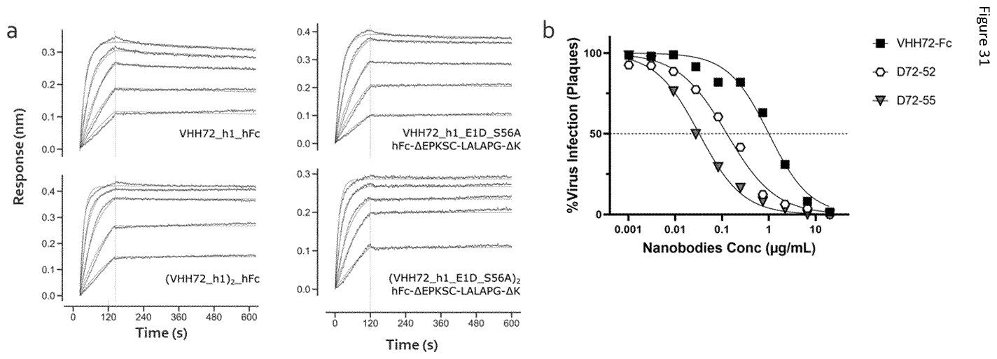

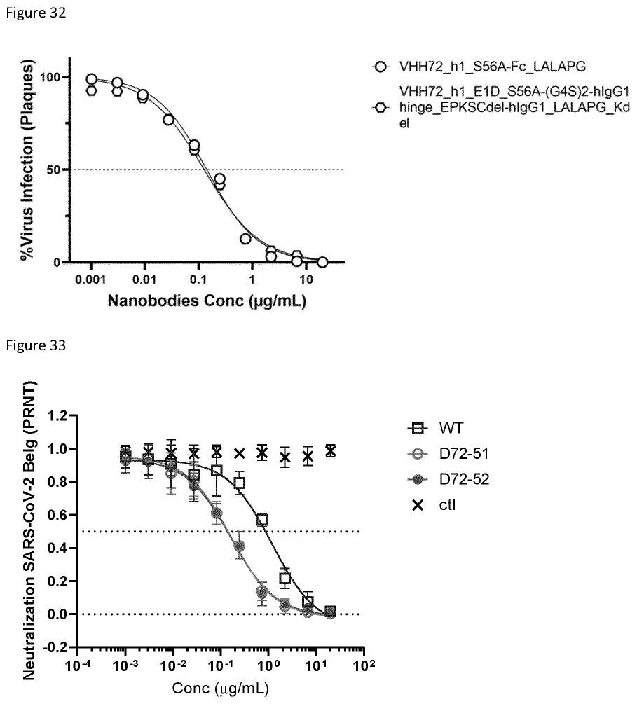

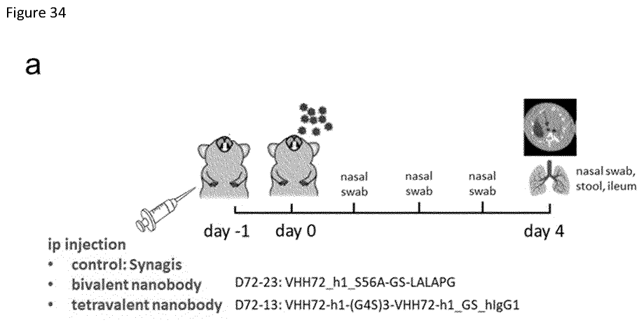

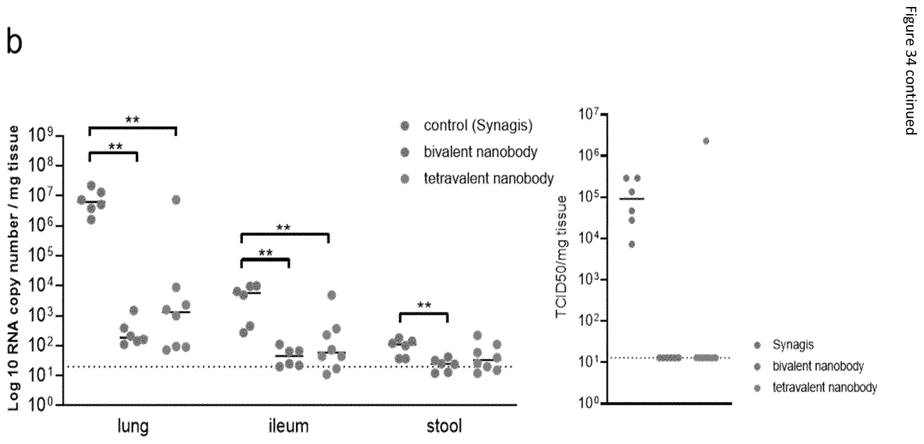

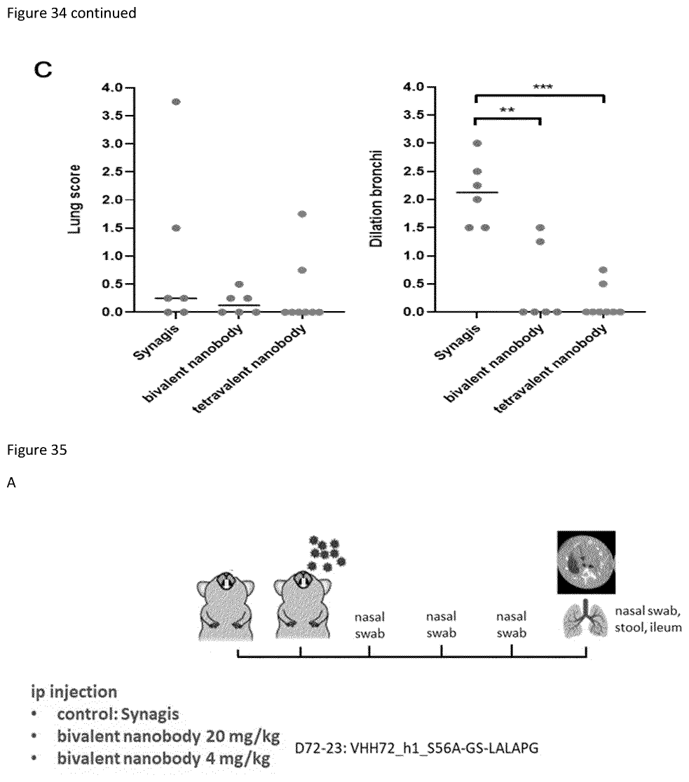

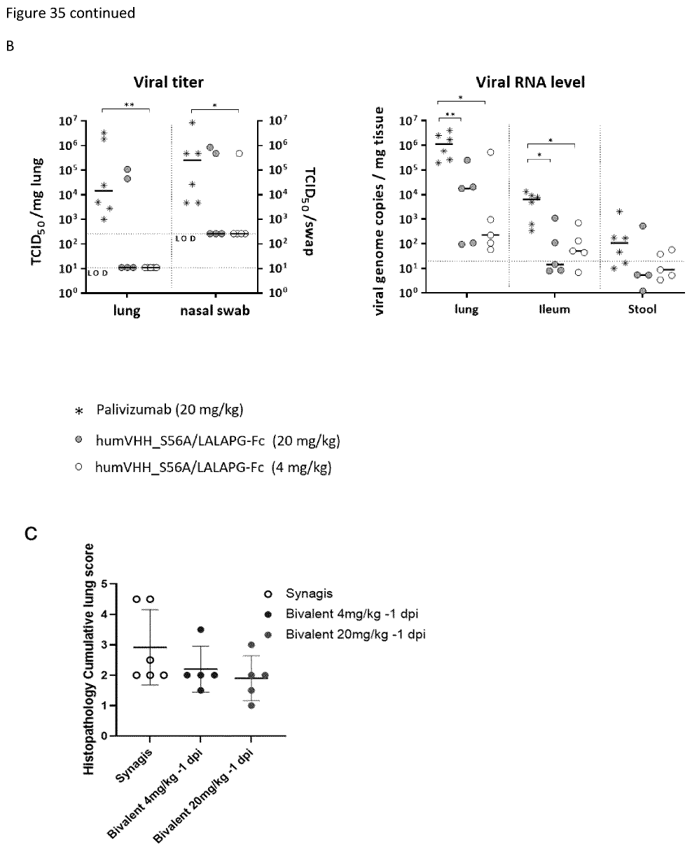

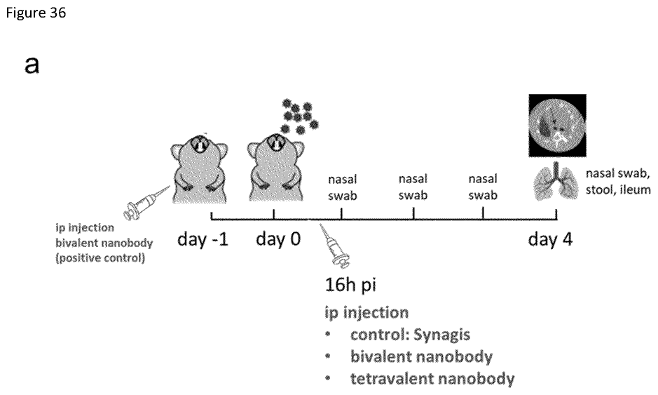

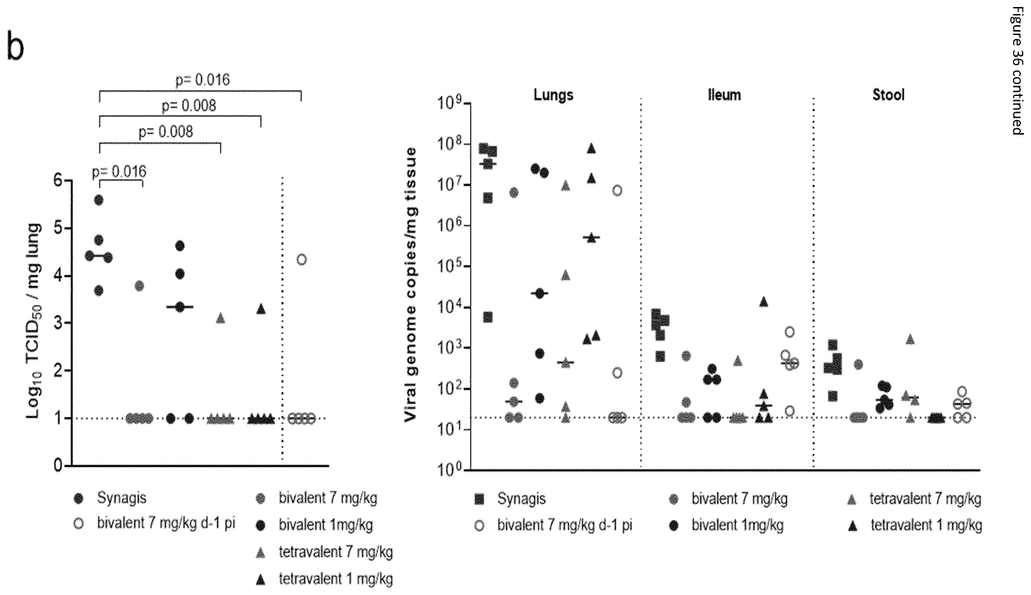

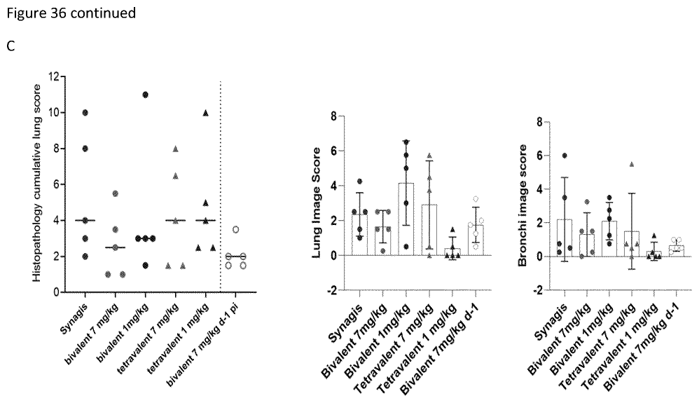

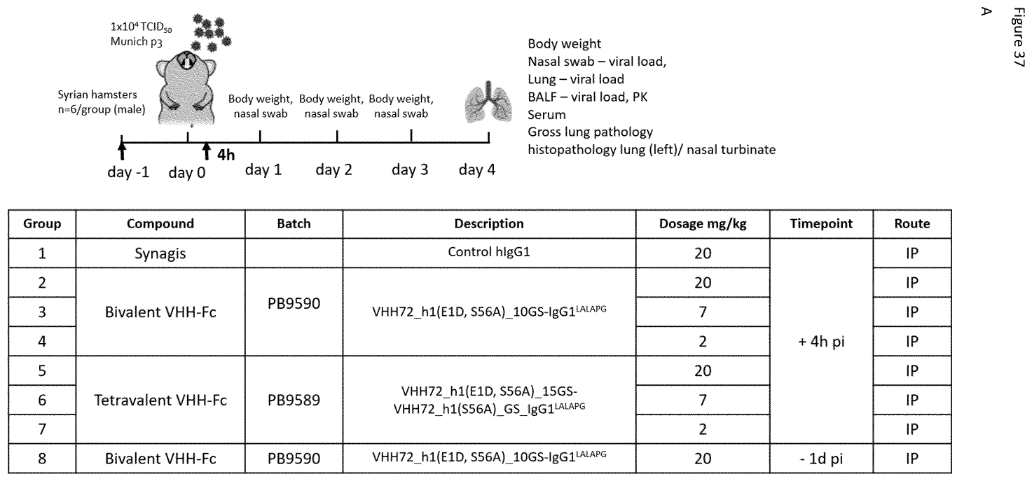

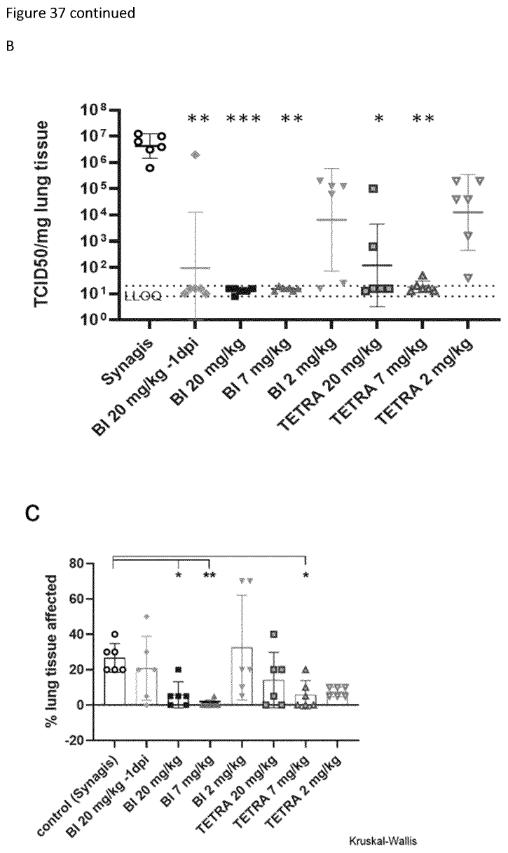

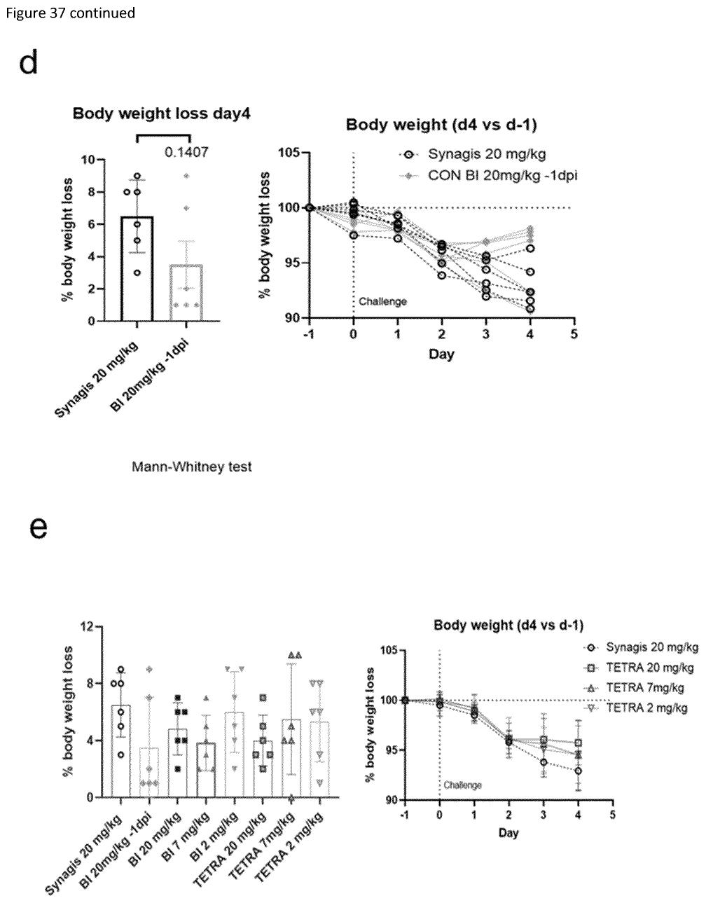

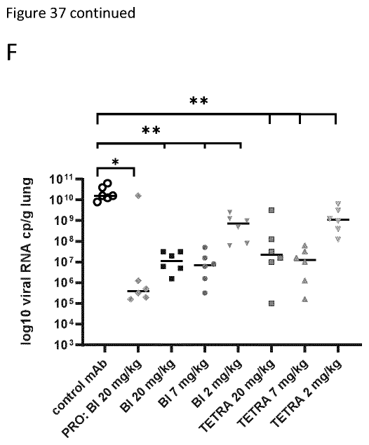

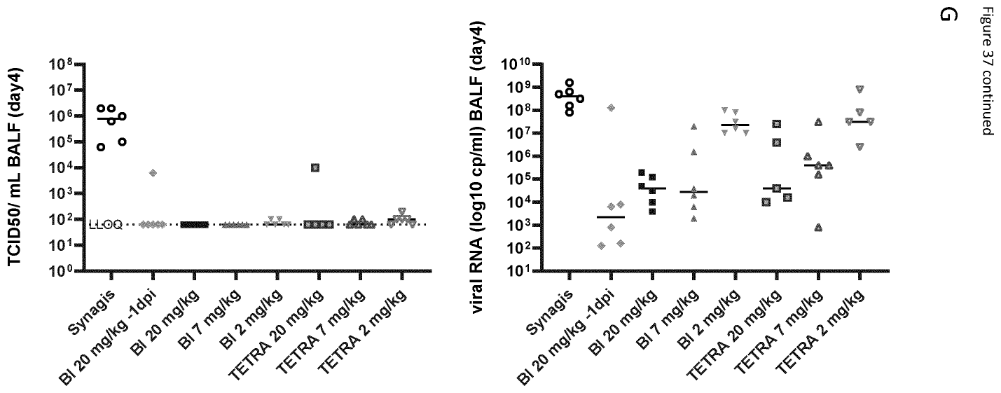

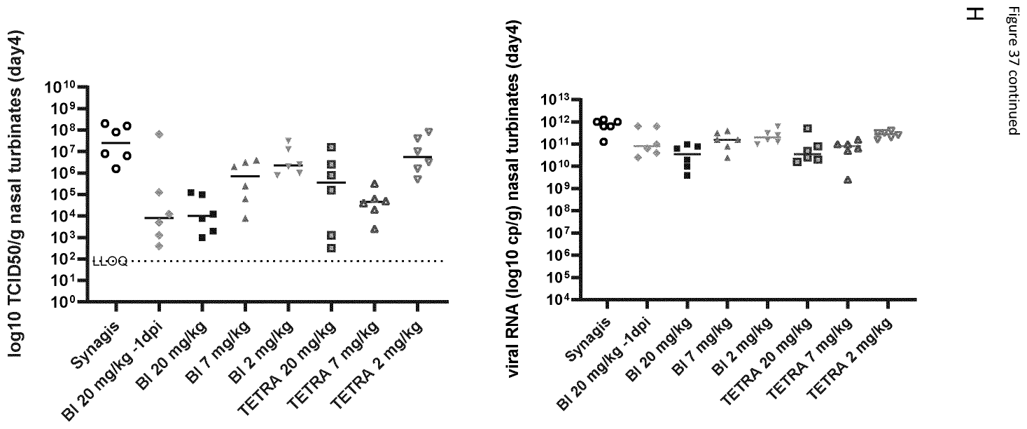

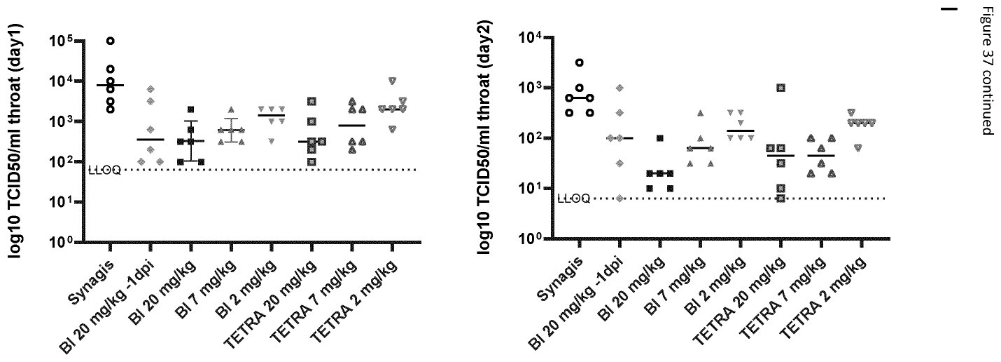

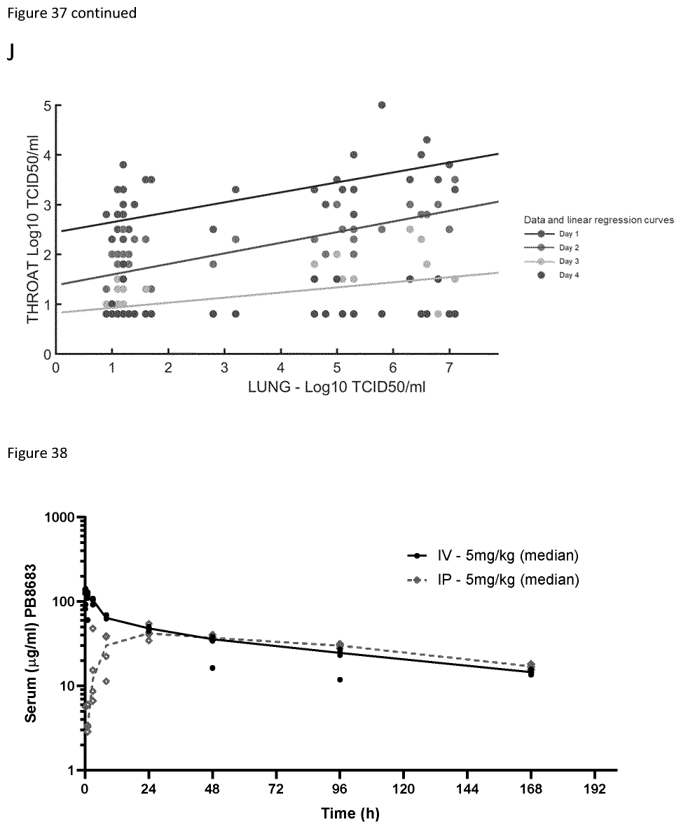

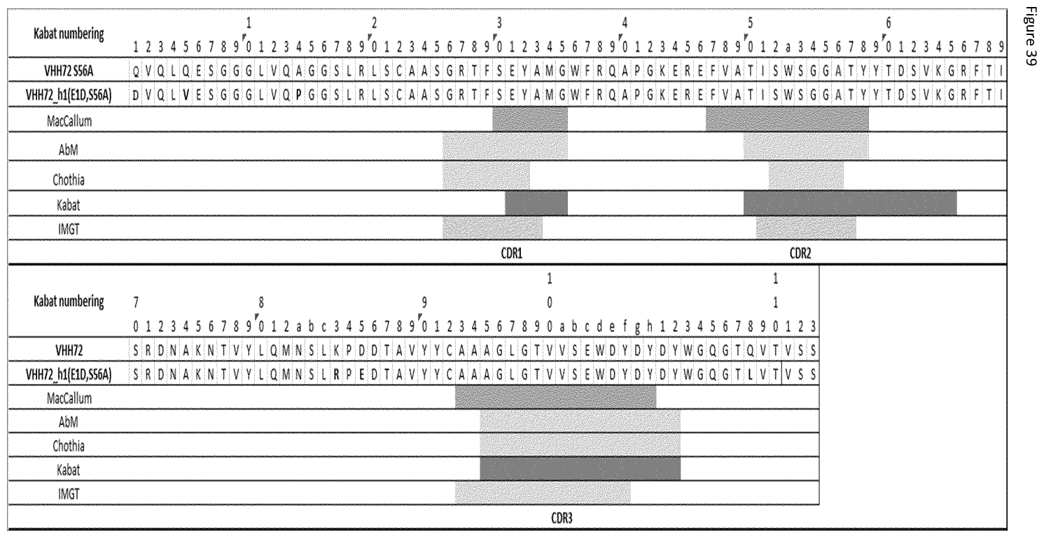

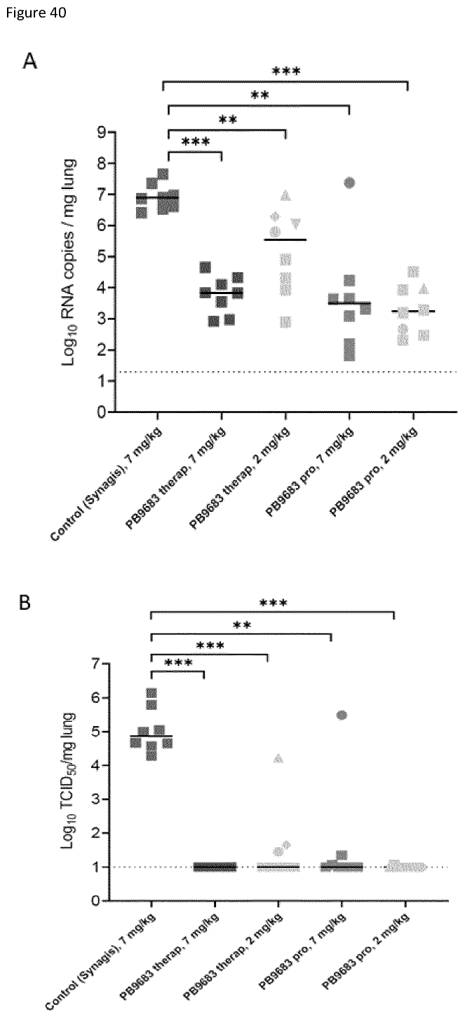

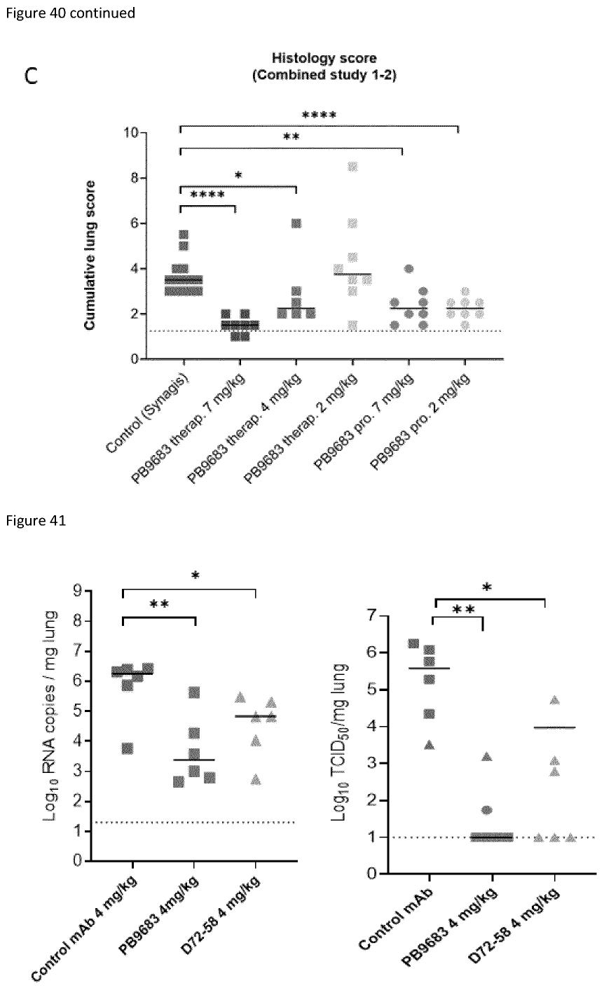

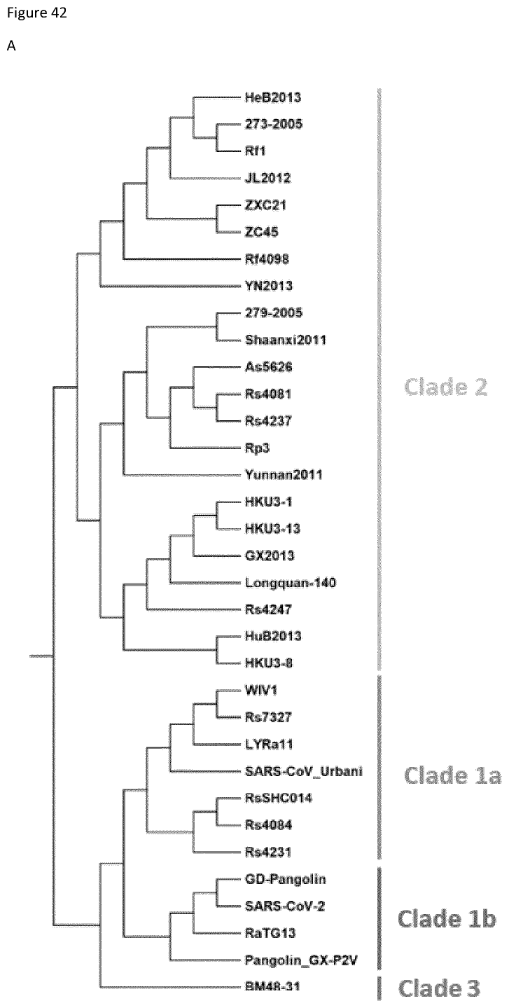

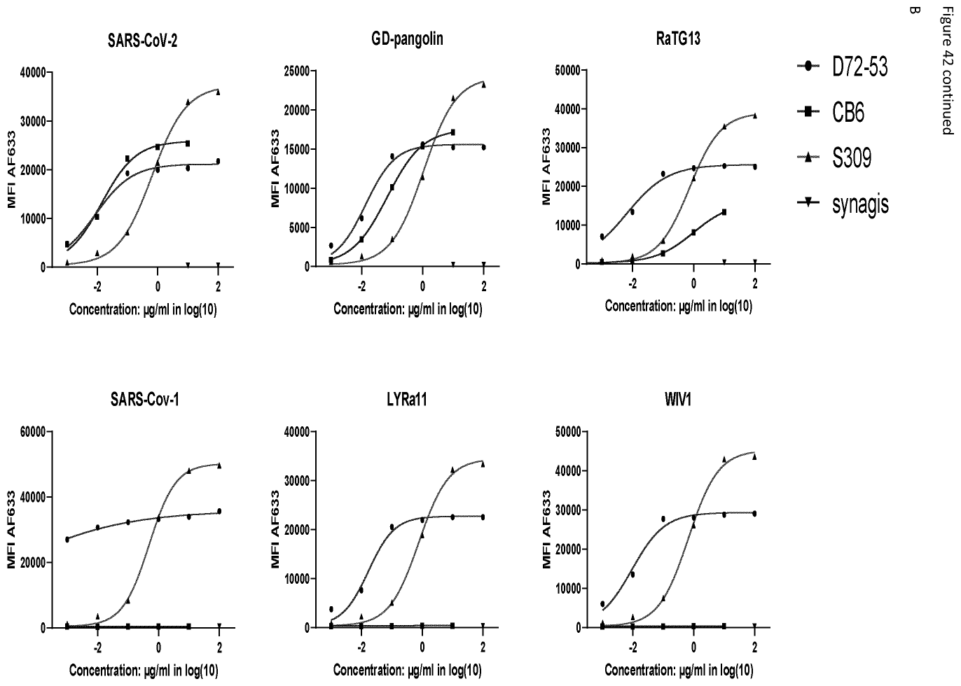

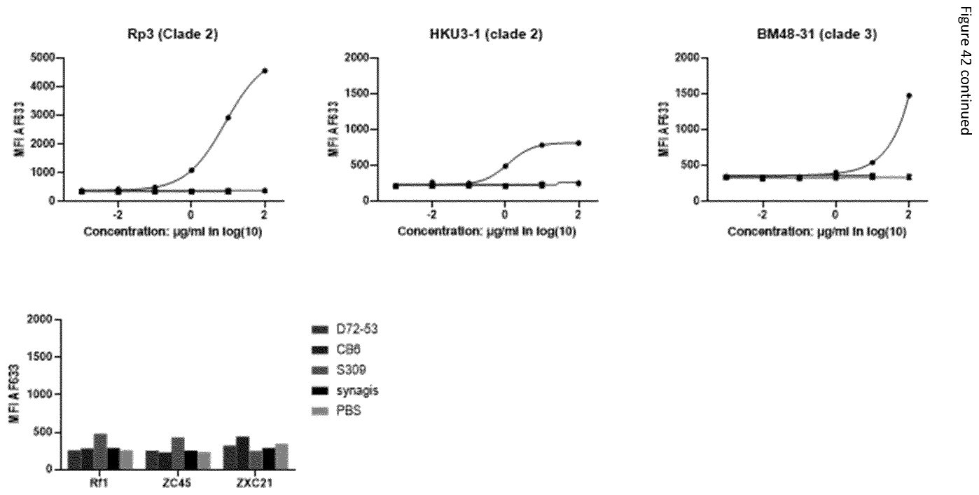

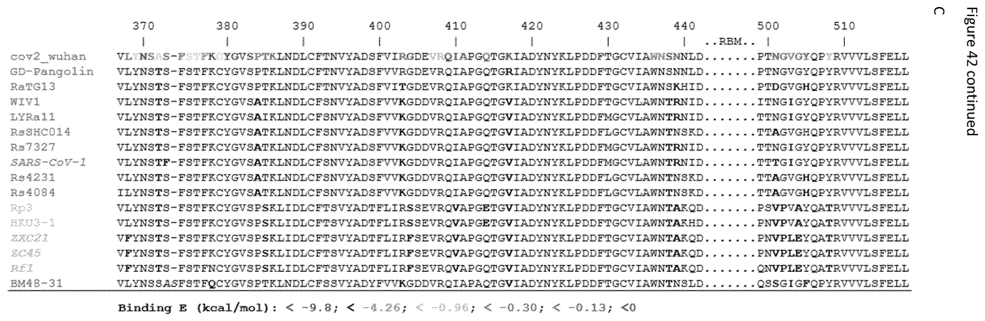

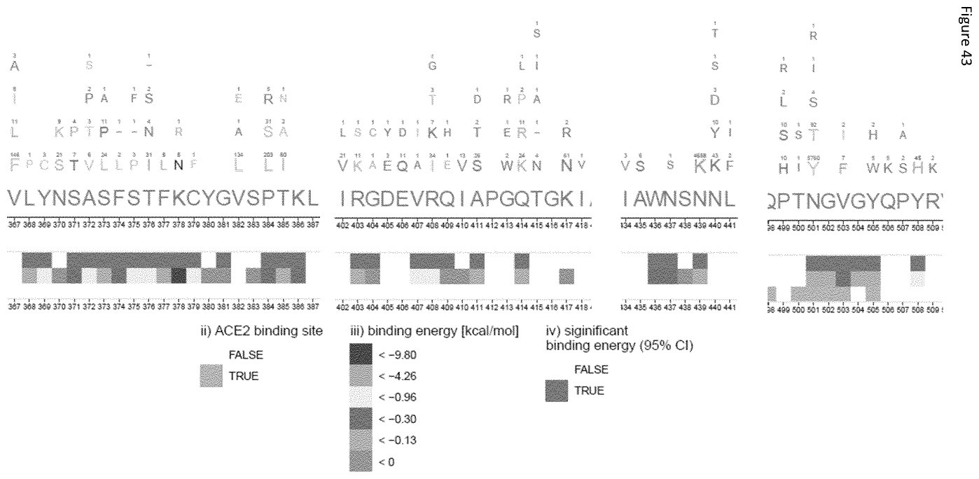

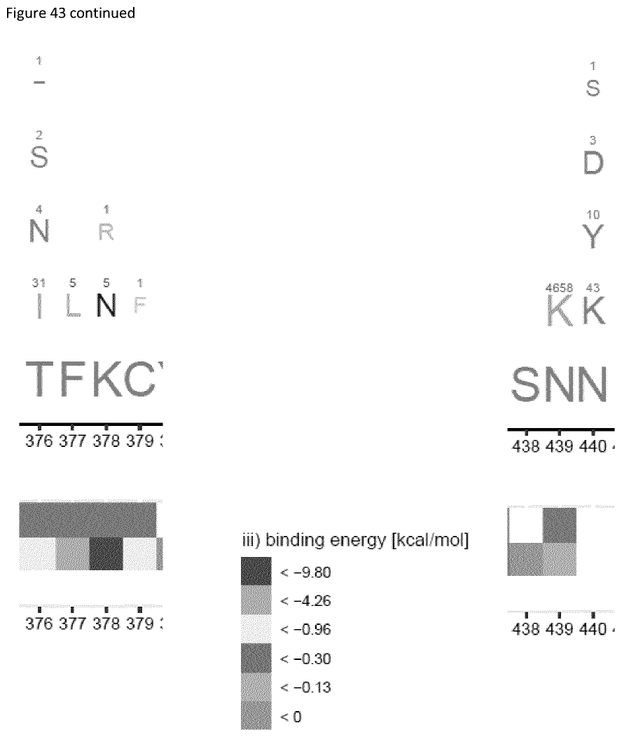

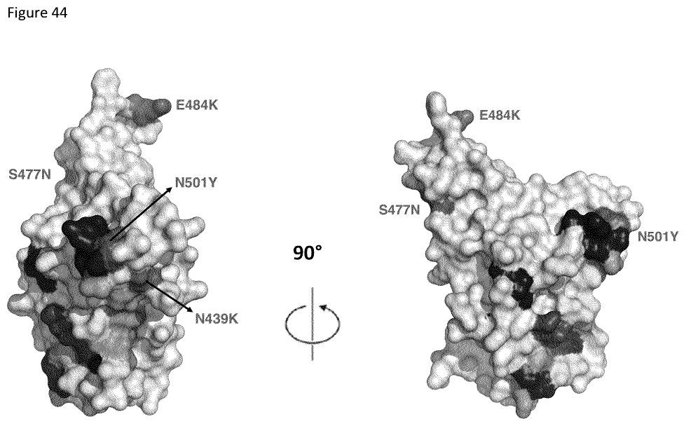

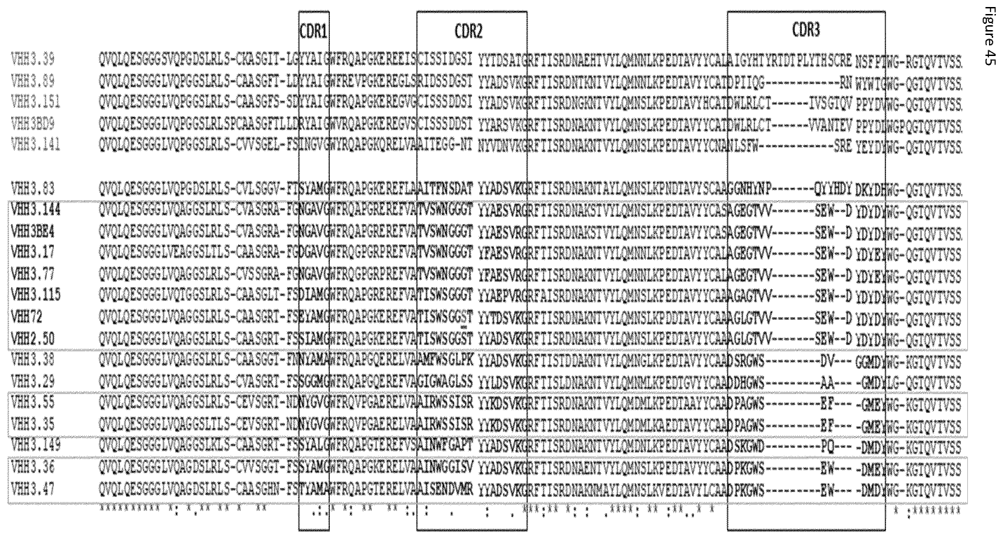

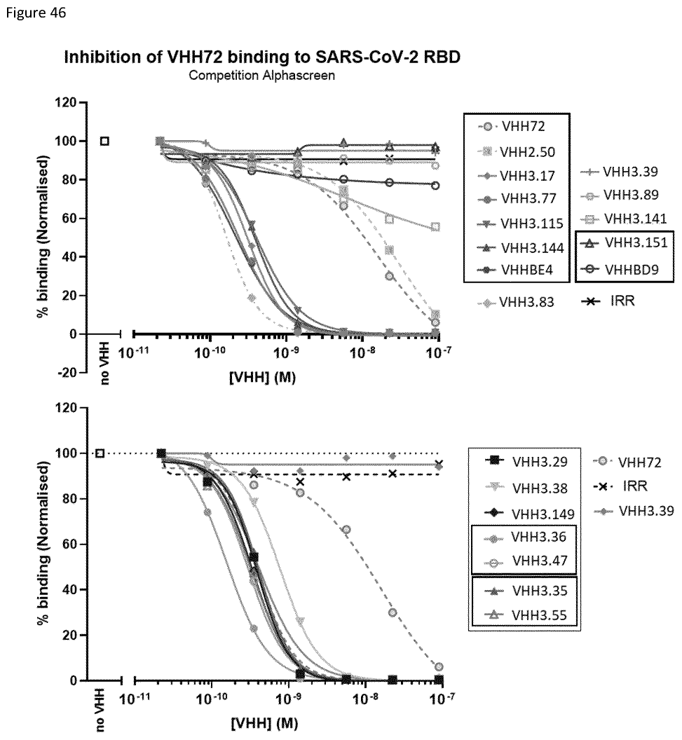

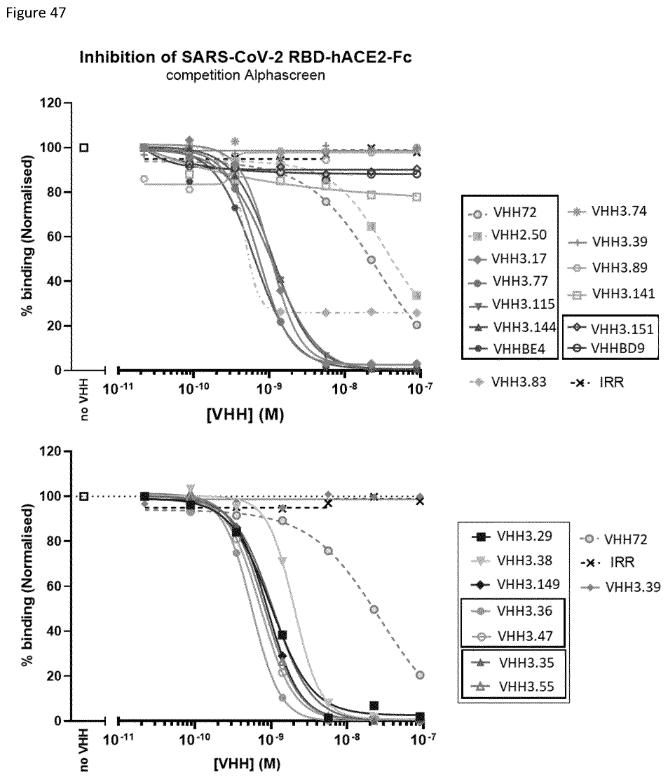

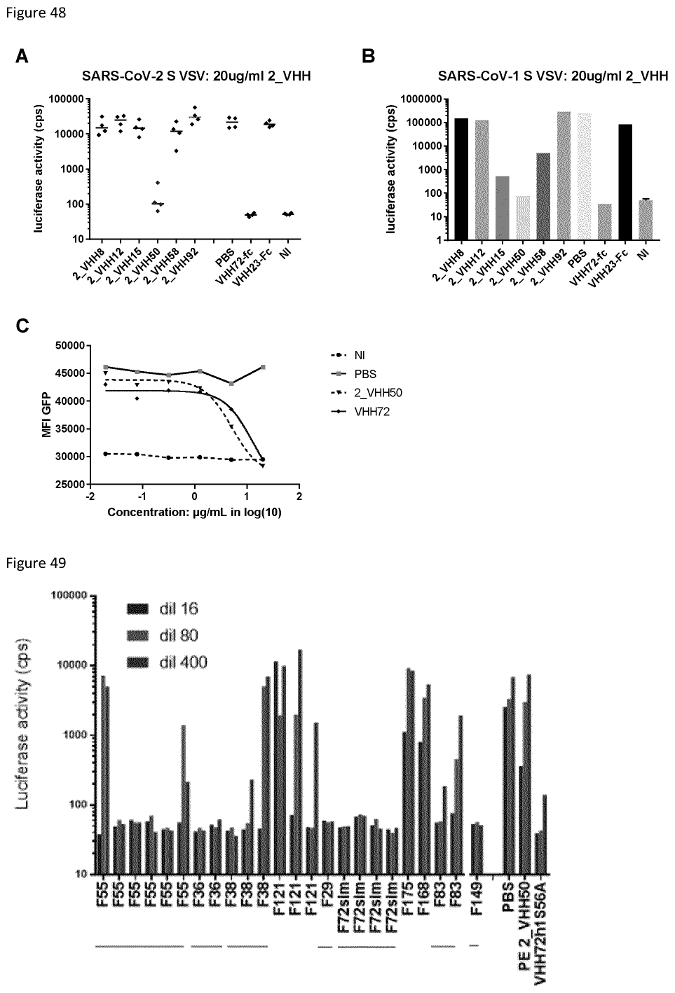

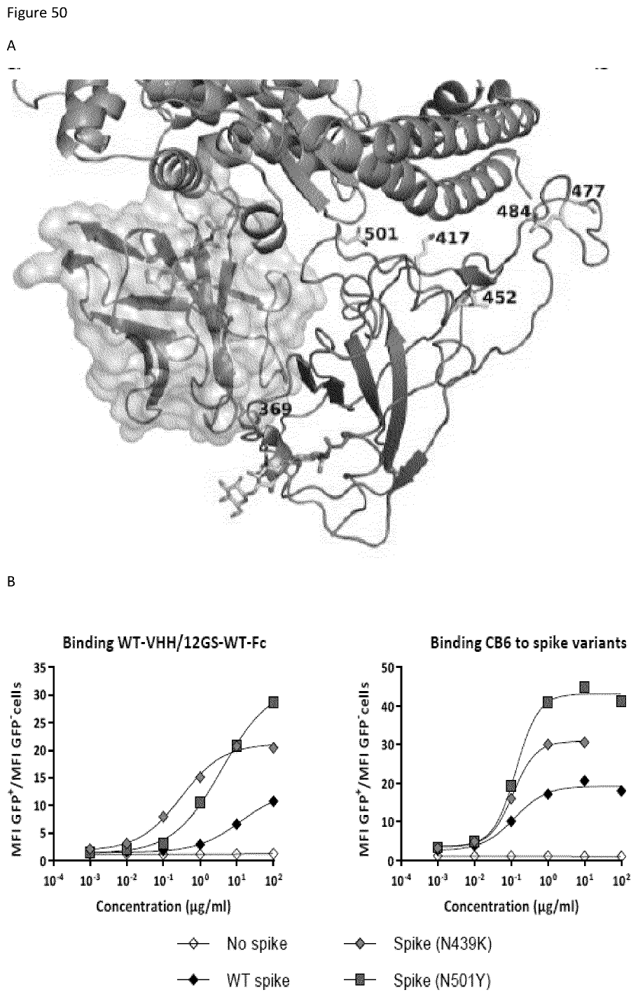

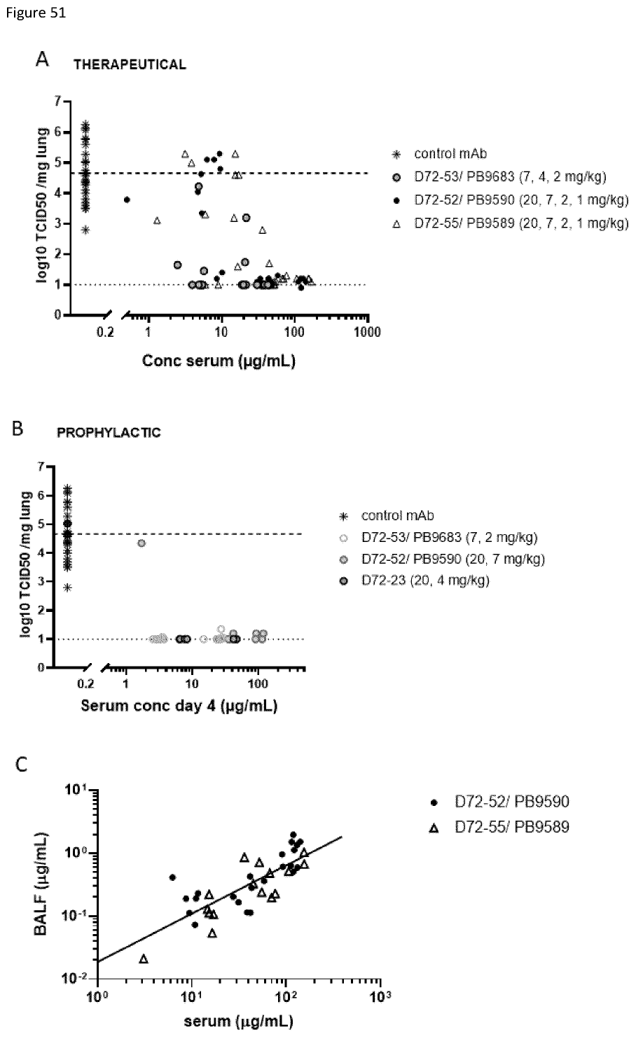

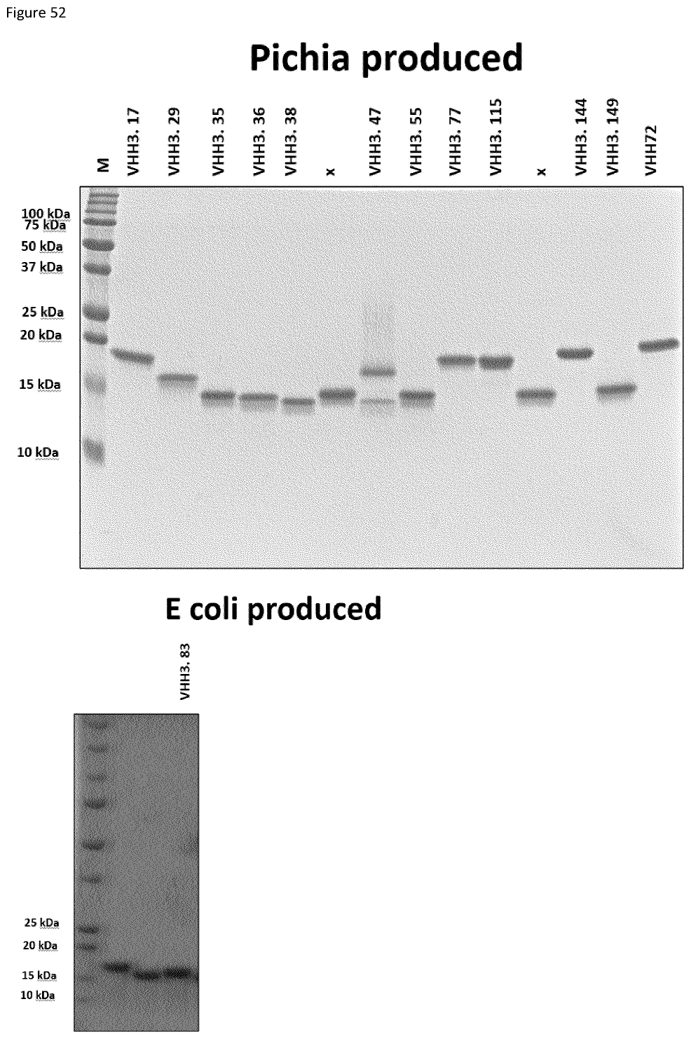

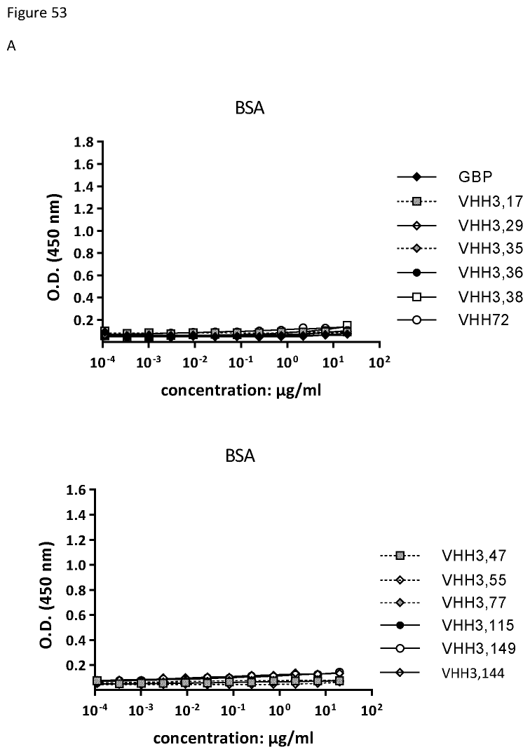

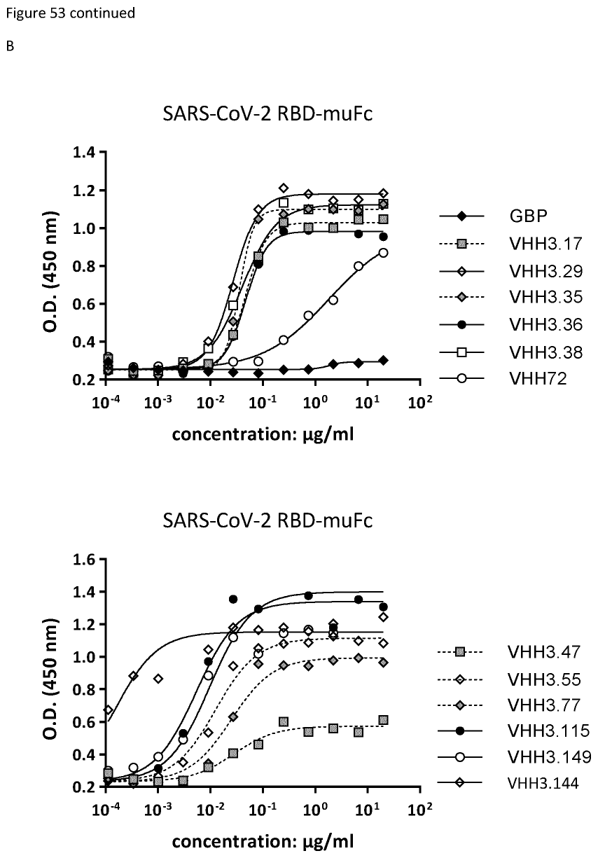

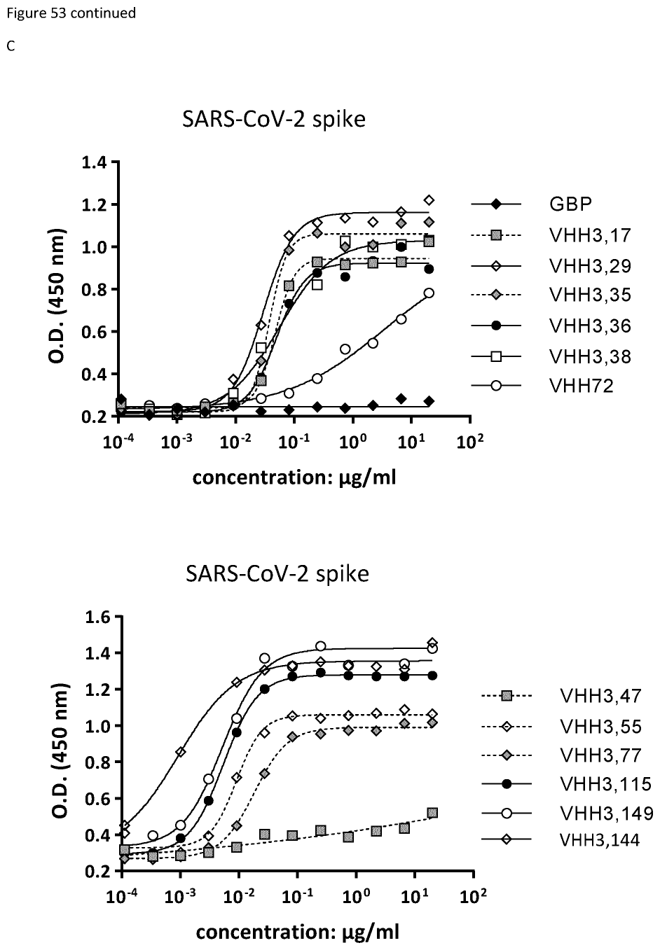

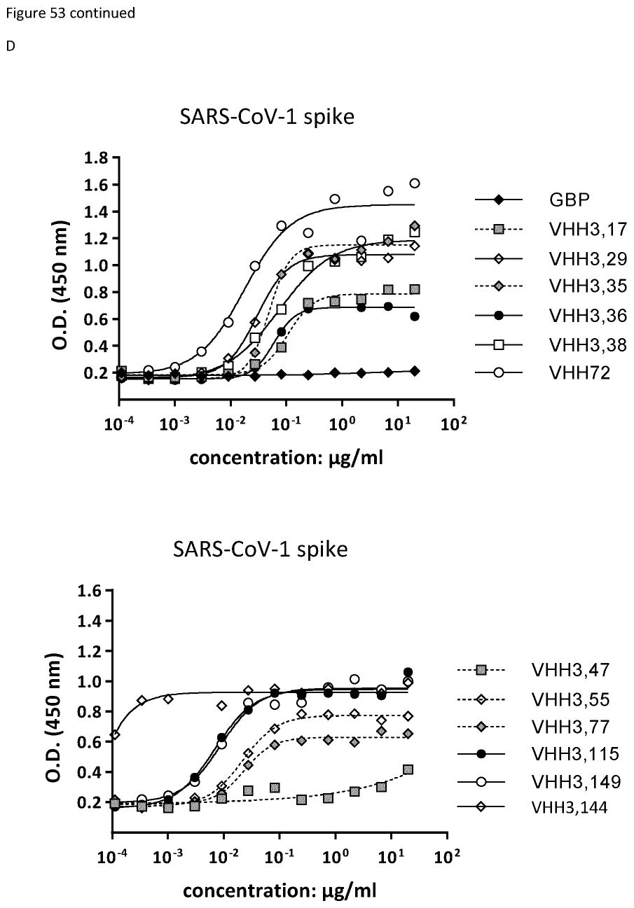

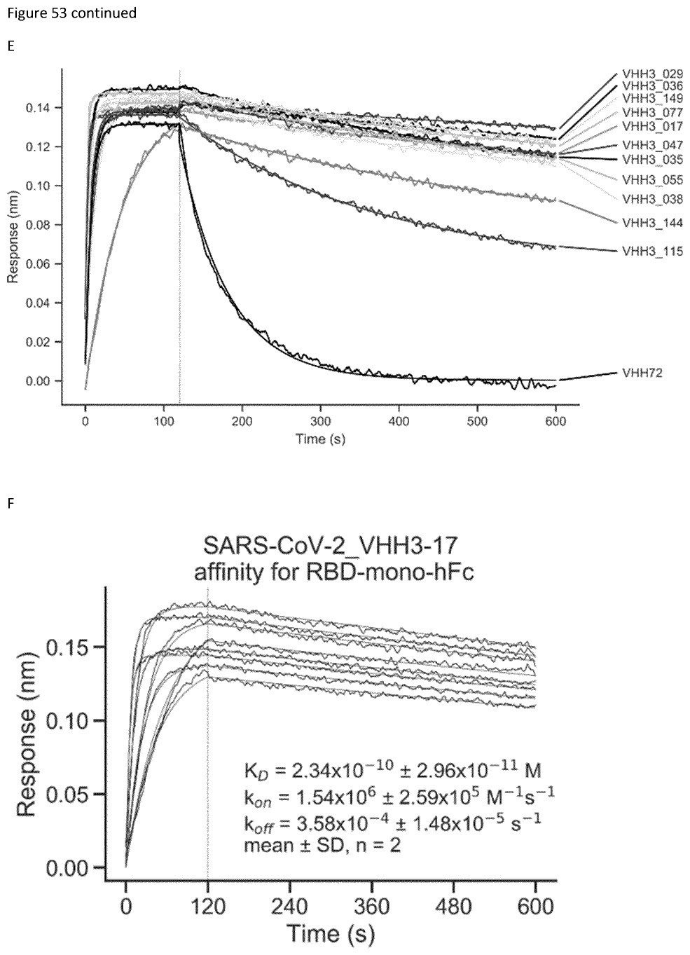

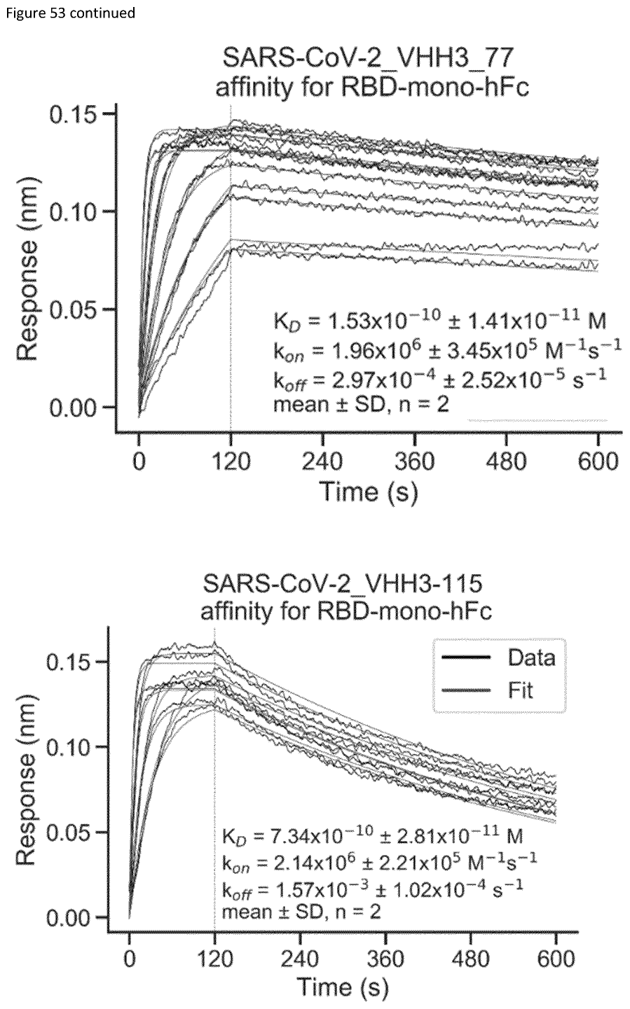

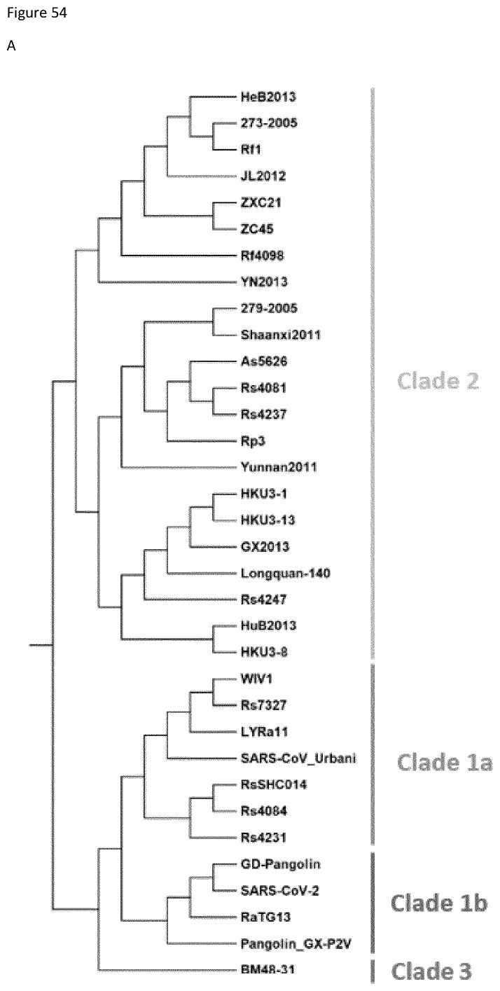

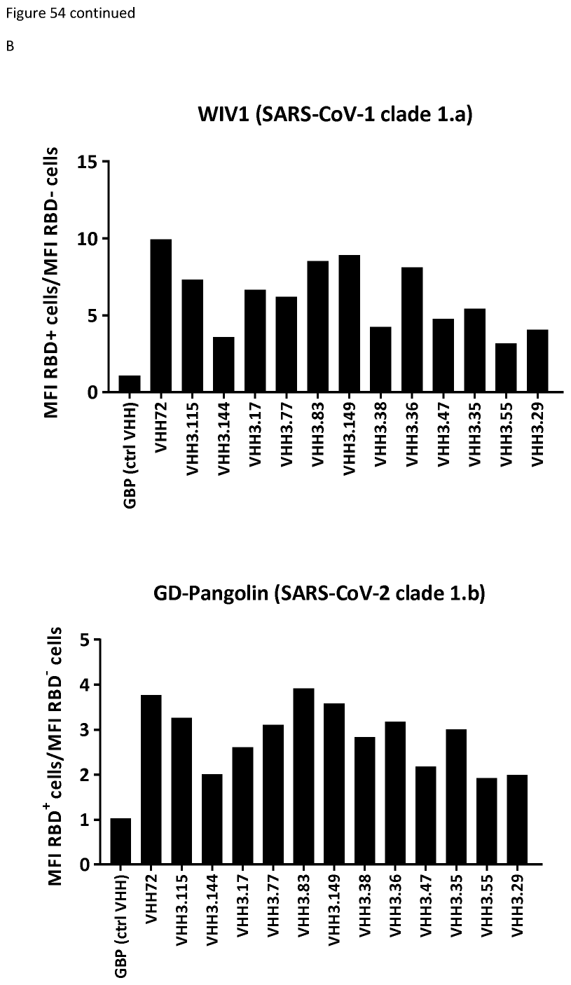

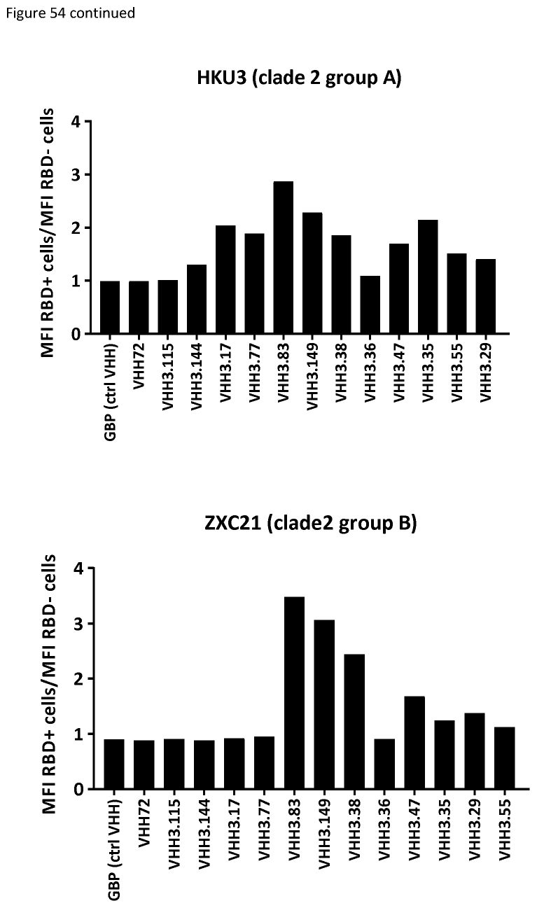

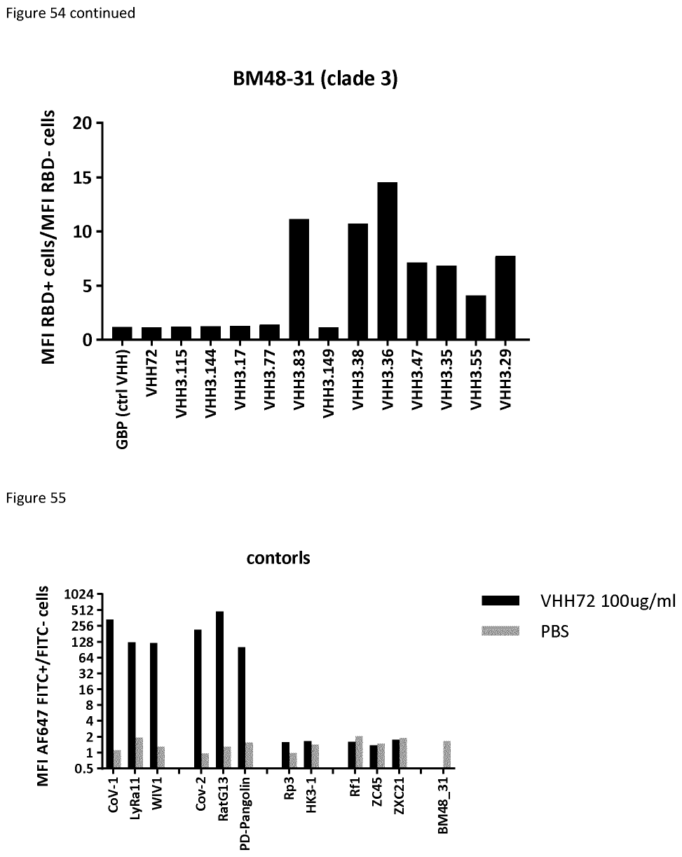

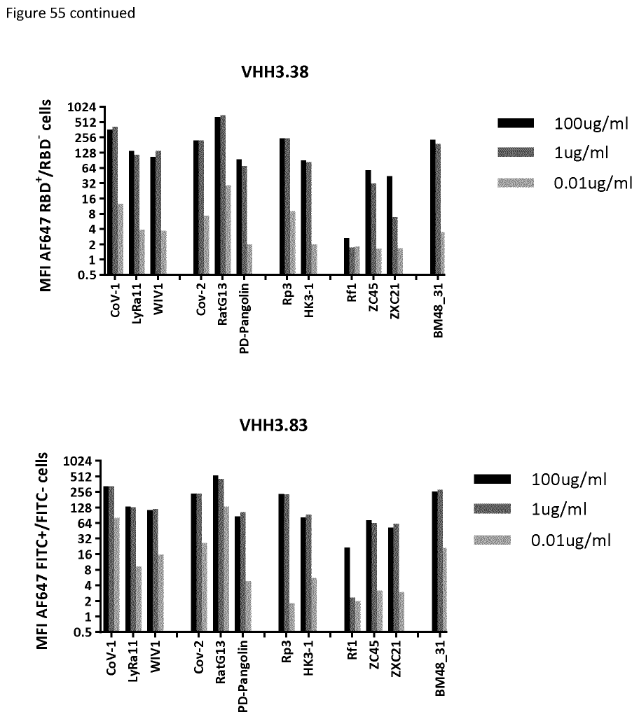

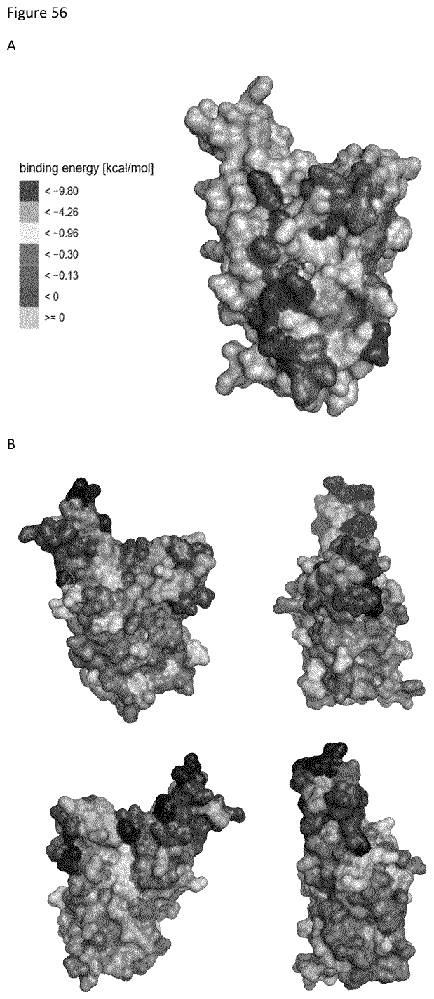

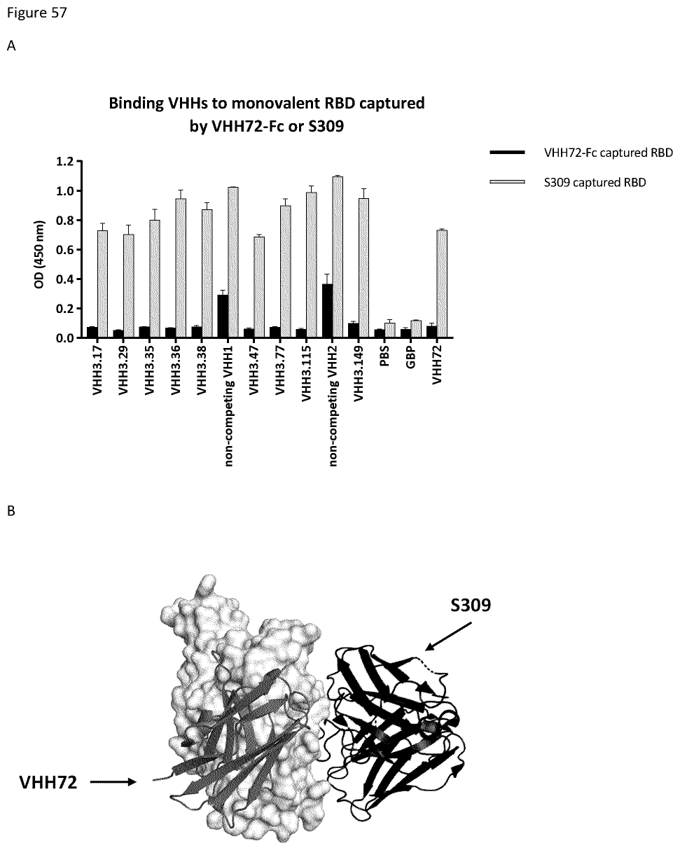

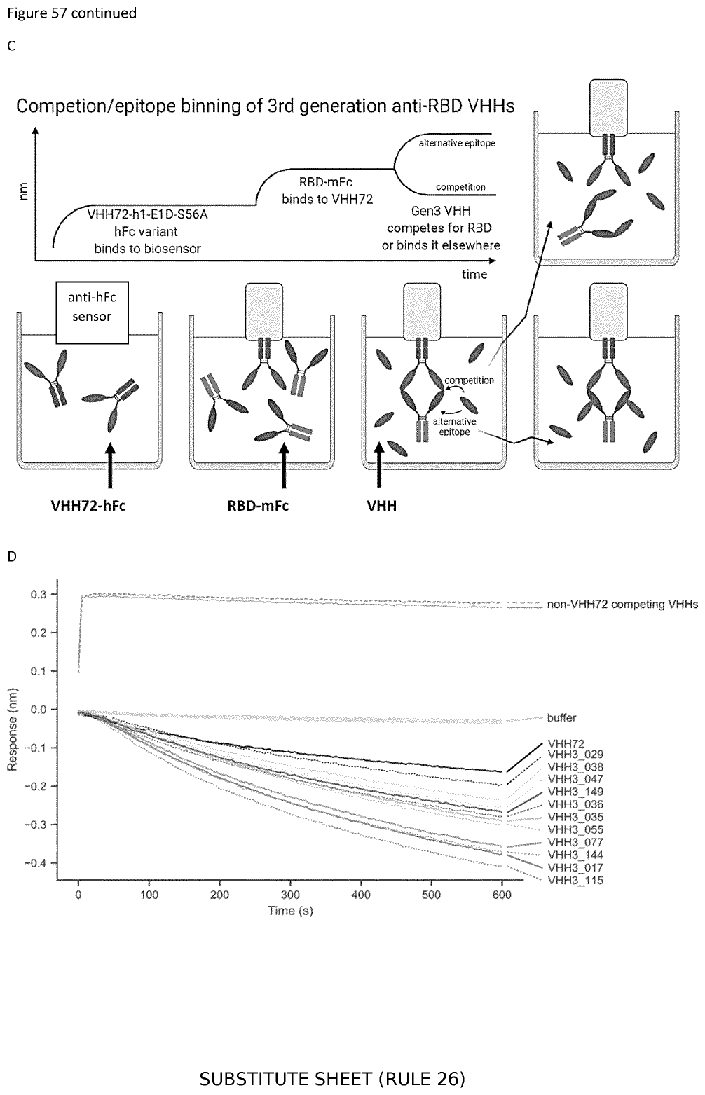

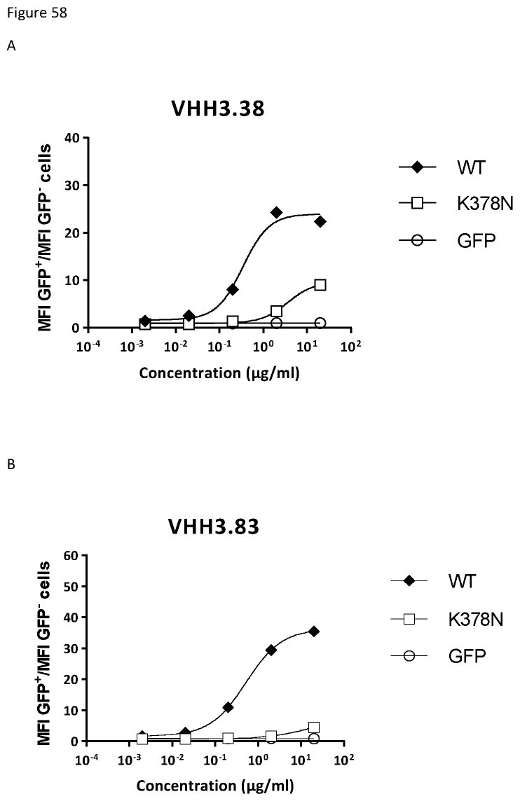

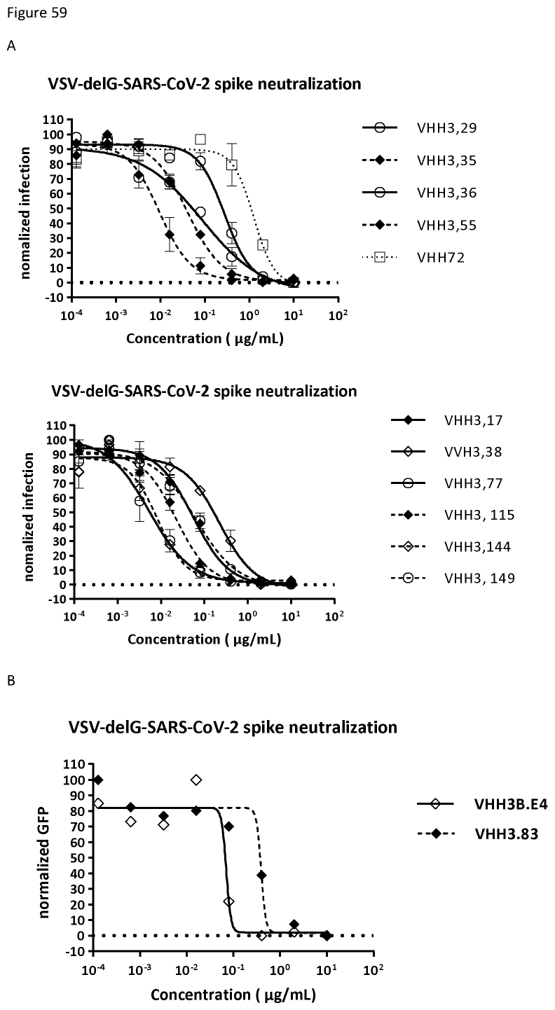

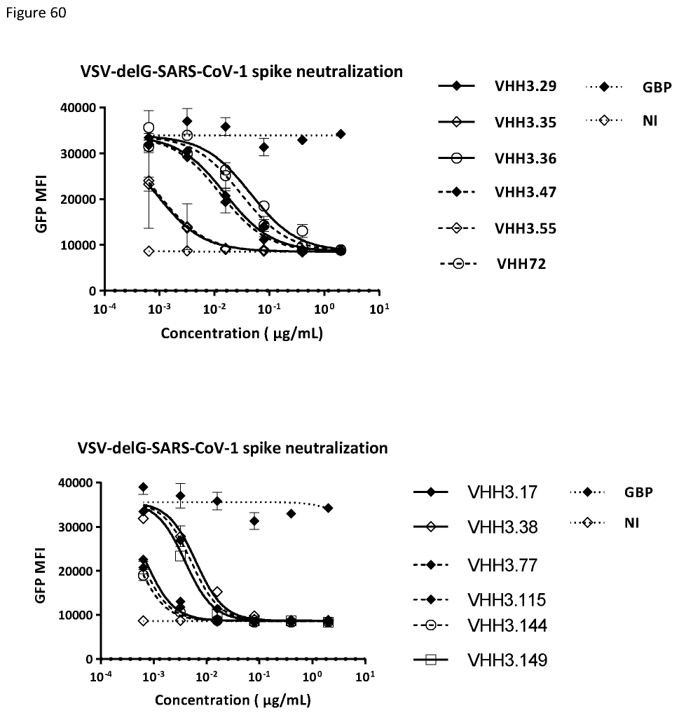

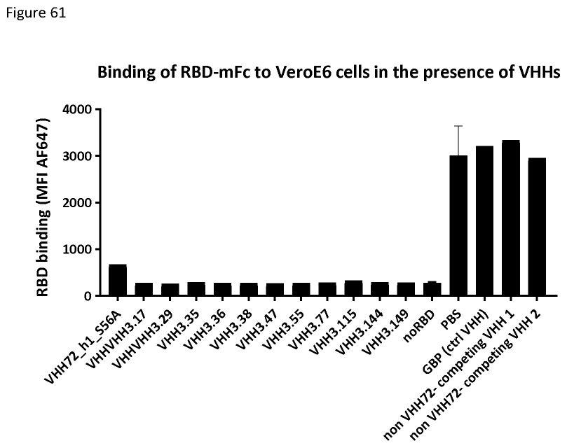

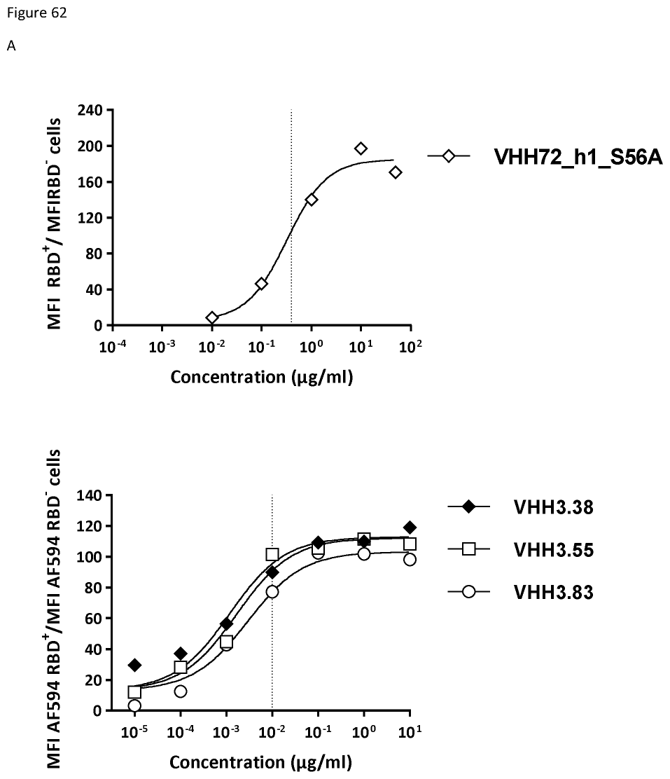

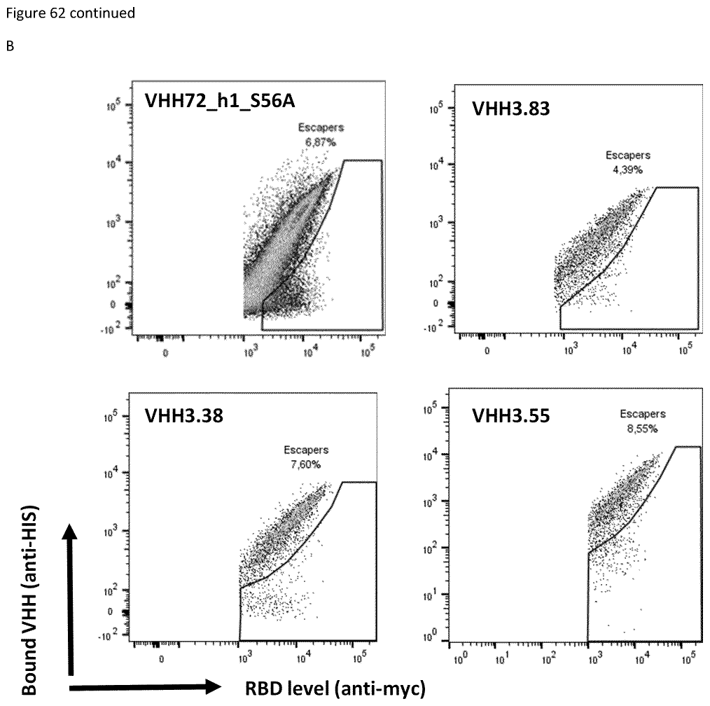

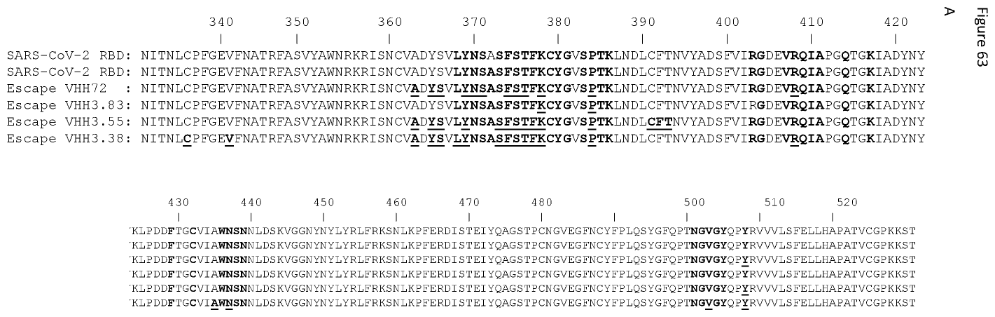

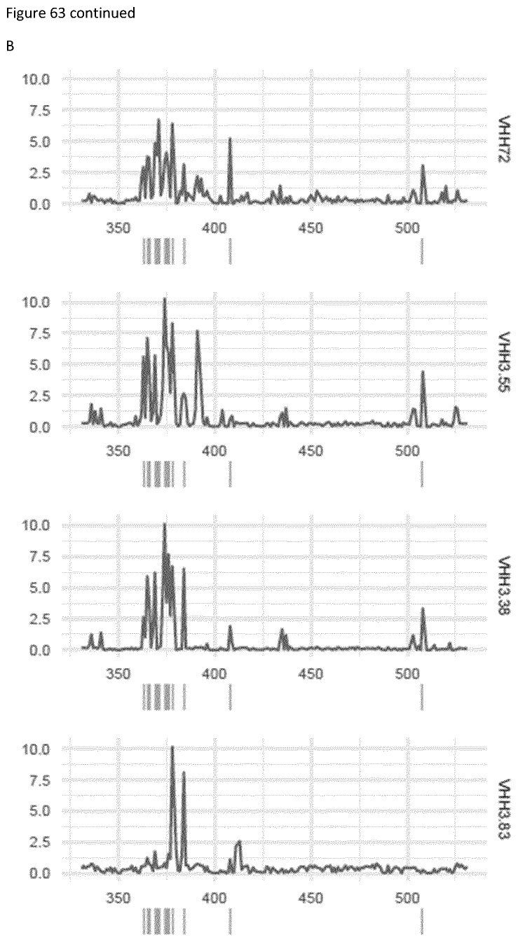

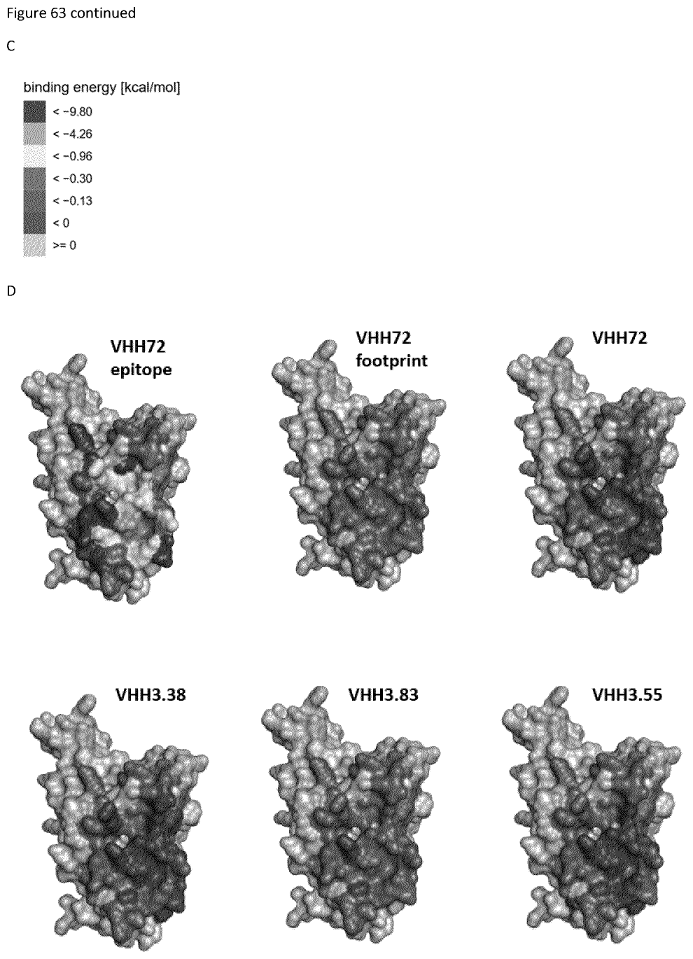

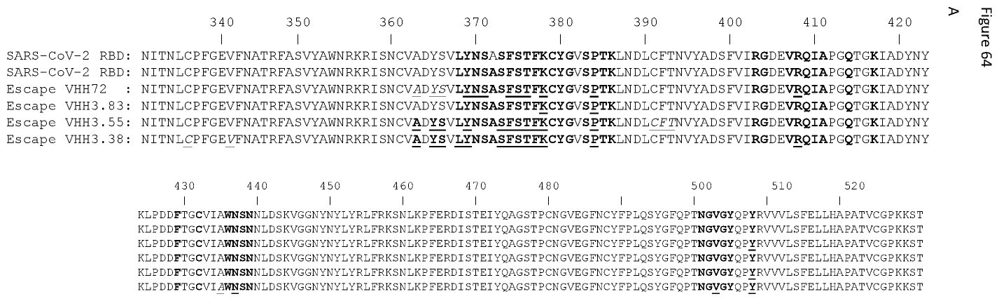

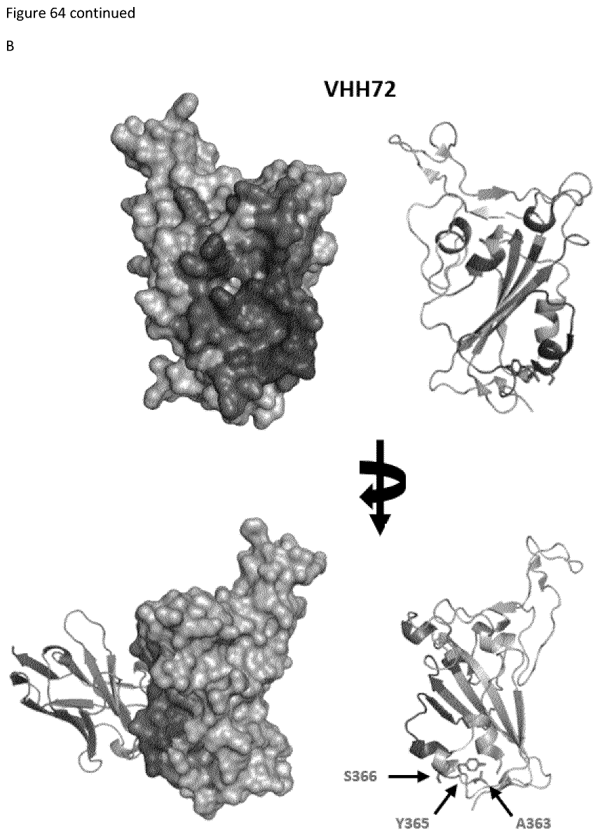

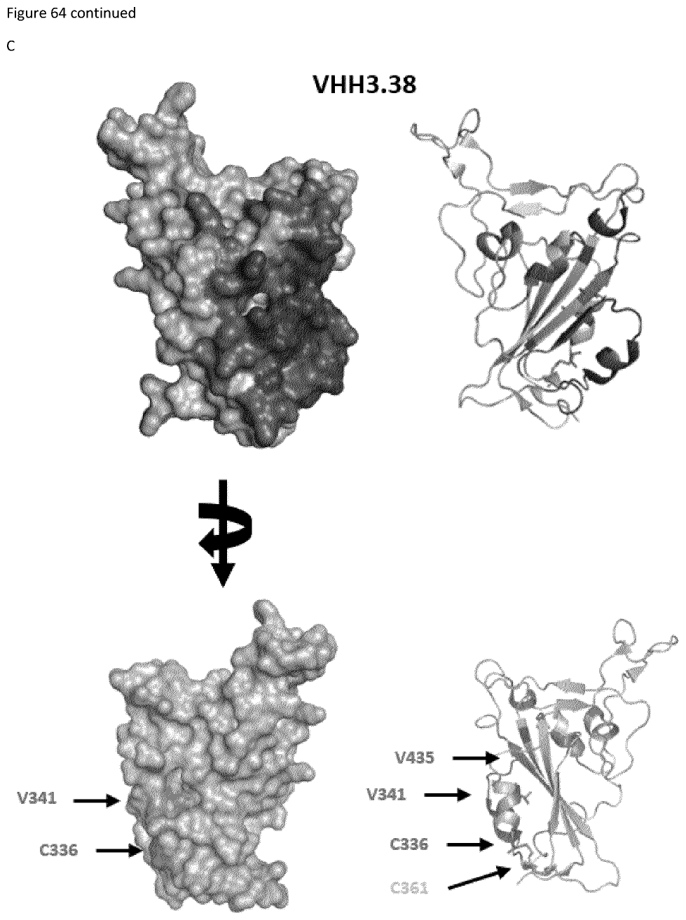

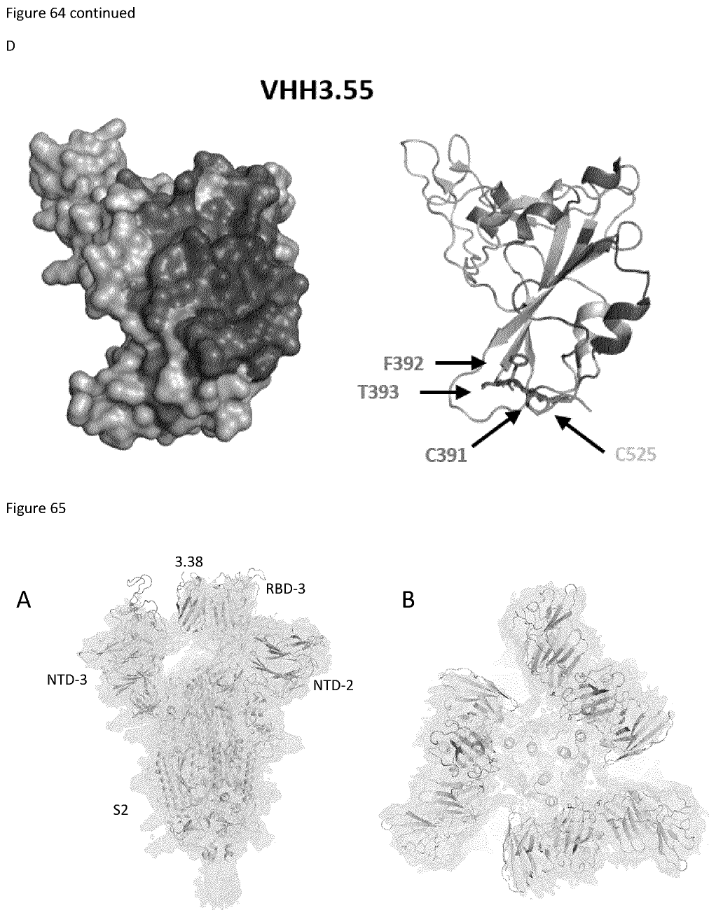

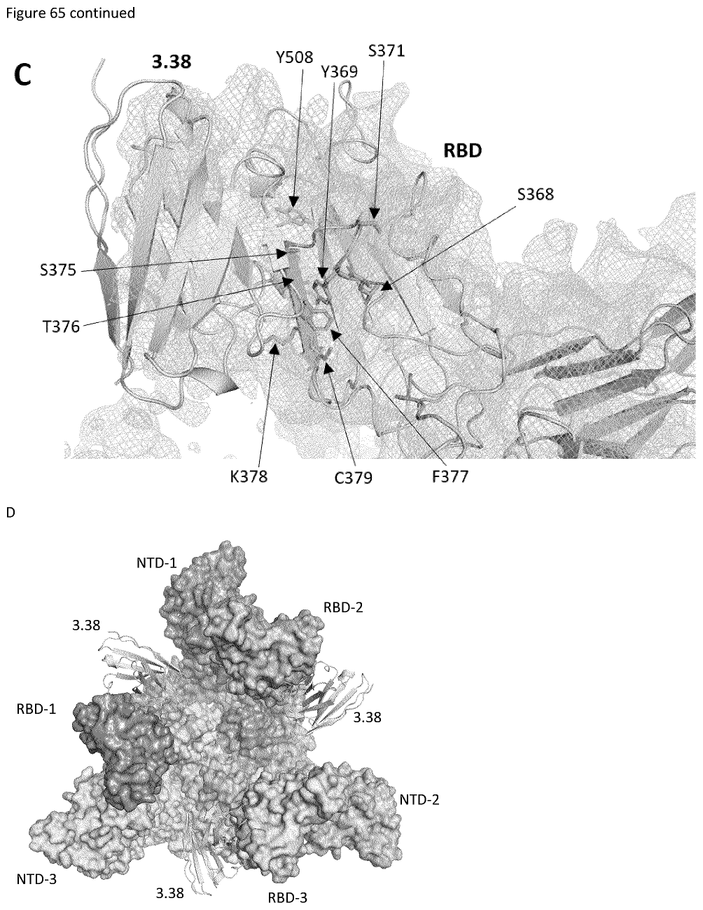

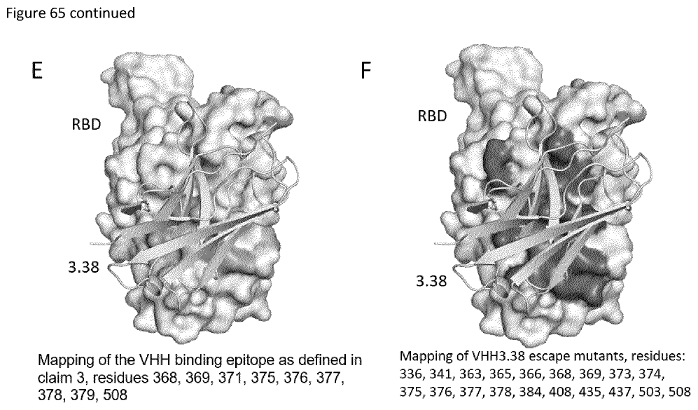

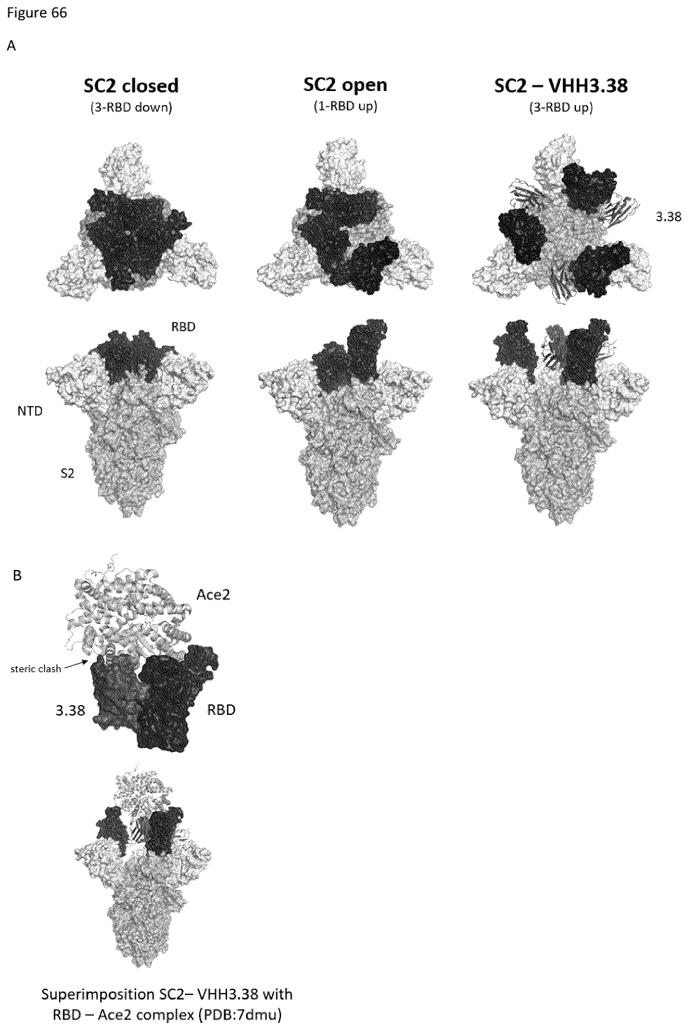

SARS VHH-72 was genetically fused to a His-tag, expressed in Pichia pastoris and purified from the yeast medium by Ni-NTA affinity chromatography. Purified SARS VHH-72 was subsequently used in ELISA to confirm binding to full length SARS CoV S and evaluate binding to the RBD or N-terminal domain of SARS CoV S. We found that SARS VHH-72 bound to full length S as well as to the RBD, but not to the N-terminal domain of SARS CoV (FIG. 1). We also determined the binding kinetics of SARS VHH-72 to purified recombinant SARS CoV, WIV1 CoV and 2019-nCoV RBD by surface plasmon resonance (SPR). WIV1-CoV is an emergent coronavirus found in bats that is closely related to SARS-CoV and also utilizes ACE2 as a host-cell receptor. His-tagged SARS VHH72 was immobilized to a single flow cell of an NTA sensorchip at a level of ˜400 response units (RUs) per cycle using a Biacore X100 (GE Healthcare). The chip was doubly regenerated using 0.35 M EDTA and 0.1 M NaOH followed by 0.5 mM NiCl₂. Three samples containing only running buffer, composed of 10 mM HEPES pH 8.0, 150 mM NaCl and 0.005% Tween 20, were injected over both ligand and reference flow cells, followed by either SARS-CoV RBD, WIV1-CoV RBD or 2019-nCoV RBD serially diluted from 50-1.56 nM, with a replicate of the 3.1 nM concentration. The resulting data were double-reference subtracted and fit to a 1:1 binding model using the Biacore X100 Evaluation software.

This SPR analysis showed that the dissociation constant of the interaction between SARS VHH-72 and the respective RBDs was lowest (1.15×10⁻⁹ M; strongest interaction) for SARS CoV RBD, followed by WIV1 CoV RBD (7.47×10⁻⁹ M) and 2019-nCoV RBD (38.68×10⁻⁹ M) (see FIG. 2). We conclude that SARS VHH-72 exhibits high binding affinity to the WIV1-CoV and 2019-nCoV RBD, demonstrating that it is cross-reactive between the related coronaviruses SARS-CoV, WIV1-CoV and 2019-nCoV.

### Example 3. The Epitope on SARS CoV RBD that is Recognized by SARS VHH-72

After having established that SARS VHH-72 recognizes the RBD of SARS S, we determined the co-crystal structure of SARS VHH-72 in complex with SARS CoV RBD (SEQ ID NO:26). Plasmids encoding SARS VHH-72 and residues 320-502 of SARS-CoV S with a C-terminal HRV3C cleavage site and a monomeric human Fc tag were co-transfected into kifunensin-treated FreeStyle 293F cells. After purifying the cell supernatant with Protein A resin, the immobilized complex was treated with HRV3C protease and Endoglycosidase H to remove both tags and glycans. The processed complex was subjected to size-exclusion chromatography using a Superdex 75 column in 2 mM Tris pH 8.0, 200 mM NaCl and 0.02% NaN₃. The purified complex was then concentrated to 10.00 mg/mL and used to prepare hanging-drop crystallization trays. Crystals grown in 0.1 M Tris pH 8.5, 0.2 M LiSO₄, 0.1 M LiCl and 8% PEG 8000 were soaked in mother liquor supplemented with 20% glycerol and frozen in liquid nitrogen. Diffraction data were collected to a resolution of 2.20 Å at the SBC beamline 19-ID (APS, Argonne National Laboratory). Diffraction data for the complex were indexed and integrated using iMOSFLM before being scaled in AIMLESS. The SARS-CoV RBD+SARS VHH-72 dataset was phased by molecular replacement in PhaserMR using coordinates from PDBs 2AJF and 5F1O as search ensembles. Crystallographic software packages were curated by SBGrid.

Crystals of this complex grew in space group P3₁21 and diffracted X-rays to a resolution of 2.20 Å. The resulting structure revealed an extensive hydrogen bonding network between SARS VHH-72 and the SARS-CoV RBD, with CDRs 2 and 3 encompassing the majority of the 834.1 Å² of buried surface area at the binding interface (FIG. 3). Although the SARS VHH-72 epitope covers a large patch of surface area on the SARS-CoV RBD, it does not obviously overlap with the ACE2 binding interface. However, if ACE2 were to engage with the SARS VHH-72-bound RBD, we predict that a sizeable steric clash would be formed between the CDR-distal framework of SARS VHH-72 and ACE2 (FIG. 3).

SARS VHH-72 binds to the SARS-CoV RBD by forming an extensive hydrogen bonding network with its CDRs 2 and 3 (FIG. 3). Ser56 from the SARS VHH-72 CDR2 simultaneously forms hydrogen bonds with the peptide backbone of three residues from the SARS-CoV RBD, Leu355, Tyr356 and Ser358. The peptide backbone of Ser358 also forms a hydrogen bond with the backbone of neighboring Thr57 from the CDR2. A salt bridge formed between Asp61 and Arg426 tethers the C-terminal end of the CDR2 to the SARS-CoV RBD. The N-terminus of the SARS VHH72 CDR3 forms a short beta strand that pairs with a beta strand from the SARS-CoV RBD to bridge the interface between these two molecules. This interaction is mediated by backbone hydrogen bonds from Gly98, Val100 and Val100a to Cys366 and Phe364 from the SARS-CoV RBD. Glu100c from the SARS VHH72 CDR3 forms hydrogen bonds with the sidechain hydroxyls from both Ser362 and Tyr494 from the SARS-CoV RBD. The neighboring CDR3 residue also engages in a sidechain-specific interaction by forming a salt bridge between the pyrrole nitrogen of Trp100d and the hydroxyl group from Thr363. Asp101 is involved in the most C-terminal interaction from the CDR3 by forming a salt bridge with Lys365 of the SARS CoV RBD. The extensive interactions formed between CDRs 2 and 3 of SARS VHH72 and the SARS-CoV RBD help to explain the high-affinity binding that we observe between these molecules.

Furthermore, analysis of available SARS-CoV strain sequences reveals a high degree of conservation in the residues that make up the SARS VHH-72 epitope (see FIG. 3). This high degree of sequence conservation in the SARS VHH-72 epitope, coupled with the high affinity that SARS VHH-72 has for the SARS-CoV RBD, suggests that this molecule may represent an attractive potential therapeutic in the event of future SARS-CoV and SARS CoV-like outbreaks.

SARS-CoV and the 2019-nCoV can both use ACE2 as the host cell receptor. However, there is considerable sequence difference between the RBD of SARS-CoV and 2019-nCoV as can be seen in the amino acid sequence alignment of these two RBDs (FIG. 5). However, remarkably, 9 out of 10 residues that are directly involved in the interaction of SARS-CoV RBD with SARS VHH-72 are identical in the RBD of 2019-nCoV (FIG. 5). This high sequence similarity in the contact residue of SARS-CoV RBD with SARS VHH-72 is in line with the binding of SARS VHH-72 to the recombinant purified 2019-nCoV RBD (see FIG. 2).

### Example 4. SARS CoV VHH-72 Prevents Interaction with ACE2 Receptor

Based on our structural analysis, we hypothesized that a mechanism by which SARS VHH-72 could neutralize its viral targets is by blocking the interaction between the RBDs from SARS-CoV and its host cell receptor. To test this hypothesis, we performed a bio-layer interferometry (BLI)-based assay in which the SARS-CoV RBDs were immobilized to biosensor tips, dipped into SARS VHH-72 or a negative control VHH and then dipped into wells containing the recombinant, soluble host cell receptor ACE2 (FIG. 4). To this end, anti-human capture (AHC) tips (FortéBio) were soaked in running buffer composed of 10 mM HEPES pH 7.5, 150 mM NaCl, 3 mM EDTA, 0.005% Tween 20 and 1 mg/mL BSA for 20 minutes before being used to capture Fc-tagged SARS-CoV RBD to a level of 0.8 nm in an Octet RED96 (FortéBio). Tips were then dipped into either 100 nM negative control VHH or 100 nM SARS VHH-72. Tips were next dipped into wells containing 1 μM ACE2 supplemented with the nanobody that the tip had already been dipped into to ensure saturation. Data were reference-subtracted and aligned to each other in Octet Data Analysis software v11.1 (FortéBio) based on a baseline measurement that was taken before being dipped into the final set of wells that contained either ACE2 or DPP4.

We found that when tips coated in the SARS CoV RBD, were dipped into the negative control VHH and then ACE2, a robust response signal was observed, indicating that no nonspecific interaction between the negative control VHH was occurring that might disrupt the association between the SARS-CoV RBD and its receptor. However, when tips coated with the SARS-CoV RBD were dipped into SARS VHH-72 being dipped into ACE2, there was only a very minor increase in response that could be attributed to receptor binding. These results support our structural analysis that SARS VHH-72 is capable of neutralizing its viral target by preventing host cell receptor binding.

### Example 5. SARS VHH-72 can Neutralize SARS S Pseudotyped Lentiviruses

To assess the antiviral activity of SARS-CoV VHH-72, in vitro neutralization assays, using SARS-CoV Urbani viruses were performed. Pseudotyped lentiviral virus neutralization assay methods have been previously described⁵⁴. Briefly, pseudoviruses expressing spike genes for SARS-CoV Urbani (GenBank ID: AAP13441.1) or 2019-nCoV S (spike protein sequence is depicted in SEQ ID NO: 23) were produced by co-transfection of plasmids encoding a luciferase reporter, lentivirus backbone, and spike genes in 293T cells⁵⁵. Serial dilutions of VHHs were mixed with pseudoviruses, incubated for 30 min at room temperature, and then added to previously-plated Huh7.5 cells. Seventy-two (72 h) hours later, cells were lysed, and relative luciferase activity was measured. Percent neutralization was calculated considering uninfected cells as 100% neutralization and cells transduced with only pseudovirus as 0% neutralization. IC₅₀ titers were determined based on sigmoidal nonlinear regression. This neutralization assay revealed that SARS VHH-72 was able to neutralize SARS-CoV Urbani virus with an IC₅₀ value of 0.14 μg/ml.

### Example 6. SARS VHH-72 Human IgG Fc Fusion Constructs and Other Bivalent Constructs

We also generated genetic fusions between SARS VHH-72 and human IgG1 and IgG2-derived Fc domains. SARS VHH-72 was directly linked to the hinge region of human IgG1. The hinge region of IgG2, was replaced by the hinge of human IgG1 to generate SARS VHH-72 fusion constructs. Additional linkers that are used to fuse SARS VHH-72 to the IgG1 and IgG2 Fc domains comprise (G₄S)₂₋₃. In addition, we use Fc variants with known half-live extension such as the M257Y/S259T/T261E (also known as YTE)⁵⁶ or the LS variant (M428L combined with N434S)⁵⁷. These mutations increase the binding of the Fc domain of a conventional antibody to the neonatal receptor (FcRn). In addition, we construct homobivalent tandem genetic fusions of SARS VHH-72 in which the two copies are separated by a flexible linker such as (G₄S)₂₋₃. The latter construct is depicted in SEQ ID NO: 12. Such tandem repeat constructs can increase the avidity and, for some other viruses, the neutralizing breadth and potency of antiviral VHHs⁵⁸.

These fusion constructs of SARS VHH-72 are evaluated for binding to SARS CoV and 2019-nCoV S and RBD binding using ELISA and SPR as described above. In addition, these are tested in virus neutralization assays using pseudotyped viruses as described above (e.g. Example 7). In vitro antiviral activity testing is also performed with a SARS CoV and 2019-nCoV strain.

In another example we fuse SARS VHH-72 to a human serum album-specific VHH as described for example in WO2019016237, WO2004041865 or WO2006122787. The resulting fusion allows the VHH to bind to serum albumin and hence provided an extended half-live.

### Example 7. VHH-72 Prevents Binding of ACE2 to the RBD of 2019-nCoV (2019-nCoV RBD-SD1)

Anti-human capture (AHC) tips (FortéBio) were soaked in running buffer composed of 10 mM HEPES pH 7.5, 150 mM NaCl, 3 mM EDTA, 0.005% Tween 20 and 1 mg/mL BSA for 20 minutes before being used to capture either Fc-tagged MERS-CoV RBD, Fc-tagged SARS-CoV RBD or Fc-tagged 2019-nCoV RBD-SD1 to a level of 0.8 nm in an Octet RED96 (FortéBio). Tips were then dipped into either 100 nM VHH-55 or 100 nM VHH-72. Tips were next dipped into wells containing either 100 nM DPP4 or 1 μM ACE2 supplemented with the nanobody that the tip had already been dipped into to ensure saturation. Data were reference-subtracted and aligned to each other in Octet Data Analysis software v11.1 (FortéBio) based on a baseline measurement that was taken before being dipped into the final set of wells that contained either DPP4 or ACE2 (data are shown in FIG. 6).

### Example 8. VSV Pseudotype Neutralization Assays

In addition, we also performed VSV pseudotype neutralization assays using a previously reported protocol to generate such reporter viruses and assess neutralization (Hoffmann, M. et al (2020) Cell 181, 1-10). We found that VHH-72 fused to a human IgG1 Fc (SEQ ID NO: 13) and secreted into the serum-free medium of transfected 293T cells, could neutralize the 2019-nCoV and SARS-CoV spike pseudotyped viruses whereas a negative control supernatant with GFP-binding protein failed to do so (see FIG. 7). The VHH-72 Fc fusion failed to neutralize MERS-CoV spike pseudotyped viruses. Purified VHH-72 could neutralize SARS-CoV but not 2019-CoV pseudotypes (FIG. 7 D-F). VHH-55 neutralized MERS-CoV but not SARS-CoV or 2019-nCoV pseudotypes (FIG. 7 D-F).

### Example 9. Prophylactic Treatment of Hamsters with VHH-72 IgG1 Fc Antibody Protects Against SARS-Cov-2 Infection

VHH-72 fused to a human IgG1 Fc (SEQ ID NO: 13) secreted into the serum-free medium of transfected 293T cells, was shown to be able to neutralize the 2019-nCoV and SARS-CoV spike pseudotyped viruses by VSV pseudotype neutralization assays (Example 8).

The SARS VHH-72 fusion construct was further evaluated for prophylactic use in Syrian hamsters, which are highly susceptible to SARS-CoV-2³⁴. Wild type hamsters were treated prophylactically with neutralizing betacoronavirus-specific single-domain antibody VHH-72 Fc¹⁰ and human convalescent plasma 1 day prior to intranasal inoculation with 2019-nCoV (also called SARS-Cov-2 herein). The viral RNA load, which is used as proxy for the quantification of viral loads, was measured in lung samples which were generated 4 days post infection (FIG. 8A, B). The VHH-72 Fc antibody was used at a dose of 20 mg/kg. Unlike a single dose of convalescent plasma, which did not significantly reduce viral load in the lungs, pre-treatment with VHH-72-Fc reduced viral loads in the lung ^{˜}10⁵-fold compared to untreated control animals.

### Example 10. Design of Variants of VHH-72 and Expression of IgG Fc Fusion Constructs in Pichia pastoris

Previously, we identified VHH-72 binding to the RBD domain of SARS-CoV-1 and also shown to be capable of binding to the RBD domain of SARS-CoV-2. The co-crystal structure between VHH-72 and the RBD domain of SARS-CoV-1 was determined with its atomic coordinates of the three-dimensional structure as provided in PDB 6WAQ. Based on the co-crystal structure of VHH72 with SARS-CoV-1 RBD and the cryo-EM structure of the SARS-CoV-2 spike in the prefusion conformation²³ several variants of VHH72 were predicted that potentially would have a higher affinity for SARS-CoV-2 RBD. Visual inspection and molecular modelling were used to generate a set of VHH-72 muteins with potentially improved binding to SARS-CoV-2 RBD (see FIGS. 9 to 11). The variants (and the VHH-72 control sequence, and the humanized variants of VHH-72) are depicted in the sequence listing of the application. VHH72 and variants thereof were cloned in Pichia pastoris (alternative name is Komagataella phaffii) expression vectors through a MoClo Golden Gate-based modular cloning system in the following constellation: Pichia pGAP promoter controlling a coding sequence consisting of the S. cerevisiae alpha-mating factor prepro secretion leader devoid of its EAEA tetrapeptide, fused to the coding sequence of the VHH-72 muteins without a start codon, fused either directly to the hIgG1 hinge and Fc, or through a (GGGGS)x2 linker to the hIgG1 hinge and Fc, and terminated by a stop codon. Transcription is terminated by the Pichia pastoris AOX1 transcription terminator. The vector contains a Zeocin selection cassette, and ampicillin selection marker and a ColE1 origin of replication for vector propagation in E. coli. These last three elements are flanked by LoxP sites.

This way, the expression of the variant VHH-72 Fc fusions is controlled by the constitutive glyceraldehyde phosphate dehydrogenase promoter. Constructs were transformed to Komagataella phaffii strain NRRLY11430 with a suppressed OCH1p activity in order to reduce N-glycosylation heterogeneity. Two clones from each transformation were randomly selected for analysis of expression of the desired VHH-Fc fusion in the yeast growth medium. Two days after inoculation of the respective yeast clones in 2 ml of BMDY (2% glucose, 2% peptone, 1% yeast extract, 1.34% yeast nitrogen base buffered at pH 6.0 with 100 mM of potassium phosphate buffer) cultivation medium in 24-square wall round bottom well plates sealed with a gas-permeable membrane, shaking at 225 rpm in an incubator at 28° C., cultures were harvested and yeast cells removed by centrifugation. A fraction of the supernatant (27 microliter) was loaded on an SDS PAGE gel (4-20% gradient) that was stained with Coomassie brilliant blue. Except for the VHH72_S52A-(GGGGS)x2-hIgG1.Hinge-hIgG1.Fc construct, expression of all VHH-Fc fusions was detectable by Coomassie staining for crude yeast culture supernatant. Based on the loaded purified reference material (GFP-binding protein Fc=GBP-Fc) we estimate that the yeast cultures expressed the desired VHH-Fc fusions at a concentration of approx. 35-50 mg/l (see FIGS. 12 to 14).

### Example 11. Expression of IgG Fc Fusion Constructs in Mammalian Cells

For mammalian expression tests, a series of Fc variants, C-terminally linked to the SARS-VHH72 VHH, were cloned into the pcDNA3.3 expression vector. These Fc variants potentially impose different properties on the chimeric antibody, such as flexibility, Fc-receptor engagement, in vivo half-life extension. Examples of constructs that were transiently expressed are shown in FIGS. 15 and 16.

Suspension-adapted, serum free-adapted HEK293-S cells were transiently transfected with the different VHH-Fc fusions. For this, cells were spun down and resuspended in Freestyle-293 medium, to a density of 3×10⁶ cells per mL. Cells were divided per 2.5 mL in 50 mL bio-incubator tubes and incubated on a shaking platform (200 rpm) at 37° C. and 5% CO₂. For each construct, a combination of 11.125 μg of expression plasmid and 0.125 μg of a plasmid encoding the SV40 Large T antigen (to boost expression) was added to the cells. After 5 min of incubation on a shaking platform, 22.5 μg of linear 25 kDa polyethyleenimine (PEI) was added to the cell/DNA mix. Five hours after transfection, an equal amount (2.5 mL) of ExCell-293 medium was added to the transfected, to stop transfection and provide necessary growth factors. Three days after transfection, the crude cell supernatant was harvested and loaded on a SDS-PAGE followed by Coomassie blue staining or analyzed by Western blot using a monoclonal rabbit anti-VHH antibody or anti-human IgG immune serum (see FIGS. 15 and 16). We noted that expression of construct VHH72-GSGGGGSGGGGS-hIgG1Hinge-hIgG1Fc_YTE was not detectable because of an inadvertent frame shift that was identified in hindsight.

### Example 12. Biolayer Interferometry (BU) Screening of Variant VHH72-hFc Fusions

The RBD binding characteristics of P. pastoris-expressed VHH72-hIgG1 Fc variants were screened via biolayer interferometry. 10 to 20 μg/ml of mouse IgG1 Fc fuse SARS-CoV-2-RBD (Sino Biological) was immobilized on an anti-mouse IgG Fc capture (AMC) biosensor (FortéBio). P. pastoris OCH⁻ cultures expressing variant VHH-72-Fc fusion were pelleted and crude cell supernatants were diluted 50-fold in kinetics buffer (10 mM HEPES pH 7.5, 150 mM NaCl, 1 mg/ml bovine serum albumin, 0.05% Tween-20 and 3 mM EDTA). Affinity for RBD was measured at 30° C. Baseline and dissociation were measured in a 50-fold dilution of non-transformed P. pastoris OCH⁻ supernatant in kinetics buffer. Between analyses, biosensors were regenerated by three times 20 s exposure to regeneration buffer (10 mM glycine pH 1.7). Using FortéBio Data Analysis 9.0 software, both association and dissociation of non-saturated curves were fit in a global 1:1 model and the decrease of response signal during dissociation was determined. Protein concentrations were estimated based on band intensity on Coomassie-stained SDS-PAGE as compared to a purified VHH-hFc protein (see FIGS. 17-21).

Similarly, the RBD binding characteristics of VHH72-hIgG1 Fc variants expressed by transfected HEK293T cells were also assessed via biolayer interferometry. 10 to 20 μg/ml of mouse IgG1 Fc fuse SARS-CoV-2-RBD (Sino Biological) was immobilized on an anti-mouse IgG Fc capture (AMC) biosensor (FortéBio). Non-transfected HEK293T cells and HEK293T cells expressing VHH72-hIgG1 Fc were pelleted and three-fold dilution series of the crude cell supernatant were prepared in kinetics buffer. RBD affinity of VHH72-hIgG1 Fc in HEK293T supernatant was measured at 30° C., with baseline and dissociation measured in equal dilution of non-transformed HEK supernatant in kinetics buffer. Between analyses, biosensors were regenerated by three times 20 s exposure to regeneration buffer (10 mM glycine pH 1.7). Using FortéBio Data Analysis 9.0 software, both association and dissociation of non-saturated curves were fit in a global 1:1 model. Protein concentrations were estimated based on band intensity on Coomassie-stained SDS-PAGE as compared to a purified VHH-hFc protein. The reported approximate k_{D}, kₒₙ and k_{off} values are the averages and accompanying standard deviation of two replicate measurements (see FIG. 22).

### Example 13. Flow Cytometry

To test the ability of the VHH-72 variants to bind to the SARS-CoV-2 Spike, flow cytometric analysis was performed using cells transfected with a GFP expression plasmid in combination with an expression plasmid for either the SARS-CoV or SARS-Cov-2 S. Culture media (1/20 diluted in PBS+0.5% BSA) of Pichia pastoris clones expressing different variants of SARS VHH-72 fused to a human IgG1 Fc with either a GS or a GS(G4S)2 linker were incubated with transfected cells. Binding of the SARS VHH-72 variants to cells was detected by an AF633 conjugated goat anti-human IgG antibody. The bars represent the AF633 mean fluorescence intensity (MFI) of GFP expressing cells (GFP*) divided by the MFI of GFP negative cells (GFP⁻) (see FIG. 23).

### Example 14. In Vivo Protection of Variant VHH-72 IgG Fc Fusion Constructs

Several variant VHH-72 IgG Fc fusion constructs are evaluated for prophylactic and therapeutic use of ACE2 transgenic mice that are challenged with SARS-CoV-2. These mice express human ACE2 and are susceptible to disease caused by SARS-CoV-2 infection (McRay, P B et al (2007) J. Virol. 81, 813-821). The mice are treated prophylactically with SARS VHH-Fc and the other fusion constructs described above 1 day prior to challenge infection with SARS-CoV-2 and morbidity (body weight change, lung inflammation, immune cell infiltration in the lungs) is monitored. The variant VHH-72 IgG fusion constructs are administered intranasally to the mice or intravenously. Viral replication in the lungs and the brain after challenge is also monitored to assess the antiviral activity of the variant VHH-72 IgG fusion constructs. In a final set of experiments the ACE2 transgenic mice is infected with SARS-CoV-2 first and treated with the variant VHH-72 IgG fusion constructs on day 1 after infection. The variant VHH-72 IgG fusion constructs are used prophylactically and therapeutically at dose ranging from 0.5 to 5 mg/kg.

### Example 15. VHH72 Binds a Conserved Epitope in the SARS-CoV-2 Spike Protein

Further investigation of the VHH72 variants for increased affinity for the SARS-CoV-1 and -2 RBD and enhanced SARS-CoV-1 and -2 neutralizing activity revealed several formats of multivalent fusion constructs with potentially increased therapeutic value. Further testing of the fusion constructs included as well, as known to the skilled person, humanization substitutions and Fcs with or without Fcγ Receptor functionality, for selecting the most suitable binding agents. Importantly, the selected molecules were shown to be expressed at very high levels in CHO cells and exhibit outstanding homogeneity and biophysical stability.

Free energy contribution analysis by FastContact¹⁴ of snapshots from Molecular Dynamics simulations with the VHH72-RBD complex indicate that the epitope has a prominent two-residue hot-spot, consisting of Lys378 which is in ionic contact with VHH72's Asp100g, and Phe377 whose main contact with VHH72 is Val100 (FIG. 24a). The epitope is accessible when the trimeric spike protein has at least one RBD in an ‘up’ conformation (FIG. 24b)¹⁰. Among the several dozens of human IgGs that have so far been isolated from convalescent SARS-CoV-2 infected patients, including those that are in clinical development, as yet only two recognize an epitope that substantially overlaps with that of VHH72, i.e. EY6A¹⁵ and COVA1-16¹⁶. The epitopes of CR3022, isolated from a convalescent SARS-CoV-1 patient¹⁷, and the humanized mouse monoclonal antibody H014¹⁸, partially overlap with VHH72's epitope.

In the three-RBD ‘down’ state of the pre-fusion spike protein, the epitope of VHH72 belongs to an occluded zone that is mutually complemental to both adjacent RBDs (FIG. 24c), that also contacts the top of the S2 domain at its helix-turn-helix between heptad-repeat 1 and the central helix. This delicate inter-RBD and inter-S1/S2 interface is important to preserve the immune-evading three-RBD ‘down’ pre-fusion state and for the conformational dynamics that permit an intermittent ‘up’ RBD positioning of one or more RBDs needed for full exposure of the ACE-2 recognizing zone^{15,16}. These functional constraints, likely severely limit viral mutational escape against natural or vaccine-induced human immune pressure on the VHH72 epitope. In line with this, the VHH72 epitope has a remarkably low level of drift. Only variant Lys378Asn, observed just twice in over 62.000 SARS-CoV-2 virus genomes analyzed, is predicted by Molecular Dynamics and FastContact analysis to impair the interaction with VHH72 (Table 1). The most frequently observed variant in the epitope is Asn439Lys, which is an analogue-reversion to Arg as in the SARS-CoV-1 RBD sequence, restoring a beneficial ionic interaction with Asp61 of VHH72¹⁰. In addition, deep mutational scanning analysis indicates the VHH72 epitope largely overlaps with a region of the RBD in which mutations may severely compromise the fold, further supporting the assertion that this epitope may be one of the most stable sequence regions on the sarbecoviridal RBD¹⁹.

TABLE 1
Reported SARS-CoV-2 RBD variants at the epitope of h1_VHH72_S56A and predicted effect on recognition.

| RBD variant | 0.5 ns | 1 ns | 1.5 ns | 2 ns | 2.5 ns | 3 ns | 3.5 ns | 4 ns | 4.5 ns | 5 ns | Average | Observed |
|---|---|---|---|---|---|---|---|---|---|---|---|---|
| Parent type | −29.5 | −30.6 | −30.0 | −30.5 | −28.7 | −30.0 | −29.5 | −30.7 | −28.9 | −29.8 | −29.8 | >62K |
| Val367Phe | −30.5 | −30.1 | −27.9 | −28.3 | −28.9 | −28.3 | −29.9 | −30.5 | −30.3 | −31.2 | −29.6 | 40x |
| Asn370Ser | −30.2 | −30.2 | −30.1 | −32.9 | −31.1 | −32.4 | −32.9 | −33.4 | −27.6 | −31.4 | −31.2 | 13x |
| Ala372Ser | −32.2 | −31.7 | −28.6 | −30.1 | −30.0 | −31.0 | −30.1 | −30.9 | −29.7 | −29.0 | −30.3 | 1x |
| Ala372Thr | −32.5 | −32.3 | −29.3 | −28.6 | −31.1 | −31.7 | −28.1 | −29.0 | −29.5 | −32.1 | −30.4 | 2x |
| Ser373Leu | −32.3 | −30.2 | −30.6 | −27.4 | −27.8 | −32.5 | −30.5 | −29.6 | −26.8 | −27.5 | −29.5 | 3x |
| Thr376Ile | −31.9 | −31.7 | −31.4 | −31.7 | −31.2 | −33.4 | −32.7 | −36.6 | −32.5 | −33.7 | −32.7 | 2x |
| Phe377Leu | −29.6 | −29.4 | −28.3 | −29.4 | −27.9 | −27.0 | −27.1 | −27.4 | −28.3 | −28.3 | −28.3 | 5x |
| Lys378Arg | −36.2 | −32.8 | −33.1 | −32.2 | −37.8 | −32.6 | −27.4 | −30.0 | −30.0 | −30.6 | −32.3 | 1x |
| Lys378Asn* | −23.5 | −20.9 | −18.4 | −23.5 | −20.3 | −23.0 | −22.9 | −23.5 | −19.3 | −20.6 | −21.6* | 2x |
| Cys379Phe | −30.0 | −30.9 | −28.9 | −27.9 | −28.7 | −26.9 | −30.5 | −29.9 | −32.1 | −28.4 | −29.4 | 1x |
| Pro384Leu | −30.7 | −31.3 | −28.9 | −30.7 | −30.6 | −30.6 | −31.4 | −29.1 | −31.9 | −31.8 | −30.7 | 15x |
| Pro384Ser | −30.6 | −30.2 | −30.5 | −28.4 | −32.1 | −30.6 | −31.5 | −29.0 | −30.1 | −31.7 | −30.5 | 9x |
| Thr385Ala | −30.0 | −32.7 | −31.2 | −30.8 | −29.8 | −30.1 | −29.0 | −26.0 | −30.9 | −29.2 | −30.0 | 2x |
| Arg403Lys | −32.5 | −31.5 | −26.8 | −29.3 | −32.3 | −32.3 | −34.0 | −34.4 | −31.7 | −28.6 | −31.3 | 9x |
| Arg403Ser | −31.4 | −32.4 | −31.6 | −33.3 | −31.0 | −32.4 | −31.7 | −29.5 | −31.8 | −31.6 | −31.7 | 1x |
| Arg408Ile | −28.2 | −29.9 | −29.5 | −31.9 | −30.9 | −29.7 | −28.8 | −31.3 | −28.7 | −31.0 | −30.0 | 8x |
| Gln409Glu | −27.8 | −29.4 | −32.4 | −28.1 | −30.3 | −29.0 | −28.3 | −27.3 | −27.3 | −26.7 | −28.7 | 1x |
| Gln414Arg | −32.7 | −33.2 | −32.8 | −32.9 | −31.0 | −30.4 | −31.6 | −30.9 | −30.6 | −31.0 | −31.7 | 6x |
| Gln414Lys | −30.9 | −31.3 | −30.5 | −33.4 | −30.1 | −32.2 | −35.0 | −32.7 | −33.7 | −30.8 | −32.1 | 5x |
| Gln414Pro | −30.1 | −32.0 | −33.1 | −34.4 | −33.0 | −31.1 | −30.1 | −29.2 | −30.1 | −28.7 | −31.2 | 2x |
| Asn439Lys** | −38.2 | −33.0 | −32.1 | −31.4 | −29.9 | −29.8 | −35.5 | −35.5 | −38.6 | −32.3 | −33.6** | 56x |
| Ser501Tyr | −31.1 | −30.1 | −31.8 | −31.6 | −29.4 | −28.7 | −27.7 | −27.6 | −32.9 | −29.5 | −30.0 | 25x |
| Val503Phe | −28.6 | −33.3 | −33.3 | −31.8 | −34.4 | −32.6 | −30.4 | −33.4 | −34.0 | −32.7 | −32.5 | 1x |
| Val503Ile | −29.7 | −32.6 | −27.8 | −30.3 | −31.7 | −30.7 | −29.1 | −28.8 | −29.3 | −31.7 | −30.2 | 1x |
| Tyr508His | −32.1 | −27.7 | −32.0 | −25.6 | −28.1 | −28.7 | −28.7 | −27.2 | −26.0 | −27.6 | −28.4 | 9x |

FastContact-calculated interface interaction electrostatic plus desolvation free energies (kcal/mol) per 0.5 nanosecond snapshots from 5 nanosecond Molecular Dynamics runs of SARS-CoV-2 RBD variants in complex with h1_VHH72_S56A, and their average. The Lys378Asn variant (indicated with *) is predicted to severely impair the recognition, whereas improved binding is predicted for the most frequently observed Asn439Lys variant (indicated with **).

### Example 16. Identification of VHH72 Variants with Increased Virus Neutralizing Activity

To further improve the binding of VHH72 to SARS-CoV-2 RBD, mutations were introduced at several positions along the paratope using a structure-guided molecular modeling approach. Since at the start of our investigation no SARS-CoV-2 RBD structure was yet available, a model of SARS-CoV-2 RBD was obtained through the I-TASSER server²⁰, which was superposed by means of the Swiss-PdbViewer²¹, to the crystal structure of SARS-CoV-1 RBD (PDB code: 6WAQ chain D) in complex with VHH72 (FIG. 24d). At or near the VHH72 epitope, only three residues are different between the RBD of SARS-CoV-1 and -2: (1) Ala372 (Thr359 in SARS-CoV-1), resulting in the absence of a glycan on Asn370 (Asn357 in SARS-CoV-1); (2) Asn439 (Arg426 in SARS-CoV-1), resulting in the loss of an ionic interaction with Asp61 of VHH72; and (3) Pro384 (Ala371 in SARS-CoV-1). Pro384 is close to Tyr369 (Tyr356 in SARS-CoV-1), for which I-TASSER predicted a different conformation: pointing upward in the SARS-CoV-2 RBD model, whereas in the SARS-CoV-1 RBD-VHH72 cocrystal structure, this tyrosine is pointing downward and resides in a groove-like depression between two small helixes of the RBD. The up conformation of Tyr369 sets it in a mostly hydrophobic small cavity of VHH72, contacting residues Ser52, Trp52a, Ser53, Ser56 (all in CDR2) and Val100 (CDR3)(FIG. 24d). Molecular dynamics simulations with Gromacs²² shows that Tyr369 can be readily accommodated in that cavity. The model, however, revealed a polar/hydrophobic mis-match with Ser56's hydroxyl function, which points to the center of the Tyr369 aromatic system. Indeed, binding experiments with the different mutants showed that the VHH72 Ser56Ala substitution gave a substantial binding improvement (see below and FIG. 25). Consultation at the FastContact 2.0 server¹⁴ of time-frames from molecular dynamics simulations indicates that the gain in binding of the Ser56Ala mutant is mainly due to a local desolvation effect, suggesting that Ser56Ala allows water molecules in VHH72's small cavity to be easier replaced by Tyr369. Many cryo-EM or crystal structures containing SARS-CoV-2 RBD have in the meantime appeared (e.g. PDB-entries 6VSB, 6M17, and 6VXX)²³⁻²⁵, the majority of which show the same upward conformation of Tyr369. We hypothesize that Tyr369, as well as its Tyr356 counterpart in SARS-CoV-1 RBD, can flip into up or down positions, and that the up position in SARS-CoV-2 RBD prevails due to the nearby Pro384 (Ala371 in SARS-CoV-1). Of note, in the I-TASSER SARS-CoV-1 RBD model, Tyr365 is also pointing upward, but cryo-EM or crystal structures always show the downward conformation as observed for the corresponding Tyr352 in SARS-CoV-1 RBD (FIG. 24d).

### Example 17. Binding Affinity Determination of Monovalent Humanized VHH72 Variants Via Biolayer Interferometry (BU)

We humanized VHH72 by mutating the framework regions 1, 3 and 4, based on a sequence comparison with the human IGHV3-JH consensus sequence, hereafter referred to as VHH72_h1 (SEQ ID NO:2), and further by the conservative substitution of Q or E at position 1 to D, resulting in VHH72_h1(E1D) (SEQ ID NO:3). The S56A mutation was subsequently introduced into the humanized variants, resulting in VHH72_h1(S56A) (SEQ ID NO:5) and VHH72_h1(E1D; S56A) (SEQ ID NO:6), after which the function, biochemical and biophysical stability were assessed of the purified monomeric VHH72 variants.

To assess the impact of the introduction of the S56A mutation on the binding affinity in a 1:1 interaction, off-rate analysis was done of humanized VHH72 variants h1 towards the monomeric viral Spike RBD protein of SARS-CoV-2 and SARS-CoV-1, respectively. Hereto the biotinylated RBD domains were captured onto streptavidin tips (FortéBio), and next subjected to distinct VHH72 variants. The S56A introduction improved the off-rate of humanized VHH72 variants towards both SARS-CoV-1 RBD protein and SARS-CoV-2 RBD protein by around 1.5-fold, with off-rates between 1.0-2.4×10-3 s-1 (FIG. 25a).

To assess the affinity of the VHH72 variants in a 1:1 interaction, the kinetic binding constant K_{D} of the monovalent affinity optimized variants VHH72(S56A into h1) were assessed in BLI, comparing binding to monomeric SARS-CoV-2 RBD protein, and dimeric SARS-CoV-2 RBD-Fc-fusion. As reference, the humanized VHH72 h1 was included. The concentration range of VHHs was between 100 nM and 1.56 nM, and results were fitted according to 1:1 interaction. Results are shown in FIGS. 25b and 26.

The S56A introduction improved the 1:1 K_{D} with 3-fold on monomeric RBD, and >6-fold on Fc-fusion. Notably, there is a clear difference in the kinetic parameters between monomeric RBD and Fc-fusion. On monomeric RBD there is a slower association rate, compensated by a slower dissociation rate (in 10⁻³ s⁻¹ range), resulting in comparable K_{D} values on Fc-fusion. VHH72 h1_S56A has a K_{D} of 3.09 nM on monomeric RBD, and K_{D} 5.26 nM on the RBD-Fc. There is a 3-6-fold improvement in off-rate of the VHH72 h1 S56A variant compared to the VHH72 h1.

In conclusion, the S56A substitution increased the affinity of VHH72 for immobilized SARS-CoV-2 Spike and RBD proteins, yielding a K_{D} 3.1 nM (Kdis 6.9×10-4 s-1) measured in a 1:1 interaction in BLI (FIG. 25b). Furthermore, the monovalent VHH72_h1_S56A competes 7 times better than VHH72-wt and VHH72_h1 with SARS-CoV-2 RBD for binding to ACE2 on the surface of VeroE6 cells (FIG. 26b). This improved affinity resulted in a significantly improved neutralizing potency of VHH72_h1_S56A as determined with a VSV-dG SARS-CoV-2 spike pseudotyped virus neutralization assay (FIG. 26c). Importantly, VHH72_S56A also displayed increased affinity for SARS-CoV-1 RBD (FIGS. 25a and 26d) and could neutralize SV-dG SARS-CoV-1 spike pseudotypes 10 fold better than the parental VHH72 (IC₅₀ VHH72_h1: 0.491 μg/ml; IC₅₀ VHH72_h1_S56A: 0.045 μg/ml) (FIG. 26e).

### Example 18. Bivalent VHH72_S56A Constructs Increase Anti-SARS-CoV Potency

The sequence optimized VHH72 was fused to a human IgG1 Fc domain and analyzed with a range of linkers and hinge regions. Genetic fusion to an IgG Fc is a well-established method to increase the half-life of a VHH in circulation, and it creates bivalency of VHH72 to increase its anti-viral potency^{10,26}.

A set of VHH72 variants were expressed as VHH72-Fc fusions in Pichia pastoris and screened for improved binding off-rates to SARS-CoV-2 RBD protein with Biolayer interferometry (BLI). Mutations introduced at position S56A improved the off-rate. The VHH72_S56A-Fc mutant consistently performed better in a subsequent SARS-CoV-2 RBD ELISA and a flow cytometry-based assay using SARS-CoV-1 and -2 spike expressing 293T cells as compared to the VHH72-Fc construct.

The possible contribution of IgG effector functions to disease severity in COVID-19 patients is still unclear²⁷. We opted to include a human IgG1 with minimal Fc effector functions in our VHH72-Fc designs because there is uncertainty about the possible contribution of IgG effector functions to disease severity in COVID-19 patients^{9,27,86}. To this effect, and as also chosen by several other anti-SARS-CoV-2 antibody developers⁸⁷⁻⁸⁸, we opted for use of the well-characterized LALA mutations in the Fc part, extended or not with the P329G mutation^{7,89,90}. So in addition to the wild type IgG1 Fc, a human IgG1 Fc LALA and LALAPG variant with minimal Fc effector functions were included in our VHH72-Fc fusion construct designs⁹. The series of VHH72-Fc constructs was expressed in transiently transfected ExpiCHO cells and proteins purified from the culture medium were used for further characterization. Compared to VHH72-Fc and VHH72_h1-Fc, VHH72_h1_S56A-Fc showed a two- to four-fold higher affinity for SARS-CoV-2 Spike (S) (Table 2; FIG. 27a,b).

TABLE 2
Kinetics of VHH72 variants as determined by BLI.

| Immobi- | | Sample | | | K_{D} | kₒₙ | k_{off} | K_{D} | kₒₙ | k_{off} |
|---|---|---|---|---|---|---|---|---|---|---|
| lized | Strain | no. | Long name | n | (M) | (1/Ms) | (1/s) | SD | SD | SD |
| RBD-mFc | ExpiCHO | D72-02 | VHH72-GS-hIgG1hinge-hIgG1Fc | 2 | 1.42E−10 | 1.83E+06 | 2.60E−04 | 1.27E−12 | 1.29E+05 | 2.08E−05 |
| RBD-mFc | ExpiCHO | D72-13 | VHH72-h1_(G₄S)₃_{_{—}}VHH72- | 2 | <1.0E−12 | 2.65E+06 | <1.0E−07 | N/A | 2.86E+05 | N/A |
| | | | h1_GS_hIgG1hinge-hIgG1Fc | | | | | | | |
| RBD-mFc | ExpiCHO | D72-15 | VHH72-GS-hIgG1hinge-hIgG1Fc- | 2 | 1.30E−10 | 2.29E+06 | 2.97E−04 | 1.84E−12 | 1.54E+05 | 2.42E−05 |
| | | | L234A-L235A-P329G | | | | | | | |
| RBD-mFc | ExpiCHO | D72-16 | h1-VHH72-GS-hIgG1hinge-hIgG1Fc | 2 | 1.02E−10 | 1.89E+06 | 1.91E−04 | 2.04E−11 | 6.93E+04 | 3.15E−05 |
| RBD-mFc | ExpiCHO | D72-17 | h1-VHH72-GS-hIgG1hinge-hIgG1Fc- | 2 | 1.00E−10 | 1.77E+06 | 1.78E−04 | 1.60E−11 | 2.12E+05 | 4.94E−05 |
| | | | L234A-L235A-P329G | | | | | | | |
| RBD-mFc | ExpiCHO | D72-22 | h1-VHH72-S56A-GS-hIgG1hinge- | 2 | 4.71E−11 | 1.07E+06 | 4.87E−05 | 3.15E−11 | 1.14E+05 | 2.84E−05 |
| | | | hIgG1Fc | | | | | | | |
| RBD-mFc | ExpiCHO | D72-23 | h1-VHH72-S56A-GS-hIgG1hinge- | 2 | 4.77E−11 | 1.76E+06 | 8.16E−05 | 2.64E−11 | 1.71E+05 | 3.82E−05 |
| | | | hIgG1Fc-L234A-L235A-P329G | | | | | | | |

Binding affinity of VHH72 monovalent and multivalent Fc fusions to immobilized SARS-CoV-2 RBD, either mouse Fc fused (RBD-mFc) or monomeric human Fc fused (RBD-mono-hFc). Apparent kinetics are based on a global 1:1 fit of the data.

This increased affinity was also observed in flow cytometry-based quantification assays using full length spike expressed on the cell surface, a VeroE6 cell-based SARS-CoV-2 RBD competition assay.

The VHH72_h1(E1D,S56A)_10GS_Fc hIgG1 LALA (batch PB9683; SEQ ID NO: 22) showed an apparent binding affinity towards full length S protein of Sars-CoV-2 expressed on Hek293 cells of EC₅₀ 45.08 ng/mL (FIG. 27 c, d). Binding to the Sars-CoV-2 RBD-SD1-hFc protein in ELISA resulted in an EC₅₀ of 47.8 ng/mL (FIG. 27e). The VHH72_h1(E1D,S56A)_10GS_Fc hIgG1 LALA (batch PB9683) competed with the binding of the monovalent VHH72_h1(E1D,S56A) sequence optimized (SO) to the SARS-CoV-2 RBD protein in competition AlphaLISA with an IC50 of 6.7 ng/mL (FIG. 27f). So, the LALA or LALAPG mutation in the Fc region of VHH72(S56A)-Fc did not change the affinity for SARS-CoV-2 S or -RBD binding as determined by ELISA, flow cytometry, and BLI.

VHH72_h1_(E1D,S56A)-Fc IgG1 with or without the LALA, FALA or LALAPG substitutions in the Fc part, neutralized SARS-CoV-2 Spike pseudotyped VSV approximately 3-7 fold better than their wt VHH72-Fc counterparts (FIG. 28). The VHH72_h1(E1D,S56A)_10GS_Fc hIgG1 LALA (PB9683) showed a neutralization potency of Sars-CoV-2 pseudotyped lentivirus (VSV) of IC₅₀ 31 ng/mL (0.37 nM), approximately 8-fold improved compared to the prototype VHH72-Fc (IC50 263 ng/mL). Constructs with alternative Fc types, such as hIgG4_FALA, hIgG1 and IgG1_LALAPG, showed similar sub nM potencies, with IC50 raging between 40-55 ng/mL (FIG. 28). The improved neutralizing potency of the S56A substitution in VHH72-Fc was also observed in a plaque reduction neutralization assay using authentic SARS-CoV-2 virus: VHH72_h1_S56A-Fc (IC₅₀=0.12 μg/ml) was 6 fold more potent than VHH72-Fc (IC₅₀=1.01 μg/ml) and VHH72_h1-Fc (IC₅₀=0.94 μg/ml), with no apparent impact of the LALA or LALAPG substitution in the Fc part of these constructs (FIG. 29). Finally, VHH72_S56A-Fc outperformed its wt counterpart in preventing the interaction between SARS-CoV-2 RBD and human ACE2 (FIG. 30).

### Example 19. Tetravalent VHH72_S56A-Fc Constructs Further Increase Anti-SARS-CoV Potency

VHHs can be easily formatted into tandem tail-to-head fusions, usually without any compromise on expression levels and stability²⁸. In addition, such multivalent constructs typically have increased target binding affinity and, in the context of viruses that display antigenic diversity, breadth of protection²⁹⁻³¹. We therefore grafted VHH72_S56A_h1 as a tandem repeat, with the VHHs separated from each other by a (G₄5)₃ linker, fused to human IgG1 Fc via a GS linker (e.g. as in SEQ ID NO:21; D72-55 sample) and expressed this molecule in transiently transfected ExpiCHO cells. The resulting tetravalent VHH72-Fc fusion construct displayed a >100-fold higher affinity for SARS-CoV-2 RBD than its bivalent counterpart (FIG. 31a). By combining the S56A mutation with a tetravalent format, the in vitro antiviral potency was further increased, reaching a PRNT₅₀ value of 0.02 μg/ml, i.e. 50-fold lower than the parental construct (FIG. 31b).

### Example 20. High Expression and Stability of Multivalent VHH72-Fc Fusions

Robust expression levels, chemical and physical stability as well as a homogenous spectrum of posttranslational modifications are important prerequisites for the “developability” of a protein biologic³². Two mutations are frequently introduced at either terminus of recombinant monoclonal antibodies that are intended for clinical use: a change of the N-terminal glutamic acid residue, which is prone to spontaneous pyroglutamate formation during production and storage, into an aspartic acid residue (indicated previously as E1D), and deletion of the C-terminal lysine residue, which is susceptible to removal by carboxypeptidase and can lead to charge heterogeneity of the drug substance³³. In addition, a truncation in the human IgG1 hinge was done to avoid possible non-canonical disulphide bond formation, as the naturally occurring hinge has a cysteine residue that forms an intermolecular disulphide bond with the constant domain of the paired light chain. The constructs of for instance batches D72-52 (VHH72_h1_E1D_S56A-(G4S)₂-hIgG1hinge_EPKSCdel-hIgG1_LALAPG_Kdel; often shortened herein to VHH72_h1_E1D_S56A-10GS-hIgG1Fc_LALAPG; SEQ ID NO: 20), D72-55 (SEQ ID NO: 21), D72-53 or PB9683 ((VHH72_h1_E1D_S56A-(G₄S)r-hIgG1hinge_EPKSCdel-hIgG1_LALA_Kdel; often shortened herein to VHH72_h1_E1D_S56A-10GS-hIgG1Fc_LALA, SEQ ID NO:22), as used herein. The RBD-binding kinetics and SARS-CoV-2 neutralizing activity with or without hinge truncation were confirmed to be similar (FIG. 32).

The VHH72-Fc variants were expressed with levels as high as 1.2 mg/ml in transiently transfected ExpiCHO cells, irrespective of linkers and Fc types. We also determined the physical stability of the purified VHH72-Fc variant constructs. Differential scanning fluorimetry over a 0.01° C./s ramp showed that thermal stability is enhanced by humanization and the introduction of the S56A mutation, while tetravalency has a minor negative effect on thermal stability (Table 3). Such a negative effect was also observed when probing the aggregation temperature of bivalent versus tetravalent formats, that is, a 7° C. destabilization was noted for tetravalent constructs.

TABLE 3
Thermal stability.

| | | | | Tₘ₀ ± | Tₘ₁ ± | Tₘ₂ ± |
|---|---|---|---|---|---|---|
| No. | Buffer | Strain | Construct | SD | SD | SD |
| D72-2 | PBS | ExpiCHO | VHH72_GS_hIgG1hinge-hIgG1Fc | | 63.0 ± 0.338 | 81.6 ± 0.227 |
| D72-15 | PBS | ExpiCHO | VHH72_GS_hIgG1hinge-hIgG1Fc-LALAPG | | 62.8 ± 0.232 | 82.8 ± 0.402 |
| D72-16 | PBS | ExpiCHO | VHH72_h1_GS_hIgG1hinge-hIgG1Fc | | 64.5 ± 0.075 | 81.4 ± 0.117 |
| D72-17 | PBS | ExpiCHO | VHH72_h1-GS_hIgG1hinge-hIgG1Fc-LALAPG | | 64.3 ± 0.119 | 81.7 ± 0.035 |
| D72-22 | PBS | ExpiCHO | VHH72_h1_S56A-GS_hIgG1hinge-hIgG1Fc | | 65.0 ± 0.204 | 81.9 ± 0.425 |
| D72-23 | PBS | ExpiCHO | VHH72_h1_S56A-GS_hIgG1hinge-hIgG1Fc- | | 64.8 ± 0.336 | 81.9 ± 0.296 |
| | | | LALAPG | | | |
| D72-13 | PBS | ExpiCHO | VHH72-h1-(G₄S)₃-VHH72-h1_GS_hIgG1hinge- | 49.2 ± 0.714 | 64.0 ± 0.354 | 79.8 ± 0.406 |
| | | | hIgG1Fc | | | |
| D72-52 | PBS | ExpiCHO | VHH72_h1E1D-56A-(G₄S)₂_{_{—}}hIgG1hinge- | | 65.5 ± 0.155 | 85.0 ± 0.544 |
| | | | hIgG1Fc-LALAPG-Kdel | | | |
| D72-55 | PBS | ExpiCHO | VHH72_h1_E1D-56A-(G₄S)₃-VHH72_h1- | | 64.7 ± 0.432 | N/A |
| | | | 56A_GS_hIgG1hinge-hIgG1Fc-LALAPG-Kdel | | | |

Differential scanning fluorimetry using a SYPRO Orange probe in a 0.01° C./s ramp. Blank-subtracted data were normalized to 0-100%. After cubic spline interpolation of the melting curves, first derivatives were plotted to identify each melting temperature (Tm). Tm values are shown as mean and standard deviation (SD) of triplicate measurements.

### Example 21. Live Virus Neutralization

SARS-CoV-2 strain BetaCov/Belgium/GHB-03021/2020 (EPI ISL 407976|2020-02-03) was used from passage P6 grown on VeroE6 cells as described³. VHH-Fc constructs were three-fold serially diluted, using a starting concentration of 20 μg/ml, mixed with 100 PFU SARS-CoV-2 and incubated at 37° C. for 1 h. VHH-Fc-virus complexes were then added to Vero E6 cell monolayers in 12-well plates and incubated at 37° C. for 1 h. Subsequently, the inoculum mixture was replaced with 0.8% (w/v) methylcellulose in DMEM supplemented with 2% FBS. After 3 days incubation at 37° C., the overlays were removed, the cells were fixed with 3.7% PFA, and subsequently stained with 0.5% crystal violet. Half-maximum neutralization titers (PRNT₅₀) were defined as the VHH-Fc concentration that resulted in a plaque reduction of 50%. Results are shown in FIG. 10. Molecules D72-51 (VHH72_h1_E1D__S56A-10GS-hIgG1hinge_EPKSCdel-hIgG1Fc_LALAPG) and D72-52 (VHH72_h1_E1D_S56A-10GS-hIgG1hinge_EPKSCdel-hIgG1Fc_LALAPG_Kdel; SEQ ID NO:11) containing hIgG1_LALAPG Fc showed PRNT50 of 164.8 ng/mL and 163.9 ng/ml, respectively.

### Example 22. Protection of Hamsters Against SARS-CoV-2 Challenge

To assess the in vivo anti-viral efficacy of our bivalent and tetravalent molecules, we pursued a Golden Syrian hamster challenge model that mimics aspects of severe COVID-19 in humans, including high lung virus loads and the appearance of lung lesions³⁴. In a first experiment, we compared the protective potential of a bivalent (D72-23=VHH72_h1_S56A-Fc_LALAPG; SEQ ID NO:19) and a tetravalent (D72-13=VHH72_h1-(G₄S)₃-VHH72_h1-GS-hIgG1hinge-hIgG1Fc) construct, which have similar in vitro SARS-CoV-2 neutralizing potency (D72-23, PRNT₅₀=0.13 μg/ml; D72-13, PRNT₅₀=0.10 μg/ml) administered at 20 mg/kg intraperitoneally one day prior to challenge infection with 2.4×10⁶ TCID₅₀ of SARS-CoV-2 (FIG. 34a). Control animals received the same dose of Synagis. Strong and significant reduction in viral RNA levels in the lungs (>4 log) and ileum (2 log) were observed on day 4 after infection in both VHH72-Fc treated groups compared with the Synagis control group, and in stool samples also for the bivalent construct (FIG. 34b). Importantly, no infectious virus was detectable in lung homogenates from any of the VHH72-Fc treated hamsters except for one (FIG. 34b). Protection was also evident based on p-CT imaging of the lungs at day 4, which showed a significantly reduced incidence of dilated bronchi in the VHH72-Fc treated animals (FIG. 34c). In a follow-up study, prophylactic administration of a lower dose of 4 mg/kg of the bivalent lead also provided significant reduction in lung viral load (FIG. 35a). In this experiment, higher variability was observed in the 20 mg/kg dose group, with 2 out of 5 hamsters having virus loads in the lungs and nasal washes that were comparable to those in the Synagis control group (FIG. 35b). These two outliers, however, had no detectable VHH72-Fc in serum at endpoint, which may have been due to a technical error at the time of injection (FIG. 35c). As a next step, we assessed the therapeutic potential of bivalent and tetravalent molecules and dosed at 1 and 7 mg/kg intraperitoneally 16 h after infection. As a prophylactic control, the bivalent construct was administered 1 day prior to the challenge at 7 mg/kg (FIG. 36a). Significant reductions in lung infectious virus (4 log) were observed compared to the Synagis control animals for all the VHH72-Fc treated groups except for the 1 mg/kg treatment with the bivalent construct (FIG. 36b). Also, strong reduction in genomic viral RNA levels (>5 log) were seen for the highest dose of bivalent in both therapeutic and prophylactic setting, with the other groups showing higher variability (FIG. 36b). μCT-imaging revealed reduced pathology in the prophylactic group and, surprisingly, in the animals that had been treated with the lowest dose of the tetravalent construct, but not in the other groups (FIG. 36c). In the 3 hamster experiments described above, we used a challenge virus derived from BetaCov/Belgium/GHB-03021/2020, which is closely related to the first SARS-CoV-2 viruses isolated in Wuhan in the beginning of the new coronavirus pandemic³⁵. Since February 2020, a SARS-CoV-2 variant virus with a glycine instead of an aspartic acid residue at position 614 in the spike emerged and has now become the dominant pandemic form³⁶. SARS-CoV-2 virus isolates with this D614G spike variant replicate to higher titers in vitro, but there is no evidence that infection with these viruses leads to increased transmissibility or disease, or that they are less susceptible to neutralizing antibodies³⁶. Given that D614G variant viruses dominate the pandemic now, we also performed a challenge experiment in hamsters with a virus preparation derived from strain BetaCoV/Munich/BavPat1/2020, which carries this mutation (FIG. 37a). To assess a dose relationship, therapeutic treatment was performed with three doses ranging from 20, 7 and 2 mg/kg IP of either bivalent or tetravalent molecules 4 hours after challenge, a time point where lung viral titers were already increasing. As control, hamsters in one group received a dose of 20 mg/kg of bivalent one day before the challenge, with Synagis serving as a negative control in the therapeutic setting. Lung virus replication was completely controlled at 20 and 7 mg/kg, although 2 out of 6 animals in the tetravalent 20 mg/kg group showed residual virus titers, with variability occurring at the 2 mg/kg dose of the bivalent constructs (FIG. 37b). Interestingly, gross lung pathology was lowest in the animals that had been treated with 7 mg/kg of the bivalent construct. Collectively, the results of these 4 challenge experiments in hamsters, using two different virus strains, indicate that prophylactic as well as therapeutic injection of VHH72_S56A-Fc fusion constructs can fully control viral replication in this stringent SARS-CoV-2 challenge model.

Furthermore, gross pathology analysis (FIG. 37C) showed that in control animals 20-40% of the lung surface showed lung lesions. Therapeutic administration of D72-52 strongly prevented lung lesions at 7 mg/kg dose, with 5/6 animals no lung lesions detectable. Infection with SARS-CoV-2 isolate (Munich P3) gave progressive loss of body weight in all groups. None of the treatments groups significantly prevented body weight loss (FIG. 37D-E).

In the lower respiratory tract, both bivalent and tetravalent VHH-Fc formats significantly prevent infectious virus spread to the lung in therapeutic setting, with full reduction >4 logs at the 2 highest doses in both formats (FIG. 37B). No infectious virus particles were observed in BALF at day 4 at all concentrations, while the viral RNA load show a dose relationship for both bivalent and tetravalent formats (FIGS. 37F and G).

In the upper respiratory tract, in the day 4 nasal turbinates, very high virus levels were observed in the control group, with dose-dependent reduction in both treatment groups (FIG. 37H). A clear dose relationship was observed in infectious virus in throat swabs taken at day 1 and 2 following treatment with bivalent D72-52 and tetravalent D72-55 VHH72 h1 S56A-Fcs (FIG. 37i). Viral mRNA copies remain high after infectious virus is cleared in throat swabs and nasal turbinates. Good correlations are seen with viral load in upper and lower respiratory tract (FIG. 37J).

In conclusion, clear anti-viral efficacy after therapeutic treatment observed in lung viral load and gross lung pathology. In general, highest reduction of both viral replication in the upper and lower respiratory tract as well as gross and histopathological changes was observed in the animals treated therapeutically with the 20 and 7 mg/kg dose of both compounds, and the animals that were treated prophylactically.

### Example 23. Hamster Challenge Studies with D72-53 (PB9683)

Finally, for the VHH72_h1(E1D, S56A)_10GS_IgG1_LALA construct (D72-53; SEQ ID NO: 22; PB9683 batch), the format optimization of the VHH72-Fc involved the fusion via a flexible Glycine-Serine linker (GSGGGGSGGGGS, or 10GS) to the shortened hinge of human IgG1 (EPKSCdel), linked to a Fc domain of human IgG1_LALA forming a bivalent single domain antibody format, and at the C-terminal end a lysine residue was omitted. This resulted in a molecular weight of 39.6 kDa (monomer) or 79.1 kDa (dimer) and an iso-electric point of 6.26 (PI). Since similar data have always been observed for the LALA or LALAPG variants, similar analyses for compositions comprising any of these variants have been performed in vivo. Golden Syrian hamsters are highly permissive to SARS-CoV-2 infection and develop bronchopneumonia and strong inflammatory responses in the lungs with neutrophil infiltration and oedema. This was therefore considered a relevant model of disease and was used to assess the efficacy of D72-53 (Batch PB9683) at 2 dose levels (2 and 7 mg/kg) and in 2 settings (treatment and prophylactic) (FIG. 40).

The SARS-CoV-2 strain used, BetaCov/Belgium/GHB-03021/2020, was recovered from a nasopharyngeal swab taken from an RT-qPCR confirmed asymptomatic patient who returned from Wuhan, China in the beginning of February 2020. A close relation with the prototypic Wuhan-Hu-1 2019-nCoV strain was confirmed by phylogenetic analysis. Infectious virus was isolated by serial passaging on HuH7 and Vero E6 cells; passage 6 virus was used for the studies described here, similar as in the in vitro neutralization test. The titer of the virus stock was determined by end-point dilution on Vero E6 cells by the Reed and Muench method. Synagis (palivizumab) which is a mAb targeting respiratory syncytial virus was used as a negative control. 6-8 weeks old female Syrian Golden (SG) hamsters of 90-120 g were randomized to the different treatment groups.

Animals were treated in a therapeutic or prophylactic setting with D72-53 (PB9683) (7, 4 or 2 mg/kg) 24 h before or 19 h after infection by intraperitoneal administration. Hamsters were monitored for appearance, behaviour and weight. At day 4 post infection (pi), hamsters were euthanized. Lungs were collected and viral RNA and infectious virus were quantified by RT-qPCR and end-point virus titration, respectively (FIG. 40). Blood samples were collected before infection (prophylactic groups) and at day 4 for PK analysis. Lung tissue sections were prepared for histological examination. Tissue sections were scored blindly for lung damage by an expert pathologist. The scored parameters, to which a cumulative score of 1 to 3 was attributed, were the following: congestion, intra-alveolar haemorrhagic, apoptotic bodies in bronchus wall, necrotizing bronchiolitis, perivascular oedema, bronchopneumonia, perivascular inflammation, peribronchial inflammation and vasculitis. A higher score indicates a more pathological condition.

All the D72-53 (PB9683) treated groups had a significantly lower viral RNA load in the lung compared to the control group (FIG. 40 A). Protection was observed at both dose groups in the prophylactic setting and the two highest therapeutic doses, while the 2 mg/kg dose group showed higher variability in lung viral RNA. Infectious virus levels in lung were reduced below detection levels in most D72-53 (PB9683) treated hamsters, irrespective of dose (FIG. 40 B).

Histology assessment revealed highest variability in cumulative lung damage score in the D72-53 (PB9683) 2 mg/kg therapeutic group, which was also not statistically significantly different from the negative control group (FIG. 40 C). The other PB9683 groups were all significantly improved (indicated by a lower score) compared to the control group. In conclusion, a clear dose relationship was observed in the therapeutical setting, where 2 mg/kg dose lost protective effect.

In a further study, an intermediate therapeutic dose of 4 mg/kg D72-53 (batch PB9683) was evaluated in comparison to the pre-lead D72-58, which is identical to D72-53 except for the S56A point-mutation (FIG. 41). Production of D72-53 protein batch PB9683 was done in ExpiCHO system from transient transfected cells, where the antibody is secreted into the culture medium. Purification was done by standard Protein A affinity chromatography followed by gel filtration, yielding a purity of >99% assessed by size exclusion-UPLC. The batch was formulated in 10 mM PBS pH7.4. The endotoxin levels were <1 EU/mg. Production of ‘prelead’ protein batch D72-58 was done in ExpiCHO system from transient transfected cells using a pCDNA3.3 TOPO expression vector. Purification of the antibody from the culture medium was done by ProteinA chromatography, followed by multiple rounds of gelfiltration (Superdex 200 μg), resulting in endotoxin levels <1 EU/mg. Formulation was in 10 mM PBS pH7.4.

All proteins were diluted to a concentration of 1 mg/mL in PBS pH 7.4, allowing the administration of volumes around 0.5 mL per hamster (weights ranging 100-120 g) for obtaining a dose of 4 mg/kg.

A validated SARS-CoV-2 Syrian Golden hamster infection model was used as described in Ref. 13 and 69. This model is suitable for the evaluation of the potential antiviral activity and selectivity of novel compounds/antibodies⁷⁰ (see materials and methods). The treatment schedule is provided in Table 4.

TABLE 4
Treatment schedule.

| | | TCID₅₀ | | | | |
|---|---|---|---|---|---|---|
| | ham- | Inoculum/ | | | Frequency | |
| Group | sters | 50 μL | Treatment | Dose | dosing | MOA |
| Group 1 | 6WT | 1.89E+06 | Control | 4 mg/kg | Once, 24 h | IP |
| | | | (Synagis) | | post-infection | |
| Group 2 | 6WT | 1.89E+06 | D72-53 | 4 mg/kg | Once, 24 h | IP |
| | | | (PB9683) | | post-infection | |
| Group 3 | 6WT | 1.89E+06 | Pre-lead | 4 mg/kg | Once, 24 h | IP |
| | | | (D72-58) | | post-infection | |

Determination of RNA viral load in the lung (FIG. 41, left panel) was done via q-RT-PCR analysis, and of infectious virus load in the lung (FIG. 41, right panel) was done using virus end-point titrations on confluent Vero E6 cells.

Therapeutic treatment with D72-53 (PB9683) (4 mg/kg), or Pre-lead (D72-58) (4 mg/kg) efficiently reduced the lung viral RNA load and infectious virus particles compared to the control Ab Synagis (4 mg/kg) in this SARS-Cov-2 hamster infection model.

From the analysis, we may also conclude that the D72-53 batch shows a difference in median lung viral load of 1.5 log for both the TCID50 and viral RNA copies readouts as compared to the Prelead batch. This is calculated based on the median values: 312.5 vs 10 (=LLOQ) on TCID50, respectively and the median values for viral RNA copies of 67406 vs 2381, resp.

As a reference to the Synagis negative control, the log differences obtained were:
Prelead vs Synagis: 1.4 log on RNA, and 3 logs on TCID50.
D72-53 Lead vs Synagis: 2.9 log on RNA, and >4 logs on TCID50.

Since the only difference between the D72-53 (PB9386; SEQ ID NO:22) and Prelead (D72-58; SEQ ID NO:17) protein is the S56A mutation in the VHH CDR2 region, the contribution of this anti-viral efficacy as log reduction in the D72-53 Lead may be credited to its difference in the S56A mutation, rather than to its humanization substitutions.

Therapeutic systemic administration of low dosage of VHH72_S56A-Fc antibodies strongly restricted replication of both original and D614G mutant variants of SARS-CoV-2 virus in hamsters, and minimized the development of lung damage.

### Example 24. Flow Cytometric Analysis of Antibody Binding Sarbecovirus RBD Displayed on the Surface of Saccharomyces cerevisiae

A pool of plasmids, based on the pETcon yeast surface display expression vector, that encode the RBDs of a set of SARS-CoV2 homologs was generously provided by Dr. Jesse Bloom⁷². This pool was transformed to E. coli TOP10 cells by electroporation at the 10 ng scale and plated onto low salt LB agar plates supplemented with carbenicillin. Single clones were selected, grown in liquid low salt LB supplemented with carbenicillin and miniprepped. Selected plasmids were Sanger sequenced with primers covering the entire RBD CDS and the process was repeated until every desired RBD homolog had been picked up as a sequence-verified single clone. Additionally, the CDS of the RBD of SARS-CoV2 was ordered as a yeast codon-optimized gBlock and cloned into the pETcon vector by Gibson assembly. The plasmid was transformed into E. coli, prepped and sequence-verified as described above. DNA of the selected pETcon RBD plasmids was transformed to Saccharomyces cerevisiae strain EBY100 according to the protocol by Gietz and Schiestl⁷³ and plated on yeast drop-out medium (SD agar -trp -ura). Single clones were selected and verified by colony PCR for correct insert length. A single clone of each RBD homolog was selected and grown overnight in 10 ml liquid repressive medium (SRaf -ura -trp) at 28° C. These precultures were then back-diluted to 50 ml liquid inducing medium (SRaf/Gal -ura -trp) at an OD₆₀₀ of 0.67/mi and grown for 16 hours before harvest. After washing in PBS, the cells were fixed in 1% PFA, washed twice with PBS, blocked with 1% PFA and stained with dilution series of anti-RBD antibodies or synagis.

The VHH72_h1_E1D_S56A-(G₄S)₂-hIgG1hinge_EPKSCdel-hIgG1Fc_LALA_K477del (=D72-53 construct) amino acid sequence is depicted in SEQ ID NO:22. CB6 antibody corresponded to the sequence in SEQ ID NO: 64-65, for the light and heavy chain (Genbank MT470196 and MT470197). S309 antibody corresponds to SEQ ID NO: 62-63, from Pinto et al.⁹¹. An isotype control antibody Synagis hIgG1 (Medimmune) was included as negative control.

Binding of the antibodies was detected using Alexa fluor 633 conjugated anti-human IgG antibodies. Expression of the surface-displayed myc-tagged RBDs was detected using a FITC conjugated chicken anti-myc antibody. The fluorescence intensity of the cells was then analyzed using a BD LSR II flow cytometer.

As shown in FIG. 42b, the binding of D72-53 VHH72-Fc antibody was shown for all clade 1a and clade1b RBDs tested, as well as for clade3 RBD of BM48-31, and some of the clade 2 Bat SARS-related sarbecoviruses (RP-3 and HKU3-1, but not Rf1, ZXC21 and ZC45), indicating the very broad cross-protection that the VHH72-Fc antibody may provide for sarbecoviruses. For other Sars-Cov-2 specific monoclonal antibodies tested herein, the binding to the RBD domain was limited to clade 1b only (CB6), or clade 1a and 1b (S309). In conclusion, VHH72_S56A-Fc binds to clade 1, -2 and -3 RBDs of Sarbecoviruses.

### Example 25. SARS-CoV-2 Spike Protein Sequence Variant Analysis

SARS-CoV-2 genome sequences originating from human hosts were downloaded from GISAID (N=322,187 genomes available on Jan. 4, 2021). Genomes with invalid DNA character code were removed. Spike coding sequences were retrieved by aligning the genomes to the reference spike sequence annotated in NC_045512.2 (Wuhan-Hu-1 isolate, NCBI RefSeq). For this purpose, pairwise alignments were performed using R package Biostrings version 2.54.0, a fixed substitution matrix in the “overlap” mode with the following parameters according to Biostrings documentation: 1 and -3 for match and mismatch substitution scores; 5 and 2 as gap opening and gap extension penalties, respectively. Incomplete genomes without spike coding sequences, or that generated very short or no alignment were removed. Coding sequences with frame-disturbing deletions were also excluded and the remaining open reading frames were in-silico translated using Biostrings option to solve “fuzzy” codons containing undetermined nucleotide(s). In the next step, predicted spike protein sequences with undetermined amino acids (denoted as X), derived from poor sequencing results (Ns) were removed. Further, full-length sequences with a single stop codon or lacking a stop signal (due to a possible C-terminal extension) were retained, while proteins with premature stop codon(s) were excluded.

The resulting 240,239 quality-controlled spike protein sequences were aligned using the ClustalOmega algorithm and R package msa version 1.18.0 with default parameters and the BLOSUM65 substitution matrix. R packages seqinr 3.6-1 and BALCONY 0.2.10 were used to calculate amino acid frequencies for all mutations occurring in the dataset at least once. Major and minor allele frequencies and counts were assigned. Effects of individual mutations on spike expression and fold were derived from Starr et al.⁷². Binding energy of VHH72 to reference and mutated RBD was estimated using FastContact 2.0¹⁴ based on 30 and 10 molecular dynamics simulations, respectively. The impact of mutations on VHH72 binding (difference in kcal/mol compared to the reference RBD data) was statistically evaluated using a t-test (mutant vs. reference RBD) with a p-value≤0.05 based on 10 simulations. Epitopes of VHH72 and other anti-RBD antibodies (by PISA buried surface estimation⁷⁴) were represented as logical vectors and clustered using MONothetic Analysis Clustering Of Binary (R package cluster). Jaccard similarity of each epitope to VHH72 was calculated (score between 0 and 1, R package fpc). Data collected for spike protein RBD (positions 333-516) was visualized using ggplot2 version 3.3.0.

Molecular modeling of the SARS VHH-72 interaction with SARS-CoV-2 RBD was performed by Molecular Dynamics simulations with model-complexes of VHH72 (chain C from PDB-entry 6WAQ) and variants, with the outward-positioned RBD from the cryo-EM structure PDB-entry 6VSB of the SARS-CoV-2 prefusion spike glycoprotein (chain A, residues 335-528) and variants. The missing loops at residues 444-448, 455-490 and 501-502 in the cryo-EM RBD were reconstructed from the I-TASSER SARS-CoV-2 RBD model²⁰ and the missing residues were added by the Swiss-PDBViewer²¹. Simulations were with Gromacs version 2020.1²² using the Amber ff99SB-ILDN force field⁴² and were run for 5 nanoseconds. After conversion of the trajectory to PDB-format, snapshots were extracted for every 0.5 nanoseconds and were submitted to the FastContact 2.0 server¹⁴.

As shown in FIG. 43, based on the calculated binding energy and modelling information, we conclude that the RBD mutant variants analyzed herein, covering most circulating SARS-Cov-2 variants, should be susceptible to VHH72-S56A-Fc. In addition, the N439K mutant variant provides for a substitution in the epitope region of VHH72, and occurs frequently (ca 2%), and based on this analysis may enhance VHH72_S56A binding, as indicated by the binding energy in FIGS. 43 and 44.

The nearest RBM mutation in recently rapidly emerging SARS-CoV-2 isolates in the distant periphery of the VHH72 epitope is the N501Y mutation seen in both the variant B.1.1.7⁸³ and 501.V2⁸⁴ variants. Molecular dynamics calculations indicated that substitutions at this position would not affect VHH72 binding (FIG. 43). This was validated experimentally in a flow cytometry assay showing equally strong binding of VHH72-Fc to wild type RBD of SARS-CoV-2 as to RBD N501Y mutant expressed on the surface of mammalian cells in the context of the complete spike of SARS-CoV-1 (FIG. 50). Binding of VHH72-Fc to RBD variant N439K⁸⁵, was also not affected (FIG. 50). Finally, we provide evidence that the D72-53 lead molecule is unaffected in its binding by currently rapidly spreading SARS-CoV-2 variants, and demonstrate its unique wide scope of binding across the sarbecovirus clades.

### Example 26. Isolation of Additional SARS-CoV-2 Neutralizing VHHs

Further to the selected and optimized VHH72 ISVD, additional VHHs were identified as potently neutralizing SARS-CoV-2 by interacting with its Spike protein. To obtain additional VHH families, the following approaches were used. VHH-72 was originally isolated as a SARS-CoV-1 neutralizing VHH from a llama that was immunized 4 times with the spike proteins of the SARS-CoV-1 by bio-panning using the same SARS-Cov-1 spike protein. Since this VHH can also neutralize the SARS-CoV-2 virus by binding to a conserved region on the RBD distant from the site that interacts with ACE2, the SARS-CoV-2 host cell receptor, but is still able to block this interaction via sterical hindrance with the ACE2 protein backbones and an ACE2 glycan, this indicates that the used VHH immune library might contain a larger repertoire VHHs that can cross-react with the SARS-CoV-1 and SARS-Cov-2 RBDs. To isolate a second generation of VHHs that can potently neutralize SARS-CoV-2 the original non-panned VHH immune library (obtained after sequential immunizations with the SARS-CoV-1 and MERS-CoV spike proteins) was panned using monovalent SARS-CoV-2 RBD (RBD-SD1-huFc). After panning, 94 clones were picked and used to test in PE ELISA using SARS-CoV-2 RBD fused to bivalent murine Fc, SARS-CoV-2 RBD-SD1 fused to monovalent human Fc, SARS-CoV-1 RBD and SARS-CoV-1 Spike protein. Multiple VHHs present in the PE extracts could bind to all four tested antigens (data not shown). Clones that were able to bind both SARS-CoV-2 antigens were sequenced resulting in 25 unique VHH sequences without internal stop codons. The purified VHHs were tested for their ability to bind the SARS-CoV-1 and -2 RBD and Spike protein by ELISA. Although several of tested VHH can readily bind to the SARS-CoV-1 Spike protein and the SARS-CoV-2 RBD, respectively the antigens used for immunization and bio-panning. However, except for minor binding for a few VHHs, the majority could not efficiently bind the SARS-CoV-2 Spike protein. Next to ELISA we also investigated the binding of the VHHs to SARS-CoV-2 Spike protein expressed at the surface of cells by flowcytometry. In line with the ELISA results the vast majority of the tested VHHs failed to bind the SARS-CoV-2 spike protein. At 20 μg/ml clear binding was observed only for CoV-2 VHH2.50, which is highly related to VHH-72, and this classified in the same VHH72 family. Next, we investigated if VHH2.50, was able to neutralize SARS-CoV-2 in vitro using a SARS-CoV-2 spike pseudotyped VSV-d virus. At 20 μg/ml only VHH2.50 was able to almost completely neutralize SARS-CoV-2 Spike pseudotyped VSV virus (FIG. 48). Further analysis revealed that the neutralizing activity of VHH2.50 is highly similar with its related VHH, VHH-72 (FIG. 45).

Moreover, a third generation VHHs were obtained by immunizing the previously immunized llama 3 times additionally with the SARS-CoV-2 Spike protein. The obtained immune library was panned with either the SARS-CoV-2 spike protein or its RBD domain. Sequence analysis of the CDR3 revealed that the VHHs that can bind the SARS-CoV-2 RBD and Spike in PE ELISA can be attributed to 22 discrete VHH families. Although the CDR3 of some of these families are related to VHHs isolated from the VHH library obtained after the first immunization series of llama Winter, only VHH3.115 belonging to the VHH3.17 family has highly similar CDR1 and CDR2 sequences to VHH-72, in addition to its high degree of similarity to the CDR3, classifying those 3^{rd} generation VHHs (VHH3.17, VHH3.77, VHH3.115, VHH3.144, and VHH BE4) within the same sequence family as VHH-72, called family 72 (FIG. 45). We previously demonstrated that S56A substitution in VHH-72 increases its affinity for the SARS-CoV-2 spike protein and its neutralizing activity. Remarkably, all VHHs that related to the previously isolated VHH-72 had a S56G substitution. All (54) unique VHHs that bound to recombinant prefusion stabilized SARS-Cov-2 Spike protein or monomeric RBD-SD1-huFc in PE ELISA and that do not contain internal stop codons were selected for further PE analysis using, including binding to cell surface expressed WT full length SARS-CoV-2 Spike protein, inhibition of RBD binding to VERO E6 target cells that express ACE2 and neutralization of SARS-CoV-2 Spike pseudotyped VSV. Binding of the selected VHHs to cell surface expressed SARS-CoV-2 Spike protein was tested by flowcytometry. We investigated the ability of VHH containing PE extracts to interfere with the binding of RBD to Vero E6 target cells that express the ACE2 receptor. Recombinant RBD-muFc was mixed with 20-fold diluted PE and subsequently added to Vero E6 cells to allow RBD binding. Binding of RBD-muFc was tested by flow cytometry, and revealed that 19 out of 54 VHHs could completely or almost completely prevent binding of RBD to ACE2 at the surface of Vero E6 cells. Only VHHs that can most potently bind to the RBD on the surface of cells expressing the SARS-CoV-2 spike protein were able to prevent RBD from binding to Vero E6 target cells. Although efficient binding of VHHs to cell surface expressed spike proteins is required for blocking the RBD-ACE2 interaction, it is not sufficient. This is illustrated by the various VHHs that can potently bind HekS cells expressing the spike protein but fail to block binding of RBD to VeroE6 cells. However not all VHHS that potently bind the RBD on the cell surface are able to block binding of RBD to Vero E6 target cells. VHHs that potently inhibit RBD binding to Vero E6 cells were mainly restricted to the VHH families: 55, 36, 38, 29, 72 and 149, wherein the VHH families are identified/numbered in view of one of its representative VHH family members (see also FIG. 45, and Tables 5 and 6).

To test if the VHHs present in the PE extracts can neutralize SARS-Cov-2 in vitro we performed neutralization assays using SARS-CoV-2 Spike pseudotype VSV-dG viruses expressing GFP and luciferase. VSV-dG-SARS-CoV-2S (VSV-S) was incubated with 16, 80 and 400-fold diluted PE extracts for 30 minutes at RT before adding to Vero E6 cells grown to subconfluency in 96-well plates. PBS and purified affinity enhanced VHH72 variant (VHH72 h1-S56A at 500 ug/ml) were used respectively as negative and positives controls. PE extract of VHH2.50, a previously isolated VHH72 variant with neutralizing activity that is highly similar with VHH72 was used as reference. Twenty hours after infection the cells were lysed and used to measure GFP and luciferase activity. Several VHH PE extracts could completely neutralize VSV-S in vitro at 400-fold dilution whereas other VHHs failed to do so even at the lowest dilution. The observation that several PE extracts, including the newly identified VHHs related to VHH72 have considerably higher neutralizing activity than the PE extract of VHH2.50, suggest that these VHHs might have superior neutralizing activity than VHH72 and its related VHH2.50. VHHs with the highest neutralizing activity mainly originate from the VHH families F-55, -36, -38, -149 and the VHHs related to VHH72 (FIG. 49). The enhanced neutralizing activity of the VHHs related to VHH72 most likely result from affinity maturation towards the VHH72 epitope on the SARS-CoV-2 Spike protein which was enabled by the additional immunizations using the SARS-CoV-2 spike protein. VHH72 h1-S56A, which was previously characterized has enhanced affinity for the VHH72 epitope on the SARS-CoV-2 Spike. The observation that the 556G substitution present in all the VHH72-related VHHs (VHH72-family members) identified in this immunization campaign demonstrates the importance of the position 56 residue (according to Kabat numbering) in the binding properties of the VHH72 family to the Spike protein.

The respective concentration dependency for respectively interfering with RBD binding to ACE2 and neutralization seemed variable among VHHs. Reasons for that may come from the fact that some VHHs can efficiently interact with recombinant RBD at epitopes that might be much less accessible in the context of the spike trimer. In addition, the performed assays might be less quantitative when using PE extracts instead of purified VHHs. Production and purification of a subset of the most potent neutralizing VHHs tested in this screen was therefore done as a next step in selection of the VHHs, as to identify which VHHs have epitopes that overlap or identical with the VHH72 epitope.

### Example 27. Inhibition of VHH72 Binding to the RBD of the Spike Protein by AlphaLISA Immunoassay

The capacity of VHHs to compete with VHH72 for binding to SARS-CoV-2 RBD was assessed in a competition AlphaLISA (amplified luminescent proximity homogeneous assay).

Selected clones from Example 26, representing different VHH families were recloned for production in either Pichia pastoris or E. coli for further characterization as purified monovalent proteins. Monovalent VHHs contained a C-terminal His6 tag, or C-terminal HA-His6 tag, respectively. Purification was done using Ni-NTA affinity chromatography, as described herein (see also Example 30).

Serial dilutions of anti-SARS-CoV-2 VHHs and irrelevant control VHH (final concentration ranging between 90 nM-0.04 nM) were made in assay buffer (PBS containing 0.5% BSA and 0.05% Tween-20). VHHs were subsequently mixed with VHH72-h1 (S65A)-Flag3-His6 (final concentration 0.6 nM) and SARS-CoV-2 RBD protein Avi-tag biotinylated (AcroBiosystems, Cat nr. SPD-C82E9) (final concentration 0.5 nM) in white low binding 384-well microtitre plates (F-bottom, Greiner Cat nr 781904). After an incubation for 1 hour at room temperature, donor and acceptor beads were added to a final concentration of 20 μg/mL for each in a final volume of 0,025 mL. Biotinylated RBD was captured on streptavidin coated Alpha Donor beads (Perkin Elmer, Cat nr. 6760002), and VHH72_h1(S56A)-Flag3-His6 was captured on anti-Flag AlphaLISA acceptor beads (Perkin Elmer, Cat nr. AL112C) in an incubation of 1 hour at room temperature in the dark. Binding of VHH72 and RBD captured on the beads leads to an energy transfer from one bead to the other, assessed after illumination at 680 nm and reading at 615 nm of on an Ensight instrument.

Results are shown in the FIG. 46. Potencies as determined by IC₅₀ values are shown in Table 5. Results indicate that 7 VHHs (families F-36/55/29/38/149) that are part of a superfamily, and VHH3.83 (Family 83) fully block the interaction of VHH72 to the SARS-CoV-2 RBD protein, indicating they bind to at least overlapping or the same epitope as VHH72. Family members of VHH72 that were identified from immune libraries after SARS-CoV-2 protein boost show enhanced potencies compared to the original VHH72, with sub nM IC₅₀ values (Table 5). A number of other VHH families, including VHH3.151, VHHBD9, VHH3.39, VHH3.89, and VHH3.141 are non-competitors of VHH72, indicating they bind a different epitope than VHH72.

### Example 28. Inhibition of the ACE-2/RBD Interaction by AlphaUSA Immunoassay

Dose-dependent inhibition of the interaction of SARS-CoV-2 RBD protein with the ACE-2 receptor was assessed in a competition AlphaLISA.

Selected clones from Example 26, representing different VHH families were recloned for production in either Pichia pastoris or E. coli for further characterization as purified monovalent proteins. Monovalent VHHs contained a C-terminal His6 tag, or C-terminal HA-His6 tag, respectively. Purification was done using Ni-NTA affinity chromatography, as described herein (see also Example 30).

Serial dilutions of VHHs (final concentration ranging between 90 nM-0.04 nM) were made in assay buffer (PBS containing 0.5% BSA and 0.05% Tween-20), and mixed with SARS-CoV-2 RBD that was biotinylated through an Avi-tag (AcroBiosystems, Cat nr. SPD-C82E9) (final concentration 1 nM) in white low binding 384-well microtitre plates (F-bottom, Greiner Cat nr 781904). Recombinant human ACE-2-Fc (final concentration 0.2 nM) was added to the mixture. After an incubation for 1 hour at room temperature, donor and acceptor beads were added to a final concentration of 20 μg/mL for each in a final volume of 0.025 mL RBD was captured on streptavidin coated Alpha Donor beads (Perkin Elmer, Cat nr. 6760002). Human ACE-2-mFc protein (Sino Biological Cat nr. 10108-H05H) was captured on anti-mouse IgG (Fc specific) acceptor beads (Perkin Elmer, Cat nr. AL105C) in an additional incubation of 1 hour at room temperature in the dark. Interaction between beads was assessed after illumination at 680 nm and reading at 615 nm of on an Ensight instrument. Results are shown in the FIG. 47. Potencies indicated by IC₅₀ values are shown in Table 5. All VHHs that were competing with VHH72 also block the interaction of human ACE2 to the SARS-CoV-2 RBD protein. Increased potencies are observed for family members of VHH72 obtained from immune libraries after protein boost with 5ARS-CoV-2 spike protein. With exception of VHH3.83, that showed partial blockade (75% inhibition), all others showed full blockade of ACE-2 binding.

In conclusion, the competition assay results confirm that purified VHHs from families F-83, 36, 55, 29, 38 and 149 bind to the same epitope as VHH72, and compete with ACE-2 binding similar to the VHH72 family members. The most potent competitors not belonging to the VHH72 family are VHH3.36 and VHH3.83, respectively (Table 5).

TABLE 5
Inhibition of VHH72 (h1 S56A) or ACE2 binding to
the SARS-CoV-2 RBD by additional anti-SARS-CoV-2
VHHs of the VHH72 family and of different VHH families,
as determined in competition AlphaLISA.

| | | Competition | | Competition | |
|---|---|---|---|---|---|
| | | VHH72/RBD | | ACE2/RBD | |
| VHH | | | % inhi- | | % inhi- |
| Family | ID | IC50 (M) | bition | IC50 (M) | bition |
| 72 | VHH72 | 1.60E−08 | 107 | 2.67E−08 | 97 |
| | VHH2.50 | 2.77E−08 | 110 | 3.42E−08 | 79 |
| | VHH3.17 | 3.16E−10 | 99 | 1.03E−09 | 99 |
| | VHH3.77 | 2.37E−10 | 102 | 7.23E−10 | 97 |
| | VHH3.115 | 4.09E−10 | 99 | 1.08E−09 | 100 |
| | VHH3.144 | 3.97E−10 | 98 | 1.12E−09 | 100 |
| | VHHBE4 | 2.12E−10 | 104 | 6.34E−10 | 100 |
| 36 | VHH3.36 | 1.56E−10 | 104 | 5.62E−10 | 100 |
| | VHH3.47 | 2.97E−10 | 100 | 7.29E−10 | 100 |
| 55 | VHH3.35 | 4.06E−10 | 100 | 1.06E−09 | 100 |
| | VHH3.55 | 3.22E−10 | 99 | 8.17E−10 | 100 |
| 29 | VHH3.29 | 3.89E−10 | 97 | 1.00E−09 | 97 |
| 38 | VHH3.38 | 7.69E−10 | 99 | 2.01E−09 | 99 |
| 149 | VHH3.149 | 3.34E−10 | 99 | 9.02E−10 | 98 |
| 83 | VHH3.83 | 1.62E−10 | 101 | 4.60E−10 | 74 |

The VHH families are identified/numbered in view of one of its representative VHH family members (see also FIG. 45).

TABLE 6
VHH amino acid sequences of the VHH72 family and of additional VHHs of different
families which compete in binding to the VHH72 epitope.

| | FL- | | | | | | |
|---|---|---|---|---|---|---|---|
| | SEQ | | SEQ | | SEQ | | SEQ |
| | ID | | ID | | ID | | ID |
| VHH | NO: | CDR1 | NO: | CDR2 | NO: | CDR3 | NO: |
| VHH2.50 | 92 | SIAMG | 111 | TISWSGGSTYYADSVKG | 120 | AGLGTVVSEWDYDYDY | 9 |
| VHH3.17 | 93 | DGAVG | 112 | TVSWNGGGTYFAESVRG | 121 | AGEGTVVSEWDYDYEY | 131 |
| VHH3.77 | 94 | NGAVG | 113 | TVSWNGGGTYFAESVRG | 121 | AGEGTVVSEWDYDYEY | 131 |
| VHH3.115 | 95 | DIAMG | 114 | TVSWNGGGTYYAEPVRG | 122 | AGAGTVVSEWDYDYDY | 132 |
| VHH3.144 | 96 | NGAVG | 113 | TVSWNGGGTYYAESVRG | 123 | AGEGTVVSEWDYDYDY | 133 |
| VHH3BE4 | 97 | NGAVG | 113 | TVSWNGGGTYYAESVRG | 123 | AGEGTVVSEWDYDYDY | 133 |
| VHH3.83 | 98 | SYAMG | 150 | AITFNSDATYYADSVKG | 124 | GGNHYNPQYYHDYDKYDH | 134 |
| VHH3.36 | 99 | SYAMG | 150 | AINWGGISVYYADSVKG | 125 | DPKGWSEWDMEY | 135 |
| VHH3.47 | 100 | TYAMA | 115 | AISENDVMRYYADSVKG | 126 | DPKGWSEWDMDY | 136 |
| VHH3.55 | 101 | NYGVG | 116 | AIRWSSISRYYKDSVKG | 127 | DPAGWSEFGMEY | 137 |
| VHH3.35 | 102 | NYGVG | 116 | AIRWSSISRYYKDSVKG | 127 | DPAGWSEFGMEY | 137 |
| VHH3.29 | 103 | SGGMG | 117 | GIGWAGLSSYYLDSVKG | 128 | DDHGWSAAGMDY | 138 |
| VHH3.38 | 104 | NYAMA | 118 | AMFWSGLPKYYADSVKG | 129 | DSRGWSDVGGMDY | 139 |
| VHH3.149 | 105 | SYALG | 119 | AINWFGAPTYYADSVKG | 130 | DSKGWDPQDMDY | 140 |

### Example 29. PK/PD Analysis Hamster Challenge Studies

For bridging from IP to IV administration, a pharmacokinetic profile after IP and IV delivery was determined in an independent study in healthy Syrian hamsters. For pharmacokinetic study, a single dose of 5 mg/kg D72-53 (PB9683) was delivered via IV or IP in healthy male syrian hamsters (n=12 group, each animal sampled at 3 timepoints). Timepoints sampled were 5 min 15 min, 1 h, 3 h, 8 h, 24 h, 48 h, 96 h and 168 h. Quantification was done using competition AlphaLISA, as described.

Serum exposure over time of D72-53 (PB9683) following a single dose of 5 mg/kg by intraperitoneal (IP) and intravenous (IV) administration in healthy male hamsters is shown in FIG. 38. Twelve animals (body weight range 90-108 g) were used per group, with each animal sampled for 3 timepoints (n=4 per timepoint). Kinetic serum profile of D72-53/PB9683 indicated a serum half-life around 90-100 hours in hamsters. After IP administration serum levels were gradually building up in the first 24 hours, reaching similar serum levels as after IV injection. The prophylactic treated animals dosed 24 hours pre infection hence had stable serum levels at the time of infection, whereas animals in the therapeutic groups (4-19 h post infection) reached stable serum levels between 28-43 h post infection (FIG. 38).

To confirm the drug exposure in challenged hamsters, the day 4 serum concentration of different VHH72 h1S56A-Fc formats (bivalent and tetravalent formats with different Fc types) was quantified. In addition, the concentration of compounds in BALF samples obtained in one challenge study were analysed. In challenged hamsters, the PK/PD relationship between lung viral load (infectious virus) and drug serum concentration at endpoint day 4 is shown in FIG. 51. FIG. 51C shows the correlation of BALF and serum for bivalent and tetravalent formats. BALF exposure follows the systemic exposure irrespective of the valency.

The PK/PD results indicate that in prophylactic setting, all doses to the lowest dose of 1 mg/kg were protective. In therapeutic setting there is a dose relationship, with animals at the lowest doses showing increased variability in anti-viral response. Across treatment groups, non-responding outliers lack detectable drug in sera, suggesting these animals were not exposed.

The PD endpoint has been transformed in a binary response variable. The viral load data in animals treated with VHH72 h1 S56A-Fc (different Fc types) were compared with the median of viral load in control group in each experiment and positive outcome were defined as viral load lower than a threshold of a 4-fold decrease in the log TCID50/mg. The application of logistic regression on the transformed binary variable allowed to define the probability of a viral knockdown as a function of serum concentration and consequently allowed to define the level of concentration (with 90% confidence interval) leading to the 95% probability of reaching a therapeutic success.

### Example 30. Purification and Binding Characteristics of a Selected Panel of 3^{rd} Generation VHH Families Specifically Binding SARS-CoV-1 and -2 Spike Protein

Representative VHHs of the 3^{rd} d generation families (see Examples 26-28) were cloned in a Pichia pastoris expression plasmid, produced in Pichia pastoris and purified by Ni-NTA affinity chromatography and buffer exchanged into PBS. SDS-PAGE and Coomassie blue staining revealed that the produced VHHs had the expected size for the following VHH families: (F, for family; numbered according to one if its representative family members characterized herein) F72 (VHH3.17, VHH3.77, VHH3.115 and VHH3.144), F55 (VHH3.35 and VHH3.55), F36 (VHH3.36 and VHH3.47), F149 (VHH3.19), F38 (VHH3.38) and F29 (VHH3.29). In agreement with the presence of an N-glycosylation site, next to the non-glycosylated VHH3.47 an additional protein band that migrated slower in the gel was observed (FIG. 52A). The correct size of the produced VHHs was confirmed by intact MS (data not shown). VHH3.83 was produced in WK6 E. coli transformed with the pMECS-VHH3.83 vector that was used for bio-panning. After purification by Ni-NTA affinity chromatography and buffer exchange the expression of VHH3.83 was analyzed by SDS-PAGE. Coomassie staining of the gel revealed a single protein band at the expected molecular size (FIG. 52B).

The binding of the purified VHHs to the SARS-CoV-2 spike protein and RBD was tested by ELISA. Dilution series of the VHHs, VHH72 and an irrelevant control VHH (GBP) were applied to ELISA plates coated with recombinant prefusion stabilized SARS-CoV-2-2P spike protein or SARS-Cov-2 RBD-muFc (Sinobiological). Except for VHH3.47, all VHH bound to SARS-CoV-2 RBD and Spike proteins (FIG. 53 A-C) with much higher affinity than VHH72. The lack of detectable binding of VHH3.47 might be the consequence of its glycosylation which might overcome recognition of the anti-VHH antibody used to detect the bound VHHs. Next to the SARS-CoV-2 spike all VHHs also efficiently bound the SARS-CoV-1 spike protein (FIG. 53 D). This indicates that the tested VHHs bind to an epitope on the spike that is conserved among clade 1 Sarbecoviruses (SARS-CoV-1 and SARS-CoV-2), such as the VHH72 epitope (as described herein and in Ref 10). Binding of the VHHs to the RBD of SARS-CoV-2 was also tested by biolayer interferometry (BLI) in which monovalent SARS-CoV-2 RBD-human Fc was immobilized on an anti-human Fc biosensor. This revealed that all tested VHHs bound RBD with a considerable slower off rate than VHH72 (FIG. 53E). For VHH3.17, VHH3.77 and VHH3.115 the binding kinetics were determined by BLI. FIG. 53F illustrates that VHH3.115, VHH3.17 and VHH3.77 bind monomeric RBD with a K_{D} of respectively 7.34×10⁻¹⁰ M, 2.34×10⁻¹⁰ M and 1.5×10⁻¹⁰ M.

To investigate if the VHHs can also recognize RBDs of clade 2 and 3 Sarbecoviruses, binding of the VHHs to yeast cells expressing the RBD of representative clade 1.A (WIV1), clade1.B (GD-pangolin), clade 2 (HKU3 and ZCX21)) and clade 3 (BM48-31) Sarbecoviruses was tested by flow cytometry (FIG. 54A). In line with the binding to the spike proteins of SARS-CoV-2 and -1 in ELISA, all tested VHHs, except for the GBP (GFP binding protein) control VHH, bound yeast cells expressing the RBD of clade 1.A (WIV1) and clade1.B (GD-pangolin) at their surface (FIG. 54B). In addition, two VHHs of the VHH72 family (VHH3.17 and VHH3.77) that bind SARS-CoV-2 spike and RBD with high affinity were able to also recognize the RBD of a clade 2 Sarbecovirus. The RBD of at least one of the two tested clade 2 Sarbecoviruses were recognized by VHHs belonging to F55, F36, F149, F38, F29 and F83. VHHs of F 55, F36, V83, f38 and F29 were able to bind the BM48-31 clade 3 RBD, whereas VHH3.38, VHH3.83 and VHH3.47 were able to bind to all RBDs that were tested in this experiment (FIG. 54B). In addition, VHH3.38 and VHH3.83 were shown to bind to all RBD's of a broader panel of clade1 and 2 Sarbecoviruses, except for the clade 2 Rf1 virus. For VHH3.83 binding to the yeast surface-displayed Rf1 RBD could be observed at 100 μg/ml, no binding or only marginal binding could be observed at lower concentrations for VHH3.83. Amino acid alignment illustrated that only a few patches of the RBD surface are highly conserved among the tested Sarbecovirsues. One of those patches is located at the VHH72 epitope, as described herein (FIG. 55).

To test if the selected VHHs compete with VHH72 or S309 for the binding of RBD, monomeric RBD (RBD-SD1-Avi (biotinylated Avi-tag) was captured on ELISA plates coated with VHH72-Fc (D72-23=humVHH_S56A/LALAPG-Fc); this is a VHH72-human IgG1 fusion in which VHH72 has a S56A substitution with increased its affinity for SARS-CoV-1 and -2 RBD as compared to VHH72) or antibody S309 that also binds the RBD core but at a site that is opposite of the VHH72 epitope (FIG. 57B). In contrast, to RBD captured by S309 none of the VHHs could bind to RBD captured by VHH72-Fc, which demonstrates that the tested VHHs recognize the same epitope as VHH72 or an epitope that overlaps with that of VHH72 (FIG. 57A). In the same assay RBD captured by VHH72-Fc could readily be recognized by 2 VHHs (non-competing VHHs) that bind the SARS-CoV-2 RBD at a site distinct from the VHH72 epitope. These data demonstrate that the selected VHHs bind to a site distant from the S309 epitope but at a site that either comprises or overlaps with the VHH72 epitope or at a site in the close proximity of the VHH72 epitope.

### Example 31. SARS-CoV-2 Spike Protein Residue K378, a Key Residue of the VHH72 Binding Epitope, is Also Important for the Binding of VHH3.38 and VHH3.83

The crystal structure of VHH72 in complex with the 5ARS-CoV-1 RBD revealed the importance of K378 for the binding of VHH72 (as described herein and Ref. 10). To test if the RBD K378 is also important for the binding of VHH3.38 and VHH3.83 we substituted the Lys at position 378 for an Asn (K378N) in an expression vector for the SARS-CoV-1 spike protein in which the RBD was replace by this of SARS-CoV-2 as described by Letko et al.¹¹. Compared to the cell surface expressed parental SARS-CoV-2 RBD, binding of both VHH3.38 and VHH3.83 to the K378N mutant was severely impaired (FIGS. 58 A and B). This is in agreement with the observations that VHH3.83 and VHH3.38 display low or no binding for the RBD of the Rf1 Sarbecovirus. This RBD has an Asn at the position that corresponds to K378 in the SARS-CoV-2 RBD. Combined with the competition of these VHHs with VHH72 for the binding of the RBD, this strongly argues that VHH3.38 and VHH3.83 bind at the VHH72 epitope.

### Example 32. SARS-CoV-1 and -2 Neutralization Potential of the Selected VHHs

To test if the VHHs, like VHH72, can neutralize SARS-CoV-2 and SARS-CoV-1 infection, the VHHs were tested for their ability to neutralize pseudotyped VSV-delG virus pseudotyped with the spike proteins of SARS-CoV-2 or of SARS-CoV-1 (VSV-delG-SARS-CoV-2-S, VSV-delG-SARS-CoV-1-S). FIGS. 59 and 60 demonstrate that all VHHs could potently neutralize both VSV-delG-SARS-CoV-2-S and VSV-delG-5ARS-CoV-1-S pseudotyped viruses.

Furthermore, we tested whether binding of the selected VHHs, similar as for the binding of VHH72 to the SARS-CoV-2 RBD, could prevent binding of the RBD to ACE2 expressing VeroE6 cells. Viral attachment of SARS-CoV-2 is mediated by the spike RBD that binds to ACE2 at the surface of target cells. Neutralization of SARS-CoV-2 by most RBD specific antibodies or nanobodies, such as VHH72, is associated with their ability to prevent RBD from binding its ACE2 receptor at the surface of target cells. To investigate if the VHHs are able to inhibit binding of RBD to the ACE2 receptor, we tested if the selected VHHs and VHH72 (VHH72_h1-S56A) can prevent binding of SARS-CoV-2 RBD, fused to a mouse Fc, to Vero cells. FIG. 61 illustrates that all VHHs could prevent the interaction of SARS-CoV-2 RBD with VeroE6 cells. This indicates that the tested VHHs, alike VHH72, can potently prevent SARS-CoV-2 RBD from binding to its ACE2 receptor.

### Example 33. Identification of the Epitopes of VHH3.38, VHH3.83 and VHH3.55 by Deep Mutational Scanning

To delineate the epitopes of VHH3.38, VHH3.83 and VHH3.55 we performed deep mutational scanning to identify the RBD amino acids that are important for the binding of the selected VHHs. VHH72 (VHH72_h1_S56A), for which a crystal structure in complex with the related SARS-CoV-1 RBD is available, was included as a reference. We made use of a yeast-display platform developed by Starr et al.⁷², consisting of 2 independently generated libraries of Saccharomyces cerevisiae cells, each expressing a single RBD variant labeled with a unique barcode and a myc-tag^{72,92}. The 2 libraries of RBD variants were generated by PCR-based mutagenesis to generate a comprehensive collection of RBD variants in which each position has been substituted to all other amino acids. The RBD variants contain on average 2.7 amino acid substitutions. To retain only functional RBD variants the yeast RBD-display libraries were presorted by FACS based on their ability to bind recombinant ACE2 (data not shown). To identify yeast cells that express an RBD variant with reduced affinity for the tested VHHs in a sensitive manner we defined for each VHH a concertation at which binding was just below saturation. For each of the tested VHHs this concentration was first determined by staining yeast cells expressing wild type SARS-CoV-2 RBD with a dilution series of VHHs (FIG. 62A). Using this approach, we selected 400 ng/ml for VHH72_h1_S56A (VHH72) and 10 ng/ml for VHH3.38, VHH3.55 and VHH3.83. This difference in concentration to reach a comparable “just below the saturation” concentration reflects the higher affinity for VHH3.38 (and VHH3.55 and VHH3.83) for SARS-CoV-2 RBD compared with VHH72 as shown above (FIG. 53). To identify yeast cells expressing a RBD variant with reduced affinity for the tested VHH, the presorted library was stained with the VHH and anti-myc-tag antibody (FIG. 628). RBD expressing cells that displayed low VHH staining were sorted, grown and used for sequencing of their respective barcodes. To identify the RBD amino acids that are significantly involved in VHH binding, the substitutions that are enriched in the sorted population were determined as described by Greane et al.⁹².

FIG. 63B shows for each tested VHH the overall profile of positions in the RBD for which substitutions result in reduced VHH binding. It is clear that the profiles for VHH3.38, VHH3.55 and VHH3.83 largely overlap with that of VHH72_h1_S56A. Escape profile analysis identified A363, Y365, S366 Y369, N370, S371, F374, S375, T379, K378, P384, and Y508 as amino acid positions that are involved (based on the average of the two libraries) in binding of VHH72_h1_S56A⁹². Except from the 3 first positions all fall within the footprint of VHH72 on RBD as defined by modeling based on the crystal structure of VHH72 in complex with the SARS-CoV-1 RBD^{10,14} (FIGS. 64A and B). Positions, A363, Y365 and S366 are located outside the VHH72 footprint. Inspection of the SARS-CoV-2 RBD structure revealed that these are adjacent to the VHH72 epitope and that the side chains of the respective amino acids are mainly oriented inwards in the RBD. Hence, the reduction in VHH72 binding by substitutions on this position most likely results from an allosteric impact.

For VHH3.38 the positions that were identified by the deep mutational scanning (C336, V341, A363, Y365, S366, L368, Y369, S373-K378, P384, R408, A435, N437, V503 and Y508) strongly overlap with those identified for VHH72_h1_S56A. The identification of RBD K378 as a key residue for the binding of VHH3.38 is in line with the observation that binding of VHH3.38 to mammalian cells expressing the SARS-CoV-2 RBD K378N mutant is severely impaired as compared to binding to wild type SARS-CoV-2 RBD (FIG. 58A). The positions L368, S373, F377, N437 and V503, although not identified in the scan for VHH72 are clearly located within the VHH72 footprint. Three additional amino acid positions (C336, V341 and A435) locate outside the VHH72 footprint. C336 locates near the lower side of the VHH72 epitope and forms a disulfide-bond with C336. Disruption of this disulfide bridge will most likely have a considerable impact on the folding of the VHH72 epitope. Also V341 and A435 locate at the level of the VHH72 foot print but at the opposite side of the RBD. Hence, also mutations at those positions can have an allosteric impact on the binding of VHHs to the VHH72 epitope (FIG. 64).

Also for VHH3.55 the positions that were identified by the deep mutational scanning (A363, Y365, S366, Y369, S373-K378, P384, C391, F392, T393 and Y508) largely overlap with those identified for VHH72_h1_S56A. The positions C391, F392, T393 locate outside the VHH72 footprint. C391 locates near the lower side of the VHH72 epitope and forms disulfide-bond with C525. Disruption of also this disulfide bridge will thus likely have a considerable impact on the folding of the adjacent VHH72 epitope. Also F392 and T393 locate near the lower part of the VHH72 epitope. Hence, also substitutions at these positions can have an allosteric impact on the binding of VHHs at the VHH72 epitope.

Only two amino acid positions (K378 and P384) of the RBD were identified in the scan for VHH3.83. Importantly, these two positions were also identified for the other tested VHHs including VHH72_h1_S56A and they are located within the VHH72 epitope. The importance of the RBD K378 residue for the binding of VHH3.83 is in line with the observation that binding of this VHH to mammalian cells expressing the SARS-CoV-2 RBD K378N mutant is considerably impaired as compared to binding to wild type SARS-CoV-2 RBD (FIG. 58B).

### Example 34. Cryo-EM Structure of SARS-CoV2 Spike Protein Bound to VHH3.38

To obtain a view on the VHH3.38 binding mode and binding epitope on the SARS-CoV2 spike protein (SC2), we determined the 3D cryoEM structure of SC2 in complex with the nanobody. Purified SC2 and VHH3.38 were mixed in a 1:1 stoichiometric ratio to a final concentration of 0.2 mg/ml and incubated at room temperature for 1 hour. SC2-VHH complexes were placed on a Quantifoil R2/1 EM grid covered with a monolayer of graphene oxide, before being flash-cooled into liquid ethane. Data were collected on a 300 kV JEOL CryoARM300 cryo electron microscope equipped with an inline energy filter and Gatan3 direct electron detector. A total of 22.000 images at 60K magnification were collected from which a final set of 24.000 single particles were extracted for 2D classification and 3D reconstruction of the complex. Three-fold rotational symmetry (C3) averaging was imposed throughout reconstruction, resulting in a final electron potential map of 4.2 Å. The cryoEM map reveals density for three copies of the SC2 protomer (see FIG. 65). In each of the protomers, the receptor binding domain (RBD (residues 334 to 527) is found in an upright position, in a similar conformation to that seen in the 1-RBD up conformation such as reported in PDB 6zgg (FIGS. 65 and 66). In absence of the VHH, the SC2 protein is found in closed conformation, where all three RBD domains are in a downward orientation (data not shown). Thus, binding of the VHH induces a transition from the closed state of the SC2 protein to a fully open state with all three RBD domains reside in an upright conformation (see FIG. 66). In additional to the density corresponding to SC2, residual density is seen along the side of the RBD, corresponding to the binding of the nanobody. The final model of the SC2-VHH3.38 complex was obtained by automated rigid body map fitting of the individual domains in SC2 and the VHH. The model and electron potential map show that VHH3.38 binds the side of the RBD, targeting SC2 surface formed by residues 368 to 380, and residues 408, 503 and 509 (see FIG. 65). The binding VHH3.38 binding epitope in SC2 is not accessible in the closed conformation and becomes exposed only upon upward rotation of the RBD into the open conformation. Binding of the VHH3.38 to the 1-RBD up conformation results in steric clash with the closed RBD conformation of the adjacent protomer, thereby inducing the 3-RBD up conformation. We observe a strong reduction (^{˜}100-fold) reduction in the particle density of the SC2-VHH3.38 complex on the cryoEM grids compared to equivalent concentrations of apo SC2, suggesting that VHH binding results in a destabilization of the complex. In agreement with this, aggregates of seemingly unstructured particles can be seen in the images of SC2-VHH3.38 complex. These observations suggest that part of the mode of action of VHH3.38 binding to SC2 is an induced loss of structural integrity in the spike protein. Additionally, when the structure of the SC2-VHH3.38 complex is superimposed with the crystal structure of SARS-CoV2 RBD in complex with the human ACE2 receptor, a steric clash of the VHH can be seen with the ACE2 receptor, suggesting that VHH and ACE2 binding to RBD are mutually exclusive. These observations are in agreement with the competition binding experiments that show that VHH binding to SC2 competes with ACE2 binding (see above).

### Methods

Molecular Modeling of the SARS VHH-72 Interaction with SARS-CoV-2 RBD.

Molecular Dynamics simulations were with model-complexes of VHH72 (chain C from PDB-entry 6WAQ) and variants, with the outward-positioned RBD from the cryo-EM structure pdb-entry 6VSB of the SARS-CoV-2 prefusion spike glycoprotein (chain A, residues 335-528) and variants. The missing loops at residues 444-448, 455-490 and 501-502 in the cryo-EM RBD were reconstructed from the I-TASSER SARS-CoV-2 RBD model²⁰ and the missing residues were added by the Swiss-PDBViewer²¹. Simulations were with Gromacs version 2020.1²² using the Amber ff99SB-ILDN force field⁴² and were run for 5 nanoseconds. After conversion of the trajectory to PDB-format, snapshots were extracted for every 0.5 nanoseconds and were submitted to the FastContact 2.0 server¹⁴.

### SARS-CoV-2 Spike Sequence Variant Analysis.

SARS-CoV-2 genome sequences originating from human hosts were downloaded from GISAID. Genomes with invalid DNA character code were removed. Spike coding sequences were retrieved by aligning the genomes to the reference spike sequence annotated in NC_045512.2 (Wuhan-Hu-1 isolate, NCBI RefSeq). For this purpose, pairwise alignments were performed using R package Biostrings version 2.54.0, a fixed substitution matrix in the “overlap” mode with the following parameters according to Biostrings documentation: 1 and -3 for match and mismatch substitution scores; 5 and 2 as gap opening and gap extension penalties, respectively. Incomplete genomes without spike coding sequences, or that generated very short or no alignment were removed. Coding sequences with frame-disturbing deletions were also excluded and the remaining open reading frames were in-silico translated using Biostrings option to solve “fuzzy” codons containing undetermined nucleotide(s). In the next step, predicted spike protein sequences with stretches of undetermined amino acids (denoted as X), derived from poor sequencing results (Ns) were removed, although single X characters, surrounded by credible amino acid sequence were allowed. Further, full-length sequences with a single stop codon or lacking a stop signal (due to a possible C-terminal extension) were retained, while proteins with premature stop codon(s) were excluded.

The resulting, quality-controlled spike protein sequences were aligned using the ClustalOmega algorithm and R package msa version 1.18.0 with default parameters and the BLOSUM65 substitution matrix. Multiple sequence alignment served to generate protein sequence logo (WebLogo 3.0) and derive conservation percentage and variability percentage values per amino acid position. Subsequently, a custom pyMol script was generated to visualize the conservation scores as B-factors of the alpha carbons onto RBD chain PDB structure modelled in complex with our nanobody. R packages seqinr 3.6-1 and BALCONY 0.2.10 were used to calculate amino acid frequencies for all mutations occurring in the dataset at least once. Major and minor allele frequencies and counts were assigned, supplemented with geographical information and collection time of their corresponding samples. Effects of individual mutations on spike expression and ACE2 binding were derived from Starr et al¹⁹. Data collected for full-length spike protein and well as focused on RBD (positions 333-516) were visualized using ggplot2 version 3.3.0.

### Strains.

Escherichia coli (E. coli) MC1061 or DH5α were used for standard molecular biology manipulations. The Pichia pastoris (syn. Komagataella phaffi) NRRL-Y 11430 OCH1 knock-out strain used for VHH-Fc screening (P. pastoris OCH1) was obtained by the deletion of 3 bp encoding for E151 in the OCH1 gene with CRISPR-Cas9⁴³. As reported before, the knock-out of the α-1,6-mannosyltransferase encoded by OCH1, results in secretion of more homogenously glycosylated protein carrying mainly Man8 glycan structure⁴⁴.

### Recombinant Protein Production in Yeast.

Yeast cultures were grown in liquid YPD (1% yeast extract, 2% peptone, 2% D-glucose) or on solid YPD-agar (1% yeast extract, 2% peptone, 2% D-glucose, 2% agar) and selected with 100 μg/ml Zeocin® or 100 μg/ml Zeocin® and 500 μg/ml G418 (InvivoGen). For protein expression, cultures were grown in a shaking incubator (28° C., 225 rpm) in BMDY (1% yeast extract, 2% peptone, 100 mM KH₂PO₄/K₂HPO₄, 1.34% YNB, 2% D-glucose, pH 6) or BMGY (same composition but with 1% glycerol replacing the 2% D-glucose).

### Modular Generation of Expression Plasmids.

The expression vectors for all the VHH72-XXX-hFc muteins were generated using an adapted version of the Yeast Modular Cloning toolkit based on Golden Gate assembly⁴⁵. Briefly, coding sequences for the S. cerevisiae α-mating factor minus EA-repeats (P3a_ScMF-EAEAdeleted), SARS-VHH72 mutants (P3b_SARS_VHH72-xxx) and human IgG1 hinge-human IgG1 Fc with or without a C-terminal (G₄S)₂ linker (P4a_hIgG1.Hinge-hIgG1.Fc) were codon optimized for expression in P. pastoris using the GeneArt (ThermoFisher Scientific) proprietary algorithm and ordered as gBlocks at IDT (Integrated DNA Technologies BVBA, Leuven, Belgium). Each coding sequence was flanked by unique part-specific upstream and downstream BsaI-generated overhangs. The gblocks were inserted in a universal entry vector via BsmBI assembly which resulted in different “part” plasmids, containing a chloramphenicol resistance cassette. Part plasmids were assembled to form expression plasmids (pX-VHH72-xxx-hIgGhinge-hIgGFc) via a Golden Gate BsaI assembly. Each expression plasmid consists of the assembly of 9 parts: P1_ConLS, P2_pGAP, P3a-001_-ScMF-EAEAdeleted, P3b-002_-VHH72-xxx, P4a-hIgG1.Hinge-hIgG1.Fc (or P4a-(GGGGS)x2hIgG1.Hinge-hIgG1.Fc), P4b_AOX1tt, PS_ConR1, P6-7 Lox71-Zeo, P8 AmpR-ColE1-Lox66. Selection of correctly assembled expression plasmids was made in LB supplemented with 50 μg/mL carbenicillin and 50 μg/mL Zeocin®. All the part and expression plasmids were sequence verified. Transformations of linearized expression plasmids (Avril) were performed using the lithium acetate electroporation protocol as described⁴⁶.

### Protein Expression and Purification.

For small scale expression screening, 2-3 single colonies of P. pastoris OCH transformed with pX-VHH72-xxx-hIgGhinge-hIgGFc were inoculated in 2 ml BMDY or BMGY in a 24 deep well block. After 50 hours of expression in a shaking incubator (28° C., 225 rpm), the medium was collected by centrifugation at 1.500 g, 4° C. for 5 minutes. Protein expression levels were evaluated on Coomassie-stained SDS-PAGE of crude supernatant. Crude supernatant was used immediately for analytics purposes (biolayer interferometry and mass spectrometry, see below) or stored at −20° C.

For protein purification, an overnight culture of P. pastoris OCH1 transformed with pX-VHH72-xxx-hIgGhinge-hIgGFc was diluted in 125 ml of BMDY to 0.1 OD600 in 2 liter baffled shake flasks. After 50-60 hours, the medium was collected by centrifugation at 1.500 g, 4° C. for 10 minutes. Culture media was filtered over a 0.22 μm bottle top filter (Millipore) before loading on a HiTrap MabSelect SuRe 5 ml column (GE Healthcare), equilibrated with McIlvaine buffer pH 7.2 (174 mM Na₂HPO₄, 13 mM citric acid). The column was eluted with McIlvaine buffer pH 3 (40 mM Na₂HPO₄,79 mM citric acid). Collected fractions were neutralized to pH 6.5 with Na₃PO₄ saturated at 4° C. Elution fractions containing the protein of interest (evaluation on SDS-PAGE) were pooled and injected on a Hiprep 26-10 desalting column (GE-Healthcare), eluted with 25 mM L-His, 125 mM NaCl, pH 6. After spectroscopic protein concentration determination (absorbance at 280 nm minu buffer blank), purified protein concentration was concentrated using Amicon 10 kDa MWCO spin columns if required, snap-frozen in liquid nitrogen, and stored at −80° C.

Biolayer Interferometry Screening of P. pastoris-Expressed VHH72-hFc Affinity Mutants.

The SARS-CoV-2 RBD binding kinetics of VHH72-hFc affinity mutants in P. pastoris supernatant were assessed via biolayer interferometry on an Octet RED96 system (FortéBio). Anti-mouse IgG Fc capture (AMC) biosensors (FortéBio) were soaked in kinetics buffer (10 mM HEPES pH 7.5, 150 mM NaCl, 1 mg/ml bovine serum albumin, 0.05% Tween-20 and 3 mM EDTA) for 20 min. Mouse IgG1 Fc fused SARS-CoV-2 RBD (Sino Biological) at 5-15 μg/ml was immobilized on these AMC biosensors to a signal of 0.3-0.8 nm. Recombinant protein concentrations in crude cell supernatants of VHH72-hFc expressing P. pastoris OCH1⁻ were estimated based on band intensity on Coomassie-stained SDS-PAGE as compared to a purified VHH-hFc protein. Crude supernatants were diluted 20 to 100-fold in kinetics buffer to an apparent VHH72-hFc affinity mutant concentration of 5-10 nM and association was measured for 180 s. Dissociation (480 s) was measured in crude supernatant of a non-transformed P. pastoris OCH⁻ culture at equal dilutions in kinetics buffer. Between analyses, biosensors were regenerated by three times 20 s exposure to regeneration buffer (10 mM glycine pH 1.7). Using FortéBio Data Analysis 9.0 software, data were double reference-subtracted and the decrease of response signal during dissociation was determined.

### Biolayer Interferometry Kinetics.

RBD binding kinetics of purified VHH72-hFc variants were assessed via biolayer interferometry on an Octet RED96 system (FortéBio). Anti-mouse IgG Fc capture (AMC) biosensors (FortéBio) were soaked in kinetics buffer for 20 min. Mouse IgG1 Fc fused SARS-CoV-2-RBD (Sino Biological) at 15 μg/ml was immobilized on these AMC biosensors to a signal of 0.4-0.6 nm. Association (120 s) and dissociation (480 s) of twofold dilution series of 30 nM VHH72-hFc variants in kinetics buffer were measured at 30° C. To measure the affinity of monovalent VHH72 variants for RBD, anti-human IgG Fc capture (AHC) biosensors (FortéBio) were soaked in kinetics buffer for 20 min. Monomeric human Fc-fused SARS-CoV-2_RBD-SD1²³ at 15 μg/ml was immobilized on these AHC biosensors to a signal of 0.35-0.5 nm. Association (120 s) and dissociation (480 s) of twofold dilution series of 200 nM VHH72 variant samples in kinetics buffer were measured at 30° C.

Between analyses, both AHC and AMC biosensors were regenerated by three times 20 s exposure to regeneration buffer (10 mM glycine pH 1.7). Data were double reference-subtracted and aligned to each other in Octet Data Analysis software v9.0 (FortéBio) based on a baseline measurement of a non-relevant VHH-IgG1 Fc fusion protein (for kinetics of VHH72-hFc variants) or kinetics buffer (for kinetics of monovalent VHHs). Association and dissociation of non-saturated curves were fit in a global 1:1 model.

### Mass Spectrometry Analysis of Intact Proteins.

VHH72-Fc protein (10 μg) was first reduced with tris(2-carboxyethyl)phosphine (TCEP; 10 mM) for 30 min at 37° C., after which the reduced protein was separated on an Ultimate 3000 HPLC system (Thermo Fisher Scientific, Bremen, Germany) online connected to an LTQ Orbitrap XL mass spectrometer (Thermo Fischer Scientific). Briefly, approximately 8 μg of protein was injected on a Zorbax 300SB-C18 column (5 μm, 300 Å, 1×250 mm IDxL; Agilent Technologies) and separated using a 30 min gradient from 5% to 80% solvent B at a flow rate of 100 μl/min (solvent A: 0.1% formic acid and 0.05% trifluoroacetic acid in water; solvent B: 0.1% formic acid and 0.05% trifluoroacetic acid in acetonitrile). The column temperature was maintained at 60° C. Eluting proteins were directly sprayed in the mass spectrometer with an ESI source using the following parameters: spray voltage of 4.2 kV, surface-induced dissociation of 30 V, capillary temperature of 325° C., capillary voltage of 35 V and a sheath gas flow rate of 7 (arbitrary units). The mass spectrometer was operated in MS1 mode using the orbitrap analyzer at a resolution of 100,000 (at m/z 400) and a mass range of 600-4000 m/z, in profile mode. The resulting MS spectra were deconvoluted with the BioPharma Finder™ 3.0 software (Thermo Fischer Scientific) using the Xtract deconvolution algorithm (isotopically resolved spectra). The deconvoluted spectra were manually annotated.

### Thermal Stability.

To evaluate thermal stability of VHH72-hFc variants, differential scanning fluorimetry (a thermofluor assay) was performed⁴⁹ Briefly, a 10 μM solution of VHH72-hFc in PBS was mixed with 10×SYPRO Orange dye (Life Technologies), and dye binding to molten globule unfolding protein was measured over a 0.01°/s temperature gradient from 20° C. to 98° C. in a Roche LightCycler 480 qPCR machine. Blank-subtracted data were normalized to 0-100%. After cubic spline interpolation of the melting curves, first derivatives were plotted to identify each melting temperature (Tm) as the peaks of these first derivatives.

### Physical and Chemical Stability Testing.

Dynamic light scattering was performed using the Uncle instrument (Unchained Labs; Pleasanton, Calif., USA). Briefly, 10 μL of sample at 1 mg/mL of sample was added to the sample cuvette. Laser and attenuator controls were set at Auto while 10 acquisitions were run per data point with an acquisition time of 10 s for each. Intrinsic tryptophan-fluorescence was monitored upon temperature-induced protein unfolding in an Uncle instrument (Unchained Labs; Pleasanton, Calif., USA). Also here, 10 μL of sample at 1 mg/mL was applied to the sample cuvette, and a linear temperature ramp was initiated from 25 to 95° C. at a rate of 0.5° C./min, with a pre-run incubation for 180 s. The barycentric mean (BCM) and static light scattering (SLS at 266 nm and 473 nm) signals were plotted against temperature in order to obtain melting temperatures (Tₘ) and aggregation onset temperatures (T_{agg}), respectively. Freeze-thaw stability was assessed by subjecting 1 mg/mL protein samples to five consecutive cycles of freezing at −80° C. and thawing at room temperature. Subsequently, these samples were checked for protein concentration and measured for any loss of protein by visual inspection, multi-angle light scattering coupled to size-exclusion chromatography, dynamic light scattering and OD₅₀₀ₙₘ measurement. Forced methionine oxidation was performed by adding hydrogen peroxide to 1 mg/mL protein samples up to a final concentration of 10 mM, followed by incubation at 37° C. for 3 hours, with final buffer exchange to phosphate buffered saline (PBS) using PD MidiTrap G-25 columns (GE Healthcare; Chicago, Ill., USA) according to the manufacturer's instructions, and storage at −80° C. until mass spectrometric analysis.

### RBD Competition Assay on Vero E6 Cells.

SARS-CoV-2 RBD fused to murine IgG Fc (Sino Biological) at a final concentration of 0.4 μg/mL was incubated with 1 ug/ml of monovalent VHH and incubated at room temperature for 20 min followed by an additional 10 min incubation on ice. VeroE6 cells grown at sub-confluency were detached by cell dissociation buffer (Sigma) and trypsin treatment. After washing once with PBS, the cells were blocked with 1% BSA in PBS on ice. All remaining steps were also performed on ice. The mixtures containing RBD and VHHs or VHH-Fc fusions were added to the cells and incubated for 1 h. Subsequently, the cells were washed 3 times with PBS containing 0.5% BSA and stained with an AF647 conjugated donkey anti-mouse IgG antibody (Invitrogen) for 1 h. Following additional 3 washes with PBS containing 0.5% BSA, the cells were analyzed by flow cytometry using an BD LSRII flow cytometer (BD Biosciences).

### CoV Pseudovirus Neutralization Assay.

To generate replication-deficient VSV pseudotyped viruses, HEK293T cells, transfected with SARS-CoV-1 S or SARS-CoV-2 S were inoculated with a replication deficient VSV vector containing eGFP and firefly luciferase expression cassettes (Berger and Zimmer, PloS One 6, e25858 (2011)^{76,77}. After a 1 h incubation at 37° C., the inoculum was removed, cells were washed with PBS and incubated in media supplemented with an anti-VSV G mAb (ATCC) for 16 h. Pseudotyped particles were then harvested and clarified by centrifugation as described (Wrapp et al., 2020 Cell May 28; 181(5):1004-1015.e15)¹³. For the VSV pseudotype neutralization experiments, the pseudoviruses were incubated for 30 min at 37° C. with different dilutions of purified VHH or with GFP-binding protein (GBP: a VHH specific for GFP). The incubated pseudoviruses were subsequently added to subconfluent monolayers of VeroE6 cells. Sixteen h later, the transduction efficiency was quantified by measuring the GFP fluorescence in cell lysates using a Tecan infinite 200 pro plate reader. As indicated in the legends the GFP fluorescence was normalized using either the GFP fluorescence of non-infected cells and infected cells treated with PBS or the lowest and highest GFP fluorescence value of each dilution series. The IC₅₀ was calculated by non-linear regression curve fitting, log(inhibitor) vs. response (four parameters).

### SARS-CoV-2 Plaque Reduction Neutralization Test (PRNT).

For the authentic SARS-CoV-2 neutralization test, SARS-CoV-2 strain BetaCov/Belgium/GHB-03021/2020 (EPI ISL 40797612020-02-03) was used from passage P6 grown on VeroE6 cells as described¹³. VHH-Fc constructs were three-fold serially diluted, using a starting concentration of 20 μg/ml, mixed with 100 PFU SARS-CoV-2 and incubated at 37° C. for 1 h. VHH-Fc-virus complexes were then added to Vero E6 cell monolayers in 12-well plates and incubated at 37′C for 1 h. Subsequently, the inoculum mixture was replaced with 0.8% (w/v) methylcellulose in DMEM supplemented with 2% FBS. After 3 days incubation at 37° C., the overlays were removed, the cells were fixed with 3.7% PFA, and subsequently stained with 0.5% crystal violet. Half-maximum neutralization titers (PRNT₅₀) were defined as the VHH-Fc concentration that resulted in a plaque reduction of 50%.

### Animals Used in Example 9

Wild-type Syrian hamsters (Mesocricetus auratus) were purchased from Janvier Laboratories. Six- to eight-weeks-old wild-type hamsters were used. Animals were housed individually in individually ventilated isolator cages (IsoCage N Biocontainment System, Tecniplast) with access to food and water ad libitum, and cage enrichment (wood block). Housing conditions and experimental procedures were approved by the ethical committee of KU Leuven (license P015-2020), following institutional guidelines approved by the Federation of European Laboratory Animal Science Associations (FELASA). Animals were euthanized by 500 μl of intraperitoneally administered Dolethal (200 mg/ml sodium pentobarbital, Vétoquinol SA). Animals were monitored daily for signs of disease (lethargy, heavy breathing or ruffled fur). Prior to infection, the animals were anesthetized by intraperitoneal injection of a xylazine (16 mg/kg, XYL-M®, V.M.D.), ketamine (40 mg/kg, Nimatek, EuroVet) and atropine (0.2 mg/kg, Sterop) solution. Each animal was inoculated intranasally by gently adding 50 μl droplets of virus stock containing 2×10⁶ TCID₅₀ (P6 virus) on both nostrils. Uninfected animals did not receive any virus or matrix.

### Virus Strains as Used in Example 9, and 21 to 23

Examples 9 and 23 applied the SARS-CoV-2 strain BetaCov/Belgium/GHB-03021/2020 (EPI ISL 40797612020-02-03) recovered from a nasopharyngeal swab taken from a RT-qPCR-confirmed asymptomatic patient returning from Wuhan, China beginning of February 2020³⁵ was directly sequenced on a MinION platform (Oxford Nanopore) as described previously⁶². Phylogenetic analysis confirmed a close relation with the prototypic Wuhan-Hu-1 2019-nCoV (GenBank accession number MN908947.3) strain. Infectious virus was isolated by serial passaging on HuH7 and Vero E6 cells¹³, with the addition of penicillin/streptomycin, gentamicin and amphotericin B. Virus used for animal experiments was from passage P6. Prior to inoculation of animals, virus stocks were confirmed to be free of mycoplasma (PlasmoTest, InvivoGen) and other adventitious agents by deep sequencing on a MiSeq platform (Illumina) following an established metagenomics pipeline^{63,64}. The infectious content of virus stocks was determined by titration on Vero E6 cells by the Spearman-Kärber method for use in Example 9, or by the Reed and Muench method⁷¹ for use in Example 23. All virus-related work was conducted in the high-containment BSL3+ facilities of the KU Leuven Rega Institute (3CAPS) under licenses AMV 30112018 SBB 219 2018 0892 and AMV 23102017 SBB 219 2017 0589 according to institutional guidelines.

### Cells

Vero E6 cells (African green monkey kidney, ATCC CRL-1586) were cultured in minimal essential medium (Gibco) supplemented with 10% fetal bovine serum (Integro), 1% L-glutamine (Gibco) and 1% bicarbonate (Gibco). End-point titrations were performed with medium containing 2% fetal bovine serum instead of 10%.

### Sera Used in Example 9

Human convalescent plasma (Patient #2) was obtained from Biobank Rode Kruis-Vlaanderen, registered under Belgian law as Biobank BB190034. Plasma donated by a healthy volunteer sampled prior to emergence of SARS-CoV-2 served as negative control (NC donor). Serum/plasma was administered i.p. 1 day prior to infection, in a volume of 1000 μl per hamster. Antibody VHH-72-Fc was administered i.p. at a concentration of 20 mg/kg 1 day prior to infection. VHH-72-Fc was expressed in ExpiCHO cells (ThermoFisher Scientific) and purified from the culture medium as described¹⁰. Briefly, after transfection with pcDNA3.3-VHH-72-Fc plasmid DNA, followed by incubation at 32° C. and 5% CO₂ for 6-7 days, the VHH-72-Fc protein in the cleared cell culture medium was captured on a 5 mL MabSelect SuRe column (GE Healthcare), eluted with a McIlvaine buffer pH 3, neutralized using a saturated Na₃PO₄ buffer, and buffer exchanged to storage buffer (25 mM L-Histidine, 125 mM NaCl). The antibody's identity was verified by protein- and peptide-level mass spectrometry.

### RNA Extraction and RT-QPCR as Performed in the Experiment in Example 9

Animals were euthanized at 4 days post-infection, organs were removed and lungs were homogenized manually using a pestle and a 12-fold excess of cell culture medium (DMEM/2% FCS). RNA extraction was performed from homogenate of 4 mg of lung tissue with RNeasy Mini Kit (Qiagen), or 50 μl of serum using the NucleoSpin kit (Macherey-Nagel), according to the manufacturer's instructions. Other organs were collected in RNALater (Qiagen) and homogenized in a bead mill (Precellys) prior to extraction. Of 100 μl eluate, 4 μl was used as template in RT-qPCR reactions. RT-qPCR was performed on a LightCycler96 platform (Roche) using the iTaq Universal Probes One-Step RT-qPCR kit (BioRad) with primers and probes (Table 7) specific for SARS-CoV-2 and hamster β-actin (ACTB), ACE2, MX2 and IP-10 (IDT). For each data point, qPCR reactions were carried out in duplicate. Standards of SARS-CoV-2 cDNA (IDT) and infectious virus were used to express the amount of RNA as normalized viral genome equivalent (vge) copies per mg tissue, or as TCID₅₀ equivalents per mL serum, respectively. The mean of housekeeping gene β-actin was used for normalization. The relative fold change was calculated using the 2^{-ΔΔCt} method⁶⁵.

TABLE 7
Primers used for RT-QPCR

| | | | SEQ ID |
|---|---|---|---|
| Gene | Description | Oligonucleotide sequence | NO |
| SARS-Cov- | Forward primer | 5′-TTA CAA ACA TTG GCC GCA AA-3′ | 82 |
| 2 | Reverse primer | 5′-GCG CGA CAT TCC GAA GAA-3′ | 83 |
| Hamster | Forward primer | 5′-CCA GTA ATG TGG ACA TTG CC-3′ | 84 |
| MX2 | Reverse primer | 5′-CAT CAA CGA CCT TGT CTT CAG TA-3′ | 85 |
| Hamster IP- | Forward primer | 5′-GCC ATT CAT CCA CAG TTG ACA-3′ | 86 |
| 10 | Reverse primer | 5′-CAT GGT GCT GAC AGT GGA GTC T-3′ | 87 |
| Hamster | Forward primer | 5′-GGG AAC TGT CAA AGG GTA CAG-3′ | 88 |
| ACE2 | Reverse primer | 5′-CCC TTC CTA CAT CAG TCC TAC T-3′ | 89 |
| Hamster | Forward primer | 5′-GGC CAG GTC ATC ACC ATT-3′ | 90 |
| ACTB | Reverse primer | 5′-GAG TTG AAT GTA GTT TCG TGG ATG-3′ | 91 |

### In Vivo Syrian Hamster Experiments as Performed in Example 22 (Challenge Study FIG. 37)

The efficacy of bivalent and tetravalent SARS-CoV-2 specific nanobodies as therapeutic or prophylactic treatment against SARS-CoV-2 infection was assessed in the hamster challenge model. The primary endpoint for the evaluation of the efficacy of the therapy was the viral load in the respiratory tract. Male golden Syrian hamsters were infected via the intranasal (i.n.) route with 10⁴ TCID₅₀ SARS-CoV-2 (strain BetaCoV/Munich/BavPat1/2020, p3, this strain carries the D614G mutation in the spike protein, which provides an advantage in fast viral entry and is now the dominant pandemic form³⁶) on day 0 of the study. Animals received treatment prophylactically (24 hours before infection) or therapeutically (4 hours post infection [p.i.]) with the different compounds at different doses via the intraperitoneal (i.p.) route, with six animals per group. Animals were euthanised on day 4 p.i. to perform necropsy. Animals were weighed and sampled daily from the throat during the study to monitor body weight changes and to assess of viral shedding in the respiratory tract. Viral load in lung, broncho-alveolar lavage (BAL) and nasal turbinate tissue and histopathological changes in selected tissues were assessed after euthanasia. Upon necropsy, broncho alveolar lavage was performed and tissue samples were collected and stored in 10% formalin for histopathology and immunohistochemistry and frozen for virological analysis. After fixation with 10% formalin, sections from left lung and left nasal turbinate were embedded in paraffin and the tissue sections were stained for histological examination.

For virological analysis, tissue samples were weighed, homogenized in infection medium and centrifuged briefly before titration. Serum samples on day 4 post infection were collected for PK analysis. Throat swabs, BAL and tissue homogenates were used to detect viral RNA.

To this end RNA was isolated (SOP VC-M098; Performing nucleic acid purification on the MagNA Pure 96) and Taqman PCR (SOP VC-M052; Performing assays on the 7500 RealTime PCR system (general method)) was performed using specific primers and probe specific for beta coronavirus E gene. The number of virus copies in the different samples were calculated using the resulting Ct value for the sample against slope, intercept and upper and lower limits of detection for the standard virus included in each run.

Detection of replication competent virus: Quadruplicate 10-fold serial dilutions were used to determine the virus titers in confluent layers of Vero E6 cells. To this end, serial dilutions of the samples (throat swabs, BAL and tissue homogenates) were made and incubated on Vero E6 monolayers for 1 hour at 37 degrees. Vero E6 monolayers are washed and incubated for 4-6 days at 37 degrees after which plates are stained and scored using the vitality marker WST8 (colourmetric readout). Viral titers (TCID₅₀/ml or/g) were calculated using the method of Spearman-Karber.

### In Vivo Syrian Hamster Experiments as Performed in Example 23

The hamster infection model of SARS-CoV-2 has been described before^{13,69}. In brief, wild-type Syrian Golden hamsters (Mesocricetus auratus) were purchased from Janvier Laboratories and were housed per two in ventilated isolator cages (IsoCage N Biocontainment System, Tecniplast) with ad libitum access to food and water and cage enrichment (wood block). The animals were acclimated for 4 days prior to study start. Housing conditions and experimental procedures were approved by the ethics committee of animal experimentation of KU Leuven (license P065-2020). Female hamsters of 6-8 weeks old were anesthetized with ketamine/xylazine/atropine and inoculated intranasally with 50 μL containing 2×106 TCID50 SARS-CoV-2 (day 0).

Animals were treated in a therapeutic setting according to the schedule in Table 4: i.e. hamsters were treated with D72-53 (PB9683) (4 mg/kg), Pre-lead (D72-58) (4 mg/kg), or control 24 h after infection by intraperitoneal administration. Hamsters were monitored for appearance, behavior and weight. At day 4 post infection (pi), hamsters were euthanized by i.p. injection of 500 μL Dolethal (200 mg/mL sodium pentobarbital, Vétoquinol SA). Lungs were collected and viral RNA and infectious virus were quantified by RT-qPCR and end-point virus titration, respectively. Blood samples were collected at end-point sacrifice and serum was obtained for PK analysis.

Hamster lung tissues were collected after sacrifice and were homogenized using bead disruption (Precellys) in 350 μL RLT buffer (RNeasy Mini kit, Qiagen) and centrifuged (10,000 rpm, 5 min) to pellet the cell debris. RNA was extracted according to the manufacturer's instructions. Of 50 μL eluate, 4 μL was used as a template in RT-qPCR reactions. RT-qPCR was performed on a LightCycler96 platform (Roche) using the iTaq Universal Probes One-Step RT-qPCR kit (BioRad) with N2 primers and probes targeting the nucleocapsid¹³. Standards of SARS-CoV-2 cDNA (IDT) were used to express viral genome copies per mg tissue or per mL serum.

Lung tissues were homogenized using bead disruption (Precellys) in 350 μL minimal essential medium and centrifuged (10,000 rpm, 5 min, 4° C.) to pellet the cell debris. To quantify infectious 5ARS-CoV-2 particles, endpoint titrations were performed on confluent Vero E6 cells in 96-well plates. Viral titers were calculated by the Reed and Muench method⁷¹ using the Lindenbach calculator and were expressed as 50% tissue culture infectious dose (TCID50) per mg tissue.

For histological examination, the lungs were fixed overnight in 4% formaldehyde and embedded in paraffin. Tissue sections (5 μm) were analyzed after staining with hematoxylin and eosin and scored blindly for lung damage by an expert pathologist. The scored parameters, to which a cumulative score of 1 to 3 was attributed, were the following: congestion, intra-alveolar hemorrhagic, apoptotic bodies in bronchus wall, necrotizing bronchiolitis, perivascular edema, bronchopneumonia, perivascular inflammation, peribronchial inflammation and vasculitis.

### PK/PD Analysis in Hamsters (Example 29)

Bioanalysis of all hamster serum and BALF samples was done using a competition AlphaLISA (amplified luminescent proximity homogeneous assay) method. This assay detects the inhibition of the interaction of SARS-CoV-2 RBD protein with monovalent VHH72_h1 (S56A) nanobody captured on donor and acceptor beads, leading to an energy transfer between beads producing a fluorescent signal. This homogeneous assay without wash steps in a closed system is considered advantageous for testing samples from virus challenged animals (Boudewijns et al. 2020 (Ref13)). From one challenge study (FIG. 37, Example 23; Munich isolate), serum and BALF samples were inactivated by heating for 30 min at 56C, yielding a 4 log-fold reduction in infectious virus. The assay is run in white low binding 384-well microtitre plates (F-bottom, Greiner Cat nr 781904). Hamster serum samples were analysed in duplicates at two dilutions (300- and 900-fold). BALF samples were analysed in duplicates at 1:3 and 1:5 dilutions, respectively. The calibration standard curve of the corresponding VHH72-h1 S56A-Fc was generated by serial dilution (1.7 fold) starting from 50 nM in diluted hamster serum in assay buffer (PBS with 0.5% BSA and 0.05% Tween20). QCs are prepared fresh on the day of assay from a different working stock in diluted hamster serum in assay buffer. BALF samples were analysed in buffer, after confirming lack of matrix effects with reference material. To each well, 5 μl of standard/QC/samples are mixed with 5 μl of 3 nM Nanobody (VHH72_h1 (S56A)-Flag3-His6) and 5 μl of 2.5 nM biotinylated SARS-CoV-2 RBD protein. After an incubation for 1 hour at room temperature, 5 μl streptavidin coated Alpha Donor beads (Perkin Elmer, Cat nr. 6760002) and 5 μl anti-Flag AlphaLISA acceptor beads (Perkin Elmer, Cat nr. AL112C) were added (final concentration of 20 μg/mL each) in a final volume of 25 μl for an incubation of 1 hour at room temperature in the dark. Interaction between beads was assessed after illumination at 680 nm and reading at 615 nm of on an Ensight instrument. Sample concentrations were backcalculated to a standard calibration curve by 4PL analysis.

### Statistical Analysis

GraphPad Prism Version 8 (GraphPad Software, Inc.) was used for all statistical evaluations. The number of animals and independent experiments that were performed is indicated in the legends to figures. Statistical significance was determined using the non-parametric Mann Whitney U-test unless mentioned otherwise. Values were considered significantly different at P values of 50.05.

Flow Cytometric analysis of antibody binding to Sarbecovirus RBD displayed on the surface of Saccharomyces cerevisiae.

A pool of plasmids, based on the pETcon yeast surface display expression vector, that encode the RBDs of a set of SARS-CoV2 homologs was generously provided by Dr. Jesse Bloom (Starr et al., Cell 2020 Sep. 3; 182(5):1295-1310.e20)³⁸. This pool was transformed to E. coli TOP10 cells by electroporation at the 10 ng scale and plated onto low salt LB agar plates supplemented with carbenicillin. Single clones were selected, grown in liquid low salt LB supplemented with carbenicillin and miniprepped. Selected plasmids were Sanger sequenced with primers covering the entire RBD CDS and the process was repeated until every desired RBD homolog had been picked up as a sequence-verified single clone. Additionally, the CDS of the RBD of SARS-CoV2 was ordered as a yeast codon-optimized gBlock and cloned into the pETcon vector by Gibson assembly. The plasmid was transformed into E. coli, prepped and sequence-verified as described above. DNA of the selected pETcon RBD plasmids was transformed to Saccharomyces cerevisiae strain EBY100 according to the protocol by Gietz and Schiestl ((Nat. Protoc. 2, 31-34, 2007)⁷⁹ and plated on yeast drop-out medium (SD agar -trp -ura). Single clones were selected and verified by colony PCR for correct insert length. A single clone of each RBD homolog was selected and grown overnight in 10 ml liquid repressive medium (SRaf -ura -trp) at 28° C. These precultures were then back-diluted to 50 ml liquid inducing medium (SRaf/Gal -ura -trp) at an OD₆₀₀ of 0.67/ml and grown for 16 hours before harvest. After washing in PBS, the cells were fixed in 1% PFA, washed twice with PBS, blocked with 1% BSA and stained with VHHs at different concentration. Binding of the antibodies was detected using Alexa fluor 633 conjugated anti-human IgG antibodies (Invitrogen). Expression of the surface-displayed myc-tagged RBDs was detected using a FITC conjugated chicken anti-myc antibody (Immunology Consultants Laboratory, Inc.). Following 3 washes with PBS containing 0.5% BSA, the cells were analyzed by flow cytometry using an BD LSRII flow cytometer (BD Biosciences). Binding was calculated as the ratio between the AF647 MFI of the RBD⁺ (FITC) cells over the AF647 MFI of the RBD⁻ (FITC cells).

Production of VHHs by Pichia pastoris and E. coli (Example 30).

Small scale production of VHHs in Pichia pastoris is described in Ref 10. For the production of VHHs in E. coli, a pMECS vector containing the VHH of interest was transformed into WK6 cells (the non-suppressor E. coli strain) and plated on an LB plate containing Ampicillin. The next day clones were picked and grown overnight in 2 mL LB containing 100 ug/ml ampicillin and 1% glucose at 37° C. while shaking at 200 rpm. One ml of this preculture was used to inoculate 25 ml of TB (terrific broth) supplemented with 100 μg/ml Ampicillin, 2 mM MgCl₂ and 0.1% glucose and incubated at 37° C. with shaking (200-250 rpm) till an OD₆₀₀ of 0.6-0.9 is reached. VHH production was induced by addition of IPTG to a final concentration of 1 mM. These induced cultures were incubated overnight at 28° C. while shaking at 200 rpm. The produced VHHs were extracted from the periplasm and purified as described in Ref 10. In short, the VHHs were purified from the solution using Ni Sepharose beads (GE Healthcare). After elution using 500 mM imidazole the VHH containing flow-through fractions were buffer-exchanged with PBS with a Vivaspin column (5 kDa cutoff, GE Healthcare). The purified VHHs were analyzed by SDS-PAGE and Coomassie staining and by intact mass spectrometry.

### Enzyme-Linked Immunosorbent Assay (Example 30).

Wells of microtiter plates (type II, F96 Maxisorp, Nuc) were coated overnight at 4° C. with 100 ng of recombinant SARS-CoV S-2P protein (with foldon), SARS-CoV-1 S-2P protein (with foldon), mouse Fc-tagged SARS-CoV-2 RBD (Sinobiologicals) or BSA. The coated plates were blocked with 5% milk powder in PBS. Dilution series of the VHHs were added to the wells. Binding was detected by incubating the plates sequentially with HRP-conjugated rabbit anti-camelid VHH antibodies (Genscript). After washing 50 μL of TMB substrate (Tetramethylbenzidine, BD OptETA) was added to the plates and the reaction was stopped by addition of 50 μL of 1 M H₂SO₄. The absorbance at 450 nM was measured with an iMark Microplate Absorbance Reader (Bio Rad). Curve fitting was performed using nonlinear regression (Graphpad 8.0).

For the competition assay in which binding of VHHs to monovalent RBD captured by VHH72-Fc or the human S309 monoclonal antibody was tested, ELISA plates were coated with 50 ng of VHH72-Fc or S309 in PBS for 16 hours at 4° C. After washing with PBS and then PBS containing 0.1% tween-20, the wells were blocked with PBS containing 5% milk powder for 1 hour at room temperature. Then twenty ng of monomeric RBD (in house produced RBD-SD1-Avi) was added to the wells and incubated for 1 hour at room temperature. Subsequently, 0.5 ug/ml of the VHHs (10 ug/ml for VHH72_h1_S56) was added to the wells and incubated for 1 hour at room temperature. After washing 2 times with PBS and 3 times with PBS containing 2% milk and 0.05% tween-20 the bound VHHs were detected using a mouse anti-HIS-tag antibody (Biorad) and an HRP conjugated sheep anti-mouse IgG antibody (GE healthcare).

### Biolayer Interferometry as Performed in Example 30.

The SARS-CoV-2 RBD binding kinetics of VHH variants were assessed via biolayer interferometry on an Octet RED96 system (FortéBio). To measure the affinity of monovalent VHH variants for RBD, monomeric human Fc-fused SARS-CoV-2_RBD-SD1 (Wrapp et al, 2020 May 28; 181(5):1004-1015) at 15 μg/ml was immobilized on anti-human IgG Fc capture (AHC) biosensors (FortéBio) to a signal of 0.35-0.5 nm. Association (120 s) and dissociation (480 s) of duplicate 200 nM VHHs were measured in kinetics buffer. Between analyses, biosensors were regenerated by three times 20 s exposure to regeneration buffer (10 mM glycine pH 1.7). Data were double reference-subtracted and aligned to each other in Octet Data Analysis software v9.0 (FortéBio). Offrates (kdis) were fit in a 1:1 model.

Competition amongst VHH variants for SARS-CoV-2 RBD binding was assessed via biolayer interferometry on an Octet RED96 system (FortéBio). Bivalent VHH72-hFc (50 nM) was immobilized on anti-human IgG Fc capture (AHC) biosensors (FortéBio), followed by capture of antigen RBD-SD1_mFc (200 nM) to saturation. Then, competition with 1 μM VHH variants (protein concentrations calculated by a Trinean DropSense machine, Lunatic chip, after subtraction of the turbidity profile extrapolated from the absorbance spectrum at 320-400 nm) was measured for 600 s. Between analyses, biosensors were regenerated by three times 20 s exposure to regeneration buffer (10 mM glycine pH 1.7). Data were double reference-subtracted and aligned to each other in Octet Data Analysis software v9.0 (FortéBio).

### Flow Cytometric Analysis of Binding to HEK293 Cells Expressing the SARS-CoV Spike Protein (Example 31).

To investigate the binding of VHHs to spike proteins on the surface of mammalian cells by flow cytometry we used expression plasmids containing the coding sequence of the SARS-CoV-1 spike protein in which the RBD was replaced by that of SARS-CoV-2 as described by Letko et al. (Nature Microbiology, 2020, April; 5(4):562-569). The latter was used as a template to generate expression plasmids of the K378N spike variants by QuickChange site-directed mutagenesis (Agilent) according to the manufacturer's instructions. Two days after transfecting HEK293T cells or HEKS cells with spike expression plasmids each combined with a GFP expression plasmid, the cells were collected, washed once with PBS and fixed with 1% PFA for 30 minutes. Binding of VHHs was detected using a mouse anti-HIS-tag antibody (Biorad) and an AF647 conjugated donkey anti-mouse IgG antibody (Invitrogen). Following 3 washes with PBS containing 0.5% BSA, the cells were analyzed by flow cytometry using an BD LSRII flow cytometer (BD Biosciences). Binding was calculated as the mean AF647fluorescence intensity (MFI) of GFP expressing cells (GFP⁺) divided by the MFI of GFP negative cells (GFP⁻). The binding curves were fitted using nonlinear regression (Graphpad 8.0).

### Deep Mutational Scanning (Example 33)

Transformation of Deep Mutational SARS-CoV2 RB Libraries to E. coli

Plasmid preps of two independently generated deep mutational SARS-CoV2 RBD libraries in the pETcon vector were generously provided by Dr. Jesse Bloom (Starr et al. 2020, Cell 182, 1295-1310.e20). Ten ng of these preps were transformed to E. coli TOP10 strain via electroporation, and allowed to recover for one hour in SOC medium at 37° C. The transformation mixture was divided and plated on ten 24.5 cm×24.5 cm large bio-assay dishes containing low salt LB medium supplemented with carbenicillin, at an expected density of 100.000 clones per plate. After growing overnight, all colonies were scraped from the plates and resuspended into 300 ml low salt LB supplemented with carbenicillin. The cultures were grown for 2 hours and a half before pelleting. The cell pellet was washed once with sterile MQ, and plasmid was extracted via the QIAfilter plasmid Giga prep kit (Qiagen) according to the manufacturer's instructions.

Transformation of Deep Mutational SARS-CoV2 R8D Libraries to S. cerevisiae

Ten μg of the resulting plasmid preps were transformed to Saccharomyces cerevisiae strain EBY100, according to the large-scale protocol by (Gietz et al. Nature Protocols 2007, 2, 31-345) Gietz and Schiestl. Transformants were selected in 100 ml liquid yeast drop-out medium (SD -trp -ura) for 16 hours. Then the cultures were back-diluted into 100 mL fresh SD -trp -ura at 1 OD₆₀₀ for an additional 9 hours passage. Afterwards, the cultures were flash frozen in 1e8 cells aliquots in 15% glycerol and stored at −80° C.

Cloning and Transformation of WT RBD of SARS-CoV2

The CDS of the RBD of SARS-CoV2 was ordered as a yeast codon-optimized gBlock and cloned into the pETcon vector by Gibson assembly. The cloning mixture was similarly electroporated into E. coli TOP10 cells, and plasmid was extracted via a Miniprep kit (Promega) according to the manufacturer's instructions. The plasmid was Sanger sequenced with primers covering the entire RBD CDS. Finally, the plasmid was transformed to Saccharomyces cerevisiae strain EBY100, according to the small-scale protocol by (Gietz et al. Nature Protocols 2007, 2, 31-34) Gietz and Schiestl. Transformants were selected via a yeast colony PCR.

Presorting of Deep Mutational SARS-CoV2 RBD Libraries on ACE2

One aliquot of each library was thawed and grown overnight in 10 ml liquid repressive medium (SRaf -ura -trp) at 28° C. Additionally, the control EBY100 strain containing the pETcon plasmid expressing WT RBD from SARS-CoV2 was inoculated in 10 ml liquid repressive medium and grown overnight at 28° C. These precultures were then back-diluted to 50 ml liquid inducing medium (SRaf/Gal -ura -trp) at an OD600 of 0.67/ml and grown for 16 hours before harvest.

The cells pellets were washed thrice with washing buffer (1×PBS+1 mM EDTA, pH 7.2+1 Complete Inhibitor EDTA-free tablet (Roche) per 50 ml buffer), and stained at an OD₆₀₀ of 8/mi with 9.09 nM hACE2-muFc (Sino Biological) in staining buffer (washing buffer+0.5 mg/ml of Bovine Serum Albumin) for one hour at 4° C. on a rotating wheel. Cells were washed thrice with staining buffer and stained with 1:100 anti-cmyc-FITC (immunology Consultants Lab), 1:1000 anti-mouse-IgG-AF568 (Molecular Probes) and 1:200 L/D eFluor506 (Thermo Fischer Scientific) for one hour at 4° C. on a rotating wheel. Cells were washed thrice with staining buffer, and filtered over 35 μm cell strainers before sorting on a FACS Melody (BD Biosciences). A selection gate was drawn that captures the ACE2+ cells, such that, after compensation, max. 0.1% of cells of unstained and single stained controls appeared above the background. Approximately 2.5 million ACE2+ cells were collected per library, each in 5 ml polypropylene tubes coated with 2×YPAD+1% BSA.

Sorted cells were recovered by growth in liquid SD -trp -ura medium with 100 U/ml penicillin and 100 μg/ml streptomycin (Thermo Fisher Scientific) for 72 hours at 28° C., and flash frozen at −80° C. in 9 OD₆₀₀ unit aliquots in 15% glycerol.

Nanobody Escape Mutant Sorting on ACE2-Sorted Deep Mutational SARS-CoV2 RBD Libraries

One ACE2-sorted aliquot of each library was thawed and grown overnight in 10 ml liquid repressive medium (SRaf -ura -trp) at 28° C. Additionally, the control EBY100 strain containing the pETcon plasmid expressing WT RBD from SARS-CoV2 was inoculated in 10 ml liquid repressive medium and grown overnight at 28° C. These precultures were then back-diluted to 50 ml liquid inducing medium (SRaf/Gal--ura -trp) at an OD600 of 0.67/ml and grown for 16 hours before harvest.

The cells pellets were washed thrice with washing buffer (1×PBS+1 mM EDTA, pH 7.2+1 Complete Inhibitor EDTA-free tablet (Roche) per 50 ml buffer, freshly made and filter sterile) and stained at an OD₆₀₀ of 8/ml with a specific concentration per stained nanobody in staining buffer (washing buffer+0.5 mg/ml of Bovine Serum Albumin) for one hour at 4° C. on a rotating wheel. Specifically, we stained at 400 ng/ml for VHH72 h1 S56A and 10 ng/ml for VHH3.38, VHH3.55 and VHH3.83. Cells were washed thrice with staining buffer and stained with 1:2000 mouse anti-His (Biorad) for 1 h30 at 4° C. on a rotating wheel. Cells were washed thrice with staining buffer and stained with 1:100 anti-c-myc-FITC (Immunology Consultants Lab), 1:1000 anti-mouse-IgG-AF568 (Molecular Probes) and 1:200 L/D eFluor506 (Thermo Fischer Scientific) for one hour at 4C on a rotating wheel. Cells were washed thrice with staining buffer, and filtered over 35 μm cell strainers before sorting on a FACS Melody (BD Biosciences). Gating was chosen as such that, after compensation, max. 0.1% of cells of the fully stained WT RBD control appeared in the selection gate. Between 150.000 and 350.000 escaped cells were collected per library, each in 5 ml polypropylene tubes coated with 2×YPAD+1% BSA.

Sorted cells were recovered by growth in liquid SD -trp -ura medium supplemented with 100 U/ml penicillin and 100 μg/ml streptomycin (Thermo Fisher Scientific) for 16 hours at 28° C.

DNA Extraction and Illumina Sequencing of Nanobody Escape Sorted Deep Mutational SARS-CoV2 RBD Libraries

Plasmids were extracted from sorted cells using the Zymoprep yeast plasmid miniprep II kit (Zymo Research) according to the manufacturer's instructions, but with the exception of a longer (2 hour) incubation with the Zymolyase enzyme, and with the addition of a freeze-thaw cycle in liquid nitrogen after Zymolyase incubation.

A PCR was performed on the extracted plasmids using KAPA HiFi HotStart ReadyMix to add sample indices and remaining Illumina adaptor sequences using NEBNext UDI primers (20 cycles). PCR samples were purified once using CleanNGS magnetic beads (CleanNA), and once using AMPure magnetic beads (Beckman Coulter). Fragments were eluted in 15 μl 0.1×TE buffer. Size distributions were assessed using the High Sensitivity NGS kit (DNF-474, Advanced Analytical) on a 12-capillary Fragment Analyzer (Advanced Analytical). Hundred bp single-end sequencing was performed on a NovaSeq 6000 by the VIB Nucleomics core (Leuven, Belgium).

Analysis of Sequencing Data and Epitope Calculation Using Mutation Escape Profiles

Deep sequencing reads were processed as described by Greaney et al. (Greaney et al., 2021, Cell Host Microbe) using the code available on the internet at github.com/jbloomlab/SARS-CoV-2-RBD_MAP_Crowe_antibodies, with adjustments. Briefly, nucleotide barcodes and their corresponding mutations were counted using the dms_variants package (0.8.6). Escape fraction for each barcode was defined as the fraction of reads after enrichment divided by the fraction of reads before enrichment of escape variants. The resulting variants were filtered to remove unreliably low counts and keep variants with sufficient RBD expression and ACE2 binding (based on published data (Starr et al., 2020, Cell 182, 1295-1310.e20). For variants with several mutations, the effects of individual mutations were estimated with global epistasis models, excluding mutations not observed in at least one single mutant variant and two variants overall. The resulting escape measurements correlated well between the duplicate experiments and the average across libraries was thus used for further analysis. To determine the most prominent escape sites for each nanobody, RBD positions were identified where the total site escape was >10×the median across all sites, and was also at least 10% of the maximum total site escape across all positions for a given nanobody.

### Sequence Listing

SEQ ID NO: 1: VHH-72 amino acid sequence
SEQ ID NO: 2: VHH72-h1 humanized variant 1 of VHH-72 amino acid sequence
SEQ ID NO: 3: VHH72-h1(E1D) humanized variant 1(E1D) of VHH-72 amino acid sequence
SEQ ID NO: 4: VHH72-S56A variant amino acid sequence
SEQ ID NO:5: VHH72_h1(S56A) humanized variant 1 of VHH72-S56A amino acid sequence
SEQ ID NO:6: VHH72_h1(E1D)(S56A) humanized variant 1(E1D) of VHH72-S56A amino acid sequence
SEQ ID NO: 7: CDR1 of VHH-72 (or VHH72-S56A) amino acid sequence (according to Kabat annotation)
SEQ ID NO: 8: CDR2 of VHH-72 amino acid sequence (according to Kabat annotation)
SEQ ID NO: 9: CDR3 of VHH-72 (or VHH72-S56A) amino acid sequence (according to Kabat annotation)
SEQ ID NO:10: CDR2 of VHH-72-S56A amino acid sequence (according to Kabat annotation)
SEQ ID NO: 11: VHH72_h2 humanized variant 2 of VHH72 amino acid sequence
SEQ ID NO: 12: bivalent fusion of VHH-72 with a (Gly₄Ser)₃-linker
SEQ ID NO: 13: VHH-72 fused to human IgG1 Fc with a glycine-serine linker in between
SEQ ID NO: 14: mouse VH signal sequence-VHH72-GSGGGGSGGGGS-hIgG1Hinge-hIgG1Fc (VHH72 fused to human IgG1Hinge region followed by the humanIgG1Fc region with a GSGGGGSGGGGS linker between the VHH72 and the IgG1Hinge region)
SEQ ID NO: 15: mouse VH signal sequence-VHH72-GSGGGGSGGGGS-hIgG1Hinge-hIgG2Fc (VHH72 fused to human IgG1Hinge region followed by the human IgG1Fc region)
SEQ ID NO: 16: mouse VH signal sequence—VHH72-GSGGGGSGGGGS-hIgG2Hinge_ERKCCdel-hIgG2Fc (VHH72 fused to the human IgG2Hinge region (ERKCC amino acids are deleted) followed by the human IgG2Fc region with a GSGGGGSGGGGS linker between the VHH72 and the human IgG2Hinge region)

### SEQ ID NO:17: D72-58 [VHH72_h1(E1D)_10GS_IgG1_LALA; Prelead]

SEQ ID NO:18: D72-1 [VHH72-GS(G4S)2-hIgG1hinge-hIgG1Fc; Prototype as used in Wrapp et al.]
SEQ ID NO: 19: VHH72_h1_S56A-GS-hIgG1hinge-hIgG1Fc_LALAPG (D72-23) amino acid sequence

### SEQ ID NO: 20: VHH72_h1_E1D_S56A-(G4S)₂-hIgG1hinge_EPKSCdel-hIgG1Fc_LALAPG_Kdel (D72-52; PB9590)

### SEQ ID NO: 21: VHH72_h1_E1D_S56A-(G4S)₃-VHH72_h3_S56A-GS-hIgG1hinge_EPKSCdel-hIgG1Fc_LALAPG_Kdel (D72-55)

SEQ ID NO: 22: VHH72_h1_E1D_S56A-(G4S)₂-hIgG1hinge_EPKSCdel-hIgG1Fc_LALA_Kdel (361AA; PB9683 batch, D72-53 construct)
SEQ ID NO: 23: Sars-Cov2 Spike protein (alternative name: Wuhan seafood market pneumonia virus (nCo2019-virus; cov2-Wuhan). Genbank Accession: QHQ82464, version QHQ82464.1.
SEQ ID NO:24: Sars-Cov1 Spike protein or Corona virus SARS Spike protein (corresponds with GenBank accession NP_828851.1)
SEQ ID NO:25: SARS-CoV-2 Spike protein RBD domain region (corresponding to 330-518 of SEQ ID NO: 23 depicting the SARS-Cov-2 Spike) amino acid sequence
SEQ ID NO: 26: Receptor Binding Domain (RBD) from SARS-CoV-1 Spike protein, corresponding with amino acid residues 320-502 of SEQ ID NO:24 or derived from GenBank ID: NP_828851.1.
SEQ ID NO: 27-61: further VHH72 mutant variants
SEQ ID NO: 62: Light chain of S309 antibody
SEQ ID NO: 63: Heavy chain of S309 antibody
SEQ ID NO: 64: CB6 light chain sequence
SEQ ID NO: 65: CB6 heavy chain sequence
SEQ ID NO:66-81: Spike protein RBD sequences from different strains, with a deletion of the RBM loop, as shown in FIG. 42
SEQ ID NO: 82-91: Oligo DNA sequences (see Table 7 methods).

### SEQ ID NO: 92-105+SEQ ID NO:111-140: see Table 6.

### SEQ ID NO:106-110: VHH3.39, VHH3.89, VHH3.141, VHH3.151, VHH3BD9

SEQ ID NO:141: CDR2 of VHH-72-S52A-S56A mutant amino acid sequence

### Aspects of the Disclosure

A binding agent specifically binding the Corona virus Spike protein comprising amino acid residues Leu355, Tyr356, Ser358, Ser362, Thr363, F364, K365, C366 and Y494 as set forth in SEQ ID NO:24.
A binding agent specifically binding the Corona virus Spike protein as defined above further comprising amino acid residue R426 as set forth in SEQ ID NO: 24.
Said binding agent, wherein said binding agent is a small compound, a chemical, a peptide, a peptidomimetic, an antibody mimetic, an immunoglobulin single variable domain (ISVD) an antibody or antibody fragment.
Said binding agent, wherein said binding agent is an ISVD comprising 4 framework regions (FR) and 3 complementarity determining regions (CDR) according to the following formula (1): FR1-CDR1-FR2-CDR2-FR3-CDR3-FR4 (1); and wherein CDR1 consists of a sequence depicted in SEQ ID NO: 7; CDR2 consists of a sequence depicted in SEQ ID NO: 8; and CDR3 consists of a sequence depicted in SEQ ID NO: 9.
Said ISVD, comprising SEQ ID NO: 1, or a sequence with at least 90% amino acid identity with SEQ ID NO: 1, or a humanized variant thereof as set forth for example in SEQ ID NO: 2 and 11.
Any of the above binding agents for use as a medicament.
Any of the above binding agents for use in treatment of SARS-Corona virus infection, more specifically for use in the treatment of 2019-nCorona virus infection.
A binding agent comprising an ISVD specifically binding the Corona virus Spike protein comprising 4 framework regions (FR) and 3 complementarity determining regions (CDR) according to the following formula (1): FR1-CDR1-FR2-CDR2-FR3-CDR3-FR4 (1); and wherein CDR1 consists of a sequence depicted in SEQ ID NO: 7; CDR2 consists of a sequence depicted in SEQ ID NO: 8; and CDR3 consists of a sequence depicted in SEQ ID NO: 9, for use as a medicament.
Said binding agent for use as a medicament, comprising SEQ ID NO: 1, or a sequence with at least 90% amino acid identity with SEQ ID NO: 1, or a humanized variant thereof.
Said binding agent for use as a medicament, comprising an IgG Fc fusion.
Said binding agent for use as a medicament, comprising an IgG1 Fc fusion, preferably as depicted in SEQ ID NO:13.
Said binding agent for use in treatment of SARS-Corona virus infection, more specifically for use in the treatment of 2019-nCorona virus infection.
Said binding agent for use in prophylactic treatment of SARS-Corona virus infection, more specifically for use in the treatment of 2019-nCorona virus infection.
Said binding agent for use in prophylactic treatment of SARS-Corona virus infection, more specifically for use in the treatment of 2019-nCorona virus infection, by administering a dose of 0.5 mg/kg-25 mg/kg.
Said binding agent for use in therapeutic treatment of SARS-Corona virus infection, more specifically for use in the treatment of 2019-nCorona virus infection.
A complex comprising the RBD of SARS-Corona virus as depicted in SEQ ID NO: 26 and any of the above binding agents.
Said complex, wherein said complex is crystalline.
A crystal comprising the SARS-Corona RBD as depicted in SEQ ID NO: 26 and the binding agent depicted in SEQ ID NO: 1 and characterized in that the crystal is:
   a crystal between SEQ ID NO: 26 and SEQ ID NO: 1 in the space group P3₁21, with the following crystal lattice constants: a=88.8 Å±5%, b=88.8 Å±5%, c=200.8 Å±5%, α=90, β=90, γ=120,
Said crystal, which has a three-dimensional structure wherein the crystal i) comprises an atomic structure characterized by the coordinates of the database entry PDB 6WAQ or a subset of atomic coordinates of PBD 6WAQ.
A binding site, consisting of a subset of atomic coordinates, present in the crystal i) as defined in above, wherein said binding site consists of the amino acid residues: Leu355, Tyr356, Ser358, Ser362, Thr363, F364, K365, C366 and Y494, or Leu355, Tyr356, Ser358, Ser362, Thr363, F364, K365, C366, Y494 and R426 as set forth in SEQ ID NO:24 and wherein said amino acid residues represent the binding agent's SARS-Corona virus RB protein, more particularly 2019-nCoV RBP.
A computer-assisted method of identifying, designing or screening for a neutralizing agent of the Corona virus RBP domain wherein said neutralizing agent is a binding agent selected from the group consisting of a small molecule compound, a chemical, a peptide, a peptidomimetic, an antibody mimetic, an ISVD, an antibody or antibody fragment, and comprising:
   introducing into suitable computer program parameters defining the three-dimensional structure of the binding site described above,
   creating a three-dimensional structure of a test compound in said computer program;
   displaying a superimposing model of said test compound on the three-dimensional model of the binding site; and
   assessing whether said test compound model fits spatially and chemically into a binding site.
A SARS-CoV-2 binder comprising an ISVD, said ISVD comprising any of the sequences SEQ ID NO: 4, 11, or SEQ ID NO:27-61, or a sequence with at least 90% amino acid identity thereof, or a humanized variant thereof.
Said SARS-CoV-2 binder comprising an ISVD, said ISVD comprising a sequence selected from SEQ ID NO: 4, 28, or 36, or a sequence with at least 90% amino acid identity thereof, or a humanized variant thereof.
Said SARS-CoV-2 binder wherein said ISVD is fused to an IgG Fc domain such as for example an IgG1 or IgG2 Fc domain.
A nucleic acid molecule encoding any of said SARS-CoV-2 binders.
A recombinant vector comprising said nucleic acid molecule.
A pharmaceutical composition comprising any of said SARS-CoV-2 binder, said nucleic acid molecule or said recombinant vector.
Said SARS-CoV-2 binder, nucleic acid molecule or recombinant vector for use as a medicament.
Said SARS-CoV-2 binder, nucleic acid molecule or recombinant vector for use to treat a patient infected with SARS-CoV-2 virus.
A SARS-CoV-2 binder comprising an ISVD, wherein said ISVD comprises the amino acid sequence of the following structure: FR1-CDR1-FR2-CDR2-FR3-CDR3-FR4, and wherein the 3 complementarity determining regions (CDRs) are selected from those CDR1, CDR2, and CDR3 regions as depicted in SEQ ID NO: 6, wherein the CDR regions are annotated according to Kabat, MacCallum, IMGT, AbM, or Chothia.
Said SARS-CoV-2 binder, wherein said ISVD comprises CDR1 comprising SEQ ID NO:7, CDR2 comprising SEQ ID NO:10, and CDR3 comprising SEQ ID NO:9.
Said SARS-CoV-2 binder wherein said ISVD comprises the amino acid sequence of SEQ ID NO:4, 5, or 6, or a humanized variant thereof.
Said SARS-CoV-2 binder comprising any of said ISVDs, wherein said ISVD is fused to an IgG Fc domain.
Said SARS-CoV-2 binder, wherein said IgG Fc domain is an IgG1 Fc domain or a humanized derivative thereof.
Said SARS-CoV-2 binder, comprising the amino acid sequence of SEQ ID NO: 19-22.
A nucleic acid molecule encoding any of said SARS-CoV-2 binders.
A host cell comprising any of said SARS-Cov-2 binders, or said nucleic acid molecule.
A pharmaceutical composition comprising any of said SARS-CoV-2 binders, or said nucleic acid molecule.
Said SARS-CoV-2 binder, nucleic acid molecule, or pharmaceutical composition, for use as a medicament.
Said SARS-CoV-2 binder, nucleic acid molecule, or pharmaceutical composition, for use in therapeutic treatment or prevention of SARS-CoV-2 viral infection or COVID19 disease.
The SARS-CoV-2 binder comprising an immunoglobulin single variable domain fused to an IgG1 Fc domain comprising the amino acid sequence of SEQ ID NO: 17, 18 or 22, or a further humanized variant thereof.
Said SARS-CoV-2 binder, consisting of SEQ ID NO: 22.
A pharmaceutical composition comprising any of said said SARS-CoV-2 binders.
Said SARS-CoV-2 binder, or pharmaceutical composition, for use as a medicament.
Said SARS-CoV-2 binder, or pharmaceutical composition, for use in prophylactic or therapeutic treatment of corona virus infection.
Said SARS-CoV-2 binder, or pharmaceutical composition, for use in prophylactic or therapeutic treatment of SARS-Cov or SARS-Cov-2 viral infection.
Said SARS-CoV-2 binder, or pharmaceutical composition, for use in prophylactic or therapeutic treatment of Covid19.
A binding agent specifically binding the Corona virus Spike protein RBD domain, which comprises an immunoglobulin single variable domain specifically binding the epitope comprising residues L368, Y369, S371, S375, T376, F377, K378, C379 and Y508 as set forth in SEQ ID NO: 23.
Said ISVD-containing binding agent, comprising SEQ ID NO:7 as CDR1, SEQ ID NO:10 as CDR2 and SEQ ID NO:9 as CDR3.
Said ISVD-containing binding agent, comprising SEQ ID NO:6 or a variant with at least 90% identity thereof and/or a humanized variant of any one thereof.
Said ISVD-containing binding agent, comprising SEQ ID NO:22 or a variant with at least 90% identity thereof and/or a humanized variant of any one thereof.
A pharmaceutical composition comprising any of said ISVD-containing binding agents.
Said ISVD-containing binding agents, or said pharmaceutical composition, for use as in treatment of human corona virus infection.
Said ISVD-containing binding agent, or said pharmaceutical composition, for use in treatment of betacoronavirus infection.
Said ISVD-containing binding agent, or said pharmaceutical composition, for use in treatment of Sarbecovirus infection.
Said ISVD-containing binding agent, or said pharmaceutical composition, for use in treatment of infection by SARS-Cov-2 virus or a mutant thereof.
Said ISVD-containing binding agent, or said pharmaceutical composition, for use in treatment of infection by SARS-Cov-2 virus or a mutant thereof, wherein said mutant comprises a mutation in the Spike protein RBD domain.
Said ISVD-containing binding agent, or said pharmaceutical composition, for use in treatment of infection by SARS-Cov-2 virus or a mutant thereof, wherein said RBD mutation comprises the N439K, S477N, E484K, and N501Y as set forth in SEQ ID NO:23.
Said ISVD-containing binding agent, or said pharmaceutical composition, for use in treatment of COVID19.
Use of said ISVD-containing binding agent, or a labelled form thereof, for detection of a viral particle or detection of a viral Spike protein derived from the viruses selected from the group of Sarbecoviruses belonging to clade 1a, 1b, 2 and/or 3 of Bat SARS-related sarbecoviruses.
Use of said ISVD-containing binding agent, or a labelled form thereof, for detection of a viral particle or detection of a viral Spike protein derived from the viruses selected from the group of SARS-Cov-2, GD-Pangolin, RaTG13, WIV1, LYRa11, RsSHC014, Rs7327, SARS-CoV-1, Rs4231, Rs4084, Rp3, HKU3-1, or BM48-31 viruses.

### REFERENCES

1. Zhu, N. et al. A Novel Coronavirus from Patients with Pneumonia in China, 2019. N. Engl. J. Med. 382, 727-733 (2020).
2. Tenforde, M. W. et al. Influenza vaccine effectiveness in inpatient and outpatient settings in the United States, 2015-2018. Clin. Infect. Dis. Off. Publ. Infect. Dis. Soc. Am. (2020) doi:10.1093/cid/ciaa407.
3. Pyzik, M. et at. The Neonatal Fc Receptor (FcRn): A Misnomer? Front. Immunol. 10, 1540 (2019).
4. Polycarpou, A. et al. Rationale for targeting complement in COVID-19. EMBO Mo. Med. e12642 (2020) doi:10.15252/emmm.202012642.
5. Cunningham, L., Kimber, I., Basketter, D. A. & McFadden, J. P. Why judiciously timed anti-IL 6 therapy may be of benefit in severe COVID-19 infection. Autoimmun. Rev. 19, 102563 (2020).
6. Mastaglio, 5. et al. The first case of COVID-19 treated with the complement C3 inhibitor AMY-101. Clin. Immunol. Orlando Fla. 215, 108450 (2020).
7. Wines, B. D., Powell, M. S., Parren, P. W., Barnes, N. & Hogarth, P. M. The IgG Fc contains distinct Fc receptor (FcR) binding sites: the leukocyte receptors Fc gamma RI and Fc gamma RIIa bind to a region in the Fc distinct from that recognized by neonatal FcR and protein A. J. Immunol. Baltim. Md. 1950164, 5313-5318 (2000).
8. Xu, M. et al. A potent neutralizing antibody with therapeutic potential against all four serotypes of dengue virus. NPJ Vaccines 2, (2017).
9. Schlothauer, T. et at. Novel human IgG1 and IgG4 Fc-engineered antibodies with completely abolished immune effector functions. Protein Eng. Des. Sel. PEDS 29, 457-466 (2016).
10. Wrapp, D. et at. Structural Basis for Potent Neutralization of Betacoronaviruses by Single-Domain Camelid Antibodies. Cell 181, 1004-1015.e15 (2020).
11. Letko, M., Marzi, A. & Munster, V. Functional assessment of cell entry and receptor usage for SARS-CoV-2 and other lineage B betacoronaviruses. Nat. Microbiol. 5, 562-569 (2020).
12. International Committee on Taxonomy of Viruses Executive Committee. The new scope of virus taxonomy: partitioning the virosphere into 15 hierarchical ranks. Nat. Microbiol. 5, 668-674 (2020).
13. Boudewijns, R. et al. STAT2 signaling as double-edged sword restricting viral dissemination but driving severe pneumonia in SARS-CoV-2 infected hamsters. bioRxiv 2020.04.23.056838 (2020) doi:10.1101/2020.04.23.056838.
14. Camacho, C. J. & Zhang, C. FastContact: rapid estimate of contact and binding free energies. Bioinforma. Oxf. Engl. 21, 2534-2536 (2005).
15. Zhou, D. et al. Structural basis for the neutralization of SARS-CoV-2 by an antibody from a convalescent patient. Nat. Struct. Mol. Biol. (2020) doi:10.1038/s41594-020-0480-y.
16. Liu, H. et al. Cross-neutralization of a SARS-CoV-2 antibody to a functionally conserved site is mediated by avidity. bioRxiv (2020) doi:10.1101/2020.08.02.233536.
17. Yuan, M. et al. Structural basis of a shared antibody response to SARS-CoV-2. Science 369, 1119-1123 (2020).
18. Lv, Z. et al. Structural basis for neutralization of SARS-CoV-2 and SARS-CoV by a potent therapeutic antibody. Science (2020) doi:10.1126/science.abc5881.
19. Starr, T. N. et al. Deep mutational scanning of SARS-CoV-2 receptor binding domain reveals constraints on folding and ACE2 binding. BioRxiv Prepr. Serv. Biol. (2020) doi:10.1101/2020.06.17.157982.
20. Yang, J. & Zhang, Y. I-TASSER server: new development for protein structure and function predictions. Nucleic Acids Res. 43, W174-181 (2015).
21. Guex, N. & Peitsch, M. C. SWISS-MODEL and the Swiss-PdbViewer: an environment for comparative protein modeling. Electrophoresis 18, 2714-2723 (1997).
22. Abraham, M. J. et al. GROMACS: High performance molecular simulations through multi-level parallelism from laptops to supercomputers. SoftwareX 1-2, 19-25 (2015).
23. Wrapp, D. et al. Cryo-EM structure of the 2019-nCoV spike in the prefusion conformation. Science 367, 1260-1263 (2020).
24. Yan, R. et al. Structural basis for the recognition of SARS-CoV-2 by full-length human ACE2. Science 367, 1444-1448 (2020).
25. Walls, A. C. et al. Structure, Function, and Antigenicity of the SARS-CoV-2 Spike Glycoprotein. Cell 181, 281-292.e6 (2020).
26. Rotman, M. et al. Fusion of hIgG1-Fc to 1111n-anti-amyloid single domain antibody fragment VHH-pa2H prolongs blood residential time in APP/PS1 mice but does not increase brain uptake. Nucl. Med. Biol. 42, 695-702 (2015).
27. Zohar, T. & Alter, G. Dissecting antibody-mediated protection against SARS-CoV-2. Nat. Rev. Immunol. 20, 392-394 (2020).
28. De Meyer, T., Muyldermans, S. & Depicker, A. Nanobody-based products as research and diagnostic tools. Trends Biotechnol. 32, 263-270 (2014).
29. Detalle, L. et al. Generation and Characterization of ALX-0171, a Potent Novel Therapeutic Nanobody for the Treatment of Respiratory Syncytial Virus Infection. Antimicrob. Agents Chemother. 60, 6-13 (2016).
30. De Vlieger, D., Ballegeer, M., Rossey, I., Schepens, B. & Saelens, X. Single-Domain Antibodies and Their Formatting to Combat Viral infections. Antibodies Basel Switz. 8, (2018).
31. Laursen, N. S. et al. Universal protection against influenza infection by a multidomain antibody to influenza hemagglutinin. Science 362, 598-602 (2018).
32. Jain, T. et al. Biophysical properties of the clinical-stage antibody landscape. Proc. Natl. Acad. Sci. U.S.A 114, 944-949 (2017).
33. Grassi, L. & Cabrele, C. Susceptibility of protein therapeutics to spontaneous chemical modifications by oxidation, cyclization, and elimination reactions. Amino Acids 51, 1409-1431 (2019).
34. Chan, J. F.-W. et at. Simulation of the clinical and pathological manifestations of Coronavirus Disease 2019 (COVID-19) in golden Syrian hamster model: implications for disease pathogenesis and transmissibility. Clin. Infect. Dis. Off. Publ. Infect. Dis. Soc. Am. (2020) doi:10.1093/cid/ciaa325.
35. Spiteri, G. et a. First cases of coronavirus disease 2019 (COVID-19) in the WHO European Region, 24 Jan. to 21 Feb. 2020. Euro Surveill. Bull. Eur. Sur Mal. Transm. Eur. Commun. Dis. Bull. 25, (2020).
36. Korber, B. et al. Tracking Changes in SARS-CoV-2 Spike: Evidence that D614G Increases Infectivity of the COVID-19 Virus. Cell (2020) doi:10.1016/j.cell.2020.06.043.
37. Baum, A. et al. Antibody cocktail to SARS-CoV-2 spike protein prevents rapid mutational escape seen with individual antibodies. Science (2020) doi:10.1126/science.abd0831.
38. Shields, R. L. et al. Lack of fucose on human IgG1 N-linked oligosaccharide improves binding to human Fcgamma RIII and antibody-dependent cellular toxicity. J. Biol. Chem. 277, 26733-26740 (2002).
39. Larsen, M. D. et at. Afucosylated immunoglobulin G responses are a hallmark of enveloped virus infections and show an exacerbated phenotype in COVID-19. bioRxiv 2020.05.18.099507 (2020) doi:10.1101/2020.05.18.099507.
40. Liu, L. et al. High neutralizing antibody titer in intensive care unit patients with COVID-19. Emerg. Microbes Infect. 1-30 (2020) doi:10.1080/22221751.2020.1791738.
41. Li, L et a. Effect of Convalescent Plasma Therapy on Time to Clinical Improvement in Patients With Severe and Life-threatening COVID-19: A Randomized Clinical Trial. JAMA (2020) doi:10.1001/jama.2020.10044.
42. Lindorff-Larsen, K. et a. Improved side-chain torsion potentials for the Amber ff99SB protein force field. Proteins 78, 1950-1958 (2010).
43. Weninger, A., Hatzl, A.-M., Schmid, C., Vogl, T. & Glieder, A. Combinatorial optimization of CRISPR/Cas9 expression enables precision genome engineering in the methylotrophic yeast Pichia pastoris. J. Biotechnol. 235, 139-149 (2016).
44. Krainer, F. W. et al. Knockout of an endogenous mannosyltransferase increases the homogeneity of glycoproteins produced in Pichia pastoris. Sci. Rep. 3, 3279 (2013).
45. Lee, M. E., DeLoache, W. C., Cervantes, B. & Dueber, J. E. A Highly Characterized Yeast Toolkit for Modular, Multipart Assembly. ACS Synth. Biol. 4, 975-986 (2015).
46. Wu, S. & Letchworth, G. J. High efficiency transformation by electroporation of Pichia pastoris pretreated with lithium acetate and dithiothreitol. BioTechniques 36, 152-154 (2004).
47. Zhao, H., Brown, P. H. & Schuck, P. On the distribution of protein refractive index increments. Biophys. J. 100, 2309-2317 (2011).
48. Tsunashima, Y., Moro, K., Chu, B. & Liu, T. Y. Characterization of group C meningococcal polysaccharide by light-scattering spectroscopy. III. Determination of molecular weight, radius of gyration, and translational diffusional coefficient. Biopolymers 17, 251-265 (1978).
49. Lo, M.-C. et al. Evaluation of fluorescence-based thermal shift assays for hit identification in drug discovery. Anal. Biochem. 332, 153-159 (2004).
50. ter Meulen, J. et al. Human monoclonal antibody combination against SARS coronavirus: synergy and coverage of escape mutants. PLoS Med. 3, e237 (2006).
51. Tian, X. et al. Potent binding of 2019 novel coronavirus spike protein by a SARS coronavirus-specific human monoclonal antibody. bioRxiv 2020.01.28.923011 (2020) doi:10.1101/2020.01.28.923011.
52. Kirchdoerfer, R. N. et al. Stabilized coronavirus spikes are resistant to conformational changes induced by receptor recognition or proteolysis. Sci. Rep. 8, 15701 (2018).
53. McLellan, J. S. et al. Structure-based design of a fusion glycoprotein vaccine for respiratory syncytial virus. Science 342, 592-598 (2013).
54. Pallesen, J. et al. Immunogenicity and structures of a rationally designed prefusion MERS-CoV spike antigen. Proc. Natl. Acad. Sci. U.S.A 114, E7348-E7357 (2017).
55. Wang, L. et al. Evaluation of candidate vaccine approaches for MERS-CoV. Nat. Commun. 6, 7712 (2015).
56. Dall'Acqua, W. F. et al. Increasing the affinity of a human IgG1 for the neonatal Fc receptor: biological consequences. J. Immunol. Baltim. Md. 1950169, 5171-5180 (2002).
57. Zalevsky, J. et al. Enhanced antibody half-life improves in vivo activity. Nat. Biotechnol. 28, 157-159 (2010).
58. Hultberg, A. et al. Llama-derived single domain antibodies to build multivalent, superpotent and broadened neutralizing anti-viral molecules. PloS One 6, e17665 (2011).
59. McCray, P. B. et al. Lethal infection of K18-hACE2 mice infected with severe acute respiratory syndrome coronavirus. J. Virol. 81, 813-821 (2007).
60. Huang, C. et al. Clinical features of patients infected with 2019 novel coronavirus in Wuhan, China. Lancet 395, 497-506 (2020).
61. Rockx, B. et al. Comparative pathogenesis of COVID-19, MERS, and SARS in a nonhuman primate model. Science (80-.). eabb7314 (2020) doi:10.1126/science.abb7314.
62. Vrancken, B. et al. Accounting for population structure reveals ambiguity in the Zaire Ebolavirus reservoir dynamics. PLoS Negl. Trop. Dis. 14, e0008117 (2020).
63. Conceição-Neto, N. et al. Modular approach to customise sample preparation procedures for viral metagenomics: A reproducible protocol for virome analysis. Sci. Rep. 5, 16532-16532 (2015).
64. Conceição-Neto, N., Yinda, K. C., Van Ranst, M. & Matthijnssens, J. NetoVIR: Modular approach to customize sample preparation procedures for viral metagenomics. in Methods in Molecular Biology vol. 1838 85-95 (Humana Press Inc., 2018).
65. Livak, K. J. & Schmittgen, T. D. Analysis of relative gene expression data using real-time quantitative PCR and the 2-ΔΔCT method. Methods 25, 402-408 (2001).
66. Drosten, C. et a. Identification of a novel coronavirus in patients with severe acute respiratory syndrome. N. Engl. J. Med. 348, 1967-1976 (2003).
67. de Groot, R. J. et al. Middle East respiratory syndrome coronavirus (MERS-CoV): announcement of the Coronavirus Study Group. J. Viral. 87, 7790-7792 (2013).
68. Chen, N. et at. Epidemiological and clinical characteristics of 99 cases of 2019 novel coronavirus pneumonia in Wuhan, China: a descriptive study. Lancet Lond. Engl. (2020) doi:10.1016/S0140-6736(20)30211-7.
69. Sanchez Felipe, L et al, A single-dose live-attenuated YF17D-vectored 1 SARS-CoV2 vaccine candidate. Nature 2020.
70. Kaptein, S et al, Favipiravir at high doses has potent antiviral activity in SARS-CoV-2-infected hamsters, whereas hydroxychloroquine lacks activity. Proc Natl Acad Sci USA 2020 Oct. 9; 202014441.
71. Reed, L. J.; Muench, H. A simple method of estimating fifty percent endpoints. Am. J. Hyg. 1938, 27, 493-497.
72. T. N. Starr et al., Cell. 182, 1295-1310.e20 (2020).
73. R. D. Gietz, R. H. Schiestl, Nat. Protoc. 2, 31-34 (2007).
74. E. Krissinel, K. Henrick, J. Mol. Biol. 372, 774-797 (2007).
75. Z. Abdelrahman et al. Frontiers in Immun. 11; (2020) doi: 10.3389/fimmu.2020.552909.
76. Shrock, E. et al. Viral epitope profiling of COVID-19 patients reveals cross-reactivity and correlates of severity. Science 370, (2020).
77. Lucas, C. et al. Kinetics of antibody responses dictate COVID-19 outcome. medRxiv 2020.12.18.20248331 (2020) doi:10.1101/2020.12.18.20248331.
78. Weinreich, D. M. et al. REGN-COV2, a Neutralizing Antibody Cocktail, in Outpatients with Covid-19. N. Engl. J. Med. (2020) doi:10.1056/NEJMoa2035002.
79. Chen, P. et al. SARS-CoV-2 Neutralizing Antibody LY-CoV555 in Outpatients with Covid-19. N. Engl. J. Med. (2020) doi:10.1056/NEJMoa2029849.
80. Libster, R. et al. Early High-Titer Plasma Therapy to Prevent Severe Covid-19 in Older Adults. N. Engl. J. Med. (2021) doi:10.1056/NEJMoa2033700.
81. Cai, Y. et al. Distinct conformational states of SARS-CoV-2 spike protein. Science (2020) doi:10.1126/science.abd4251.
82. Piccoli, L. et al. Mapping Neutralizing and Immunodominant Sites on the SARS-CoV-2 Spike Receptor-Binding Domain by Structure-Guided High-Resolution Serology. Cell 183, 1024-1042.e21 (2020).
83. Leung, K., Shum, M. H., Leung, G. M., Lam, T. T. & Wu, J. T. Early transmissibility assessment of the N501Y mutant strains of SARS-CoV-2 in the United Kingdom, October to November 2020. Euro Surveill. Bull. Eur. Sur Mal. Transm. Eur. Commun. Dis. Bull. 26, (2021).
84. Zahradnik, J. et al. SARS-CoV-2 RBD in vitro evolution follows contagious mutation spread, yet generates an able infection inhibitor. bioRxiv 2021.01.06.425392 (2021) doi:10.1101/2021.01.06.425392.
85. Thomson, E. C. et al. The circulating SARS-CoV-2 spike variant N439K maintains fitness while evading antibody-mediated immunity. bioRxiv 2020.11.04.355842 (2020) doi:10.1101/2020.11.04.355842.
86. Arvin, A. M. et al. A perspective on potential antibody-dependent enhancement of SARS-CoV-2. Nature 584, 353-363 (2020).
87. DeFrancesco, L. COVID-19 antibodies on trial. Nat. Biotechnol. 38, 1242-1252 (2020).
88. Yang, L et al. COVID-19 antibody therapeutics tracker: a global online database of antibody therapeutics for the prevention and treatment of COVID-19. Antib. Ther. 3, 205-212 (2020).
89. Shi, R. et al. A human neutralizing antibody targets the receptor-binding site of SARS-CoV-2. Nature 584, 120-124 (2020).
90. Xu, D. et al. In vitro characterization of five humanized OKT3 effector function variant antibodies. Cell. Immunol. 200, 16-26 (2000).
91. Pinto, D. et al. Cross-neutralization of SARS-CoV-2 by a human monoclonal SARS-CoV antibody. Nature 583, 290-295 (2020).
92. Greaney et al., Cell Host and Microbe, 29-1, p44-57 (2021).

1. A binding agent that specifically binds an epitope of Corona virus SARS-Cov-1 Spike protein as set forth in SEO ID NO:24 comprising amino acid residues Leu355, Tyr356, Ser358, Ser362, Thr363, F364, K365, C366 and Y494, wherein
said binding agent comprises an immunoglobulin single variable domain (ISVD) comprising 4 framework regions (FR) and 3 complementarity determining regions (CDR) according to the following formula (1): FR1-CDR1-FR2-CDR2-FR3-CDR3-FR4 (1); and wherein
the 3 CDRs are selected from the group consisting of CDR1, CDR2, and CDR3 regions as depicted in SEQ ID NO: 1, SEQ ID NO:4, SEQ ID NO:27-61, or SEQ ID NO: 92-105, wherein the CDR regions are annotated according to Kabat, MacCallum, IMGT, AbM, or Chothia.

2. The binding agent according to claim 1, wherein the epitope further comprises amino acid residue R426.

3. The binding agent according to claim 1, wherein the binding agent also specifically binds to an epitope of Corona virus SARS-Cov-2 Spike protein as set forth in SEQ ID NO: 23 comprising residues L368, Y369, S371, S375, T376, F377, K378, C379 and Y508.

4. The binding agent according to claim 1, wherein said binding agent competes for binding the epitope of Corona virus SARS-Cov-1 Spike protein with ACE2.

5. The binding agent according to claim 1, wherein said binding agent is a peptide, a peptidomimetic, an antibody mimetic, an immunoglobulin single variable domain (ISVD), an antibody, or active antibody fragment.

6. The binding agent according to claim 1, wherein CDR1 consists of SEQ ID NO: 7, SEQ ID NO:111-119, or the sequence SYAMG,
CDR2 consists of SEQ ID NO: 8, SEQ ID NO:10, SEQ ID NO:120-130, SEQ ID NO:141, or the sequence TISWSGGGTYYAEPVRG, and
CDR3 consists of SEQ ID NO: 9, or SEQ ID NO:131-140.

7. (canceled)

8. The binding agent according to claim 1, wherein the ISVD comprises SEQ ID NO: 1, 4, 27-61, or SEQ ID NO: 92-105, or a sequence with at least 90% amino acid identity thereof, or a humanized variant of any one thereof, as set forth for example in SEQ ID NO: 2, 3, 5, 6, or 11.

9. The binding agent according to claim 1, wherein said ISVD is fused to an Fc domain.

10. The binding agent according to claim 1, which is a multivalent or multispecific binding agent.

11. The binding agent according to claim 10, comprising a bivalent ISVD, or a humanized variant thereof.

12. The binding agent according to claim 10, wherein said ISVD is fused to an IgG Fc domain in a monovalent or multivalent format.

13. The binding agent according to claim 9, comprising a sequence selected from the group of SEQ ID NO:13 to SEQ ID NO:22, or a humanized variant thereof.

14. The binding agent according to claim 1, having a sequence consisting of SEQ ID NO:22.

15. A nucleic acid molecule encoding the binding agent according to claim 1.

16. A recombinant vector comprising the nucleic acid molecule according to claim 15.

17. A complex comprising the Receptor binding domain of SARS-Corona virus as depicted in SEQ ID NO: 25 or SEQ ID NO:26 and a binding agent according to claim 1.

18. A host cell comprising the binding agent according to claim 1.

19. A pharmaceutical composition comprising the binding agent according to claim 1 and a pharmaceutically acceptable carrier, diluent or excipient.

20.-30. (canceled)

31. A pharmaceutical composition comprising the nucleic acid molecule according to claim 15 and a pharmaceutically acceptable carrier, diluent or excipient.

32. The binding agent according to claim 9, wherein said Fc domain is an IgG, IgG1 or IgG2 Fc domain, or a variant thereof.

33. The binding agent according to claim 11, wherein the bivalent ISVD, comprises the sequence SEQ ID NO:12, or a humanized variant thereof.

34. A method of treating a subject with a coronavirus infection, the method comprising: administering the binding agent according to claim 1 to the subject.

35. A method of treating a subject with a coronavirus infection, the method comprising: administering the nucleic acid molecule according to claim 15 to the subject.

36. A method of diagnosing a subject with a coronavirus infection, the method comprising:
obtaining a sample from the subject.
introducing the binding agent according to claim 1 to the sample, and
determining whether the binding agent binds to components of the sample, wherein the presence of binding indicates the presence of the coronavirus infection.

37. A method of in vivo imaging of a coronavirus in a subject, the method comprising: administering a binding agent according to claim 1 to a subject and obtaining an in vivo image identifying locations of binding of the binding agent.

38. A method of lowering viral loads in a subject after infection of the subject by a coronavirus, comprising administering the binding agent according to claim 1 to the subject prior to the infection.

39. A method for detecting a viral particle or a viral Spike protein from a virus selected from the group of viruses belonging to clade 1a, 1b, 2 and/or 3 of bat SARS-related Sarbecoviruses in a sample, said method comprising allowing the binding agent according to claim 1, or a labelled form thereof, to bind to the binding site of the RBD of the viral Spike protein.

40. The binding agent according to claim 6, wherein said ISVD comprises a combination of CDR1, CDR2 and CDR3 selected from the group consisting of:
a. CDR1 consisting of a SEQ ID NO: 7; CDR2 of SEQ ID NO: 8 or 10; and CDR3 of SEQ ID NO: 9,
b. CDR1 consisting of a SEQ ID NO: 111; CDR2 of SEQ ID NO: 120; and CDR3 of SEQ ID NO: 9,
c. CDR1 consisting of a SEQ ID NO: 112; CDR2 of SEQ ID NO: 121; and CDR3 of SEQ ID NO: 131,
d. CDR1 consisting of a SEQ ID NO: 113; CDR2 of SEQ ID NO: 121; and CDR3 of SEQ ID NO: 131,
e. CDR1 consisting of a SEQ ID NO: 114; CDR2 of the sequence TISWSGGGTYYAEPVRG; and CDR3 of SEQ ID NO: 132,
f. CDR1 consisting of a SEQ ID NO: 113; CDR2 of SEQ ID NO: 123; and CDR3 of SEQ ID NO: 133,
g. CDR1 consisting of the sequence SYAMG; CDR2 of SEQ ID NO: 124; and CDR3 of SEQ ID NO: 134,
h. CDR1 consisting of the sequence SYAMG; CDR2 of SEQ ID NO: 125; and CDR3 of SEQ ID NO: 135,
i. CDR1 consisting of a SEQ ID NO: 115; CDR2 of SEQ ID NO: 126; and CDR3 of SEQ ID NO: 136,
j. CDR1 consisting of a SEQ ID NO: 116; CDR2 of SEQ ID NO: 127; and CDR3 of SEQ ID NO: 137,
k. CDR1 consisting of a SEQ ID NO: 117; CDR2 of SEQ ID NO: 128; and CDR3 of SEQ ID NO: 138,
l. CDR1 consisting of a SEQ ID NO: 118; CDR2 of SEQ ID NO: 129; and CDR3 of SEQ ID NO: 139, and
m. CDR1 consisting of a SEQ ID NO: 119; CDR2 of SEQ ID NO: 130; and CDR3 of SEQ ID NO: 140.
